SEQUENCE LISTING (694 sequences)

SEQ ID NO: 1 - DNA, 5425 bp, Homo sapiens
cccgggccaccgcctccgcccggctgcccgcccggactgtcgcggcccgcggtggcgacggcggccgctgcaaagtttccccggcggcggcggcccgggggcgcatcctcccgcaactgtcaagcgctggcggcggaaatgatgaggcgctggccattttccgagcccgggtttcctgcctgagccccgctcgagcgagccgcgagcgaggagccggcgggcgggagaggacgcgcccagggcgggggcccgcccgccccctcgggatttcgagggcccgggggcgcgcgacgccatgggccggccgggcccagagctcctgtctctcagcccggccgcaccacctgggtctccgccatgaacgggcctgccctgcagccctcctcgccctcttccgcgccctcagcctccccggcggcggccccgcggggctggagcgagttctgtgagttgcacgccgtagcggcggcccgggagctggcccgccagtactggctgttcgcccgggagcatccgcagcacgcgccgctgcgcgccgagctggtgtcgctgcagttcaccgacctcttccagcgctacttctgccgcgaggtgcgcgacggacgggcgccgggccgcgactaccgggacacaggccgtgggcccccagccaaggccgaggcgtccccggagccaggccccggccccgccgcccctggcctgcccaaggcccgcagctctgaggagctggccccgccgcggccgcccgggccctgctccttccagcactttcgccgcagcctccgccacatcttccgccgccgctcggccggggagctgccagcggcccacaccgctgccgcccccgggacccccggagaggctgctgagacccccgcccggcctggcctggccaagaagttcctgccctggagcctggcccgggagccgccacccgaggcgctgaaggaggcggtgctgcgctacagcctggccgacgaggcctccatggacagcggggcacgctggcagcgcgggaggctggcgctgcgccgggccccgggccccgatggccccgaccgcgtgctggagctcttcgacccacccaagagttcaaggcccaagctacaagcagcttgctccagcatccaggaggtccggtggtgcacacggcttgagatgcctgacaacctttacacctttgtgctgaaggtgaaggaccggacagacatcatctttgaggtgggagacgagcagcagctgaattcatggatggctgagctctcggagtgcacaggccgagggctggagagcacagaagcagagatgcatattccctcagccctagagcctagcacgtccagctccccaaggggcagcacagattcccttaaccaaggtgcttctcctggggggctgctggacccggcctgccagaagacggaccatttcctgtcctgctacccctggttccacggccccatctccagagtgaaagcagctcagctggttcagctgcagggccctgatgctcatggagtgttcctggtgcggcagagcgagacgcggcgtggggaatacgtgctcactttcaactttcaggggatagccaagcacctgcgcctgtcgctgacagagcggggccagtgccgtgtgcagcacctccactttccctcggtcgtggacatgctccaccacttccagcgctcgcccatcccactcgagtgcggcgccgcctgtgatgtccggctctccagctacgtggtagtcgtctcccaaccaccaggttcctgcaacacggtcctcttccctttctcccttcctcactgggattcagagtcccttcctcactggggttcagagttgggccttccccaccttagttcttctggctgtccccgggggctcagcccagagggtctcccagggcgatcctcaccccccgagcagatcttccacctggtgccttcgcccgaagaactggccaacagcctgcagcacctggagcatgagcctgtgaatcgagcccgggactcggactacgaaatggactcatcctcccggagccacctgcgggccatagacaatcagtacacacctctctgaccagtgaggaattccaggcctcaacagctgcccttgaggagcacaggcagaagtgtgaacttgtgaatgtaattgatctttccttccttccagagaaagatttaagggacactgttaactgctcgtgccagtttggaagtgacccttctattaggcctgttgaagggccctcctgtaggtttcatctatccacctggctttctccttattgtttacagatgtagttcttgttagaggatgccgctagctcctgcccggggtccctatgcccagtccccgttactcttagagaaaggagttggggtgagggccagagctggcagtggaaacttgttctctttttcactgacactgtcacagcggatgacagactttctacggggaggagggggggatcatcaggaagcccagaacactaacaagcggttctcccatctaccgtcagtccacatggcaggtctgctgtgtccacaccacagatgaccacatctaatcctgcttctactctcagctttaggacaaaagctctgtcagaggcacaagctgaaggtcaaaaatgatttaaaacattttacctcagactaatttctttaaaggattcaggttcaaaacttaaccactgcttatttcagtgcactgtttcaactaacacccatgctatttttgtagtcagaaacagctatgcaaaccctacctaatttacagtctgagccagcatgctggcttgtctactgcatcctcgggacagtcacctgccactgagtggccactgtccttcctaaatgtcaagaagtgaagtatgtcaccctttcagggaaattcaggcaattactgaaataggagggtggcaagaacagttctatcctggtgccttacgaataaaaaactggattctggtttacagcagctttacagtgatagttaaattaactggggctaggggaagagcaagcaaaaagggaagaaggactcctaggccctttctagtaaatccttcagcaacaaggctggcttggtgccctccaagcatctaatggcttattaaattatcccacaagtgggttttaggctccttttttgagccaaaatggaagctgggaatctggtgccataactaatgagaaactcctttaatagcccacaatcagtgttctgttctagctggctactgcttcactggattgagaatctatctatctccttgcacacatgggcacacacaatctccaccatccagggaggtcctgaagtcaaatctctatctatacaagtgatacaattcatagggggctggctcctcccagaacctgtctggaggctcagaaacgggggcagtgacagtggagtcagctgctcttgggtgccagcagagccattcagtacaacccccaggctcacagcagtggcttctaggaaactgggagtttagatcagctttacagatacatcgatcagaggctaaaatgaaacctcagcctaaaactcataggactgactgcctgggaggagggttaggtctgcttcttccacttatacttagtctctgtgctccaagaggtcaaatttttgcttctagaatttccttggggtctttcagagggtgggggaacaaacccctatgcacttttcttttttttttttttgagatggagtttctcttgtcaaccgggctggagtgcagtggtgcaatcttggctcactgcaacctccaccttcctggttcaagcgattctgcctcgacctctcaagtagctgggattacaagcaccagccaccatgcctggctaattttgtatttttagtagagacagggtttcaccatgttggccaggctggtctcgaatgtctgacctcaggtgatccacccgccttggcctcccaaagtgctgggattacaggcgcgagccaccgcgcccagcctacaccacttttagtaccaacactcttgggtgatttcatggaccctaaagcagacctgacactgatccagatttgcagtccatttttaaggacacctgtctttatttcctcaaagtcaagcagctttctctggaaaatgaatgctaattagtgtgaaccaaaagagtaagtaagagtctgaagtttttttaaaggagaaagcttattatggaaagtcactggtcctcccctccgcacaggaaaggtacccagtagataatgaaccaaattaagttccctccctccagccagaagttaaacatctgggatatgacgtcttcatgccaggggcactcatttcttagcagcctctctacatacatctctcaggtggtgccaagaggcacaccaggtagagcaaacttagcagctctgactaacaggctgcaaagtgcaagttcagattctgtggcagagatttggaaggcacccacctccagactgcttcccgtccaagttaccaggacagctcaaaaacatgctgacagaaaactcccatggctctaggaagaagtgacactaagccaacacctttctttatgtgggagcagaatcagctgatgaaggggtgggcagcagtgtggggcaggcaccccactggctgcagctagcccaccataggcacagcacatcccaccactctccttccagtcctgaccaggccccagccggcaacttctaccgagagccatggctcaacaccaaactggacagtagacatcatgatccctccagttagctctaattacagaccccaccagtacagcttgacagctcccggcaccatcccttccttcatctgacttattgaacttttacaaactaacagtcaccagcaccaaagaattaagtcaactaacctgccttgaattttagaccagcaatccatatggctttatctggtataaatcttctgcctttgatcatttctggaccgtaggaaaaaggaatagcaatcattaaaatcttgggccagagaacactatttttacataacagtttcttaacctaaagtcaaggccttggactcttccctgagggttgcctgagattccttcatgctttctattcaggactaagtcccttactgcaaatgtgttagctctaacatctcccacaagctagaggaacttgcgagtatattaacaaggacacatctgacatcctgtgtttggttagaatatacagcacattgtgataacataaagtggattcatcttgtatcattataggcagaaggtatttggcaaatttttatgtattgttttatgtactgtacaagtaacttattcttgaataatgcaaattttgctataatgtacaaattgctatatgtgaattaaaaagttttcagaatcttgaaaaaaaaaaaaaaaaaaaa

SEQ ID NO: 2 - DNA, 3088 bp, Homo sapiens
ccccttttccagaatcacttgcactgtcttgttcttgaatgagaaaggaagaaaagagcctcccattactcagacccgtgtaaacattattccccccaggagaaaatggtgttattcaaatgaatcataataaaatagcctctaaacagtttctaagcgggagcctccgtggaactcagcgctccgctcctcccagttcctaagaggtcccgggattcttgagctgtgcccagctgacgagcttttgaagatggcacaataaccgtccagtgatgcctgaccatgacagcacagccctcttaagccggcaaaccaagaggagaagagttgacattggagtgaaaaggacggtagggacagcatctgcattttttgctaaggcaagagcaacgttttttagtgccatgaatccccaaggttctgagcaggatgttgagtattcagtggtgcagcatgcagatggggaaaagtcaaatgtactccgcaagctgctgaagagggcgaactcgtatgaagatgccatgatgccttttccaggagcaaccataatttcccagctgttgaaaaataacatgaacaaaaatggtggcacggagcccagtttccaagccagcggtctctctagtacaggctccgaagtacatcaggaggatatatgcagcaactcttcaagagacagccccccagagtgtctttccccttttggcaggcctactatgagccagtttgatatggatcgcttatgtgatgagcacctgagagcaaagcgcgcccgggttgagaatataattcggggtatgagccattcccccagtgtggcattaaggggcaatgaaaatgaaagagagatggccccgcagtctgtgagtccccgagaaagttacagagaaaacaaacgcaagcaaaagcttccccagcagcagcaacagagtttccagcagctggtttcagcccgaaaagaacagaagcgagaggagcgccgacagctgaaacagcagctggaggacatgcagaaacagctgcgccagctgcaggaaaagttctaccaaatctatgacagcactgattcggaaaatgatgaagatggtaacctgtctgaagacagcatgcgctcggagatcctggatgccagggcccaggactctgtcggaaggtcagataatgagatgtgcgagctagacccaggacagtttattgaccgagctcgagccctgatcagagagcaggaaatggctgaaaacaagccgaagcgagaaggcaacaacaaagaaagagaccatgggccaaactccttacaaccggaaggcaaacatttggctgagaccttgaaacaggaactgaacactgccatgtcgcaagttgtggacactgtggtcaaagtcttttcggccaagccctcccgccaggttcctcaggtcttcccacctctccagatcccccaggccagatttgcagtcaatggggaaaaccacaatttccacaccgccaaccagcgcctgcagtgctttggcgacgtcatcattccgaaccccctggacacctttggcaatgtgcagatggccagttccactgaccagacagaagcactgcccctggttgtccgcaaaaactcctctgaccagtctgcctccggccctgccgctggcggccaccaccagcccctgcaccagtcgcctctctctgccaccacgggcttcaccacgtccaccttccgccaccccttcccccttcccttgatggcctatccatttcagagcccattaggtgctccctccggctccttctctggaaaagacagagcctctcctgaatccttagacttaactagggataccacgagtctgaggaccaagatgtcatctcaccacctgagccaccacccttgttcaccagcacacccgcccagcaccgccgaagggctctccttgtcgctcataaagtccgagtgcggcgatcttcaagatatgtctgaaatatcaccttattcgggaagtgcaatgcaggaaggattgtcacccaatcacttgaaaaaagcaaagctcatgtttttttatacccgttatcccagctccaatatgctgaagacctacttctccgacgtaaagttcaacagatgcattacctctcagctcatcaagtggtttagcaatttccgtgagttttactacattcagatggagaagtacgcacgtcaagccatcaacgatggggtcaccagtactgaagagctgtctataaccagagactgtgagctgtacagggctctgaacatgcactacaataaagcaaatgactttgaggttccagagagattcctggaagttgctcagatcacattacgggagtttttcaatgccattatcgcaggcaaagatgttgatccttcctggaagaaggccatatacaaggtcatctgcaagctggatagtgaagtccctgagattttcaaatccccgaactgcctacaagagctgcttcatgagtagaaatttcaacaactctttttgaatgtatgaagagtagcagtcccctttggatgtccaagttatatgtgtctagattttgatttcatatatatgtgtatgggaggcatggatatgttatgaaatcagctggtaattcctcctcatcacgtttctctcattttcttttgttttccattgcaaggggatggttgttttctttctgcctttagtttgcttttgcccaaggcccttaacatttggacacttaaaatagggttaattttcagggaaaaagaatgttggcgtgtgtaaagtctctattagcaatgaagggaatttgttaacgatgcatccacttgattgatgacttattgcaaatggcggttggctgaggaaaacccatgacacagcacaactctacagacagtgatgtgtctcttgtttctactgctaagaaggtctgaaaatttaatgaaaccacttcatacatttaagtattttgtttggtttgaactcaatcagtagcttttccttacatgtttaaaaataattccaatgacagatgagcagctcacttttccaaagtaccccaaaaggccaaattaaaaaaaaaaaaaaaaaaa

SEQ ID NO: 3 - DNA, 6247 bp, Homo sapiens
agtggctggggagtcccgtcgacgctctgttccgagagcgtgccccggaccgccagctcagaacaggggcagccgtgtagccgaacggaagctgggagcagccgggactggtggcccgcgcccgagctccgcaggcgggaagcaccctggatttaggaagtcccgggagcagcgcggcggcacctccctcacccaaggggccgcggcgacggtcacggggcgcggcgccaccgtgagcgacccaggccaggattctaaatagacggcccaggctcctcctccgcccgggccgcctcacctgcgggcattgccgcgccgcctccgccggtgtagacggcacctgcgccgccttgctcgcgggtctccgcccctcgcccaccctcactgcgccaggcccaggcagctcacctgtactggcgcgggctgcggaagcctgcgtgagccgaggcgttgaggcgcggcgcccacgccactgtcccgagaggacgcaggtggagcgggcgcggcttcgcggaacccggcgccggccgccgcagtggtcccagcctacaccgggttccggggacccggccgccagtgcccggggagtagccgccgccgtcggctgggcaccatgaacagcagcagcgccaacatcacctacgccagtcgcaagcggcggaagccggtgcagaaaacagtaaagccaatcccagctgaaggaatcaagtcaaatccttccaagcggcatagagaccgacttaatacagagttggaccgtttggctagcctgctgcctttcccacaagatgttattaataagttggacaaactttcagttcttaggctcagcgtcagttacctgagagccaagagcttctttgatgttgcattaaaatcctcccctactgaaagaaacggaggccaggataactgtagagcagcaaatttcagagaaggcctgaacttacaagaaggagaattcttattacaggctctgaatggctttgtattagttgtcactacagatgctttggtcttttatgcttcttctactatacaagattatctagggtttcagcagtctgatgtcatacatcagagtgtatatgaacttatccataccgaagaccgagctgaatttcagcgtcagctacactgggcattaaatccttctcagtgtacagagtctggacaaggaattgaagaagccactggtctcccccagacagtagtctgttataacccagaccagattcctccagaaaactctcctttaatggagaggtgcttcatatgtcgtctaaggtgtctgctggataattcatctggttttctggcaatgaatttccaagggaagttaaagtatcttcatggacagaaaaagaaagggaaagatggatcaatacttccacctcagttggctttgtttgcgatagctactccacttcagccaccatccatacttgaaatccggaccaaaaattttatctttagaaccaaacacaaactagacttcacacctattggttgtgatgccaaaggaagaattgttttaggatatactgaagcagagctgtgcacgagaggctcaggttatcagtttattcatgcagctgatatgctttattgtgccgagtcccatatccgaatgattaagactggagaaagtggcatgatagttttccggcttcttacaaaaaacaaccgatggacttgggtccagtctaatgcacgcctgctttataaaaatggaagaccagattatatcattgtaactcagagaccactaacagatgaggaaggaacagagcatttacgaaaacgaaatacgaagttgccttttatgtttaccactggagaagctgtgttgtatgaggcaaccaacccttttcctgccataatggatcccttaccactaaggactaaaaatggcactagtggaaaagactctgctaccacatccactctaagcaaggactctctcaatcctagttccctcctggctgccatgatgcaacaagatgagtctatttatctctatcctgcttcaagtacttcaagtactgcaccttttgaaaacaactttttcaacgaatctatgaatgaatgcagaaattggcaagataatactgcaccgatgggaaatgatactatcctgaaacatgagcaaattgaccagcctcaggatgtgaactcatttgctggaggtcacccagggctctttcaagatagtaaaaacagtgacttgtacagcataatgaaaaacctaggcattgattttgaagacatcagacacatgcagaatgaaaaatttttcagaaatgatttttctggtgaggttgacttcagagacattgacttaacggatgaaatcctgacgtatgtccaagattctttaagtaagtctcccttcataccttcagattatcaacagcaacagtccttggctctgaactcaagctgtatggtacaggaacacctacatctagaacagcaacagcaacatcaccaaaagcaagtagtagtggagccacagcaacagctgtgtcagaagatgaagcacatgcaagttaatggcatgtttgaaaattggaactctaaccaattcgtgcctttcaattgtccacagcaagacccacaacaatataatgtctttacagacttacatgggatcagtcaagagttcccctacaaatctgaaatggattctatgccttatacacagaactttatttcctgtaatcagcctgtattaccacaacattccaaatgtacagagctggactaccctatggggagttttgaaccatccccataccccactacttctagtttagaagattttgtcacttgtttacaacttcctgaaaaccaaaagcatggattaaatccacagtcagccataataactcctcagacatgttatgctggggccgtgtcgatgtatcagtgccagccagaacctcagcacacccacgtgggtcagatgcagtacaatccagtactgccaggccaacaggcatttttaaacaagtttcagaatggagttttaaatgaaacatatccagctgaattaaataacataaataacactcagactaccacacatcttcagccacttcatcatccgtcagaagccagaccttttcctgatttgacatccagtggattcctgtaattccaagcccaattttgaccctggtttttggattaaattagtttgtgaaggattatggaaaaataaaactgtcactgttggacgtcagcaagttcacatggaggcattgatgcatgctattcacaattattccaaaccaaattttaatttttgcttttagaaaagggagtttaaaaatggtatcaaaattacatatactacagtcaagatagaaagggtgctgccacggagtggtgaggtaccgtctacatttcacattattctgggcaccacaaaatatacaaaactttatcagggaaactaagattcttttaaattagaaaatattctctatttgaattatttctgtcacagtaaaaataaaatactttgagttttgagctactggattcttattagttccccaaatacaaagttagagaactaaactagtttttcctatcatgttaacctctgcttttatctcagatgttaaaataaatggtttggtgctttttataaaaagataatctcagtgctttcctccttcactgtttcatctaagtgcctcacatttttttctacctataacactctaggatgtatattttatataaagtattctttttcttttttaaattaatatctttctgcacacaaatattatttgtgtttcctaaatccaaccattttcattaattcaggcatattttaactccactgcttacctactttcttcaggtaaagggcaaataatgatcgaaaaaataattatttattacataatttagttgtttctagactataaatgttgctatgtgccttatgttgaaaaaatttaaaagtaaaatgtctttccaaattatttcttaattattataaaaatattaagacaatagcacttaaattcctcaacagtgttttcagaagaaataaatataccactctttacctttattgatatctccatgatgatagttgaatgttgcaatgtgaaaaatctgctgttaactgcaaccttgtttattaaattgcaagaagctttatttctagctttttaattaagcaaagcacccatttcaatgtgtataaattgtctttaaaaactgttttagacctataatccttgataatatattgtgttgactttataaatttcgcttcttagaacagtggaaactatgtgtttttctcatatttgaggagtgttaagattgcagatagcaaggtttggtgcaaagtattgtaatgagtgaattgaatggtgcattgtatagatataatgaacaaaattatttgtaagatatttgcagtttttcattttaaaaagtccataccttatatatgcacttaatttgttggggctttacatactttatcaatgtgtctttctaagaaatcaagtaatgaatccaactgcttaaagttggtattaataaaaagacaaccacatagttcgtttaccttcaaactttaggtttttttaatgatatactgatcttcattaccaataggcaaattaatcaccctaccaactttactgtcctaacatggtttaaaagaaaaaatgacaccatcttttattcttttttttttttttttttgagagagagtcttactctgccgcccaaactggagtgcagtggcacaatcttggctcactgcaacctctacctcctgggttcaagtgattctcttgcctcagcctcccgagttgctgggattacaggcatgtgccaccatgcccagctaatttttgtatttttagtagaaacgggtttcaccatgttggccagactggtctcaaactcctgacctcaggtgagcctcccaccttggcctcccaaagtgctgggattacaggcgtgagccactgcattcagctcttcttttctttagatatgagagctgaagagcttagacacattttgcatgtattatttgaaaatctgatggaatcccaaactgagatgtattaaaatacaatttttggccgggtgcagtggctcacgcctgtaatcccagcacttggggagggcgaggagggtggatcacgaggtcaagagatggagaccatcctgaccaacatggtgaaaccctgtctctactaaaaatacagaaattagctgggcatggtggcgtgagcctgtagtcctagctactcaggaggctgaggcaggagaatagcctgaacctgggaatcggaggttgcagagccaagatcgccccactgcactccagcctggcaatagaccgagactccgtctccaaaaaaaaaaaaaatacaatttttatttcttttactttttttagtaagttaatgtatataaaaatggcttcggacaaaatatctctgagttctgtgtattttcagtcaaaactttaaacctgtagaatcaatttaagtgttggaaaaaatttgtctgaaacatttcataatttgtttccagcatgaggtatctaaggatttagaccagaggtctagattaatactctatttttacatttaaaccttttattataagtcttacataaaccatttttgttactctcttccacatgttactggataaattgtttagtggaaaataggctttttaatcatgaatatgatgacaatcagttatacagttataaaattaaaagtttgaaaagcaatattgtatatttttatctatataaaataactaaaatgtatctaagaataataaaatcacgttaaaccaaatacacgtttgtctgtattgttaagtgccaaacaaaggatacttagtgcactgctacattgtgggatttatttctagatgatgtgcacatctaaggatatggatgtgtctaatttagtcttttcctgtaccaggtttttcttacaatacctgaagacttaccagtattctagtgtattatgaagctttcaacattactatgcacaaactagtgtttttcgatgttactaaattttaggtaaatgctttcatggcttttttcttcaaaatgttactgcttacatatatcatgcatagatttttgcttaaagtatgatttataatatcctcattatcaaagttgtatacaataatatataataaaataacaaatatgaataat

SEQ ID NO: 4 - protein, 16 aa, Unknown
  source: /note=“Description of Unknown: RFGF peptide”
AAVALLPAVLLALLAP

SEQ ID NO: 5 - protein, 11 aa, Unknown
  source: /note=“Description of Unknown: RFGF analogue peptide”
AALLPVLLAAP

SEQ ID NO: 6 - protein, 13 aa, Human immunodeficiency virus
GRKKRRQRRRPPQ

SEQ ID NO: 7 - protein, 16 aa, Drosophila sp.
RQIKIWFQNRRMKWKK

SEQ ID NO: 8 - DNA, 5420 bp, Homo sapiens
ttttttttttttttttttttcaagattctgaaaactttttaattcacatatagcaatttgtacattatagcaaaatttgcattattcaagaataagttacttgtacataaaacaatacataaaaatttgccaaataccttctgcctataatgatacaagatgaatccactttatgttatcacaatgtgctgtatattctaaccaaacacaggatgtcagatgtgtccttgttaatatactcgcaagttcctctagcttgtgggagatgttagagctaacacatttgcagtaagggacttagtcctgaatagaaagcatgaaggaatctcaggcaaccctcagggaagagtccaaggccttgactttaggttaagaaactgttatgtaaaaatagtgttctctggcccaagattttaatgattgctattcctttttcctacggtccagaaatgatcaaaggcagaagatttataccagataaagccatatggattgctggtctaaaattcaaggcaggttagttgacttaattctttggtgctggtgactgttagtttgtaaaagttcaataagtcagatgaaggaagggatggtgccgggagctgtcaagctgtactggtggggtctgtaattagagctaactggagggatcatgatgtctactgtccagtttggtgttgagccatggctctcggtagaagttgccggctggggcctggtcaggactggaaggagagtggtgggatgtgctgtgcctatggtgggctagctgcagccagtggggtgcctgccccacactgctgcccaccccttcatcagctgattctgctcccacataaagaaaggtgttggcttagtgtcacttcttcctagagccatgggagttttctgtcagcatgtttttgagctgtcctggtaacttggacgggaagcagtctggaggtgggtgccttccaaatctctgccacagaatctgaacttgcactttgcagcctgttagtcagagctgctaagtttgctctacctggtgtgcctcttggcaccacctgagagatgtatgtagagaggctgctaagaaatgagtgcccctggcatgaagacgtcatatcccagatgtttaacttctggctggagggagggaacttaatttggttcattatctactgggtacctttcctgtgcggaggggaggaccagtgactttccataataagctttctcctttaaaaaaacttcagactcttacttactcttttggttcacactaattagcattcattttccagagaaagctgcttgactttgaggaaataaagacaggtgtccttaaaaatggactgcaaatctggatcagtgtcaggtctgctttagggtccatgaaatcacccaagagtgttggtactaaaagtggtgtaggctgggcgcggtggctcgcgcctgtaatcccagcactttgggaggccaaggcgggtggatcacctgaggtcagacattcgagaccagcctggccaacatggtgaaaccctgtctctactaaaaatacaaaattagccaggcatggtggctggtgcttgtaatcccagctacttgagaggtcgaggcagaatcgcttgaaccaggaaggtggaggttgcagtgagccaagattgcaccactgcactccagcccggttgacaagagaaactccatctcaaaaaaaaaaaaaagaaaagtgcataggggtttgttcccccaccctctgaaagaccccaaggaaattctagaagcaaaaatttgacctcttggagcacagagactaagtataagtggaagaagcagacctaaccctcctcccaggcagtcagtcctatgagttttaggctgaggtttcattttagcctctgatcgatgtatctgtaaagctgatctaaactcccagtttcctagaagccactgctgtgagcctgggggttgtactgaatggctctgctggcacccaagagcagctgactccactgtcactgcccccgtttctgagcctccagacaggttctgggaggagccagccccctatgaattgtatcacttgtatagatagagatttgacttcaggacctccctggatggtggagattgtgtgtgcccatgtgtgcaaggagatagatagattctcaatccagtgaagcagtagccagctagaacagaacactgattgtgggctattaaaggagtttctcattagttatggcaccagattcccagcttccattttggctcaaaaaaggagcctaaaacccacttgtgggataatttaataagccattagatgcttggagggcaccaagccagccttgttgctgaaggatttactagaaagggcctaggagtccttcttccctttttgcttgctcttcccctagccccagttaatttaactatcactgtaaagctgctgtaaaccagaatccagttttttattcgtaaggcaccaggatagaactgttcttgccaccctcctatttcagtaattgcctgaatttccctgaaagggtgacatacttcacttcttgacatttaggaaggacagtggccactcagtggcaggtgactgtcccgaggatgcagtagacaagccagcatgctggctcagactgtaaattaggtagggtttgcatagctgtttctgactacaaaaatagcatgggtgttagttgaaacagtgcactgaaataagcagtggttaagttttgaacctgaatcctttaaagaaattagtctgaggtaaaatgttttaaatcatttttgaccttcagcttgtgcctctgacagagcttttgtcctaaagctgagagtagaagcaggattagatgtggtcatctgtggtgtggacacagcagacctgccatgtggactgacggtagatgggagaaccgcttgttagtgttctgggcttcctgatgatccccccctcctccccgtagaaagtctgtcatccgctgtgacagtgtcagtgaaaaagagaacaagtttccactgccagctctggccctcaccccaactcctttctctaagagtaacggggactgggcatagggaccccgggcaggagctagcggcatcctctaacaagaactacatctgtaaacaataaggagaaagccaggtggatagatgaaacctacaggagggcccttcaacaggcctaatagaagggtcacttccaaactggcacgagcagttaacagtgtcccttaaatctttctctggaaggaaggaaagatcaattacattcacaagttcacacttctgcctgtgctcctcaagggcagctgttgaggcctggaattcctcactggtcagagaggtgtgtactgattgtctatggcccgcaggtggctccgggaggatgagtccatttcgtagtccgagtcccgggctcgattcacaggctcatgctccaggtgctgcaggctgttggccagttcttcgggcgaaggcaccaggtggaagatctgctcggggggtgaggatcgccctgggagaccctctgggctgagcccccggggacagccagaagaactaaggtggggaaggcccaactctgaaccccagtgaggaagggactctgaatcccagtgaggaagggagaaagggaagaggaccgtgttgcaggaacctggtggttgggagacgactaccacgtagctggagagccggacatcacaggcggcgccgcactcgagtgggatgggcgagcgctggaagtggtggagcatgtccacgaccgagggaaagtggaggtgctgcacacggcactggccccgctctgtcagcgacaggcgcaggtgcttggctatcccctgaaagttgaaagtgagcacgtattccccacgccgcgtctcgctctgccgcaccaggaacactccatgagcatcagggccctgcagctgaaccagctgagctgctttcactctggagatggggccgtggaaccaggggtagcaggacaggaaatggtccgtcttctggcaggccgggtccagcagccccccaggagaagcaccttggttaagggaatctgtgctgccccttggggagctggacgtgctaggctctagggctgagggaatatgcatctctgcttctgtgctctccagccctcggcctgtgcactccgagagctcagccatccatgaattcagctgctgctcgtctcccacctcaaagatgatgtctgtccggtccttcaccttcagcacaaaggtgtaaaggttgtcaggcatctcaagccgtgtgcaccaccggacctcctggatgctggagcaagctgcttgtagcttgggccttgaactcttgggtgggtcgaagagctccagcacgcggtcggggccatcggggcccggggcccggcgcagcgccagcctcccgcgctgccagcgtgccccgctgtccatggaggcctcgtcggccaggctgtagcgcagcaccgcctccttcagcgcctcgggtggcggctcccgggccaggctccagggcaggaacttcttggccaggccaggccgggcgggggtctcagcagcctctccgggggtcccgggggcggcagcggtgtgggccgctggcagctccccggccgagcggcggcggaagatgtggcggaggctgcggcgaaagtgctggaaggagcagggcccgggcggccgcggcggggccagctcctcagagctgcgggccttgggcaggccaggggcggcggggccggggcctggctccggggacgcctcggccttggctgggggcccacggcctgtgtcccggtagtcgcggcccggcgcccgtccgtcgcgcacctcgcggcagaagtagcgctggaagaggtcggtgaactgcagcgacaccagctcggcgcgcagcggcgcgtgctgcggatgctcccgggcgaacagccagtactggcgggccagctcccgggccgccgctacggcgtgcaactcacagaactcgctccagccccgcggggccgccgccggggaggctgagggcgcggaagagggcgaggagggctgcagggcaggcccgttcatggcggagacccaggtggtgcggccgggctgagagacaggagctctgggcccggccggcccatggcgtcgcgcgcccccgggccctcgaaatcccgagggggcgggcgggcccccgccctgggcgcgtcctctcccgcccgccggctcctcgctcgcggctcgctcgagcggggctcaggcaggaaacccgggctcggaaaatggccagcgcctcatcatttccgccgccagcgcttgacagttgcgggaggatgcgcccccgggccgccgccgccggggaaactttgcagcggccgccgtcgccaccgcgggccgcgacagtccgggcgggcagccgggcggaggcggtggcccggg

SEQ ID NO: 9 - DNA, 3088 bp, Homo sapiens
tttttttttttttttttttaatttggccttttggggtactttggaaaagtgagctgctcatctgtcattggaattatttttaaacatgtaaggaaaagctactgattgagttcaaaccaaacaaaatacttaaatgtatgaagtggtttcattaaattttcagaccttcttagcagtagaaacaagagacacatcactgtctgtagagttgtgctgtgtcatgggttttcctcagccaaccgccatttgcaataagtcatcaatcaagtggatgcatcgttaacaaattcccttcattgctaatagagactttacacacgccaacattctttttccctgaaaattaaccctattttaagtgtccaaatgttaagggccttgggcaaaagcaaactaaaggcagaaagaaaacaaccatccccttgcaatggaaaacaaaagaaaatgagagaaacgtgatgaggaggaattaccagctgatttcataacatatccatgcctcccatacacatatatatgaaatcaaaatctagacacatataacttggacatccaaaggggactgctactcttcatacattcaaaaagagttgttgaaatttctactcatgaagcagctcttgtaggcagttcggggatttgaaaatctcagggacttcactatccagcttgcagatgaccttgtatatggccttcttccaggaaggatcaacatctttgcctgcgataatggcattgaaaaactcccgtaatgtgatctgagcaacttccaggaatctctctggaacctcaaagtcatttgctttattgtagtgcatgttcagagccctgtacagctcacagtctctggttatagacagctcttcagtactggtgaccccatcgttgatggcttgacgtgcgtacttctccatctgaatgtagtaaaactcacggaaattgctaaaccacttgatgagctgagaggtaatgcatctgttgaactttacgtcggagaagtaggtcttcagcatattggagctgggataacgggtataaaaaaacatgagctttgcttttttcaagtgattgggtgacaatccttcctgcattgcacttcccgaataaggtgatatttcagacatatcttgaagatcgccgcactcggactttatgagcgacaaggagagcccttcggcggtgctgggcgggtgtgctggtgaacaagggtggtggctcaggtggtgagatgacatcttggtcctcagactcgtggtatccctagttaagtctaaggattcaggagaggctctgtcttttccagagaaggagccggagggagcacctaatgggctctgaaatggataggccatcaagggaagggggaaggggtggcggaaggtggacgtggtgaagcccgtggtggcagagagaggcgactggtgcaggggctggtggtggccgccagcggcagggccggaggcagactggtcagaggagtttttgcggacaaccaggggcagtgcttctgtctggtcagtggaactggccatctgcacattgccaaaggtgtccagggggttcggaatgatgacgtcgccaaagcactgcaggcgctggttggcggtgtggaaattgtggttttccccattgactgcaaatctggcctgggggatctggagaggtgggaagacctgaggaacctggcgggagggcttggccgaaaagactttgaccacagtgtccacaacttgcgacatggcagtgttcagttcctgtttcaaggtctcagccaaatgtttgccttccggttgtaaggagtttggcccatggtctctttctttgttgttgccttctcgcttcggcttgttttcagccatttcctgctctctgatcagggctcgagctcggtcaataaactgtcctgggtctagctcgcacatctcattatctgaccttccgacagagtcctgggccctggcatccaggatctccgagcgcatgctgtcttcagacaggttaccatcttcatcattttccgaatcagtgctgtcatagatttggtagaacttttcctgcagctggcgcagctgtttctgcatgtcctccagctgctgtttcagctgtcggcgctcctctcgcttctgttcttttcgggctgaaaccagctgctggaaactctgttgctgctgctggggaagcttttgcttgcgtttgttttctctgtaactttctcggggactcacagactgcggggccatctctctttcattttcattgccccttaatgccacactgggggaatggctcataccccgaattatattctcaacccgggcgcgctttgctctcaggtgctcatcacataagcgatccatatcaaactggctcatagtaggcctgccaaaaggggaaagacactctggggggctgtctcttgaagagttgctgcatatatcctcctgatgtacttcggagcctgtactagagagaccgctggcttggaaactgggctccgtgccaccatttttgttcatgttatttttcaacagctgggaaattatggttgctcctggaaaaggcatcatggcatcttcatacgagttcgccctcttcagcagcttgcggagtacatttgacttttccccatctgcatgctgcaccactgaatactcaacatcctgctcagaaccttggggattcatggcactaaaaaacgttgctcttgccttagcaaaaaatgcagatgctgtccctaccgtccttttcactccaatgtcaactcttctcctcttggtttgccggcttaagagggctgtgctgtcatggtcaggcatcactggacggttattgtgccatcttcaaaagctcgtcagctgggcacagctcaagaatcccgggacctcttaggaactgggaggagcggagcgctgagttccacggaggctcccgcttagaaactgtttagaggctattttattatgattcatttgaataacaccattttctcctggggggaataatgtttacacgggtctgagtaatgggaggctcttttcttcctttctcattcaagaacaagacagtgcaagtgattctggaaaagggg

SEQ ID NO: 10 - DNA, 6247 bp, Homo sapiens
attattcatatttgttattttattatatattattgtatacaactttgataatgaggatattataaatcatactttaagcaaaaatctatgcatgatatatgtaagcagtaacattttgaagaaaaaagccatgaaagcatttacctaaaatttagtaacatcgaaaaacactagtttgtgcatagtaatgttgaaagcttcataatacactagaatactggtaagtcttcaggtattgtaagaaaaacctggtacaggaaaagactaaattagacacatccatatccttagatgtgcacatcatctagaaataaatcccacaatgtagcagtgcactaagtatcctttgtttggcacttaacaatacagacaaacgtgtatttggtttaacgtgattttattattcttagatacattttagttattttatatagataaaaatatacaatattgcttttcaaacttttaattttataactgtataactgattgtcatcatattcatgattaaaaagcctattttccactaaacaatttatccagtaacatgtggaagagagtaacaaaaatggtttatgtaagacttataataaaaggtttaaatgtaaaaatagagtattaatctagacctctggtctaaatccttagatacctcatgctggaaacaaattatgaaatgtttcagacaaattttttccaacacttaaattgattctacaggtttaaagttttgactgaaaatacacagaactcagagatattttgtccgaagccatttttatatacattaacttactaaaaaaagtaaaagaaataaaaattgtattttttttttttttggagacggagtctcggtctattgccaggctggagtgcagtggggcgatcttggctctgcaacctccgattcccaggttcaggctattctcctgcctcagcctcctgagtagctaggactacaggctcacgccaccatgcccagctaatttctgtatttttagtagagacagggtttcaccatgttggtcaggatggtctccatctcttgacctcgtgatccaccctcctcgccctccccaagtgctgggattacaggcgtgagccactgcacccggccaaaaattgtattttaatacatctcagtttgggattccatcagattttcaaataatacatgcaaaatgtgtctaagctcttcagctctcatatctaaagaaaagaagagctgaatgcagtggctcacgcctgtaatcccagcactttgggaggccaaggtgggaggctcacctgaggtcaggagtttgagaccagtctggccaacatggtgaaacccgtttctactaaaaatacaaaaattagctgggcatggtggcacatgcctgtaatcccagcaactcgggaggctgaggcaagagaatcacttgaacccaggaggtagaggttgcagtgagccaagattgtgccactgcactccagtttgggcggcagagtaagactctctctcaaaaaaaaaaaaaaaaaagaataaaagatggtgtcattttttcttttaaaccatgttaggacagtaaagttggtagggtgattaatttgcctattggtaatgaagatcagtatatcattaaaaaaacctaaagtttgaaggtaaacgaactatgtggttgtctttttattaataccaactttaagcagttggattcattacttgatttcttagaaagacacattgataaagtatgtaaagccccaacaaattaagtgcatatataaggtatggactttttaaaatgaaaaactgcaaatatcttacaaataattttgttcattatatctatacaatgcaccattcaattcactcattacaatactttgcaccaaaccttgctatctgcaatcttaacactcctcaaatatgagaaaaacacatagtttccactgttctaagaagcgaaatttataaagtcaacacaatatattatcaaggattataggtctaaaacagtttttaaagacaatttatacacattgaaatgggtgctttgcttaattaaaaagctagaaataaagcttcttgcaatttaataaacaaggttgcagttaacagcagatttttcacattgcaacattcaactatcatcatggagatatcaataaaggtaaagagtggtatatttatttcttctgaaaacactgttgaggaatttaagtgctattgtcttaatatttttataataattaagaaataatttggaaagacattttacttttaaattttttcaacataaggcacatagcaacatttatagtctagaaacaactaaattatgtaataaataattattttttcgatcattatttgccctttacctgaagaaagtaggtaagcagtggagttaaaatatgcctgaattaatgaaaatggttggatttaggaaacacaaataatatttgtgtgcagaaagatattaatttaaaaaagaaaaagaatactttatataaaatatacatcctagagtgttataggtagaaaaaaatgtgaggcacttagatgaaacagtgaaggaggaaagcactgagattatctttttataaaaagcaccaaaccatttattttaacatctgagataaaagcagaggttaacatgataggaaaaactagtttagttctctaactttgtatttggggaactaataagaatccagtagctcaaaactcaaagtattttatttttactgtgacagaaataattcaaatagagaatattttctaatttaaaagaatcttagtttccctgataaagttttgtatattttgtggtgcccagaataatgtgaaatgtagacggtacctcaccactccgtggcagcaccctttctatcttgactgtagtatatgtaattttgataccatttttaaactcccttttctaaaagcaaaaattaaaatttggtttggaataattgtgaatagcatgcatcaatgcctccatgtgaacttgctgacgtccaacagtgacagttttatttttccataatccttcacaaactaatttaatccaaaaaccagggtcaaaattgggcttggaattacaggaatccactggatgtcaaatcaggaaaaggtctggcttctgacggatgatgaagtggctgaagatgtgtggtagtctgagtgttatttatgttatttaattcagctggatatgtttcatttaaaactccattctgaaacttgtttaaaaatgcctgttggcctggcagtactggattgtactgcatctgacccacgtgggtgtgctgaggttctggctggcactgatacatcgacacggccccagcataacatgtctgaggagttattatggctgactgtggatttaatccatgcttttggttttcaggaagttgtaaacaagtgacaaaatcttctaaactagaagtagtggggtatggggatggttcaaaactccccatagggtagtccagctctgtacatttggaatgttgtggtaatacaggctgattacaggaaataaagttctgtgtataaggcatagaatccatttcagatttgtaggggaactcttgactgatcccatgtaagtctgtaaagacattatattgttgtgggtcttgctgtggacaattgaaaggcacgaattggttagagttccaattttcaaacatgccattaacttgcatgtgcttcatcttctgacacagctgttgctgtggctccactactacttgcttttggtgatgttgctgttgctgttctagatgtaggtgttcctgtaccatacagcttgagttcagagccaaggactgttgctgttgataatctgaaggtatgaagggagacttacttaaagaatcttggacatacgtcaggatttcatccgttaagtcaatgtctctgaagtcaacctcaccagaaaaatcatttctgaaaaatttttcattctgcatgtgtctgatgtcttcaaaatcaatgcctaggtttttcattatgctgtacaagtcactgtttttactatcttgaaagagccctgggtgacctccagcaaatgagttcacatcctgaggctggtcaatttgctcatgtttcaggatagtatcatttcccatcggtgcagtattatcttgccaatttctgcattcattcatagattcgttgaaaaagttgttttcaaaaggtgcagtacttgaagtacttgaagcaggatagagataaatagactcatcttgttgcatcatggcagccaggagggaactaggattgagagagtccttgcttagagtggatgtggtagcagagtcttttccactagtgccatttttagtccttagtggtaagggatccattatggcaggaaaagggttggttgcctcatacaacacagcttctccagtggtaaacataaaaggcaacttcgtatttcgttttcgtaaatgctctgttccttcctcatctgttagtggtctctgagttacaatgatataatctggtcttccatttttataaagcaggcgtgcattagactggacccaagtccatcggttgttttttgtaagaagccggaaaactatcatgccactttctccagtcttaatcattcggatatgggactcggcacaataaagcatatcagctgcatgaataaactgataacctgagcctctcgtgcacagctctgcttcagtatatcctaaaacaattcttcctttggcatcacaaccaataggtgtgaagtctagtttgtgtttggttctaaagataaaatttttggtccggatttcaagtatggatggtggctgaagtggagtagctatcgcaaacaaagccaactgaggtggaagtattgatccatctttccctttctttttctgtccatgaagatactttaacttcccttggaaattcattgccagaaaaccagatgaattatccagcagacaccttagacgacatatgaagcacctctccattaaaggagagttttctggaggaatctggtctgggttataacagactactgtctgggggagaccagtggcttcttcaattccttgtccagactctgtacactgagaaggatttaatgcccagtgtagctgacgctgaaattcagctcggtcttcggtatggataagttcatatacactctgatgtatgacatcagactgctgaaaccctagataatcttgtatagtagaagaagcataaaagaccaaagcatctgtagtgacaactaatacaaagccattcagagcctgtaataagaattctccttcttgtaagttcaggccttctctgaaatttgctgctctacagttatcctggcctccgtttctttcagtaggggaggattttaatgcaacatcaaagaagctcttggctctcaggtaactgacgctgagcctaagaactgaaagtttgtccaacttattaataacatcttgtgggaaaggcagcaggctagccaaacggtccaactctgtattaagtcggtctctatgccgcttggaaggatttgacttgattccttcagctgggattggctttactgttttctgcaccggcttccgccgcttgcgactggcgtaggtgatgttggcgctgctgctgttcatggtgcccagccgacggcggcggctactccccgggcactggcggccgggtccccggaacccggtgtaggctgggaccactgcggcggccggcgccgggttccgcgaagccgcgcccgctccacctgcgtcctctcgggacagtggcgtgggcgccgcgcctcaacgcctcggctcacgcaggcttccgcagcccgcgccagtacaggtgagctgcctgggcctggcgcagtgagggtgggcgaggggcggagacccgcgagcaaggcggcgcaggtgccgtctacaccggcggaggcggcgcggcaatgcccgcaggtgaggcggcccgggcggaggaggagcctgggccgtctatttagaatcctggcctgggtcgctcacggtggcgccgcgccccgtgaccgtcgccgcggccccttgggtgagggaggtgccgccgcgctgctcccgggacttcctaaatccagggtgcttcccgcctgcggagctcgggcgcgggccaccagtcccggctgctcccagcttccgttcggctacacggctgcccctgttctgagctggcggtccggggcacgctctcggaacagagcgtcgacgggactccccagccact

SEQ ID NO: 11 - DNA, 6379 bp, Homo sapiens
agctgctaactcgctctagtagctgtggtcggggctcgggaccggctgccatcttagtctagggactgaggagtcgccgccgccccgagtcccggtaccatgcatttcacggtggccttgtggagacaacgccttaacccaaggaagtgactcaaactgtgagaacttcaggttttccaacctattggtggtatgtctgacagtggatcacaacttggttcaatgggtagcctcaccatgaaatcacagcttcagatcactgtcatctcagcaaaacttaaggaaaataagaagaattggtttggaccaagtccttacgtagaggtcacagtagatggacagtcaaagaagacagaaaaatgcaacaacacaaacagtcccaagtggaagcaaccccttacagttatcgttacccctgtgagtaaattacattttcgtgtgtggagtcaccagacactgaaatctgatgttttgttgggaactgctgcattagatatttatgaaacattaaagtcaaacaatatgaaacttgaagaagtagttgtgactttgcagcttggaggtgacaaagagccaacagagacaataggagacttgtcaatttgtcttgatgggctacagttagagtctgaagttgttaccaatggtgaaactacatgttcagaaagtgcttctcagaatgatgatggctccagatccaaggatgaaacaagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcacaggaaaatctgccctagacaatggacctcagatagcctatgttcgggacttcaaagcaaaggttcagtatttccggttctggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaattcatagacacgggtttttctttaccattctataagcgtatcttgaacaaaccagttggactcaaggatttagaatctattgatccagaattttacaattctctcatctgggttaaggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcagaatggtagctgagtggaggttgtctcgaggtgttgaagaacagacacaagctttctttgaaggctttaatgaaattcttccccagcaatatttgcaatactttgatgcaaaggaattagaggtccttttatgtggaatgcaagagattgatttgaatgactggcaaagacatgccatctaccgtcattatgcaaggaccagcaaacaaatcatgtggttttggcagtttgttaaagaaattgataatgagaagagaatgagacttctgcagtttgttactggaacctgccgattgccagtaggaggatttgctgatctcatggggagcaatggaccacagaaattctgcattgaaaaagttgggaaagaaaattggctacccagaagtcatacctgttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttagatttatctcccagtgatttctactcagcgtttccagaaatcaggtctgcaaatgactagtcagaaccttgcttaacatgagattttaacacaacaatgaaatttgccttgtcttattccactagtttattcctttaacaacaatattttatgtgtgtcaaaagtctcacttgggagtagtgtttttttcttttagacattctgcagacatgcagggaagtcctttggtaactgcaatatacaagattttcctattaagcctcttggtaagaggcatttgttaaaagtgcaagcttactcctgcttctggggatgtgagcaaaattcgggcttgtgttctccctctcattttagtctgacttgactattgtttttcctttctggcgcatgaatccatacatcattcctggaagtgaggcaagactcttgcatctctacaaagtagttttgtcaatttgaattcagggaaaagttggtcacagcctgcaaatgacttcatttggaagtctgattgtttcagttgcctgacaaatactacactttacaaacaatgttaacactgtgattccttcattgttttaagaagttaacctagggccgggcatggtggctcatacctgtaatcctagcactctgggaggccgaggcaggaggatccctttagcccaggagttaaagaccagcctgggcaacatagggagaccctgtcttttttttgggcagcgtggtgggggataaataaataaaaggaaaaaaaacttagcctagaattagaattaatttaattgaattcatctaaagatgtctctggtgatttttatatgttccgctatataattgatgctttatagttttatcataatccaacaacttcagttatatttaattattgttaaggagtttaagactagaaagactagagtgctttctagtccaaatagaggtcagtgaaacagcttttgacatcagattttcatttgagagggagagctgtggtactggctaaaaagaaaggaagataacatccagtaaccacaggaatatattctctgtgaattaaaagtcttcaaagttatcatttctctgacatatgttggagtagtcatttccattctttacattgtcatgaactggattgataaccctcatctgcaatattttcacccctaaaatttttaacagggtttcctttttttctcacgactatttaagtttagattgctccattattaactgattaatgcactttgaagttctctggaattaattattttaacttggcctagcttcgactgtcaaggtggctgttataaatttgacttcattggcagtggatgaagcctaagccagctgagtctctatcatagctgaaccctgaggacagcctcatagctcatgtatcagggacttttgccacatttcagaggcatagcatgaacaagtaatattaagccaagaataagcagcagaaccctgttccatatggaaaaaagaaaaacaattttttgtccctaatgttcttccttttacatcctggaacaacaataaaaacatttttttaaacttgtctactgtaagatactgccatcataaagcagagacttacatgagtgaaagggttgcctcatcaagcagctcagtgtaaatggggaggctaggctctccccagccctatggtttttttatttcatgtaccccaggaaatactgtgtggtttctaaaagccctggttgttaaaagtagggactctgcctttttgttggtagggagaaaaaacgctattgctttgtcttacagagcgaatgtctgccaactacccgttcattatataagtctgaacttggtaatatatggctaatgaagattaagccctctataaagacttcctgttgaggtgaattctcatactgaaatgtagttacctacaatatttactagagatttatgaaattaaattaagagataatgtaagaaaatacatttttttggttctatataatgcttcatgattcatttagggacctagaaatattgtgtgaaaatatataaatatcacccaaaaggctttctgccctatatttttaaaatacagaatagttatatttgaagtagccctggccctagttctatagggcttggctatttaatatttttatggaagaagtgtttagttctggaaaaggtaaatgcttgtatatatttttgcagcctgggatctccctactccattttttcctttaatttaagtggccacatgtatatgtcttccctgctgtgttaggaaaatgggggctggatatcccaagaatcagaggttatataaaaatactgcaaatagaccgcagacataaatatctaccaaatgctatcttaaattttggtccaaactgaacatatggaaatagatttattgtaagtatttacttagagctttttcttaaatctgaactaacttgcttttagaagtctttttctttgtaagcattgtaaatgctaataaatcctgttaatttttttttttttttttttttttttttactgcatgttacattgaaataaaaacaaagtaaaatgagcaattgccttattgttctgctcttttgcagggggtgggggttggggaagacagcattccacagcctggtcaggagggaaggacctcacttgtttcatcaaatggagtggtttaaaactgatcaagcagctcataataatgaattcctgctctgtcttttattatcttatgtcagagtgcagcaaaatctcatttcataagacttacaataactctatttaaaattcaggtattgtactcgggaggctgaggtgggaggatcccaggagttcaaatcctgcctgggcaacatagcaagaccctgtctcttaaaaaaacaaaaagacatattcaggtactgatttttcatcaagagattgacataccaatgatcagaaattttagtgaaacttgactcaactatacacaaagttctcatcctaaatgaactatttcagtcaagcccactctaccactttttacttatctggttaataatccgaaatgttaccggggttgactcacggaattaaatttttcctctatttttatatcctttcctgttgtggatgtaacttacctttctaaactcctttagagttaacgacttctatttagtattgttggtatctccagcccccctcctttttttgtttttcttttagttatttactttgtttgtataatttgctttcattaactaaaaatggaattggcagcactggattgtatattactgtattaagagtacctttgttttcttgatctgttacttttaatactcaggtaatgctacaaatattacagggagtcaaatgcttttataaagttccttcagtggttgcctcacttgcgttttcctacaagatgtaagactgtttataattaaacttttttggtccttc

SEQ ID NO: 12 - DNA, 6379 bp, Homo sapiens
gaaggaccaaaaaagtttaattataaacagtcttacatcttgtaggaaaacgcaagtgaggcaaccactgaaggaactttataaaagcatttgactccctgtaatatttgtagcattacctgagtattaaaagtaacagatcaagaaaacaaaggtactcttaatacagtaatatacaatccagtgctgccaattccatttttagttaatgaaagcaaattatacaaacaaagtaaataactaaaagaaaaacaaaaaaaggaggggggctggagataccaacaatactaaatagaagtcgttaactctaaaggagtttagaaaggtaagttacatccacaacaggaaaggatataaaaatagaggaaaaatttaattccgtgagtcaaccccggtaacatttcggattattaaccagataagtaaaaagtggtagagtgggcttgactgaaatagttcatttaggatgagaactttgtgtatagttgagtcaagtttcactaaaatttctgatcattggtatgtcaatctcttgatgaaaaatcagtacctgaatatgtctttttgtttttttaagagacagggtcttgctatgttgcccaggcaggatttgaactcctgggatcctcccacctcagcctcccgagtacaatacctgaattttaaatagagttattgtaagtcttatgaaatgagattttgctgcactctgacataagataataaaagacagagcaggaattcattattatgagctgcttgatcagttttaaaccactccatttgatgaaacaagtgaggtccttccctcctgaccaggctgtggaatgctgtcttccccaacccccaccccctgcaaaagagcagaacaataaggcaattgctcattttactttgtttttatttcaatgtaacatgcagtaaaaaaaaaaaaaaaaaaaaaaaaaaattaacaggatttattagcatttacaatgcttacaaagaaaaagacttctaaaagcaagttagttcagatttaagaaaaagctctaagtaaatacttacaataaatctatttccatatgttcagtttggaccaaaatttaagatagcatttggtagatatttatgtctgcggtctatttgcagtatttttatataacctctgattcttgggatatccagcccccattttcctaacacagcagggaagacatatacatgtggccacttaaattaaaggaaaaaatggagtagggagatcccaggctgcaaaaatatatacaagcatttaccttttccagaactaaacacttcttccataaaaatattaaatagccaagccctatagaactagggccagggctacttcaaatataactattctgtattttaaaaatatagggcagaaagccttttgggtgatatttatatattttcacacaatatttctaggtccctaaatgaatcatgaagcattatatagaaccaaaaaaatgtattttcttacattatctcttaatttaatttcataaatctctagtaaatattgtaggtaactacatttcagtatgagaattcacctcaacaggaagtctttatagagggcttaatcttcattagccatatattaccaagttcagacttatataatgaacgggtagttggcagacattcgctctgtaagacaaagcaatagcgttttttctccctaccaacaaaaaggcagagtccctacttttaacaaccagggcttttagaaaccacacagtatttcctggggtacatgaaataaaaaaaccatagggctggggagagcctagcctccccatttacactgagctgcttgatgaggcaaccctttcactcatgtaagtctctgctttatgatggcagtatcttacagtagacaagtttaaaaaaatgtttttattgttgttccaggatgtaaaaggaagaacattagggacaaaaaattgtttttcttttttccatatggaacagggttctgctgcttattcttggcttaatattacttgttcatgctatgcctctgaaatgtggcaaaagtccctgatacatgagctatgaggctgtcctcagggttcagctatgatagagactcagctggcttaggcttcatccactgccaatgaagtcaaatttataacagccaccttgacagtcgaagctaggccaagttaaaataattaattccagagaacttcaaagtgcattaatcagttaataatggagcaatctaaacttaaatagtcgtgagaaaaaaaggaaaccctgttaaaaattttaggggtgaaaatattgcagatgagggttatcaatccagttcatgacaatgtaaagaatggaaatgactactccaacatatgtcagagaaatgataactttgaagacttttaattcacagagaatatattcctgtggttactggatgttatcttcctttctttttagccagtaccacagctctccctctcaaatgaaaatctgatgtcaaaagctgtttcactgacctctatttggactagaaagcactctagtctttctagtcttaaactccttaacaataattaaatataactgaagttgttggattatgataaaactataaagcatcaattatatagcggaacatataaaaatcaccagagacatctttagatgaattcaattaaattaattctaattctaggctaagttttttttccttttatttatttatcccccaccacgctgcccaaaaaaaagacagggtctccctatgttgcccaggctggtctttaactcctgggctaaagggatcctcctgcctcggcctcccagagtgctaggattacaggtatgagccaccatgcccggccctaggttaacttcttaaaacaatgaaggaatcacagtgttaacattgtttgtaaagtgtagtatttgtcaggcaactgaaacaatcagacttccaaatgaagtcatttgcaggctgtgaccaacttttccctgaattcaaattgacaaaactactttgtagagatgcaagagtcttgcctcacttccaggaatgatgtatggattcatgcgccagaaaggaaaaacaatagtcaagtcagactaaaatgagagggagaacacaagcccgaattttgctcacatccccagaagcaggagtaagcttgcacttttaacaaatgcctcttaccaagaggcttaataggaaaatcttgtatattgcagttaccaaaggacttccctgcatgtctgcagaatgtctaaaagaaaaaaacactactcccaagtgagacttttgacacacataaaatattgttgttaaaggaataaactagtggaataagacaaggcaaatttcattgttgtgttaaaatctcatgttaagcaaggttctgactagtcatttgcagacctgatttctggaaacgctgagtagaaatcactgggagataaatctaaatgagaacattaatttacttacactcagataactctctaaagagccattgtccaattttacaagatgtgcaacaggcagagaaggacaaacattgcaaataagttcttgcccattcatggtgcaagttctcagaagttactcttgtccaaatccttctgtttcttctatggcaaacaacagcttttccttcagttgctcatagctcttgtatggtggcaggtccaggcgattaaaacaggtatgacttctgggtagccaattttctttcccaactttttcaatgcagaatttctgtggtccattgctccccatgagatcagcaaatcctcctactggcaatcggcaggttccagtaacaaactgcagaagtctcattctcttctcattatcaatttctttaacaaactgccaaaaccacatgatttgtttgctggtccttgcataatgacggtagatggcatgtctttgccagtcattcaaatcaatctcttgcattccacataaaaggacctctaattcctttgcatcaaagtattgcaaatattgctggggaagaatttcattaaagccttcaaagaaagcttgtgtctgttcttcaacacctcgagacaacctccactcagctaccattctgatgtattcctctttattttcttctgttacaagaatattgccaccattaggtttcagatcatgactcttaatttcacctagaatttctttgtcaacggagaagtacatttccaaatcacattcctcaatattgttttccttaacccagatgagagaattgtaaaattctggatcaatagattctaaatccttgagtccaactggtttgttcaagatacgcttatagaatggtaaagaaaaacccgtgtctatgaatttcccatggaacagagccatggcaataaatctgccaataaaacgaaaatatttcaggtgatctggattgatgtaagaagcggggtttatctgcaagcagtagttatccttccctgcatattcaaacaggcaatacattgggttcaacacttcatgtgacaaaagaaagaaccattctcttgctacacctccataatctaaaccttcttctcctggaaaaatcacccacaaacgtcttcgcagatcttggggactgaagctcattatctgttgaaaggaatcctcaaacaatgtttttcttgtcactgtaatctttatgtgctgtggcatggccagttgctgacaccagaaccggaaatactgaacctttgctttgaagtcccgaacataggctatctgaggtccattgtctagggcagattttcctgtgcggggatctatataggtggtagttcttctattgtggtccacaaaatatggaattccatccactgtgaatctcatttcccaaccttcaggtaagggcttttcatttaattgaccttgacttctggggtcttcccattgtgtaattcgtgtgttgtggttgacgaaatatactctgccattgctgtctgttctcttctcccatccaggtggcaatggaccaagaggatcaaattctttactttgtgatgtagcaaataaatcttgattcccataaatgaatctctggttaaactgctgcattgctccttgaagctgactacgctgtagctgccattgttcatagttccggacggattccagtgttggcctctgccacgttgttgttcttgtgaaatggtcaacataataaatacgtcccatgttgtcaacccgccgttcccagccaggaggtagaggttctggtctatcccatgttgttcttttctcaacatgatctacatagtaaactcgcccgtgctggtccactctctgctcccaaccaggtggcaagggagcttgagttacaggatttaatggcctagggcctgagcctccagatatagtaagaggaattattaatccagatgttgctccttcagatgtatttgtatttgtatttgtcggcggcagagagcctgtactagacccatcactttcagaagtggcagatggtgaaccattgacagatgctggtctacgtggggtgggtggtggtggtcgtgaaggtcttggaggtctagaaggtttaaaaccaccatttgagagtgatggagaattattcccactgactctcctattttcaccagctcctgcatcttcagggtcatctgatccatttgtgctcactcttgtttcatccttggatctggagccatcatcattctgagaagcactttctgaacatgtagtttcaccattggtaacaacttcagactctaactgtagcccatcaagacaaattgacaagtctcctattgtctctgttggctctttgtcacctccaagctgcaaagtcacaactacttcttcaagtttcatattgtttgactttaatgtttcataaatatctaatgcagcagttcccaacaaaacatcagatttcagtgtctggtgactccacacacgaaaatgtaatttactcacaggggtaacgataactgtaaggggttgcttccacttgggactgtttgtgttgttgcatttttctgtcttctttgactgtccatctactgtgacctctacgtaaggacttggtccaaaccaattcttcttattttccttaagttttgctgagatgacagtgatctgaagctgtgatttcatggtgaggctacccattgaaccaagttgtgatccactgtcagacataccaccaataggttggaaaacctgaagttctcacagtttgagtcacttccttgggttaaggcgttgtctccacaaggccaccgtgaaatgcatggtaccgggactcggggcggcggcgactcctcagtccctagactaagatggcagccggtcccgagccccgaccacagctactagagcgagttagcagct

SEQ ID NO: 13-19 - skipped (7 entries)

SEQ ID NO: 20 - DNA, 47907 bp, Homo sapiens
ccccttttccagaatcacttgcactgtcttgttcttgaatgagaaaggaagaaaagagcctcccattactcagacccgtgtaaacattattccccccaggagaaaatggtgttattcaaatgaatcataataaaatagcctctaaacagtttctaagcgggagcctccgtggaactcagcgctccgctcctcccagttcctaagagtaagtgatcctcttggcttttatttctttctctttcctgctggtggctgggggtggcggtggcgatgggggggaggctgatgttgctggacttgtcgctgatcttgtcaccttttgtgtactgtttctggggtgtgaggaggcgtttgctccctttccttctttctcctgctctctcttctcaggagagaggaccgcgagagggaccgggtcgcttttttgttcgtggagatccccgctttccgccaaaccccatccttccgatctccccaggctaaaactccggggccggtccccttgtcctttctctttgtcttgtttattatagctgcctttcttcccggctcttccaatttgcttgtcatttgcatacctttcacttctccttttttaaccccagcagaggaccgggaactgggaggaggagagagggaggtgggggggcgctctgttactttcgtctcaaaacgctgtcgaagccgaattgtggaaatccggcttggaggggagcggtgatgggtcccgggaaacgcgcgcggcgcccctcttccgagctcctggacccagggctgggtcaagttgagtagggtaaggcggcaccgggaggctcggggggtcgcgtggcggtgggattgggacaccagcacgaggaggaccggaggatcgcgggccgggtaagagtagggggttcttgggcagcagaaatgggaggcgatgaatctcccagccatcgctggcagactatggtgttgggcagcttcggtctggtctcgtctgggtggtacctaccgttttgccccagttaggaggactggggagggaggacaggagaggtgagagtaattgttactgggaagactagtgaggagggcgggaagagggagggaagagctgctatcttgcctgagcagatcaggagggggacgcagtgggcggggggagacatcacccaaagtccagtttagcaagttgttgattcttctggtgtgccagcccgttactccccctgctgaagctgaaggttggtggagtgatggagcgtggggatggtaaaggaggagtaagtagctttccacagactcccaggtctctggccccttcccagcttcttgggaaattgagagccctccaggcagacagagaacagaactagaaggaggggtggtgcttagtcttaaatagctcaaggaggcaggttggagtgtgaaactgctgttcttggcaacccagaaggctactctgcctgggggaaggctggaaactcacctgcttgtttttatttttccgagaagatctgtgctgtctccttgagcttataaaaacagaggaagcacagggtggcctcctcgcaaagtcaaggctagaagactcccttctcctgttctcttttccactcatgccctcccttatttaaaaaaaaaaaaaaaaagaaagaaaagaaaaaaaaaagaactcatttcctttcctaacctaggtaggcagaaatctattagcagagtgcgcatgggcagggcctgacaggtgtgttgtgtcaagaaagacaggtgcaaatttcctctgtgtctgtgtgtgtctgtacagctctagaccacaatgcttgctcgagggttggagaggtttatgaatttatggttgtcctggttaataggattgtctgggctaatgggaattgggctgttgttcttttgagccctgccatgtgagttcttggggtggggggtgggggcaagttggtatgtgtttgtttatttttcttaaggatattggcagtctactgctgaggctgtgtcccaggcttctgtctgccagtcagcccaaagcacccccactttaggcagcaggtggagggagactgacttttcctttgcttcctaccagtttatgcctatctcccaggtctgtgcttggcagagagagagagagagagagagaactgtcgtgtgtgtgtgtgtgtgtgtgtgtgtgtgtgtgtgtgtgtgtttgtgtgtgtgtggtgtatgctttggatagcaatgagtggtgtgtaactgccaagaattccaaagtcagtttgaaagtgttactgttgttaaagcttatctttttaagcatgctttctccttgcccagaaagaataggtatgtacataaactctttcaagtcatatgttaaataatctcataaagtagaatgagcctgtcattgtcccagacatgtgccaaatgtcctagatatgaatttgatggagaaagaaaatctcaagtacatgagaaggtaactgtgcttttctattctgatgcaagatgtgagaagtcagttctacagggaatttcttgcaagaacttctgagtatttccaaaatgaaattttttgtgtgtgttgagggaggaaaacgagagtattcacattaacttgtccatgggttaaaacatggacatgtatatgtaatagtaaaataggtgaagctaaggactgtggcttgatgtgtgaggaaagttgttgggaattcaatgtaagcactatatctggcttcttaaaacttgaccttttaaaattatctttaaacagactacttctgtagactgagttgcacaggaataggttggttggcaaatggtttttgctcattggctttgtgtttgggtagttattgtttccatgaaaatgagatcgtatgtgtcatttattctgtagacttcaacattaacgtccccccacctcccaaacacacacacacacacccaatactttccttggatgcttttgaagttctttggtaattaaaatgtcatctatgcctatgttcatttgctttatttttaataggggttatctgtgcttggcacttattgatattttatgtgtccattatgcagaattctatttagtttaatcaccaccttgtgggaaaaaaaagtcatgcatacataacatgcatctttgttctcactttattcatttcctagcatcattcctctataagcagcacatgctatcttaaaacctaagctggcttattctgtaagttgccagacttcctctttatttgtttaaaactcaaacaggcctcttttcatgaatgtcttatatcattttagggattgtcttgaatttgcagtgttaatataagaagttttaggtttcagattaacaaaagaaattataaaatgtgactgatgttataatatgaaaatagattgtgcatgatgtatcattataggattttaattaagtacctgtgtaacttggaaaggaaccatatacataaggaatttctcagacttattgcctgtgcattctcaaaggacatttagagagttcaattttctgcaaaaagaaaaaagtgtattttcttaagattatttcacactctgtcttatttacctatctgataagttgttactttttaaacaagtagaaattaatattttaggcatgtctcagaaaatgttctgtgttcattttgcaggtgaaaagtgtgtggaatttttgatgggatgggagaatcttaaatgaaatcttaaatgatttgagaagtatattatgacaggaaatttaaaaacctgataacgcaatcttagttaatttaggtattaacttatgtcaagtgagttcttcaaaataaatatcaaaggttttcttaacctgatagggagcagaaatatctccaatatctctgaagaaaaagttgctaattagcagaaacaaattcttgaatgtagtgaaggggacaatttaatgattcaggggctacttaaatcagaccatctgatttttcccctttgaatcactaatttccagattgatttgaaatattctttgttaatgatatcctatttgaaatttcataaccaggttgacccaagtagattagaggcccatacaaagatgattttctaaaagaagtcaagtgtaggcttgcacaatttcttcaaataattttatcaacaaagacagatcatctaaataatccaagcaggaaaccatgccaaccttacactctccctgcctcataaaagatttgtctgaactatctggataattaccgtaatgaaacacttctttgtccagaatctggactccagatagatgcagtaaaagttgaatcctcctccccgaaataacttctttattaaagtagagcacttaaccactttatacttcacgctgcagtgttcctttgaaattctttactgaaaattctttcctcctaaccttaagtcatcagtttccttagaattttgcatgttaaagagaatgtcagataattcagatattaaaggagactcttttggagtagttaaaacctgttttgattatacctggatgtttattcttctaatatctttttctgggaggaatctgctatgttaagatatgcattgtataagaattactaaagcatttgtgtaggttatatacgaagtgatgcaacaaaatatttaatgatgaaaaactctatatagactttcacattaattaaagaggggtttacaggaatagagtaagtgtatccgatcaataatacatttgggttcaaattctcatcagtatttttctgcatccttgctgatttggacatccaccagtgttgatcaaaagcttcatattgcctagtgaaactgaaaattaatgttaaaatgcaaatatgatatgcatcaataataattgcaggtgaaacatgatagcttaatacatatcttgagaaataaaggagtttaaaaaatatcaatgataaagtcattccatggcttcctttaaattctgaactggaatatcatggaagcacttgggaaatgtttttaagagatttaatttatattatggtaacgtaacagtacattttcttatgtggtaaatatattcatatagatatcttgtttatgaaatgtgatgctaataaagtgctgtgtcaaccggttattattatttaatcatgcctatagcttccatgggttatggttccagtgtgtgctaccactatacttttatttctaaattaaatctaagctatatggagagatatatttatttgtgcctattaatataatgccttgtcctggattatataatttatcttatttttcccatttgttttgtcttatttgttatgttccagctggacattttacaacaagacctaaaagtatttaaattcttttagcccaagacagatacaaatcgttatttaatctaaaaatgttgactgaaatagaattacaaaattagtttagtttggtgaatatcaagggagttatatcttgttcttaacagactccacaagcatttctttccaccttaggaagagcacagccctcctcttggctccagcatggggcagggatgcagctgttgatacctaggctagatgagaggaagtgcagttgacgcagaggtaaatggcagttggaaaaggaaggatgcctggggatgaccttgtgctcatcagcgacaccagtctgtcctttccaagcctctgtggcagagctgctcttcccacagcaaggatggcaggaggaaagtccagtttgggtgttagggtgaacagggagagaaaaaatactgcaaaaagtttgtttgacattttgattggagatccatgtgctttgcaggtgatagtcaagagaaaaggatttgcatacaaatagaaaagatgtaaaatttaaaaataagggcaataagctctattttggggaaggtgatatacacacagaaaaaagtcttccttgtaaccgccccccatgcaagtgtttctttgattaacagagctttgaaatgattcatcctttttcttgtctcagcctctccttgttctttctgtcatctgacagctaacctgatttatcagatctaatgtgtttgtgtagtatttgtcactgcatttttgtattcctgaaaccaattttattattagtgtttgaaagggtctcaatcattctgaattcaattttgaacccaatgttgtagttcttgagaactccatctccattctaagttcaggaaattttatcctgaagcatgcaaaaagtatttcattctcaagcatgcaaatatatatatatatatatatatatatatatatatatatatatatatatatataaagaggtatcattttgctttcatgataccctaaagcaggctcttttaaaatgttttatctttctatagaaaccaggagcaaagatttcatgaggaaatcactgtcacttaaaaaaatatacatattgttgccatctaagcattgagcattttcttgatttttacaggttatttcatgctgaaattatgcctatttgcatggatagtcattctttaaagctagccacagatgcagtcctagggagcacgtagatgtttttacaggtgaaccgaaagagatgggagccgttccagacactctgcatgctgcctttggcaatggaccctgttattgtgaagatgtgctctgttaagcaaacgtgaagtttaatattagataaacccaacgtgaaaaaaattttcattttcttcataaaatgttaattataaacaaaaagatgtgacatcttatatgtctacaaaatttgggattagcatcactagttaataagttacacaatgtcaagtgccttttatgaaattcaaagaaggatgttctctttttatactgtgtttccaagaaacaatggaagttcatatacaaagaaatatttccctttctcacacatttgatggacattattttctttcttctttatatatcttctttcagttttttctgtttttttttttcctttaatttggcacaggaaataaggttcacaaatcctgtatgttaaagagtttctttgggcattggacatattattttggcagatttaaacagaaggaaactagtcctgaagatatatttatctttatctcggtcaataacttattattcctcatattgatttctaaaatgtggtaacatccttgttttgcagtgaatccaactttgtaataatttgtcattaaaaggacattatgaaaatgtataaatattcttatagttacattaagatatatcaacagatatcatcttcacctatgattttacaagtaaaaaatgcatagctaagctaaataagcagacttataaaatgactattgtgcatttatttcaatgctaaactgaccatttatgtttgaaagatgctgctgctaagggtgttctccttcccattttacatatgacaaaaatattgtaaaattcaagaataaaagctctctattatatatttgcatttattttagagtccttttcctttaatagcgttaaaaccacactaattgtaatgcagaaatgcaatttttcatgtgaatttctcatagtctcaaaatttaaccttatttcttaagtatagagcagtttcatcttccttataatatgaatctcaatgcccaaaatttaatcaattggttgtcagaggctgtgttcttataatctactgtttcttctgaagataaacagtatcattttaggcatttgtgagagagaatcatattactggtgcttaagcagtttttgcttaatttttttttaatcttaatccatcttaaaccagtggagcagaaatatttaaaaatgtttcatttcaagcagagtgcataataaattgcaataattgtaatgtgccataaatcccagagcctatgcattttgcatttgattcaggattgaggtcaggaaatttggagaaatttaaagaaaatgattcatcagtccttttgttctgttggccagggtcccgggattcttgagctgtgcccagctgacgagcttttgaagatggcacaataaccgtccagtgatgcctgaccatgacagcacagccctcttaagccggcaaaccaagaggagaagagttgacattggagtgaaaaggacggtagggacagcatctgcattttttgctaaggcaagagcaacgttttttagtgccatgaatccccaaggttctgagcaggatgttgagtattcagtggtgcagcatgcagatggggaaaagtcaaatgtactccgcaagctgctgaagagggcgaactcgtatgaagatgccatgatgccttttccaggagcaaccataatttcccagctgttgaaaaataacatgaacaaaaatggtggcacggagcccagtttccaagccagcggtctctctagtacaggctccgaagtacatcaggaggatatatgcagcaactcttcaagagacagccccccagagtgtctttccccttttggcaggcctactatgagccagtttgatatggatcgcttatgtgatgagcacctgagagcaaagcgcgcccgggttgagaatataattcggggtatgagccattcccccagtgtggcattaaggggcaatgaaaatgaaagagagatggccccgcagtctgtgagtccccgagaaagttacagagaaaacaaacgcaagcaaaagcttccccagcagcagcaacagagtttccagcagctggtttcagcccgaaaagaacagaagcgagaggagcgccgacagctgaaacagcagctggaggacatgcagaaacagctgcgccagctgcaggaaaagttctaccaaatctatgacagcactgattcggaaaatgatgaagatggtaacctgtctgaagacagcatgcgctcggagatcctggatgccagggcccaggactctgtcggaaggtcagataatgagatgtgcgagctagacccaggacagtttattgaccgagctcgagccctgatcagagagcaggaaatggctgaaaacaagccgaagcgagaaggcaacaacaaagaaagagaccatgggccaaactccttacaaccggaaggcaaacatttggctgagaccttgaaacaggaactgaacactgccatgtcgcaagttgtggacactgtggtcaaagtcttttcggccaagccctcccgccaggttcctcaggtcttcccacctctccagatcccccaggccagatttgcagtcaatggggaaaaccacaatttccacaccgccaaccagcgcctgcagtgctttggcgacgtcatcattccgaaccccctggacacctttggcaatgtgcagatggccagttccactgaccagacagaagcactgcccctggttgtccgcaaaaactcctctgaccagtctgcctccggccctgccgctggcggccaccaccagcccctgcaccagtcgcctctctctgccaccacgggcttcaccacgtccaccttccgccaccccttcccccttcccttgatggcctatccatttcagagcccattaggtgctccctccggctccttctctggaaaagacagagcctctcctgaatccttagacttaactagggataccacgagtctgaggaccaagatgtcatctcaccacctgagccaccacccttgttcaccagcacacccgcccagcaccgccgaagggctctccttgtcgctcataaagtccgagtgcggcgatcttcaagatatgtctgaaatatcaccttattcgggaagtgcaatatccttttattttcccctcgaggaaaaaacaaaccaaaaaaggtttcccaaaaggttgggtttacacaatatctagagtaatgtagattagtatcttcttaagaaggcaacctttcccattattcaaaggaataggcttttatcagcatgcgtgtgccattcctgattgcagaaaagcttaaaactaagccaacatctttgcagcttccacaagttgttcactgccttgaggagctcctatttaatatgtgctttctcagcagtgttttttttctgctgttcttcctgcattatcttcttatccctatctcttaaaaaaaataaagaagtagatttagagatgagaaaacagtctcattgtaaatactgattgaattctctcagatattttttaaagatggtaagtttaatagaataaggagaaaagtcagttttcagatccctaagatcccataagaagaattctcagtgtaaaccatctgcaaggcttctggtccgtttaaagacagcccgatgaaatcttaggaagagcgctttacaagtgggaggttgaggaggaagaaaaatggatgtgggtggggagttagtctctctttcatctttaagtgagacttttttttttaaggaaatatacaggtactgatttattcagacagcatcggtctctctcccgttcacccaaggtctgttctttgggtctggtgcagctgcctctatgcatgattaacctctgttcagccatacacagaaatcttttgtcccaacatacacaaagcaaattattttggaaagcgagagagcacaattaaatataaaactcagctgtattcgacttaaaaatggctctttttatgattcttttaaattctgaaactgacgtttatgtagagataacagttatatttttttattaggcctatcccgaactccagctatttttaactgaagatttttttttctctctgtatatcggttctttctgtaaattttttaaaaatcttgtggtcgttggtcttttgggagtagtaaaatagtagcatttgggggcaggtggaggcatgtttcttatataataaacagatggatataaaatttagcaattaagttggctgtgactaaatttaggattttgagcaattgtcttgatgactagagattgacattttcatatctaagcccactccagaggctgccacgtaagtgcaaagtcccagctattggtggaaatatgttttcctggttagtggaggtcgtacttcaagccacctctcaggataatagtgtagatttctgatagggtgaactactagggccctaatcatgagtcctgcttgggcagttaaacatggagtctctcttatactgagcaagagaagaacattgtaacagaaagggaagagaaagatgtgggagatttctacatatacgtagaaatggagttttagcttggttgttgatttcacttggaccttttgaagatctaaaattcaatccaccagccatgaatcaaagctgcaccaagcaccatgccttacatattataagcaggcagtaaatattgatcaaatgattggaatatcgctgttggtgatgagaaaggcaaagtaagaagacacaatggcttgaatggtttttgtgccctttgcaaaaagagcatcttcagaggttcatgtaaggctaatgtctagggctaagaccccattgcaccccagagatctcttaacttcattttgaaccaggtagttgtgatagtgggttctttctgtctctctctctctcttacacacacacacacacacacacagacacacacacacagagtaaagtgacatgcgtgccaattttggtgaatatttaaagatttaatgccaggtttcaaaactcctgtaagtccacactaagctctttagttcaagatgccagtttatggtttttctttaaattagacttttcattataaccagatcattataattatggctgtgctttttgtttttagtcttctaggaaaaaaatcttttagattgctttaagtgttggctatgttcattgtctcaacctctccaaatccccggaggaattttgaggatttgaattgaaataagttccttttattttgatacatatcaaaggctttaaagaaaatatagttgcttcttcttcagaggcatgacttctcctttcttctatcaacataactttctgtcgagcggtgattctgttgggaaacacccgtgttcatgtgaaatgttagttgctcacactcagaattgtttctttcatatagctaaataatgtcggcctctcgtggcaattagtgattacattttccaccttttggccttctatgctcctattcttttcccccctctactattaatacattgcacttttaaccatttatctcattggtatattatttctcaggaagagtaagataggcaaacaaccttttctatagttcccacaattctgaaaccagtgaggatctgttggtttgtagagagattgggcccacttttctcctgtctctacctctgtatggcagtgtgttcttcccttgatttaactgttagtgtgtaggcaaaattctcaagcttttactttgaagaaatatctgggaatcacagtgagtgatgtcttacttcaattttagggatacggggccatatatgatccggttgtacagttattcctcgaaaagatcaatagaaatgggcagaaatgtaatgaaatggtacaactgtgattgctattattatgttttaatttttcgttcatggctttccaaactgttatatataatttaatttttcaggaaaaattatctcccactccaaaaggtaccatctgttttttgaacaaagtagctaagataagaactattaagaacaccagcttatcaggtcaacccattctacattcaccacattaaacatatatgttctgtaggatagaacacactacctcattatcccatctagtagaagggaaatagtgaatgtgtatgcaagttaaactgaatttcagtgcacctgctccaagggctcatgtcttggattttaaaaatatgttcagtatctttgcaaatgaatctgtttaatcaaatattaagttttattcaaattccaaaagaaacagtcagccaattgcttttcttcatgatgttccttgtcattcatcctctttgcatctcaagaaaaatagcctagtttaggccccaaacatttgcatgcacccagttaaagcacaagaggagtagtataagccgttaagacgtgcaggtgaagaaattgagcctgttctctgaaacagccggctttttctactcaacttttagggagaatgttagaaagacttgaagtttagaaaggaaaatggtttagtaatttgaaattaaaatccaaccaggaaccatagattagaaatgaatttctgaaatttgaaaccatccacagaaattgatcttatacatttttagaagtcttgtggaggctatagtacttatattagctagagcaaaacatgtagattaaagactaaaagactttgggctcctacactacccccctcccctgaaaaaaattataaagtaagtaaattaaaaaaaaaaaatccctacactacacagccctccgattatggtgaacttcctagtgggagttacgacttgctctatcactgtcattatgtgagagagtttagatcttttctccccattttagtttctagggggaaaacctcttagaaacttagcaaattagggaataaggcagaactaaaattctttaggtttcaaatgttttggaaaatgtaagtagtctcaacccatttgctgggaactgcagcacgtacaatctctagctacaatccagagtttagctggaaaaaaagaattttcttcctccgctttcacagcttattattctcccatttgcctttttgctgcctccgctgctcctcccgtggctgctgtttaggtaaggttatattgtacttggtaaacagacaacacttaggttctcaggttgtttgaacactgctttacgttcagctgcagtaccctgcttctctgatcttttatattcccgagcagatgtctttcattaatttatggatttatcatcttttcttttttttttcttttttcttttttttttttttttacacctggcagctgtctcaagtttcaacagttattgtctattttgcattacacatagaattgaatgtcatctgtcttcacaaagctatggctaagagaattgaggcacagccacatgagctgctgggacagatcttgtttgcgttccatcccccctcaccccactcccctttacctccttaatatttatttgtgctcattttctttcctggccttgaatggagcttagctcgtgttcagtacagctgtatgtttactgaatctattccatcatgagtcattgtgcgtgtgtaagtatcctggaaacagctagtgctttcttggaagaacagttgcttttcagcacaagcacttaaaagggaaattaaccaattggtcagttcagatttattttgaggagaaaaaaaggattatctaactgttgccttttaaatgtttcattagttatttttaatagtttattagaaacatatattttatgggaattttatcttaattacacaataagcaagagataaagattaattctgtgttccatttcaactgatcagttccaagtattaccaacaggaaacattttaaagcaaaaatgaacttgagaaatccaaatcagaataattttttgttagataaaaagcctctaaatactgatcaaaataaaatggatattttactttttttagataaaaagaacaaaaacatcttagcataaattagatgtattaaaagcttcaggaagttttggtagctcagtgcccatctaagaaacacagaaaaacactttgtattttgtatgacaccaaattttaaaagatttgtgacttccaattaaatgcatgacgttgtcttaatgtagccatctgaaagaaaagattagaacccagatctgagagtgtctgtcaaagtttggacttgcctaaaactcttatcacaaggcagtcgcagacagcttgcaactattatttcacttatccatttggacagatggtcctgaagtgtgctgggctcctttagtcttctgtatcagtctaatggaggttactggagggcctttcagccctctccttggcacaagaagtatgtcagtcataaattatcgtctttgtaatcattaaggatctcaaacaaaaacacaagttcagttaagctgctttggcttacagatataaaatcaaaatttctttctttagtgtttattttcagtttaacaaaaaataaaaaaataaaaaacctgcactacttaacttttctatttacagaccaaggtgatctttttaaaattgcatgggatattaaagggaatgttaattgaacaaattctcagcagaatatttggttaaacaccctgttataagtagtcaagagcttatccatattaatttgattatgcttctctagtaactttctggtttccctccattcttaagattagtcacgctagacttgatgaaggtcatttggaaaattttacctttcctaaatatctgtgtttatttgacatttctgcctaaggggtgaaatttttgttgggtagttgtgtgagtgtgtttgtgtgtgtgtttgcacacacaagcacactttcttttcttttttttcttatttttcttagacactcttctaaaagaaaatccttagagaagcttctaggaagggcccttaattgaccttgtgggggaccacattgattttctccacgtgcatcttcatttctgataaattataaagccattaatttgctgaggaaatggcagggccaggctgcggcacagatgtgaccagagccatcccagctctgagtctgctgaggagtgccaagaatctgggggagaatcaggaagcctggattgttatggttagcctcacattctcttgggaactgttttagttgctgctgtttacagatctaaaaggtaatgatgtttccagataaataggccttcttattttgggtaagtggccatttattgatctgctaacccacatgtattgatttgttagccccaactactgcgtcactctcaaaggagttaactataaatccaagacaggcaaattgtatttggttttggaccattgctttcacaaaagcaacagccccctccctgtcctctccatgccaaaactactcttcccaagttttagctattatttaaaaggaaaaacaattaaaaggatataataagataaaaagcaagtgagtcaagatgctccattagattaacactaaaaggtaaaatgtgaaacttgcatagcagtgttcaaaataatgcattttatattttcatgtacattagtagaataatttgctttaaactgcagagtgtggagagaagaacaaacagaactgtaattgcaaggaagaaaaaaaaacctcttatgacaagagttgtgtagtacatgttgggtgcatttgtctccttagcaacaagtgaatgtatagatagcctaccgacctaaagcaaggaaaatattttgccatcctcaccctaaagtagccaagattctgcaactcaattgtgcatcctcaccattgcatgtggcaacctctgacaggcgacggtcactgagcaaatggcagcaagttagcaatggatgccatagccagtgtcatataccttccagcactcccaccgcagcttgatggacccccagactctatggaggtggggactggagggagggaggtgggagtccttgtgcttacagaattgcttttccttaaccaattgcatcctacatgcaggaaggattgtcacccaatcacttgaaaaaagcaaagctcatgtttttttatacccgttatcccagctccaatatgctgaagacctacttctccgacgtaaaggtagggactttttttattcttaattttttcattttctatgcatgtggcagtaatttgaactcccggaagttaatggagatgaatgtggaattggtttattcctacacctgtgttataattgatttaatgcacttgtctttttgtctaaaggtgtgttaagcaaagatgccacttgtgtattaagattggaagactggtgttaataagttgcatgggtttccaatgtagtctgaaaaacttagcctctgtctttatatgtttgagtagcttctttgaagaaatttcagctggtaatggatgggtgtgctttagagaatgttttttccctcccctcagcaacagtaaactgtttctgtttttgtttctgttggtttccccatatttgtgcttatgaaagcaaactctagcacctctttttccccctgtcgaaaaggagcgtacattgaaattctctatgcagtagctgcttaaaaacaaaagtgatgattgtctcttatttacaacttaatttgttgttgatgtagagtacactgagcataaggagaatgaataaagtgacagattcaggacacattattcaaatgaggatatgaaagctgtcggcctacagctgcagcctccctcattctacagaatattgggacctcctggttctctctgtgtgtgtatgcgtgtgtgtgtgtgtgtgtgtatgtgtctgtgtctgtgtgtgggttttaagtaattgtttgcatcaacttgatgttgtgttaatcatctgtaactttttaaaacatagattgggttttgatgatgataatgacacacatggtatcattatcccaggaacttgataaacactacattagctgagattagtttattaggggtgggtgttttttccccactcctcccctgcccacccccatatgtacaagttcttctttctgccatggagaactcacaagctgccaaaacacactcgctcttccactgctccccgcacgcagcttgttttgtgcttgatgcccaagtggcttcattggccccattttgcaggccaactcatttcagtttccttcactggtgttttatttggccttataagaaaagttctgttttccctcctgtttgcttttgaattgtgtatcaacttcagccttttatctttctccttccctggctgtgctccttaagtggaaggcttgttttctccttgttcagcaccagcaaactgggcaagatggggaggcagggaaagtccatcacgtaaatgtctggataagactaagtgagcacaaacaaggctgagtgacacagaggccaggaaaagggtttgggctttgtagaggacaatctagaatacacaaattgaaggcaatttgtcacctggttgaggactgaccagcttctagagtctagtagaacctggtaaagtttgtcttccagggaatcctcccaacattttagttctaggaggggacatggaggacagggagaaaagggttattgtgtgcacatatgtgtgtgtgtgtgtctgtgtgtgcagatgtccatgttactcattccttttagggcaatgatcttcagtgttgtgaaataataatgacaataacttatattctttgcatagcaattttcacccagaagtaggccaaagagctttaccaactgcacacataggtgtcactcacccaccacggaaacacagccacctggagggtgggaaacagcagccattctgagccaacactacccaacagtagacgtcaatattagaaacaatcattttttgtgagagttcaagcatgcgtgcatgtgtgtggtgtgtggtggcaagtggggaagattattgatctgtagctttataaataccatgcaatacaaaccaacaagaaactgttcccattcctctagaatgcccctagcaattcagctttgcaaataaccactgactctgtgtagataacaatggaatacctgggtgaatattttattttcaaaagcactaatattcagattgttgattctatccataccttacccatactggaagagaaggctgttaaagtatatgtgagtctggttactaccaattatccactgtaatggaggggaaacagtagaacatatcaggcaaagcagaaaatcactgaaggtcacttctcttttatttttggaaggaattatacatttttaactttcctaattatgttttttctttggttagtaataaatgaatttgtatttcttgagcttacactgatgagagtagaaagccatgcaaagaaagggaaaggtagtccaggcaatgtggtccagagactttccagaaaacaatggcagagcattctgggatttcttcaatattaaggataatcacagatgtgaatattgacaatgtatacacacacatatgtgcatgtgcatgggttcacaatacacatatacatatatacacatatctatagcttgacattgacatacagatagacaagtgtgtctatttatttgcaaggctgaaagaaatagatatttctttatatatgaatatacaatccaaacttttattttggccaggattcaagaaatcactagagaaattggggaagagaacttagggtcttctcagaaatgaaacctgcatcatttatctggaacaagatatatgcatgtatctatggaccatgtaatgcttgttataatgacatgaggctctacttggtcatggccacattcatctaggagaaaattcctaactttagtaaaatgtactctttcaaataataaagttattttattcaattttttttttttgagacggaatttcactcttgtcacccaggctggagtgcaatggtgcaatctcagctcactgcaacctccacctcctgggttcaagagattctcctgcctcagcctcccaagaagctgggattacaggaatgtgccaccacgcctggctaatttttgtattttttttagtagagacggggtttcaccatgttggcgaagcttgtcttgaactcctgacctcaaatgatctgcctgccttggcgtcccaaagtgctgggattacaggcatgagccaccgcgctcagccctcatattttatttagtgatcataagttcattttgcaagcaaaaacaaaaaacaaacaacaacaacaacaacaaaaaaaaccaggagaaaaaaatgtgagcagaaaatatcttgtttcctgaatatggtataacgtaatggtccatcaaagccacacttggaggatagagctagatggggtaaatcctctgacttgctctagaaggtgagtcatgccaaagtggtgcccactcctttgtatttctccttaggaatggacacagtgcttaactctccacaaatgacttccacctgggtaagaggtaaatgcttttcaattaccttggaacgaaagaggtagagggaaatcatacaattcagagatgttggcatggcgagagttcttcttctacaggggtgatgtatatgaaggatgaaaccagggccgacctagtttaactcctagagcaagaatctaaacaaagttctatgttctcacagagagccaacttaattccctcataatgacatttagccaaacaaaaagctcagctcatcggggctacaaatcctttgagaaggacaagtggacaaatgtgagagagctgccagggatcgatgggccgcaccagctccctgttcactactgggtgctgattttaatgtacaaactaataactcttagaccactaagtacagcagattcagtgtcattttagctttgaagaacagacgctcacagcttttcaagccggcagtgttaaatgatgtatctcattccctccaccccttgagtcaactgctgcctagccagattaaggtgtcagattgatttgttttatacatcttttgaccatgctcattgaatatttaggaagtttcttcagcccatattgaggctgagatgtcccgtgggaagcattaatcaaagtcacagagactcgtacactgtggaaacacagcctctttattgtagcgattagtttttgcagtaacacattaacacactacagagctttcctttatagaacaattgatccttttcttgtaagccactacagaatgagggaaattaactctttaaagtttaatactttttctcccccagtgtgaatatctagaaaagcgggggcttgcttttgcttttagccggcgactaaaactgaacaaattttagttcacttctcctggagggaaaccctgttccttaggctgttgggctggtcatttcgcttgcctcatgtttggggagtctgttgtttttgtccattctttctctctggtatttccattctccaacaataagctttaaatctccctttatgtcccattcgtaaataatggcaagtgcacttacttttttgtcctccccattaggtcattcgtgaccattctagaaaaaaaatacccttctatttttttcctctacagtactcttgtccatatgagacaatgtcttgtaacaatgcagaagcctaatctccatgtcaaagcaattttcattccccagtgcacagcctgctatcattttgtaatgttttgtttcttattctaaaagaattaaaaaggaacagtaagccgtcacgggggcctgtagtccttatctcagtgtctggaaatttggacagtgtattttactgctgagataaaatggaaagaactccaagttcagcaaatcgtaatgggtttaagttctattgaaatcggcaaccagaagatcagataatgggggtccttcagttgtctttttaatcgggttccccgcgaggctgaatagagacagagcagacacacagagtgaaaatataattcttggataggttaagtacatgtttgaactcttgcaagcagaagcgatttgctgatgacttaatcattttctggtcaattatctgtaagggcccttgcaactccatggcaattatgatgcaagttggccttttgggagaaacaccagtctctctgcttctgtttccttgtgacttccattctctgccataaattttcattcatttattatctttgctagtatagaaacaactttctgtgtagtaattagagccccaatacacactttagctgtcatcttgttggagtctggatgttctcatggcctgtgtttgataagtgctctttgttgatttttgatgaatgtacatctttttctgggggcccagggaaggggatgcctgtgatgacaaaaggcagggggttgtctgtcagcccgcctgatatagagctatggatttattggttttgacttggcaagttgagactcatctgtcctttacgtgagcagaggactgtcaataaggatggtatcatttgcagtgcatccagaaagacatcttcatttcaaaggtcatcaggaaaccttggtaaacaaagttttaaggcctaaccatgttatagtaacttggcatttaaaaaaatgtaataaagctcctgtctatgccatctgtgtactgtgtcctaaccatgcctcccaaatggcagagataccaagggagggggacatgggtcttatccaatgctggcttcaggaagcaggtgaacaggcaccaggagctgaccagacctcaccagacatgaatgccgtgggcaaacattaagtggaatcacagttggatggacatgggaatcactcattgccaaaaaaataagcaaatgccaactcctcccattttgtgggaaggccatttgtctgcattgaagggggctgtaatgcggtgatacaaatcctcacttaaaaaaaaaaagtatatcaaactagtggtagagtcatgtggcacatcacctctggtacatgggagtaacaacacttccaggattctatggcttcaatgaatgtccataagaagtatataaatgcaagttgttctactgaaagatgaagaacaatggttaaaaataaagatgttcggcttaaggaaagtctgatttagaatgtgacttttccacttgaaaggtagagggttgtgatatgatttccattactgacaggtttttataatttcttgtaagtatattcttcctcttgcctctcttgccaccattttggtggagttaaatacgtatctttccaagtaaagaagggacgggaacattaaaaatgcttcagacacttaaaaaaataaatgaagaaaatggcaatgttcttatccttttcaacatttaaatttaacagttcaacagatgcattacctctcagctcatcaagtggtttagcaatttccgtgagttttactacattcagatggagaagtacgcacgtcaagccatcaacgatggggtcaccagtactgaagagctgtctataaccagagactgtgagctgtacagggctctgaacatgcactacaataaagcaaatgactttgaggtaggaactaatctttattttttggtcatctcccttttccttttttaaaaaatttattttctttagaaatgtacccaaatctgtttttgtgttggtttcgcatacaagcatcccccaatagagtaacaggtagagctgtgatgaggagcttccatagtccccattggaatcatgaggctctgacccactgccattttttccccattccctggcttttcagcttgtgtggaagactcatttggccacagaaaagggaactgtagaatccaaagaaaaatggcagcaagcagcaaagacagagtgattcattttccaaggaagaggtccctactccaatagacctttttcatatttaggttctgagaggtcaatgagctgatacatgctatgtgcaatggtagctaccaatgttattttcttaaaaagtctagaaacgttgatgggggagtgatcatggtttctgactttgacatttagtccctttgtggaggaaatggtatgataatttactaagtacatagcataagagatccattgacatctttttttgggattttgtttctgtttttgttctttttggaggagagactcgtgtgttttgcctaagtgtaccttcacaagcatgctgctctttgtacaaacactctcatacacacttatatatatctgtgacgtgtatattctagatccacacaaagcagcatagagaattcccagaaagcaatatccatgcaacaatgaaagatgtgtggctatgagtaaggcatttctttatgggctaatgtggtgcctcagcaaacagttttcatcacaacgtgatgactctctgtgagacaacactagcaaatctcccagtactcacaaaggcattttgctgagccctgctggctgaggcaacagtagttggaggtgggaacatggcaagaattctgcaggctgaactccctgatgatgagatcagacaggctgtggcttgacaaagttggtccatttcttgtattatcttggctagatgctgtgccatcttgagggtaggaattttttctccaacgtctgtgtgcacttggaccttatgttaatattcttgctttcttcttgtagataggtatccaggaatacccaggaagttccaaatttcaaaggaaagaggacaccttggcctcgctctgtcaattaaggggtctgacccctagtactcttcctgcttgcccccctcctttttttcggctcttgtccctacagttcttggcaatgcagaccagttatagtggcttataaagaattgaatatggaagctcagcaatggggaagtcatagtttttctttgaaagtttgagtagttatagtgtaagctacctatttgtctttgctctctaagactaatatattttttgccaaatgtgtgataaatgaagtttgggtggtgtgtgtgtgtgtgtgtgtgtgtgtgtgtttgctaaatacattaaaagtgagaattcttcgtgtactgctccactattttaaaatctgtttttaaagtctcagttgtaatagagcactggctcactataatgacagagcactagcaggcttcttctaaagctgaagaatatgattatggctaaccattttaaagaaatctcattaagagcatcttttctcccctgcctttctgctaagcctgttgccctaaaccttaagctaagagacttctgtgtgctagtgaattatttacattacatgatgacataagtatctgtttggcagcatacatcaagcttcatgaaagaattgcccaagattcatgagatgacttctgcatttttgctatataaaatacccaagaggacaagtccttaaagtgcgcacgagggttttcgggttgcttaaaccttacctggttggaatttaatccgctacccacaggccaggggccaaaatgacacaaacaggggatggctggcatcaggaggtacccgacaagctgctccatttagcatcatctaaatcctctttaatatgattaacatctaatatttctctctttgtgaatcatatccacttccagccaggccacctctcctttatctgcagtgtctattttaagactgcttcactgcaaggagtatggggcccgggcaggaattttgtcacttctcatgtgacttcggacagttattggactattctggatctgattcctccttcagtgaaaagaagggaagaaagcaggaccatgcagtgtgtcctgccccctctactcacacacttacacatccatatgcacacacgcgtaccgaccaccacacataatcctaatatcacgaaatcgtttttcttttagcctctcggtctggctcatttactgacaaaagtttcagataaggtgagcccttcttttccgtgcctttgtgcatggaggtcactgcttaagtgagatgcttaaaaagccaccgttcttatcgtggtagctttgctagtgtgggccgtggctgagagccaaaagtagatccggcaccttcagctgaatacctccactgatactgtgtgcacggctttacttttgtatttaagtttctcctcttaaggtcaagtaaaatgaacctatagtttaagtattagcaagtgaagaggatggcaaaatggagaactgtgctacaaacagagctaaaccatggtagagggactttgaagctacgtctacacggtgccccaagatccagtcgattccaaggaatcgtgtcacccagcttagtaggagctggtcaaacaataaaatgtcttattgattgtattcccagacttctcaatcaattgttgggaacaataataaaatagctaacatttattgactgtttactaatgacctaggcactcttctaagtgttttaccaaaatagggcttatttaatgtgggtaataataatgacagtgataccaatataataacaagaaaaacttcagtttgcccaaagctttactattcttcaagttattctaactgggcagaggcagatcgagccagggagagagaaggaggtttgacgtctcttcactactactttattccttctttctctcctctaccccttgtcttctctcagccttctactcccatctctgcctctgtcagaagcttgctagtggcacctttgtcactgcttagcaccacctccgtccagcccctgctgctgatggctctcaaggctggagaggctgctgacccctggcctacaggaaaataaagcagatggggaaagtttatcagcagcgaagagggagtggcttgcctgctctcctctcctagaccctgcatttcctggcctttatgagtacaggaccttctaagtggcagtagagcttgttctgccttttgtatcagtttacacaattgccagaattcttggcacggtgtgcagacttagggtggtgagcgtttgagaagacccaagggatgtggaagaagacacccaaggggaaaaatacgaaatacacttttagtttgtgctaaagggcagaagcttggccatatcacaccgggtggggtgtcttgcttctgtgcgtgagtgtgtgaggcacgcaggagaggggtgtgtaattatgtgctgtatccttcatttctgctcctcacatttaatgagattggcaacaataaatttgtctttccaggtgtgatggtatatatttctatgcttcattctcacttcactttgaagggcttccaaaaaaaattttatgggcagaaagagcaagtttgggattccttcccagtttttaaatcatactgatacttgtgactttaggggcgtatgagttggattttatcgcttttgttgttttcctcacaactgtggcaggaaaagaagatgacgatctctgtcagtttctgaggctggtttacctgttttgcaaagagctccaccgagacaactaacttgtgtaactcacaaaggttaattgcacaacgtaaggagccaaaagacatagcagctatatgtgcagctgcgaaaggcagaatcatccaaaggttggagggtttgttaccgcctgagtgtaggttgagaaaagaatgtgccagattccttcatccagtcacattgagctctctttctcattccagggtaccgggaggtagtgtttcccacgccatggtaagccacacatccctcctgggcccctcagtggctagtcattcacctgtaggcagggtctaagtttccagtaagaatgacagatctcccctatcctcgctaaaggcccaggtttggggatggaaggcttcaaaataaattgaatagggaacttgattcactcattagtggccttatgaatgccattttctaaggtactaatacctcactgggcagatgctccatcttagagactgtgggtttgacatttttctgggtgacacatgacagggaagaagggtacttccgcacacctttgaatgtgttttcttactttcctcttggaaatagaaaataaaaaacaacaccccaccccacccccaacacacacacacactaatacatacacacttgctgaatatgttctctaccccatacctacccttttcttaacctactcccactttcaatagaacccacatttcagaagatttaatatatttggaagacttttattcgcattgtcatctctttaaagaaaaatgaggacaggtggatttaggaagcgcttccctctgctccaaatagatccttaaatatgagtgatcgtttagaaaactggcacatgagtgagagcctttcactgctgttgcagtcttttggcctcaaagctgctgagccgtttaaataatcgcataacacactcttggtgggtggcgaggaggaaaagaaacccttaccatttcttcccttgccagtcccaccgttgacaagccaaattgatcttttaagagatcaaatgaatgttctctaaatatatgtacacacatggctgcctggaaacgtattccttccacagaatgattgcctgaaatttgaaggagagcgcagtaaagacaccaggttggaagtggggttgaagggctagggggtggagtggaggtagaattctatgcgtgcatgaggcttcacttttgtacactgtccttttgggattcaaggtgttcatcagtataatgaagcgggcccattgatttatcatctatttggtaatgtcattgcatttttagctccctgtgtcttttttgtcattgggttacattcaagcacagtaagatcaactttaaaacctccttactcaacagctttattagttatagcattccatgacctttctcaacattcttaaagaaaaagatacagtgtaatgtcgctttactttgcttattgtcctttgttggggtgaacaaagcattttctacagtggctatatcacataattatacagctttcaatagcagtgtcttggcacatatcaaagttcagaggagcctttagaaaaaaaaaaagatgttttgtggcagcctagggagggtctcatctttccttcagaaaatagttcaaggctcttctgtcaagcttccctacttagagctttttctcctcctgcttcataaagtttaaaggggattcagtggagttctatgatctatttcctttgaaagattgttcctcggcacagagaggccctttgacttcaagagttcacagattcatgtctttaggtatcatatgtctgaccttatcagttactccatttaatgtaggagaaaaagtctcaactctttgtgtttgtctgttttgcctctgtgaaatgatttggtgaaaagaccatcctttttaacacaccactgagaggccgtttctgactgtaacctaccctgtggcttttctctctttaaaaaaaaaaaaaatcgtccttgtgttttgtgtatggatgagttcacagtgagaatagaattatacaagggcaggcgcacacacaaaaaaatctttgctttcctccctcacctcccgcacccccccacaaatgatctattggctctctcggcggctgtaccccaacaggcgaagccatttagcaaacacagaggtagcggctgtggtgctgggacagtggtgggttttcccttgcttcgacctacccctaaggccttcataattaattgtccttcagcgatgaggaaagttcagaaacagtgtgtggagtgatgcctattgtctgatattcagttctccttgccttggttctttttcttcatcccacaaagggttatcaatgggagaaagagagcaagttctcttctgagagctgctggtggtggctgtagctttcagtgggatgttatcattgtgttcagcccatcctggattaaatgtctgaagaagttctaacaaccttttgaaagacagcctgtttatttcgcctagatgaaacaaattcatttagcaaaccaaagcttgttcgaagttggccaccccttttcacatggcagataacattatagatcaaatttcttcatttttccccccgcaggatgttatttaacttgaactgtttggttctttgtcagtcacagggcagaaattttaatgactattcactcactgctcttaaatacatcaatattaatttacaataatacagtttttgctaacatcctttttgatgaagcgtagacgtttaatacttgaaagcagataattagtttaaaaatattgtttctccttcaatgactgccttcagccaatcttcaattctatcttgtaagatgatgtgaaacaaacgcattttgtcttcctgcaccccccaatttttggctgagatacaaaataaagatgcagtgtggagagagctatttgagaagggtaggaaaaagagaaccgtctattaatgatcattatactactgttcctgttaaatagggtgaagccaagaaaaacaaatataatcgttcttccgaggagagcagttgaactagtaaatcacagaggtttaaaataactacattgtagtgttcatgacaacttcaaggctgaagggaaccatatttaaaggcaatctctgtgtctcttatagcagtttcttttggaggaagagaccgacaggatggccagaatcaattctgccccctttgctctttgaaaacaatttcacaacagaccttttggtatttaaagagaacctgtatatggaagttgacacaactaatatagtcataccaaaaagggggtcataaaaaattaaagttcttcttatgaatctttcatgagaagcaatgaaaagggacactagtgtagccaagttctttgtgctacaagctcttcttccgggctctgagctattgttctttcagctcctcaaacagactttcactttcaaactgacaaaagtcacttaaaagccagacagctgtactaacacacccaccttactgagcaagagccactggcaggtgacaaggcctgctgagagaccttgttgaaaatgagcaggggtgactttctcgtgccttaacgttgcttttgcactcactttgagatggcccattgactgctctttttgcccccccaccccaaaacaggctccccaaaatatgttgtgcattttctttgcagtgtgcaacattgacatccgtgatcatatttctgccttacacctgtgtggctaggcacgggttctgggaaatttgtgcccttctagcagaagacagggagtttgactcacaaaactcctgctgcctcttttccttttgcccctccattcagttcaaatctcacttaaggttttcagatttctgttgcctcactagggttggatagaaaacacccaccaaagatgggtgcaaacctcaccttcggatttaagatctaggcagagatcgttaggtgggtagtcctgcctgcatcccgaccctcagggcagcagccgtcgtgggccatgggaggcctccctgtgtgcgcattacaggcctcccctcccctgtcaccttgtgtacagtctggtctgtgacactgatggtgattatgtcattattttgctctgggggccctggcacatctgcagagcccaagcacatcttctttgttgcgttggcaaatgtcccacgccgcaaatgcttcattagccctgctgccggcctccttgccagacgcctgtgcccaaatcccggcttctttttgctccgttcttttgtgtagctgatgatcatgtattcatcttcctggttcttccccattttcctcgacttctgaactccagatgtcccagttttcttgcccaaatcactccgaagtctacaatgcgaaatgaagtgactctttacccttgaatccttccccactcctgaccacctttcctactttttttcccccaaatgaatagtgactttgaatagctcgccaccatgaagactaacgttttcaaacttgcaatctgaaaagacaccaagtgattgcttccagtttatgatgagagacagggttagaatgagtttggcattattagatattgcttattatctgtgtgccttcctcctccgtccccactctgcccccctcactatttccttggatcctttatttgcacctgtgcattgccacattttaccaattttctgaaagcactttgaaatgtgagtacagaaaatactcttcatgcctcgctgtgcacgttacagtcttctgaaggttcctttctctaagtgaatcttcatctccactctaccctctcccaaaaccactgccccctccttctgccccagccctcaacaatgacctactattagatacttacagtgattaacacttggctgttttggaaacagctaaaacatttctctctctaaagttttattctatatatctaacagagccacagcttttgtgaaggtgtactggtttctacattagctgcagtaaattttagagcttaatatcttgggctgtgatggatactacataattggtatgtttaattttcccttaaatttgaattaattgatctgtgttagcatattatgagcagcttttccaatagagtttaactagtttttaaattctctaactactgcaacataaaatgatttaaatgtctccatctttgagcaaaccataagattttagttttcaggtgtagttaaaggagttaagtgtatattttatggaaatcatggttagatcactgccatgaattgtaatttgaaattcaagacaaagactctgttaagggttaaagaaaacttcctcagaggaatgagttgccacattgtaccgggttgctgagattttcaaatacctatcaaagaggggcacaagaatatgcatgttgcaaatattaggaccaatgtagccaacaaggtgagaagagaggtggtcagatcaggcgggtgggctccccaacccattgtcagccctgtgcagggagcatattgggagaggctggtacctgtcattgaatcatttttcaaaaggctcgagatatatccaaaatattcctaacctcccagttgcccaccattatggttttatcacccatgagttttacttaaaccttttttaaacttaatctcattgtcagaatataccactccttaagataataattctctaagtgtattacctgctgggaaaatactatcttctttttacggctctaaacgtgattcccctagaactccacagggatagcccttgttataatatcctgggattgtgaagagggttgtgtccatattctccatttcctttctgattttacagactttgatcattactccctcttaatcttcatctctccagattaaggagctctaatcctttttaaaagcctaatctcatacagtaagtgggctgccctggatcattttagctgccctgctgtaatgcgcttccagcctgactgtgtttttctgagggacagttacagttactaactcacacagcagaactccaggtgtgggcagtcatgccacggtttggtgatggtgccttgtgcacacccaatgggacttttttgattaccccaaaagtttatcctcagaagctggaattcttgagttggatctcagtagtgcttattggttaaaatgatcctatgagaccagctgatcagactcttggcaaatactctggcaaatatgattgtgtctataggacatacccagccaaatagaaaataggcagatccaccctgccctccagatgttttcagtgttcttgtagatcaagcactggggtatttgacatcatgaggagatagccttagtcttgaacttgagtctataataatgacagctctgggggaaagctccagtttctgctttatttgatgttattctcaggcaggcaatgaaatgttcacctgcaagtagtcaatattttatataaaacatccccttgaaatcttacaaagaaaatgctttggggagtctttccactgtcagtggtcctggatcaataccgttgtaggacttacagcatggactctccagccaggccctgggatcaaatcccagctctgctgctttctagcagtgaaaccctggcaagtgtcttaccctgcctgtacttcagtttccttatctgtaaaataggggatgtaatagtgactacttcacagagtgttgtgagaattaaatgaatctacacaattgtattagcacaaagtaagtgctgtataagcattcacatttattcatttgcagagccaagtaaatgttaccttgttgctgtgacatctgtggtccaattattgcaccatttcctgctgaccctaaataggaaagtaaacaaacgggcaatgagggagctctcatcagaattggaacatatattcaacgtaaaactggttttcacaagagcaagtgttcctgctctgaatgtggctgaaaaggcgacactagcctggaacagctccaggactctggggtcatccgttccagatgagaaggacacgatgagatgctgggggtggtggaaggagcactggcctggagggtctggctctggccatacctgcctcattgtggtctactgtgctcaccttttggaaagtgataagattaaattcaagagtttcattctagctctgaaattttgtgactctagagtagaggggcagtttcattctagctctgaaattttgtgactctagaatagaggggtattctgcattctctaaataaagtctcttttgagtcttggtcatgttgcaaagctttaagcagtgagtatagaggccctgggaatccagatggcttccatgtgaggccccttctaccctggtgactctgctgcagcttaattatctcagtcaaaatctccagggtgcccattttcgttttctcccaaggccctatttgcagatctgaatctcaacagtgcccttggagacatggcaattcccttactgggattatagagactaatttttcaaattcatacacaatttattgactgaattggcactatcattagacttgctgctcactttatttgttgccttggccagggtggccaaacaatgaggaaatttgtcagtgaagccctcatgccattgggttttctcacacattccatgcaggcctcaacacagactatcagcatttataatatgcattaacttctatataatgtacgtctcctctctttcagagcagaattggctatgtttttttttttattcttttatttttttatttttttgagacacagagtgttgcactgttgcctaagctggagtacagtggcatgatttcagctcaccacaacctccacctctcgggctccagcgattctcctgcctcagcctcccaagtagctgggattacaggtgtgcatcactatgcccagccagaattggcagttttagatgatataactaccttccctactaagcctacttggtagtgtttgcaaaagcaacaccacccttttctttaaatattccccaaatgatagtaatatagatcatgaaagtcttttcccttgagattgttttgtatgtgtgagagtttgtggttgggaggtattgagtcctcatacaagccatttggatatgtattcttcatatttcttatggctattgcacctaagttctgttttcttaaggctacattaacattttaaattagaatatggtgctaaaagtgactttcagtaaaaggtaatgtattccctgagaacaagtaaatacttgggcagggagggatggtttgagtagaggtgaaaacagagaaatgatgggaagctgaccatatgtagaagaagctgaaaggtcatggtttcaaggccactgtgtttcctttcatttagagcatccacttttaaagatttatcattttcagtgacctgaaggcgtacaagataatctgtgtagatacctgaaactgcctttcaacaaggccagtcctaggtattgacagcatcctaggttgtcccaccctaaacattacctcaagtcccattgggtaggagtctagtggacttccaaaagcccccgagttcattctgcaatctgcctgtctttgcaatctatttacctgtcttgaaaaagggattccaaagcccttcacaagctcttaagtagcatttgaaatacagcccatccttagttttgcaaagggtgattgcagagaaagacaaatagaattccctggaaatacagaatagaatttctctgacagaacaaagatcttgcagtcaaaaccaagggatgggattgaggccaataatccccatcctttcctaaagcaactcggatattatttggggtgtcataagctattgccagcagagtgccagcatcccccatgaacttgtgttctctgaagctctgtctgatttcctaccatctgtatcacaagcgctttctttggtgtttactatgagcaatccctttctcatcacaacctgcctgaaccccacttcctaacagcttctccctaggctccttactcacattgctccatcaatagcaatacagggcacacagactagttttaatattagcctaggcaaagcttaattatgaaggtaaagctgtggcagaaaacaatcacgtaatacattctcgaacgaaacaggagtaactgtggattatctgtgccccagcttcccttcatgcaatattggagtgtttgtgctatgttgtttttggataatgtcccatccaagaatggcaccaagcttggccctgcttcttttaccacctcacccagtaattgtagcaaaagttaaacttcaagggctgtcagcttgtcttgaactcagacaccaatggcaccaaatttacggggctgacttaaaggggaatttgttaacactacaaagtgactggtatatgattgcagggcttatttttccacctaagtattgagctgatttgtcagatgtgtcatgaagcagggatacattcctctgtttagcacatttaaatatgtactggcaggaaagctcccaattaaacgttcctaatcagagcagggtaagactgaagtcttcctggtccttgaccaccacgtgtgtggtttattaactctgttcccgtagacataggcagccttaactccatcgggggaatggtctggccttacaggtcgaattcaagtgaatcaatcgaactatcctccaagatagagcagaatgaaagacccaggatcagtgcagaatgaaagaccattaggcctctagaaaagctgttagccctcaagtttggctaaaagcaggggctggcaaagtatggcctatgggcagagctgcccctcaatctgtttttatggcttcaagctaagaatgacttaaatttttaaacagttgtaaaaaataaggagaatatccaacctagaccaaatatggcccacagagcctatgtatttattacctggccctttactggcaaattttgctgaccaccggctgaaggttttttctcttctgtgggacatgaactctctgagattccttctagttctgaagttccaaaattctgtgattcctttttttttttttttttgagatggagtctcactctgtcacccaggctggagtgcagtggcatgatctcagctcactgcaacctccgcctcttgggttcaagcaattctctgcctcagcctcctgaatagctgggattgcgggcgccagccaccacgcccggctaatttttttgtatttctagtagagacggggtttcaccatcttggccaggttggtattgaactcctgacctcatgattcacccgcctcagcctcccaaagtgctgggattacaggcgtgagccaccgcacccggccaattccatgagtctttgatggaatagtcttggtccagctcttacctgaacagcctaccagatgagcaatttctgcacagtgcttccagttgtttttaagatcttaacagtatctgtgtagtatctcagggggagagaatgaggtattaggttttagtttttgatgctttttccttgattttgcttgcatatttgtttgtttgtttaaacttggaatcactttttaagacctatgcagagtttgggagagaaggaaaatttgcttcatcgcgaccaataatgtgacaattatgtttcctaacacgtataataccaagacctccatgtgtgagcaaataaactagccacttaaagcacgttcactgaccaaatttcagccccacgaaataattttgacagtctctcatagacatttgtcattctgctcctagcaagctagtactatcttctactggggctatggaagagatggttttacttaccttgatctctacatgcagaattgccaatggaatacttacataatttaaaatgtatgcacaatttattaaacgtagaatagaagatgttaagacatccttttctattacctgaaagtcacaattattcgaaatgctcaaatctagaacattgttgataattatataatattttaacaacacatatgttatcaacatcataatgctgtagaaattttattgtgaattttgtattttctaaatactcttaaaagacaaagactcaaattcaggtagaaaaacaaagaagatactcagggtgtatctctgcccttcattcattgctgtggtcagagaagtctgtgtgaggggtttggccggtagcagccccccagatccgtacactgcagaccaaaattcagctcctgtgatgcttttccatggagtttccctgtcaattcaaggtagatcctcaacctccctccttggcagtttgcatgtgactgttcattctttttattacatttcctccagggggccattttcaccatgtcatatctgtttgctatcagcatttataagggctggtgtggcattggaggatgtcaagtggtctgacttggaagtgtactgccacaaactccatgtaggtgacaggaggagagacctgctttcccgttgccactttttggattatccctgcaactctttccgtctggctgacaaaaaccttggggctattgggtggctcatcacttctgctccttctctagcctttccctgggtttgcttcccccaacccccacaccccctcgcacattaacatgacattgcctggtgagcacagaagagagcagcttccaccagctgaaacctctgatctcaaactcactagagagtttggcttcgggattttggcaagaaggccgattgcccatcaggtcagcatgaataaagatttctttcttcccttcttttttaaagtcaagcatcaaccgaaactgctcccaaagctctgtctctcaagacaatttaacccctttcacctaagtacattttctattttgaatgcatggtactttgttttattcttttcctgtgagatgaccaagaaatctactatatgtaaaatttgaaagccaagtcaattctaaaccaggcttatcatttttaaagtatgtttatccagctttgtagtaggaacaagcagactgtttgaaggccacatacttttcaaaccctggttgcaacacgtctgccccgttttgaaactgtctttatctagccgagaaaacgaaaatctatttgacaaagtggcactctggccagtttatcttgcaatatggctttagctcactgagtctattgatttccttaaattaatgtttacagaatgctactgaattttgctcaacagaacattgttctttcgaagctttatatatatatatatataaaagagatacagactgttattgccatgtgttcctttgtttagaccaaggaaacatagtttttaggtttttttttttcttaagacagccttgaactatagccacttcctacaagcatttacttttcacatatttaaacagcaaaacatgtaactagaaagtgggcccaaactgcatgggtattagacgaatctaatcctcagtgttcctgaaagctgaatgccacctggagcatcagagggagaaagcctttagtcctaagcccagatgttgctggagaaccttcctctgcctcatttggggtaactcggcaggcacccgaaagcaacttcacagccagtgctcctggatcctgctagtttttccaaacacaagcatcctaataaaattcaaacaccatttagctgtttgggaactctaaatataacatcttgccctttgaccacggtgctcagtgttcaatacacaaaacctaatctctaaagatgattttaaaactgaccttcccagagaagtacacgtatccattcagctacgaacagtgcagaaaacaggattttgactcataattatgaaatggccaaaataaaacttagggaacacaaagcaacttttctcaaccggttgactcagccaacaaactcacccaagcgaacctcctcagagcacctctcaaaacgatgctttgcagacatttattaatcacagtgaatgcttcccaggaattagggctcctctttaaaatctcaaacttgtaaaccaccttatatttggatgatattttatgcttcccaaagtgcattcatgttttcttttccatttgatcctcccctggaatgagagggcactggaatagaatctcaggattcactgtgtatagcatcctgcaccattccttctcttctggagggcctgttagtccccggctgtacacacaggataaatgcatgcatgactgcaaagggagacccttagtaaccacatcttgtgaccatattttacagctccatgattcctcttttcagcctctggcaggagagtttagtgtgagtgagacagtgaagaggagcagcaataacgtatctgttcttggcttttcatctgataatctctatgaggagttactaaagcatctgagtttatccatttaagtccactctgtctgcagtgtaagtccccagcttgtgccactgctgtcaggagatgagtctctccttgatcgatatttacttaacaaacagcagggatgggagagtttgtttagaggaatcatgtgcactctagggtgaatgaatgctcgggaaagtacttcaactatttgtctccttccctaagatttttgtgtacgtgtgtgtgcacacacgtgtgcagatgcccattctctttttaacttctccaaagacacttcgaagtcatctagaaaaatacctcgctatgtatgattggtacatcattataccgttaaggagctaatgatgcagatgcagtttttctaacccagcaaagtttggttcttcttttgtgctcttatatagagcacaaaagagactcttaggataaactaaatgcacaagcatctacctttgacccctttcagatgagtggaagggaagaaaatacggatggaaacaataaaagcagtttgacaaggcagctcttcactatgtatttttgatggcattacctatatatttttaaaggcccacagggacaaaaagtaactttctccaatttttcagagctgcttcagcattagatatatttaactctactactgtatatgaattccacggtgtgaaaattgagagagcactgttctttcgagttccctgaaacaattgcttgaaggctcaagtcagcctcttgaatgcagttgacttggaggcatctggggctagatcgaggggttttgtttctgggtgtggggagaggctggggggtggctggggagttatttatttatttgattttgtgaatcggagttgtaaaagccatctgaaatattcatgcagaatagtctgagaagcccgtttctgttttatttaccgcacagtagaacagccacagcggattagttctacaatacccgtaacaaaagcccaacagctgatgcatgtgatgttaggaggtgacaaaacagttaaagtatgctgctggctacaggcaagcagtcagcagatgcagacaaaagggtttgtgacaagaataactctctctccaaggcgagcagtgaagagtatccaaaataccagtacccttttctccttgacattgtcttcttacagtcagcattttattgcccttttatagtataaaaaaaaatggaggaggaagaagaaggaaaacccacacacaaactaattcaccaaaatactaggcaggattgtactttcccattcgctagccatgcctgccagtacacgtgtccttttccatttctccatcgaagcaagtttgaaaaaaaaaattagcttaaaagatcagctataaagatgatttcccttgaaaagtttgtaatctattgataggcttgataggccattggagcctttggttacgggttggggggtgggtggccagggaaagaagtcgatgcctggtttgttttctgtccatttcagtgaagatcatttcagtgatgaaatgaggccagagggccaatttttaaaggggattgaggagggaggagtgtccatggagaactgagcaaggggcaaggtttaggtcccccgcaagaggctgatgaatgagcttacggacggttcagaggtgtgaaaaatgagcttctctgtctccagaaaataggagaggctgtcttctttttaacctttgtaattccccttctattctctgtgacattcattcagctgccaagagcgtttggcaaggtttgggccagcgagcacacttccagtgaccgctaaccttggtatgtcctgacacttatgatgagtatctgcaggacacagaaggcaggcagcctgctatgtcaggcttttattatgtactgcagaggctagggacagtcagtttaataaaacaaatcatccttgaaggtaaagcaactgggaagaggaggaagacaggagaaaaatgtgtctttgccactcattccgatggaaaaaaaaaagaacagcaaaacaaccacccacccaacacaccgtgtgtgtgtgtgtgtgtgtgtgtgtgtgtgtgcgcgcgcgcgcattcgcgcacgcacacacacgcgcaacccagctgtggactgggcagacttgaaaacctcctctcattttctgcatttcatggaagcccagaaggctcttgtttgctctgaggagactcaagtctgtgatgaaattggtagaagctgatagccaacccccttcaaatttatgcatatcttcaagtacctcattactttatattcttctccaaatatcaaggcaagaccatctggggtgacgttcctatattgggatgcctttttatcaaaacaaagtttccactctcctctcctgaggaacgctgggcaaagcagctcccacaatagcctcagagttccagccaaagactttggaagccttttgttttttccctgtggcatgtccaaaggcagggccttctcccctcctccgcccgccctccccagccgcctgcattgtcttgcattccagtgacttgattgactgttaccacctgatgctgaggagatactctagggttcattctgcagattgttgggttctattaaaagaaacctagataagggattacttgtcactaagggattttctgcagatgtttattggtgatgggaaagccattaggtgtgaagaggtgcagaaaaatatggacaacatcattctgataagactggtttctaagatgctcccacaaaacatcagaaagtaccccctattattctgttaaatggagctgggtgttttcaagcagaggtaaaggtctctttttccatgggtgatgtttctatgtgtggatgaaattcactggaaccctctcagaagatcagttgctacccaaaagtgtacctctgggagccaccaaacacatgagttgctccagtagttcagtatctcattacaactttcttttgtccagtccagtccattgcatgagtatcacctcaaagtaagcactatattaactaatcattttatttgttcacaaagaattcatttcttcccaaatataaaccaataaccaaagtctcctccagggcatcttttataccatttccatttattttgaagttactagattctctgtggtttttcaagattacagaggcacagcttttcaaggttttggtgcctcatataaatagtagaaattgctgaaaaagcattaaaagggagccagcatcgtttaatgcaaagacaccttacctcacagtaatctcttcatctcatcatttcttcatctcatacaatctcatgctttcttcatctataaagtgatgatttctgagatctattcgaactctttgaattctaccttactttaccattattttaaacttctttttttttttttatttttgagatggggtctcactgtcacccaggctgtagtgcaatggtgcaacctcagctcactgcaacctccgccacctgggctcaagccatcctctcacctccacctcccagtagctgggaccacaggcatgtgccaccacacccagctaatttttttgcatttttggtagagacggggtttcatcatgttgctcaggctgaagcttccctttattaagtattgttaaagtattaagtaactgccactctagagcaatatggagtaaagcagaaggcaagatctcactatgagctatttaccaaataactttgcaaaagatactctgctgaggctccttatctagagacaccttatgatgaggtaattgaaagtacataaaagtagataaaaagttaaacagcatcaagacacaaatgcaaaaggtgataaaggataacctatgattgccaccacaagaaaggaatatttaaaacagattaaaacccactaaaaaccattaacaagcatgacgaactataaaaatgatgaagaggagactgcatacaacccccaaagaagttgccttgttctcatgcaaatcctacaactacacttccctccctcccctgctgctgatgttctagatgtacctcttctctctcctctgacagtcttgaacaatgcctgcccttcccctgtccctggttccccagacctcctgtgcagttcttggtgtgggcagggcttccggccttctctggcttctctggggcagctgcccacaccttcacccctcaaagctctctgccatgtcatgctgcatccctgagtgctcaaggaacatagaatttcactgaggctgtattgccgttggctgatgaaaccacccttcttgaaacgtttattttaataaatgcctataattggccaggtgcagtggctcacacctgtaatctcagcactttgggaggccaagacgggcagatcacctgaggttgggagttggagaccagcctggccaacatggtgaaaccccatctctactgaaaatacaaaagttagccaggcgtgatggcacttgcctgtaatctcagctactcaggaggctaaggcaggagagtctcatgaacccaggaggcagaggttgcagtgagccaggatcatgccactgcactccagcctgggtgacagagcaaaactccatctcaaataaataaattaataaatgcctatgattatgtttctgtagcatttggctaacagctcccaatccaaggagtgagagtgggcagttgctccgcttcactgttctccagccacattccctccctcagtgatgctcatttgatagaatgtggaggattatctttgggggtggaggtgactgtgctagaaaagattgcttcacgaatttttatttgtataatgtgagtgggagggctaagctctcctccaacaaatactcatgtatacaagacatttgggaggaaatcacccaaaggcctgtagaaaatccacatgaattctcagcagagaatggcccttgaggtgtatgggtttgcacattcatggcggacaaggcggcactttgaaggattttccaggcaacactgggaattatgtcctaagaaatgggccagtgtgaaagtctttaggagggtctgataaaaatgtaagcttaagactgattggccccaaaaggagtccctttcatttttttctgcagagttattacatttctttataaacaacaattaacttgccatagggaacaatgaacttctttgtccaattttaaacgtgaaaaacagtgatgtcgggtgatgattctggttttctttaccagttactactattgttaaaaagtacattgcacccaaggtgggaagaaagagatgaaacatgttcaacattacactacttcctttttactttggtacgtggcatgtctgaacttagatgaaatgtctttcatctcttgtatatgcgtagataaatatggctacatgtacacctatgatacgtttatgtcctcatacgtctgcacttaatgtaaaaatgaaactttactggtgtataagtaccccactaaaagaaatctactaagtgtcaatgtgtacttggaaaatcatgagttcatggattattctgtgattccattatgttggtgtggggatagatagaccatgctgtactataagtaacttccaaagaacactaaataagtacatcagtagctactgctttccttagtcaagagatcagattaataagtaattaagagaacacacacacacacacaacacacatacatattaattgctgtggaagaaaagccttaagaaattggggttctaaaatgaatatttggggaatgtttattttggatgataaggaccttgaggaatttccttaccctctctgagcctcagttttctattgtgtaactgggataataacaccccttagagagattgggagaactgaatgacataattcacattcagtacataaaacatagcctggcaagtagtaaatactcgaaaaaagttagtttgtattattattattatcagctgaataaatcactctcttatggagcaattctaatctcaaggttaagtagtttctgatgtaatattttaggatcagttttgtgacttcatgttaatattattattttactcctttatgtatatagaatactttatattgcagattaatatacaacttagcatctgagtcaacaatcctctgagacaaacagataactgagattttagaagattttcttcatttaaagcttgggtttaatttataaagaagcccaactatttgttattctattttgagaacgtattttgttttcatcatggcaatcaaaaagaaataggattcaaattctgaaaaaataattggagactttcttctggatagcacttatttaataaagtgaggaatcccaaaagtcacatcccatattcctatcctaatatccacaatgaaatcccagtttttcaataggtctgcgttggatctttcatacactcttcttaaaacaaagctgtcaaccccacatcacaatgcttctatatataatgactttacattaaaagaatagaagccagctatttttagaaaatgcaggtgccatgtaagcccctttctgcaagaatgatcttagctcagtttccttggaataactgtagacttgaaactgaaaactttattaatgccattgtctccttgtatcagcaggttccagagagattcctggaagttgctcagatcacattacgggagtttttcaatgccattatcgcaggcaaagatgttgatccttcctggaagaaggccatatacaaggtcatctgcaagctggatagtgaagtccctgagattttcaaatccccgaactgcctacaagagctgcttcatgagtagaaatttcaacaactctttttgaatgtatgaagagtagcagtcccctttggatgtccaagttatatgtgtctagattttgatttcatatatatgtgtatgggaggcatggatatgttatgaaatcagctggtaattcctcctcatcacgtttctctcattttcttttgttttccattgcaaggggatggttgttttctttctgcctttagtttgcttttgcccaaggcccttaacatttggacacttaaaatagggttaattttcagggaaaaagaatgttggcgtgtgtaaagtctctattagcaatgaagggaatttgttaacgatgcatccacttgattgatgacttattgcaaatggcggttggctgaggaaaacccatgacacagcacaactctacagacagtgatgtgtctcttgtttctactgctaagaaggtctgaaaatttaatgaaaccacttcatacatttaagtattttgtttggtttgaactcaatcagtagcttttccttacatgtttaaaaataattccaatgacagatgagcagctcacttttccaaagtaccccaaaaggccaaattaaaaaa

SEQ ID NO: 21 - DNA, 47500 bp, Homo sapiens
agtggctggggagtcccgtcgacgctctgttccgagagcgtgccccggaccgccagctcagaacaggggcagccgtgtagccgaacggaagctgggagcagccgggactggtggcccgcgcccgagctccgcaggcgggaagcaccctggatttaggaagtcccgggagcagcgcggcggcacctccctcacccaaggggccgcggcgacggtcacggggcgcggcgccaccgtgagcgacccaggccaggattctaaatagacggcccaggctcctcctccgcccgggccgcctcacctgcgggcattgccgcgccgcctccgccggtgtagacggcacctgcgccgccttgctcgcgggtctccgcccctcgcccaccctcactgcgccaggcccaggcagctcacctgtactggcgcgggctgcggaagcctgcgtgagccgaggcgttgaggcgcggcgcccacgccactgtcccgagaggacgcaggtggagcgggcgcggcttcgcggaacccggcgccggccgccgcagtggtcccagcctacaccgggttccggggacccggccgccagtgcccggggagtagccgccgccgtcggctgggcaccatgaacagcagcagcgccaacatcacctacgccagtcgcaagcggcggaagccggtgcagaaaacgtgagtgtcccgagcgcgtcctcatcgcgggggctgggcgctcaggcacgcgggtgcgggaggcagccccaccccgcccccaaatccctgcgatcctgggattaggtccattcccggcactgcccgtggaatcgaggtttggaggccggctgggagacagaggacttggattctcccgttttcaatgattgcattttaaaaattgtcctcccacccatcctcttcccccgcaccagtgcatacacaccctctcacatttgcaactttgttctttctgatttggtcctgatttgttgacagctctgatctgccttgggctaatggattagttagtataaacctgtcttgaaatctccttccacttttcatatccgcatgggttaggttagaggacggtcttaattctcattcttcgtagagtttggtaaggcgagatgttttaaagagctaaacacatgctgacttttacgaaggggctgagtgcctctaaaggtactttttggtccccaccaactggctttgtgttgcgctcatatttacttatttggcagcaattttgtagcgctcatcgtgaccatatcggtaaagtggatagaagtggtagcaggtgggagtggatgcaacggacaggatacggtggaggatttgtattcttcagacatctttttcggaagcttgcttatcacttttggggtggctttagggtctgtaaaataaaagatttagaactgcatttgagagaggttttcattcttaacatctacctctaattcttttccccttcaaagttttcttctagttgagtgagtacaaacttgcttttgtcgttgtcggttgattttggtggaatatttgtgtgagatggccaattttttgtgtgtgtcagaaaaaactatccgtgctcaaaacatttcctgttgtgaggtggttgtattcatgtgtttattccaatttttttatttctagatctctgagttactttacctacatgtgtttcaagttttcgtaaacaattttaagaactcttaaacgtcttctcagaccaaatcagaagtgtgcattttgcttagactccaactatcactaatgggaaccagaaaaataacactttaagatttacatttaaaaccagttattggtttaaagtttcattctaaaattttagtttcctttcagaatttcagttatcatagttcttcagaagtatacttatgaattggcattaacagaggataataatctataaatagactgatgaaaatttggaagttttatggtacagtgtgtcttaagggaatattgtcaaaccagcagttttctaccttggcagtttgagacatcggttattaatacttttaattttgtagaatggtttcttatattaaagtatgtttgtcttatttgacattgtcattgatggttaaagttgggaggcatcccaaatgtctgttctatgtgtttattaatataataatacacttagcatcatatataataggggtgtcatgggttttatgtgtatggtgagaaaacatgaagatctccctgatgtgtttttaagatactgggtttcagttagggtgttatcaaaggacctggatggctttgtaaacaaaaacaaatgccataattttgtattcattacacagtttaacatttatggattttggttattttgcgtaaaatgtactttttattagattatcctaatattatacttacttagtttttaatatacttgtattaatttcttataatataacaaaagtattttttccattcacccacattttgttagcatagatgtttatcagatctctgctgagcaccgtatattgcttctaatcatacagatgccttaaatattaccagcttgctctgtaagaataggctagtgctggcattttcattatcaagtgaaagtgtttatataaattatttttacctgaatgaaaaattgttaactttataatgcagttaatcaaagtttattttagaaatgaagtcattaaacctagtattaaatttgagttttaaaattaaactaccagtttgtggcctaatgagccccaaaacatgccaacattggggaaaatagatcttgttttgagctgaaaatgctatccagacctacttaactttgacctcttaaatgagagccattttattttatttacagataaaatatgctgtagcattattataggactgtttaaaaagacagccaattaaatataaattttatggaatataagtattccataaaattttggagtaaagtaggtgacaattgtaacctgaagctctttcaatcttgagttgaatttattaagaaaaatcaaccccccctagtggcggctctcacattgaaaattatctttattagtaggaaatacattttaaatgtgttcaatttaatcccactgaagtatctccaacatagtagactctcaataaatatttcctttctttttggagtaatgaaggttgggaaattaaaggctctgaagcaactttacgtccaattcatgttcctaccttcaagaagaaaatatcaaagataactttcattatagaatctactataactttgcttagactgttgccccatacttagtgaccctactcagaaaaagagaacaaatcctgtgtttattttattctctttggaaatgaaagtgtttatttattttgtctcatgaagatagtcaaaaaggccattatataattaaatagaatgtttttccctagtgataattttgtatatatttttgtatttttaaggttttaatcataaagaaaaagttgtaatggatttcagcttacctgtacatagaaagttagcactgaatgttggcaaataataggtgttgagaaaaggggatctacactttaaaatgataccagctgtagtggtgattaagccattatgaagtgtcagaactggaagtcacttctgagccttagctgatctttctttaaaatgcatactgataaaaacttttacttttattaacatattaataatataaaagcattgtcttgctattctttttaaatgtcatgaatttcatatatgtaatactgaattgtattacttacataatactcattaaatgttttctataaatttatggtgccatttttaaaatagcatctaaaataaaagaatgggtcttttcatttacacaattatttctccatcatttttctttccaatagctttagatagcatatacatagtagttatggacacttgcttattagaactatgaaatgaagtaagtgaaagtgtttagttataggtaacattaaatggcaaaaagaggaaacaggtataaattgaaagcactggatctaaactaaaaaatgaggtctagtttcttcactgttacagatctgggcaagtttcttgaccaataaagcacgacagtcagcacctgtactgctactctcacaactaaaacagaacaaaaaaaaaatatatatatatatataaactgtgaagactgctaaagatacaaatgaaacagaaacgtaaattctattcttttagcttacattatcacagaacaatcaattcgcccgtgaaacagtaagaggcagtatacgattatgttctcaattacggctacggctgaaatgtgctacttcactggtgaataaatttacatagtacgtgcttactcatgtatttaattaagacagttacttagaaataaataccatacaaatcttttacaacagtacctttctacttttatgcttcttttaaaattgatattttgctagtggcttaatatgacaaaagatgactgtttattagcacttattctgatacaagcttgcccagttctttacatcattttcttcatttttataatagtactaaagaaaggtagtattatttaagtttatagatagggaactgagattcaaattcagagttgctcagataatgagaaagagtttggatattggatcccaggtctttttgcttcaaagcttgtgttcttactgacttgtgttcgtactgtccaggtgaacagattcagtgatgcatacagtacagccatatcctagggattttcactgtttcttctcagatagtgttatggctgaaataaattatttgtcatttcccatacataatttgtacttgctcatccccatacttgtgtttttacgtcctttcctcttagagtcttttctcctgtcttattgatttgctacccacctcccttcctctttaaaacattgtttttaaaggctgcataatactctgttgtactaatattctataatttttttaactgtttttcttttgggaggaaattttattcccaaccatttaatactttaatatttcagagaatacccttgggacgtgtaaattttaatgcaaatttatttttttaggaaaaaattcctgtaagggaattttgggatagaaaagtatgtgttgttttaaggttgaaccaaatcgtacactccaagagatagtatcaaattaagaaaaatttactaattacattccatcatcaatctggggatgtgactatatctttgcatttttgcttaaaaaatattattgtcaatttcataggcaaaagtgtggttggtaattaaagttatatgttgggatcatggttatgtataggagaagcacttgtatttgcagattttctacttagggtgtcctttacgtattcatttgttttgtcttacatttttcagtcatcttttagtttgttctttttacttctttctattgaaatattgttttaaaatgtattcctattaaataatactggcagattttaaaagtcaattatatatgtggcacatcttaagtatagcatgtgaggccttatcttttgatgtcttactgtggtagttagggattttagtgctgtcttgtctcactttaacctttttcaccttcctgcatcctcccaatatattttatcacaattttggattatatacacattagtgttgtattatttttgactgtatattgtttactgtttagctaagtaatatactgtgattatatttgccttcctgttctatgctattattgatttcttttctaccatcttcctgtttattagcatctgggtttagctgtcacttaggttcatcggagacagagtttgtgtttctggctcctgcttttcttgttttggtgtggttttctggaagagaagggtagttgcagaaagctagatattctgccgtcacagaaacagaagtgtcttgcctgtatttaagacccgatttagatgccacatctccatgaactcttttgcagcacctgtgttactgagcaatatattatgcttgattttgccatgttataaaatatatttcctcctagtaggaaaaagaatgtctttaaagataggactattatttgtctttgaaactaccagagcttcccagtacagtaccatgcacttagtacatatttgagatgtactaatttaacaattaactaccacttaaaatttttttggtaatgttttagtgattctggtaaggcagatacattttatgcgggttcgagggggaataaaaatgtgtgtttgcgtagaagaagggggaggggaaacaatgagttttccaggattatggaactagtagtggtttctaggccatatttcattttcctgatcttccctgctaatgtcttgtgctagaaactatgctgtttatatctcatcatctctcctcaggtatagcaaacaaaagggaaaggaaagaactcatcacaattatagggcagcagcaggagggtggcagaagacttctgactcattaaaagtggcttccagccagaattctcagaagtttttgtagatgtttttgactcatatagggaaataaaatatccagctgtgttcagcctgacaatgaagttggggtctatttgtgctatgtgtcacctccattacattaagcacatagtttatgattgtaaatatttgaaatttatattcagagataaatacggatgcactgaagctgggggaaggaaatgaagaatgaatggggttaagagttgggctttattgggtgtaggtagtatggtagggggattttattgaggaatacttcagatttcctagctgttagatggagagtagcaccatagtgctgagaagcgggtgcccaactccaggggaatgttgaatggaagggcacagggtcaggtacagggactggaaataaagataaacaagttatgggtaagtagaccaacgcagtaagagtagattaaatcaccccgaggaaaaatccgatagagcaaactcagtactgtgagaggcaatctcttgttaaaaaacgtcactttgatctcataggattactgaaaccatatctttgcatgtcattttgaggttttataatgaatattatataaaacagttatatttatgagtaaacttggttaattttttcagctttgcttcaaacactggctacaaatgatgaacctcatagacttttttgatggagatgttaaaaaggctttcaagatagcattcagactggcattgggcatactgttgggcctgttaccaccagcagttaaagcttgcagggcttctaaacagaagcatggcatgcagtcagctggagtcttgatttcataacaattttacagattgggacaagatttttgaggtaaaggtttcaaaaaataataagttactcctttgcctgaccaacatcatacggtctatttcaggagatgtttgaactccagagctcccaaatccaatcttacagggatgtttatttatatttcaaacaggcttttccttaaataatggttagcagtgtcctttcttaaagattcgaagacaatacaaaagtaaaaatattagcgtaatccaagtcaagtgcagttaatgtaagattactctatttttaattgttctgtatatcttcaagatgtatggatagatattaaatattttaacaaaatctgtacatttctcacaaaatcagtgaaaaagtgggtcaaatgcattacctttgttagctttaacaatctgacattttaaaagcttcaaatccagtacatctgaattgttaagttctgagggtacttcttagccctgaccttaattcctctgtgtatttgactttcttcctctgtgtatttgactttcttctatgacctccaaaatattctaccctggtttttttccgtctatcatttcttctctgtgtctatgcctcttccatagccctatacctaaatgttggtattactgaggtctatcctcaagccacttttttccttttctggtacagctctcattaggaaatgtagttgttcttctgagctacaaatcgcagaaccatttgcaacctctcctcactcaatggtttcctcttccttacttcctatagtgagttagtttagttcagtgaacatttgagtctggcttaaatctctcttcaagccagtcctttcttaacctcactgtcactgcctttggtcaggttgtgatcattccttttcttggcacttttggtcagcaaacattaattgagaatccactctattatgctgggcattaaggatacaaaaatgatttaagcttacatagaaataagggtgttaggcaaacaagtataagcaattatgttacttgacaatggaaatttgctctcatgttaaatattttgtaagacacatagtaatgcattagaaatgtagacaattttgcattatgaaaagcaagcctcagaatctagtgtgaggctgaatattgcaaagagccagcacaagacaggcagcagtaccacatggagggatacctttatgttactattagttctccctgtgtcatagcatgttgccttgtttcagcgtttgtgtcactctcttctctccagtgtttcataatcattactggaaagccaccaagagtgcaggactggattgtgtacacactttctatttctgtgaagttgtttgtatggaatattccttaatggaatcattatattagacatttgtataagaagtcaaatggggcagagttgctaatccattgtgaggtaggtcagggaggacttcatgaaagtgatctacttgagcggggacttgaaaccaatgggacataacaaggaggttgtatgaaagcactaaaccacatgttgtaactgtaagtaatttcttagctctggggtagaatagacaaaagcggcttcaatagaagaaatgaagccagaggggtaaatgaagctagaaagatcatgaaggattttgtgtgctaagctaaggaacatggacagttcagagctactgaaggagtttaatcagaggagtgacacagtcagatttatgttttagaaagaagttcctggcatcccggaatcctaacttcagcgaaccctgccctgctttctgtatactctatcaccagagtcagcttggtgaaacacaggctgatcttgtaaatctcagctcaacacttaagaacagatgcctaagtccgatttctatcgttactttgctgtctcctctatcttgaacatttttgcatcagacacccacatagctcacttcattttactcatgtctgtcctgaaacatcacatcagagagaccttcctaggctgtccaaaccagcatcacgtctgtcaccccttagtttcttaccttgctttatttttcttcttagcactcaccgccctaaaagttatatgtttatttgtttatttctccatcctccacctaggatgtaagctccatgacaacaggagcttttgtatattttgtttacctgtaagtgttctgtgctgggcacttgataggtgttcagcatttgttgaaagaatgtggccccaaccatccttcttcttttctctctttcaccctgtgcctctcccttcatgtgggggaattcttacagttcactaatccttagttatgcctcttcatgaaatcctgtccctcactcaagatccatctcagataccaccttttctgtaaaatcttttttcaagcagttgctgcttctgcttagctcccataacactttttgaatagttaggagaaaagtgtttacctgggctgtacaggatgtgatggatataaataaatacttgtcagcaagttattaattaaccagtagagaattacacaatgaggtagacaagcattataggtaaagtttgaaattcagttggttaagtggagaagtgaagtaattaggtgtgacttgaaatttgtttataaatgcaatatgattaggggaaaatagctaggggaaccatcaccaatttctattatatacacagttgtacagacaacctggagaaggtatctgttgatacatacataaacacacacacacacacacacacacacacacatcattttaccgatgtattaccttttttgtagaagtatttacttttcatcttctttctgcctaaagtattacttctccacatacatcactgaaaagaaattatctagaaatgttttgattgggtcatattaatagcactatgcccaggccaatttctactttacaaaagctgatataattccatttgaattgtcaaaaatgtctttgttgtaagaaaacgctagataattactcttctggctaacaggaaggtatagaggtataaggtataaattactttagtttcccagttcagcgtgggtatctgatgatttttaaattcactttaagtgattaatgtctgctgtgaaatcctggctggggtctttgtgtgaagttattcaacttttcttttctgaactcatgttcctctttgacccataagtgtctttattctcaggtgaaaatcatgctaaattgtttagggttccgttgaactttctaattgagacacaagctacgaaagctaataattaagtataattattgtctacatgtaaaagcaaaagttgctggtaatcaccattaacctctaatatttgccaacaagaaatgtaaatagcaaaagtcaaatttattcttcttttaatatttgaggaccacattagatggctggaggaccatctgcaggtgtcagaggggctttgggacattatctctaatgagttgaaagtaaataaatacagaaagctggtgatatgaagataaatactttatttagggttgtccttgtttattgacaacgtcaatgatgctgagatgcttcctgtggaagatagcaaaggaagtatgattttcatataatttatcacaaatttctctttttaattttccaccaaacaatggctaatgcatttgagaggggaaaaaaatcccagtagatctgttaattgggaatcactgtgctacaaatgcttgaagtataagcaaacattttcctatgatgagttcataagctttttaatgaaatattcggaagaatttaacccattccctgtttttcttaagatgtgaaaggaagattttagaaagacttacgtaaactttaagtagactttaaaagtttgttgtgttagagaaatatttgaggagatgttataatgcaatagaaatttttgctttatattttttaaaggattttttatggtattttgtttgtttttcagagtaaagccaatcccagctgaaggaatcaagtcaaatccttccaagcggcatagagaccgacttaatacagagttggaccgtttggctagcctgctgcctttcccacaagatgttattaataagttggacaaactttcagttcttaggctcagcgtcagttacctgagagccaagagcttctttgatggtaagacagaagggtttaatttgtctacaataacgtataaaaaatacttgtactagatatagctgtttctgtgttaataactacaaaaattagccgtatttggaaaatttattgctgtatagtaaaatttcagtggcaaagccagatttataagtcttaaaatcaattttccagttttattcacccaacagatgcagccactgaaatgatgcatggtatcattctttgaaaatcatatatttagaagttacactcaaattgattatgtcaaattccagtttcccttctacttcaatatttaaaagtcttttttaaaaaaaaagaaaagataaaacaccatcttgctggattttctgtgttttcatagacatgtcattttatttatccctccaaacttacgtatatttctaatctttttttttttttttgaggcagacttttgctcttgttgcccaggctcgagtgcaatgacacgatcttggctcactgcaacctctgactcccgggttcaagcgattctcctgcttcagcctcccaagtagctgggattataggcgcccaccaccacgcccagctaattttttgtatttttattagagatggggtttcactatattggccaggctgctctcaaactcctgacctcaggtgatctgcccgtctcagcctcccaaagtgctgggattacaggcatgagccactgcgcctggcctatttctaatattttacattaattgtataccgactgcaagaaatataattaaaacatgtttttcactagaaagaacatttgattgtaagagtcaatagaatttgatttcattctctattgtgtcactgaaaactgtttgtcattaaccaaaccaggtagcatccccgtatctcagttttctcagcagtaaaggttggggttttgagctaggcagctggatgatatttgttacctttaaattgcaatggaagtttttgtttgcaaatgatgtttcagtgtattttctcatgtaaaattaatttttgatttatcttttatctgtctataaggcagaaatagattttttaaggaaatggtctttttaaaatatcatataattttactaaccatagcaagttaatgataaaatctcagtgggctatgtcagtcaatgcttctcatacattaatgtgcttacaaatcacatagggatcttgttaagatgcagattcttgttcagtagctttaggtgtgtccctagattttgcatgtctaacaagctccgagatgatgctgatcctgctggtctaggtcactaggaagtgcctagacaacacgtttatggatatttgactggtaatttgtgtttagaatattgcaacagttaatttccctttagatttatgtttacataatctgatgtgtttcgtctgagcatctttttattgatttgcttgcttgctctcccgtctttcttttggacaaaagctatttagtttaacagttgtggtctgggcaatatacagatttgaggatagtgatttcaggaaagattatatatttcaaagtttcatagtacacaaattttctctgacctatataaaaagtgtttgaattttcagttgcctctataccaaacccttcatcttgggagaagagttacgcagtattcagtgatggtctgtaagtatgtgtgtattagctatagtcatttagctgacatagttggaactagaactcaggtttctagattccttatattgtttttcctgaccaaataatacctacttttttctgggaaccttcgcataggaaagcactgaggattttttcctagacctgtggttcttaaaagtgtagtatccagacaggcagcatcagcatcacctgggaacttgctagaaatgcaaattattgggtccaaccctagacctgtggaatctaaggtggtgggacctttggtgatagggctagatgcatattgaagtttaggagccactgctgtagattgtaacttcaggcctcctcccagcgtcacggaatgctgtcctgatccctagcatttactcacatgctgtgcttagtgcttgaccatgtggtggcagattctgtgatgtgtaagacgtggttgccacccttagagattctggagtctagaagccaaggaggcattattctctgactctgataaagctttattgacttgagtcattttacctttccatttcttgttcctttgtaatggggaatcaaattatatatgttaaaaagaaaaaaaaagtataggtttttcctctgtttagaagtagtacatatgtgtgtatctagacagatacatataaatgtgtgtgaaacacacatatataaacagatatatttctatatttaatgtctacattatgtgtgtatgtgtgttcatgtatgtatacacacagacacatacatgattagataagtgtcctattattcagtgcttcaagtaaaggagatctcacagtctatcttataaagcatgtcatagtttaacacagggttcatcacggaattgttaaaaacttttttctcagctcttcattaatgagtttgtctgttttttattctggttcagtgtatttgacagtaggatcttattacctatttgcatgattccttcatcaactacattatcattatctttaacgttagattcttttcaaactgcattgtgcataagaattacctagaaagctttttaaaatgcaaatttggaggcatgcccctgctgataacaaggaagttctgtgtaaaacccaagaatcagtattttaacaagcatgttacggagtctgaggcagtgatttagcctagccttgattatgccctgaacaagttaatttttgtttctttgccttcagaatgacaaatgcaaacatacaaaatttaaagaagtctatgaatatttttgcttgtgttatagtggtgtacattcagcctaagcagtttcttagattgtttgatacagaaacatacagttagatggaaaatgaaaataataaggttttttttttaatccccaaatttcctccagactaccagtggtctggaaaggcaatgcaacatttcagagcacacagagaatgaaattataagggtgacccttatgaactgaaaaaatggtagatgcaaacgaagagatttgaaacatgctaacaatgattaggatttttgtgtgcagttggacccactcttccactattagggaaacagtatagcgaagcgaagggaagctcaaagtgaataacaggatgcaaacagggggtgtgtaaattcctgcggaaagaacaatataaacgagtaatcatacaatcttgttagaaaggaaactcaagttttactgtaacaatagatatttttccaagaaaatacatctatttattgcaagtttaaataatccaacttggatggatcttcttaaagtttggaaccatgactatactgctgctgctgtctgaaaagcaacactataaagttttaagtactacattaatatttatccatttctaattaattaaaacatattgaaatataactttaaaatacttgaacaaggcttagcaatttttcagttataaagtcgaaaatagtttctttgacatgatttaagcattacatttaaatatattatctctgagatggagggtattttaaaaatttaaataacaatggttacaatcaataatagtttattaatagagactaggatttgggtggttacaatctaatctttgaaactggttgatgtgaagagaaaggattgtttcctcctatatcatgtctctcaaaattgttaaacagtgtacactgggcttgttgatcaatctaatcagcattgtaatgtttcattcctaatttcagcatatctttaatacaatttttaatgagatttccttactattttttgaaatgcccattattatgtaaatattttgttcaaattattctgtttttatacagctgccccaaaattgagaattcgggaatgactacttttgttttctgtttacatacaaacacctaatgagcctgacaaaatggagcttagggatccgagtttctgatttcaaagtcttgcatttgcagctgtcatagggaatagtagtattctagttatttttaaaacaaggtaatgttagggaaaatctaagtcttgaatcattatacatgcttaaaataatcaccattttataaaatgtagaattatttacatgttaaaatattttcattcattaagcctaaaaatgctgtgggaagagaggataatagttatattatgtgatgatctaacatttatttgaccccattgtttatcatggatctttcttgactttattttaaattaaaaattacaagtgctctgttcttggtcttccttttgcagatgctttaacagttcattcccagttcattaggttttccaaagagagctattgtgtttattcatcttcagcttgtgcatttctctgaatttcagttgttttggtatctctctagatcagaacattattcttgaaaaacaagtatacgtacatgtgtatatgcaaagaacaagcttgaacaagcagtaaaaatgaagtaatgcacttctaagtgttgatgttttataaaatctaatagatttgtatgtctgctttttcattcatgagaattctataatttcatctgctttgttgtaattttaaaatttctgtgtgtcaagtaatttaccaaagtttcagtatgatagtatcatagcaaatgttatttcttaattctaggactatccaaagtaggtttgtttgcataacagtttagcacatttgaaatcaccaaataaagtatttgttgaatttgatttacccatggcattatccatcttaaatggtgatttaactgttatcttttttcctaattcacattttaaaacatgaaactctaattacaatattcatgataactttcatagctaaaactatataaacctaggttcttttaaaattttaatttatggaacccctgcagatttaaacatgaataaatacaaagaatagtagcgcattcttattgaatgaatatacaaatcctggctctcatatttaatattgtgacctctttggttcaggaatttaatctctcttttcctcagatatcccaatttacagtataagatttattagattatatgaataaattaatatgatagaactttatataggatacttttatctctgcttttgtttgctttgacaactattttgaagaacacagtgacaaatatctgagattcaaacaatctaaactcattgtatcatatattatgtattttgctgctttcctgtctcttttaaatttactgatttaatggatttctctttgatcattacctagtcttgcatggttctagaatccttgcttatttggccatgaacaggtcattagtcatcattttttttctttttagggagactaccgtgggctaacattaaaatcaccaactttcagcaattttccttttttagaaaattttcaaatacagacatttaaaaatctgtagttaagtcttaagatatttcgtgagggtttgtgatctggttaatggaaatgtacaccttcatgttatagtgttttataaaacatattcatatataatattaattatattaaaatctgaagaaatttttatttggtaatatttgtatattatttttaaatcttgctcttacagccttttgacttttcaatcaatgaattttaatgagtaactcaaattcgtttatttgcttatggctggagatttagattttagagtcattttatcgtcagaataacattttaagccattaaagtgaataaaatattccagggggagaacatgtagaataagaggaaaagtaccaaagataagatgttttaaggatgagcaggggtagtaaaaagtgaaatctgtggagtatgaaagtatgctaaggtgatagagttgacttaagatactttcatttgatgtgttcttaaattggagagccagagagaaaactgtacatgaaagtgaaaactgaaatgaaatgaaagtgtaaactgaaatgaaatgaaagtgtaaactgaaatgcaatgaaagtgtgtacagaaaaataataacactagagaaggtgatttctgttccttgtttgtccatagaggccaaaaagccattcagttgttagcagtgggagattaagagggagacaaatgtctgggctctgtggctcatgcctgtaatcccagcattttgagaagccaaagcaggaggactgcttgaaagcaggagttcgagaccagccaggacaacgtagtgagacctcgtctccatacaaaataaaaaattagctgggcttggtggtgtacttctgtatcccagttattcgtgacactgaggtgggaagatatcttgagcccaggatgtcaaggctgcattgagccatgatcgtgccactgaacttcaggagtgagactgtttcacacacacacaaaagacggagacaagtagtctgatgggattggttgacataaagaaccttaatgaattgtacctactgaattcttcatgtattttctagcctgaaattttctcatacaattgtcatatttacagagcattctgttgtcagctggatttttttttatcgtgtctaatttctgtgttatggagacatttcatctctttatctcagcatggtagagggaggttgtgttttgtgattaaagcactgacagcaggagctgtggatgctagttcagttaacgaatcttgtgttttttgatagactttaacctcactgttccacagggtcttcatcttctcagcatgtttgataaagatgcttgcttgccctgatatattaggtttgtagtaaattctaaaaatgagagatctgtgaaaaatttttataattaaaattgttactgttatggaaatcatagttatttcctgcagaatcctgatatttcacttgttttagtggagaattttgtttttttttttttttttgaatatgcataagggcctttacggacctggtattaatatcaattgagaatccctttgtttaattaatttcatattttttatttagagatggaaatttgttaataaactgtttttccccttttgatataatacataataatttacttacatatactgttttcttttatcgaaagtggcacatattccaaatctggacacttggaaaagtgtgatagcttgctttggtgaggatagaaattttgggcattgtatgctacttgctttattgggatacatgtcaaattttctgttattgcgctatgtcttaagataggtgcttcagcttttgtgtttgtaaaattgttattatttcccgaagaagttcacaataataactgtgctgaacagcatggaatgataattttaagaagtatgagggaattcaatccagaacatacgtgagttatttcctaaatcctgaggttctaagtctctgtttctcaaagaatggcacctcattctcagcatcactggggaacttgttacctatgcaaactgtaaagccctatcctagacctactgaatcagaaattctagaggtgtggctaagcattgtattttaagccttccaggtgattctaatgtctgtgaaagcttaaggatcactgccctcattgagtatttacattatgttacttaaaatgcaatttaaatactactcttgtatatatgaaaagctaagtgcaactcattccccactgaaaaggtcaattaactgctcttgttccattaatgcagagatttgtcatatagttatagaggaattgtataaaactgagtgcgtaccaatgagtaactggaagaatgttaaacatctttggaagtcagtttttatgtaagagtttggatagaacaaacttttaaggactaccctgccaaagtagtaaaggggatggtaaatattatgaaattaattgaatttatagcagcagttagggactaaaaggggatatgctttccctggcaagatcatctttctgtttaaatgcaaactatagtaacaaaatacaaaatatagtttaccactattcggtattcctcatgaatttttttttccttgaccaattcaaagcatgtgatatgaagagatgtggcatttagggagtaggggaaatctgagcttagatgtctaaggaaggaaagtaccgtattaggaaacatggatatggttaaatgcatgtgggaataaaacttgagtcatctgtggtatgaccatttgataatgctgtgatgtatttgcactctaaattccatcatttgttagattaaattcttatttgtattgtacgactccttcatagatctttcaatgaggaaatagtggaaatattttggtggtttactatcttaacattctactgaatgttcagagtctgattctcacactttggaaagtggtacatatcctcactgatggggaagattttaacaaatgcccattacggtacagcagatacttcccagctggtgacctattttcagatactacattttgtttctgtggactgtcataaatttaattccagtggtgtaatatttaaagtgtcgttatgcatataatttatgtaacaatttacagttttgaagtatagttgacccttgaacaatgcattaataacatgaacagccgattaatgtgtcttttgtatatgtattatatactgtattcttaaagtaggccagagaaaagaaaatgttattaagaaaatcataaggaagagaaaatatatttactgttcattaagtggagtggatcatcatacaggtcttcatcctcattgtcttcatgttgagtaggctgaggaagggttggtcttgctggcttgggagtggcagaggcatgtaagtggacttgcatagttcaaacccatgttgttcaagggtcagctgtactttcaaatgtgtcatatagttaccatagtaatcctattagatactaattactgctttccacttaaagatgataaaaatactcagttacctccttaggggcactttaattgtaagtttctcatcatggtttgagggtgtataaagtcagcatagacttcgttacctctgtccttgattgtcagtgattgattccaggcattaccctggtattgtacatgttattttaatcctcacaagaatttgtctcaagaggtagcattgtcctcatttaaagatgaggaaactaaaggtcaaaggtagttagcaaagtgggcggtggctaataatagctcacacactgaggtctggctacctgagtttttgtctaagctctgccacttaaatctctatgactctgaacattttctcaatctccttaagtctcacctttctaatatattgaaataacagtgtcagtctcatagaactattatgaagattaaataggatataatacgtggaaagtgctaggtgtacagtgtgtgcccaatatcacatatgagtactaagtaaggtcactccaatggtaggaagcagctcttgtgacagtgtctcatgtcactttcttactcattgttttctttctaatcagcacaagcaatatatacatgaaaatgagtagtattgatagagcctctagtaatttactaagttagaggtagtaggcatgagagcaagaaatagaagaaaaacttcatgcaggggtagacattacatagtttaggctgaaaggccatgacataaaatgtttcctgaaaaagcacagcagtgtttttttaaatgattttaaaagtccacccttctcagactcatctttgcacaatatttacttcaattatttaccagtgattaaccttaaacacaagagcaacagaaatatttgcatatttgtttttggtagaaacctccagtgatcattccgccaaatgaacttgtcacttttcctacttatgtagtagttacacatattatctatcatccttccaacatttggaattataagcattgttgtttagaaagaatttgttctttttattttttctctacttcaagttcctgtaatactttaattaaaccctattctggtacttagtatttttgacattgttttctagttatttccatagcttatatcccctacttaaagacagggtctgactcaagtgtctaattacattggctaatactcccagtacaagcccaagaacaaattataaattactctaagatgaatggtctctttcattgcttagtcatcttctcagtgatcaagtagttatctcttaatatttatgtttttctactgccagtgactctaggtggaactaatatcaattgtgttgattaaatattaattttaagagattccaaacaataatttagatatataaagtctgacactgttattgtccgtttttaggcttatttcataatgtaagggtttggctttgttgttttctttttaaaagcaagtgttactgatgcttcataccagagcttttagaaaagtaacagaatatagttgactctgacgttgtatggttttagtgcattcaagcaaatgtggccattagtaagcaagcaacaacataacttaccgaatagaaagggagaaaaggccattctctcttctgccattctctctgtgtacagattaaacatttcctttggtcttgatgaactgttaggtcagtttacctcaaactgttttttggcctaaggaaatctgttctctggccttttgaagttacattcagagaccgtgcttttggctgtttcttgcaaagagatatgttttaacttctgcaaaggccagaatggcatgagattctggtgtgacacattcactgatgatttaccagaaaatacactgcaaatatgaaagactgcagacaggttttatatgtggaactctgctcagactgagagaaagtctagttattagtaaatctctagttttttcagagctggattattttgtcacataaaaatatagtgagataaatgttaagaggactgccagagtcagttttatttaatggtttctcttattcaaatatcacaggttaaaagaaggtcagaaagtggcaagtatctaaaagattatgctttataaccaagcaatccataagctctgagaaatatttctactaattacagctactgtttgttgaacagtattgatccactatgtgtcagaaactttacatatattctcattactcttcacaacagccctgcaagatgtgctttaaaatttttttttttaattaccacctcacatattaggagacaggctcaaataactcccacttttagtaaatgtcagaggtaaggtttgagcccaattctgtcagttaccaatatccacatgcttttaagtgagataatatttgaaagtgcttggcaaaatgtttaagtggtagctgttattatttctattcagctgcgcttcctcccagactataaatacatacataccacacacatacacacacacacacacacacacacagtgaggaaaaaaaagaagagatagcattataacatgacacctccaatagtaagcatcctatcatgccatcagactttccttcacatatataatccacaagcagttaaaagctataccggaaaagccactttacaagatgtaatacctaggagtaaatataaagatacctgtgcacatcttttttgtagacaactttaaaacattctagcaggacaaaatatagacttgaacaaatggaaagaaatgtatgttcttggatagaatgattcaagattataaagacattaattctccatgagttaatttataaatttaacatgaacctattaaatagtcacagtaattaacagtttttacccaaatgctggacaagaagattccaaacatcagaaatccatctctcaataaggtgtatagttgctttttttctcctcaccaccaccagttccctaatatttcaataaatacataaaaatgttggattcatttttataggttacattatggagaactttagtctcctaagaaaggaggtaggagtggagaggagtgtttgtaacagtgttgtgcagttctttgaaactcctaagttttgtgacaaaattatatcgtgaactcatcaaaaattatttttaacaatctgtcagctgacaacttgactaaacccttcagttttgttactagcatagaataaggagctacccactggcaacaggattacttacctagtatttattatttaccttgttttagccaaatactaaaaataccagtggatgccagtatttcaaattgtctttgtttggtgttcagaagttttctattatagctctttactcttgcttacttttaaaatcattgtttttcctttttttccatagttgcattaaaatcctcccctactgaaagaaacggaggccaggataactgtagagcagcaaatttcagagaaggcctgaacttacaagaaggagaattcttattacaggtaaattttagtaaatatagtttcttacactaaggacagttgtaaatggaaaatgaattaataagtcttttagtaattccctgtttacttaggatttgctcaatgttttttgccactttcatctgaactgcgatagtgaatgatgcttaatacatgaaaaactggaattatagtcatgcaccatataatgatgtttcagttaacaacagaccacatatgcgacagtggtcccgtaagattataccatattttttactgtaatttttctagtttagatacaaaaatgcttaccatgtgttaaaattgcctacagtattcagtatagtaacatgctgtacaggtctgtagccttggagcaataagttatactgtatagcgtaagtgtatggtagactataccatctaggtttgtgtaagtacactctatgatgtttgcatgatgatgaaaatcacctaaccacatatttctttgaatgtgtccctgttaagtaatgcatgactgtatatgaagtagattagttagaagaccggtcaaccccttctgttgtattaatatttcttcaaacagcaagaactcctagagtattttagaggttctaatttaagatgttgaatttatatatatttttatggaatgtatctctattaaaattgttaaatgcatttcttaaatagtttaaaatagttcttaacccaaatttcattttgccaatatgttataacttacattttattttagcaactgtttgtgacttattaacatactaaagattttctcaacaactccaatccctatttttatgcttcagaatttagtttctaatttgtctatgaaagcattttgcatctttttttactcttttttttagaattgatgccatgattgattctcacttaaaattctcagatacttattttgttgaaaaaagatagaataaagatgtgtgccatgtgtgctgtcaccgctatgtgatcattatcagtttcacccactgaactgactagaaaaacttcaccattgttacaaaaagtaaccctaagtttcggtaactccactagttcattcagtggatggtgatgtgcctaataatataacatttttattacaattgtgtaatcacattcaaagtcctgttagcaaaaattaacttatgttaaacaaagtaaatctattaaagcatgagtcctcccctagccccaactgaggatcttcatgtatatgtaaacccaaacataaagactccttattcaggtcataggaaagcattttaataattgctatttaagatgattaaacacacctcttgggttttctactaagggcctaatgaaatcccgtatttcaggtgaaatagacagatatgtactgattgacgtaacacttggcaagttggtttaaattatgggccatttttatttttttcccaaaacatttaggcaaattgtgtacataccctatgtcagggaaacagaataacaataatatgaatgcaaagctgtaaagctataattatatggaatatatatattttaaattatctgcaaattggtaacccttaagaagttaaaataaacttacctgtttatattgaataccatttgtagattgctcttttaataggaagactcacattctctatgattttcttaggattgtgcttatatgttatctttttatgaagaagatgactaaaaacattatagatatgtggaaatggctgtttgattataacaattgcataatgacatctgactttatattgcaaccgacggttatcagccaattaccactacagtgtgaaaggtgttaccagaggttctgtataccatatggcctaaaacttttgaggagaaatattttcctttgcttaacaaaggcatatgaagaaaatgttacaaaaatttttattaaaggccaggtgcggtggctcacgcctgtgatcccagcactttgggaggccaaggcgggcagatcacgaggtcaggagttcaagaccagcctggccaacatagggaaaccccatctctactaaaaatacaaaaaattagctgggtgtggtggcgggtgcctgtaatcctagatacccaggaggctgaggcccgagaatcgcttgaagccaggaggcagtagttgcagtgagcagagatcacaccactgcactccagcccgggtgacaatgcgagactccatctgaaaaaaaaaaaaaatttattaaaagtttaagacatctttctaggttagcttcaatttattactattttacaaggaataaaaatgaaacaatgtgaataactgtgtctttatttacttcacatgtttatttcataagtttaaaatatatctcttgctatttaatactgtattgttaaaatcttgcagctttgtttttcagtgacaggagttgaaattttatataacagtattgcagtttctctgaaataataagtgagcttaacaaaattttcagataggttgagtgaatctttgtgactaaaagaatgatgatctcttttatttaattagcatattaattccttctgttaatttattgagagaaatcaatacaagtaataaccttgggtctcctggtacttgagatcatttgagatatataaaaattgatgtgcatctatcgtttctagatttttatttctgataataacatttgtctaaagagatttgataactgtacttaaattatccttctgtcttctggcatgtacaccaatgagtttattggaatgtgactaaatttgtttcactgtttgaaaaagtatgatgttctacaaaataatatgaatgtgatcaatggataatcttccctagaagcagatattttcaggatgaatcagtgcaatgacggatatatagtgttactgccaagagaatttaagaatatccaattttgaatgtgttagtcattaactattcaagttcttatgggtagaatcaccattgtttatcctaagagaattgccaggtattacatatcatatttgcagtcaaaaacctgagagcctgcttgtggttaaagggacatggatggagccggaggccattatccttagcaagctaccacaggaacagaaaaaccaaataccacatgttctcccttataagtgagagctaaatgatgagaacacatggacacacagaggggaacaacacacactggggccgtttagaggctgcagggtgggaggagggagagaggatcaggaaaaataactgataagtactaggcttaatacctgggtgatgaaataatctgtacaacaaaccctcatgacacaagtttacctatgtaacaaacctgcacttgtaccactgaacttaaaagtaaaaaaatagaacaaaacagtcagaaccatgcaccaatttgggatcgataacacataaaggcagcatatcatgttatttaagtgagtacgctttggagccagactgaatttatctcctggatcagccattttctggctatgtgaccttgggcaagttacttttaaaggtatagtccttgacctcagtttcttctattgtaaaactggaacatggcacttacctcattgggtttttgggagaagaaaccactcaaatacataaaaacatttagaaaaaggaatactaaataatagttgttatctttgtagtcaagttcttagaagtataatatagatcaagaatcttgaaggtggaaagtaattggagtggaaagagagacagtcactgtatttcatttatttcatctgataatgaagatatcgactggactatatgtgagcagcataagcactagggcaaagacttgttaactcttaaatcccagtacttagcatacatatctcctcttgggtatgtaatagtttattacgtgttgaataaatgaatcactattatttatatagctctttatagttctcaaagcatggtgatataaatctttatttgctcatttctacaaccatataacattgtaccttattacactgagtgctggctctgtgccaagcattgagttaggtgggatatcgtcattatatctacctgatgaagtgccagtttttgaaataacactgaagcctaaaaagcagtaaatagcttaaccaaagccacacagctagtaatagacagagacaggagtccagaccaaatctgtctgactacacagtacaattcttaaactaaaccatactgtgtttctccttacatgaaaaacagggtgttagaagacattagctgtgatcacatagtaaagaatagatcaagtaccaaattgaactcaaggagaaaatagtgagggcttgtagaattaaaatatatgataataataaagtgatctatgatactattgttatccaagaggagaataaaggtattagttgatattggacttgaattatccagtaataggaaaagaaactataacatccaaactaaagagtatgggctaataaaaaataatggacccaaagtaggcaaaacagagagaaaaagaaacattatacatgtgaatgtgctgtgaacaaatagaaataacacatataaaatgataaatttaggtatgtgttcataatgacattaaattttagtggactaaattcactgtatgcttgctttacatgtgtattatagtttaattacatgtatattttatatatatagtgttttcctgtaaaagtcttaaaatgttctgtgacttaaaatgttagcatcagtgcttgaaatgtctttgtacaagaagagttttctattaatgttcccagtttaagaatctaatcaacatctttgcctcctgtttccctctaaaaaaaagcagtgtgatagactatagtgaatgatgtctagtttgttttttaaaaaatatatacaatatatttaaaagagaaaatctgaaacatgaagaagatgaattccatattctaccaaagttattctgattgagcattaaatttagccatttttccatagacaaatcaaaaggtatgaatagatatttcttagtccaggagtgtatgttttggctgtgtttgtgaaatgtgacaattttaactattttgaagagaagaattttcagagataaaagtaataacctttatctgatggtcaatattaagtcatattactaattttagaacttcctttccttgtaggctctgaatggctttgtattagttgtcactacagatgctttggtcttttatgcttcttctactatacaagattatctagggtttcagcaggtaagtatatattatttatatcatttatattattatatcatttactttttatatatatttaggacacagttggccatttgtatgtataaagtatcaatatatgctgtagaatacagtatgtatttgtagctaataattttttctaatattaataatttactgcactataattcctttaaacctttatgtgattcatggttactgtgtttcttctttcgcttatcaagaatcttatatataagaaactttcaggcacgtagggtctaggggagaagatacaaaattatttgctatcaaataacttatggatagcagaggatatatgacaactatgaaatgaatgagatgctgttaaaaagtacaagtaaaatcctatgatttatacctttaattctgatttatttggattatggaagttaccctaaatcaaatggtatgtaagctgacctaaataatgtgtaacaggtgggtggtcaagaaaacataggaatattgaagtactgtaagcattagactggaagtcattgaaggaaacagacgtgtctgacttgtttgatgttattgaatgaacagtgaatagatggaattataaatatttgatatattggaagggcaagaacatcttttatggtttgagaacagattgtaacatttgaatgcttgctattctcatatatctggttctcaaattgtactgtgcacaagaatcacctgaagaccttgataaaacttggtcatccctgtgagccccattgtcagatattctgaatcagtagttcacagtggggcccatgaatttgcatttctattaagctcacaaggctgctggtcttaataacagttatatagactagagcttcaactatattatatacccagtggaaaccactgaagggttttgttacagggggtgtgtatatttcaacttgtgtttcagatgcaacttttggaaattgtgtaatggttggcatcaagatgatttaatactagaagattaggttcctaggaagatttaagtgtagatgactaaagtagtggtatctagctactactgctaccacaaagtctaaactcctgatttcttgcattctaaagaaaatacattaatgtccatagaaaagcctgtttgaagcctaatgtattatataactgtcttaatcaggatttgatattgtgatttatattagtaagaaaatcagagagattagccattttccaatccatttttttctcgcatatacacatggatttttttcctaaaagaatattcatatctgacgcaagtggtagattaagacaccatttttaaaactgcaagttatgactcgctcatgggtatgcagtcgagacagtggatcactatcaaactgttttttagttgtttaggatagaataaaataatatagaatacacagtgttatctcatgtaataatggtaagtgttttgtgaaacttttccatgtgtttatgtctgtgcacacacctgtgacagatggaaatgtattttttatcataacttgcagttaaaaaaaaaggaaaactctggtttaagcactaaggactcagcaccgtagtaaatggtgagaggcaagagaaggaaagtgggtttaatatggtaaaggcacaatcccttgctcttgtggagtctacttggagtgaaaattttatatgaagaaaattttagagaacagtgaaggaaatacataaagaataccattaagcttagagaagtcaaagaggcctggggtggttaggaaaagctgcatggaagagataagaatctaaaaactaacaaatgaatatggaacataaacagattttaatatgttgcatttaaggaatggggatgtgacttatttgagcaagagcattcatgttgggaactgatgacaagtaacttttggcttatctgttgtagtgccagtctctttagctttttctaccttaggttctatcttaggtgactagggaattttaggaatcattcaattcgtattcatcaccactagcaagcacccactaatctaaataggctttaaaattaatttagccatattttttaatcagtccttttgttgtattccttgtatcttttttctttagtctgatgtcatacatcagagtgtatatgaacttatccataccgaagaccgagctgaatttcagcgtcagctacactgggcattaaatccttctcagtgtacagagtctggacaaggaattgaaggtaagaattgatggtacaaaaaatagtgttggtagtttttaaatatgagtctgtgaaaggaggctgggaacctgtagggtcatagaactccatgatagggaagtagtggaaagatgtaaagtctgcaatcataggccacagctaggtcacaaagagcctgatgagctaaggaatattttggcaaatgagagccatttaatgagtttcaggcaacagagtattgccatagtcagtaatgtattttataaaaataagtagtaccttcatgaaagatggaaggaaggtacaaggttgatacctgagtgcatgtaaattattgtgtttaaaatgcaccaaagaaaaaagtaagctagcatatgcaagtataaaaatgcttttctcaatgcaaaaaatgtttttggaaatttgttaaagtgttaagaaaacaaaaatgcattagaaaatatgaaaattgttgacagtgatagctcaataaaaatttaaaaaattatgcaagttctatctgataaaagtgatatttaataaacttttagtttcttgtcattagctcttttgaaaatgatttttttgtattcagaacacagactccagtttagaaactaatacaaattttacctattcaagtgcttaattttacagcaaaatggaaagtaaattttgttttgcctttatttctacagaagccactggtctcccccagacagtagtctgttataacccagaccagattcctccagaaaactctcctttaatggagaggtgcttcatatgtcgtctaaggtgtctgctggataattcatctggttttctggtaaggtacaaaattttatgatactggcttttactattgttacaataaaagcttgaggcaaatttaatttagcaaaatataattcagcagagaactattcccaaatcaggcaaccctcagaaccagagagcttcagagagctcacttaaaaacatgggctggagcatttatgaacagaaaatgcaagtgaggtacagaaatggcttgattgattacagcttgggatttgccttatttgaagatgatctgattagttggccacctgtgactgactgagactcagctatttggttacaaaagtttaattctactgttaggctttccgttagcttatatagtaaatgaagttgcaattccttggtaaggacttaaggagcagaggcatcttcagcccaaatttagtttaacattaagttatagaaaaaacaagccaattttctttggttatctttgatttcagctttaaaaatctgctaccaaataatttgataaaggaaagtaacattttaaagtaattatttctgattttgactatcttctataactcagaaaactgttctttaggatatagtaacaaaaattgagtgaaataacacgaagccttgtattttgactattttaggatttttaaggtatgagagaattatgtagcccttttttaataggaggatgcttaccatcttgggattgaatgcctgctagacagagaaattatggcctttccatatgtctcaaataactgccacatagaaaaacgattgtatattcatgattgtggaatcatcatttgataggagatgctgttttcttgttacaaagtctgaacacctgcataggagttgactgtagggaacagcttaggattcctcagagtggacttgtctgttatctcattgtatccttagctcatgttaccaagtacaggagaagaaaatacaaagtatattgtatattctttttgtgaataacaaggaatttgaaacagataatcaaatcttaaattaacattaccaagcaatgatttttgtttatgtgtttatttctgaagaggattttgctgacccagaattagaattatgctttgagacagttttcccagtgttattgctcatgaacaattagtatgtagagtcatgtcctgcataatgatgctttggtcaataacagaccatgtatatgatggtagtcccatgagatcataatggagccgaaaaatccctatcacttaatgatgtcatagcattgctcacatgttgatggtgatgctgatggaagcaaacctgtgttgccagtcatataaaaagtatagcacatacaattatatatgatacataatacttgaaaataaaacaataataaatcactatgtatttactattaatcattattttactttatttttaaatgtattatacttttaattattttattttaatcattattttagaataaggttaactgtaaaacaggctcaggcaggtccttcaggaaatattccaaaagaaggcattgttatcacaggagatgacagctccatgcatgttattgcccccgcagaccttccagtgggacaagatgtagaggtggaagataggatcattaatgatcctcaccctgtacagacctaagctaatgtgtgggtttgtgtcttagtttttaacaaaaaaagtttaaaaagtaaaaaaattaaaaattaaaaaatagaagaaagcttatagaataaggatataaagaaagaaaatgtttttgtatggctgtacactgtgtttaagtgttattacagaagagtcaaaagttaaaaatgcaaaagtttatgaagtaaaaatgttacagtaaactgtgctgggattacaggcataagccactgcacctggccaaaaggaaaatattttttataaattcagcgtagcctaagtgtatagtgtttataaactctatagtagtgtacggtaatgtctcaggccttcacattcactcaccactcactcactgactaaaccaaagcaactttcagtcctgcctaccttgtttacaataagtgctcttatacaggtataccattttttgtcttttatactgtatttttactgtatattttctatgtttagatacacaaatacttaccatcatgttacaattgctttaagtattcagtacgatagcatgctgtacaggtttgtagcctaggaacaaaaggctatactgtatagcttagatgcgtagtaagctataccatctaggtttgtgaacctaccctctgatgttcacaggataatgaaattgcctaaggacacatttcacagaacatgccccttttgttaaatgacatgtgagtgtatgtctgaggtggaatatctgtgtgcaggagggccttttcctccttgtgctgctattattttgatgtcagaaccatcccttggactggttgatattgttttaagcaattaaatatatcatgttcatgtttaagtgcctttgttttccctgtcttctaaatttcttccatatctttactcagtagggctctgcattctttatatgttttcatgatgattcattttagtgtcttaataaaattatattttgcctctatagaaaagattatgaactatgcagtatgctatgagctgtgatatagttcatgtatgcttgaaaatgaatgtttactttaggactttatcaccattcaggatatattgattgcctggtgtgcaaggagctgtgtgaggtgaaaatgatagggtaatggcaagcttggaaaatgatataggttttcatgttatgaccattaggaacaaggagtggttcacttatccctttagaatatttttcatattactctttgagatagatttaatgggcgtcccatggagagaggagtgaaggaaactaacagttctcagcttcaggaatatttatggaattatcaagaaccctagaggcagccataatggagccttttaaaaaggcactattttatattgttaattttaatgaacttttttgttgttgttgcttttttaaggcaatgaatttccaagggaagttaaagtatcttcatggacagaaaaagaaagggaaagatggatcaatacttccacctcagttggctttgtttgcgatagctactccacttcagccaccatccatacttgaaatccggaccaaaaattttatctttagaaccaaacacaaactagacttcacacctattggttgtgatgccaagtaagtgagactttttcacttttattttattggatgtacattatgtttcagtaagtctctcttagcatgtaaaacatacagtgtatgtaatattgtttattattagattggctattatcgtacattcttccagtgtagccttttacagtaaaataatttaatcaggctcacaaatcatctttttaatttttgcaatacttgtctcaaatgatgatgttcttatatattagtaaggtttattcttctgatgacatttatgttggtttgttttacatttttaaatggcacaaatgttatttgatttctagccccagaagttttagtaactttcagaaaattgtgagcaggagtctgatattaaacatttatatcatatcaaacatatcagtgcttttctcctttctaatgtgggaatgcagagaggtaattagaaaaaccttcttccctaaaaatagattataaaaacattccagatttttattaaagatgttctgttacccatacatctgctctattagattcatttttagcatactaatttccatggatttattcaggaaaagaaaataggctgaaactgaaatttgcaatcataagcaaagaagatcagtttacatggatgtgtttaatttcacatctacttatgtgaaattctaaatgtgaactaaaacatattgcagaaactagcgtaaaaccaatgaatttatcttggttatttcatttatgttaaatcttaatccattcttattttacctttttttattttaaacagaggaagaattgttttaggatatactgaagcagagctgtgcacgagaggctcaggttatcagtttattcatgcagctgatatgctttattgtgccgagtcccatatccgaagtaagttgtagttccttatgaacatgtcagaagaaaacggcatatactgttgtacatgtttcaaattcttacgtaatgtaaagtgtttaagtaaagtatatggataaacttctacttagtaacatatcattatgattaaccagggttacactctttgttcattaacttgtctacacctgccatgtagaaatattgttgtttagttattaaaaaatgaccattttggcccatgaagttttgcttgggaattttttaattacatcgttatcaattcataaatctatgttaatattgtaacaataatagtgttaataattctagtttaaaattataaagtattaggctgtaggaaaatattccctatttaaaatgaaaataattttttccccaacttatgttctctctatactttcctgtgttactagaattgtttggtacataatcttccctttaacatttaaaataataaatttaaaacaactctgaaaaactatttccatgccttttattattttttgtttaaaatttattgtctttggggataaaggaaatacatccagaactatgtcacaagagctttgttttaggaataatctttactatattgatttgggggtttgataatttaattttttaattttattttagtgattaagactggagaaagtggcatgatagttttccggcttcttacaaaaaacaaccgatggacttgggtccagtctaatgcacgcctgctttataaaaatggaagaccagattatatcattgtaactcagagaccactaacgtaagcacaaataatgtttcctgttttaacagttttgttttcataagtcctcttatgtgaaagcataaaaataattcaagcagactttagtctgtaaataaaaattgaaaagtttaattcatctagaaagaagagcacaggtgagagacattgagaagatagaattgacgaacttgattgagagctacatgtgcagagctatagaaagaataataaaactctagggttttatcttgggcatatgaaagacacatgaaaagctagtgtcattggtaaaaaagaagaaaaatataacaatgctttatttagaatatattctctaaacccagcagtgattttaagagtttcttacacatgctgcagatgagtcctttatcaaatatttgtattgcaaatgtttcctcccactctggagaattgatttttgtctgtagcttgaggtaaggattgattccccccagccattatacgtatacccaattgaccagtaacattgaaaagatgatttaccgagaaggtcattgtttcccggctgcgctgtattttgatcaggggattttgtctgtggttctctgtctggattgtgttgttctattaatagctccattagtgcagttgtctattttgtctagcattacactgtcttagttattctatctttattatatatttctatgtcagagtgtgtcattttgtttgttttgccttttttgttctccttcaagattgccttggttcttctttgccctctataattccatatacattttagaaagcagttgtctgaaaatctgttgagattttgagtgcaattaccttgaaactgtagatcatttggggagaattaacatctttttaatactgaatctttgagcccatgaacatgatgaatgtatgtatgtgtataagtacccatggaagtacatgagtatgtgtgtaccatatgtatatgagtgcatgggtatgtgtgtacctctacaaatttgtcacttctcccttgatcaatgtttctagaaggcactttttttaattagattttattagtctttattaggatccaacttttggtttattgatttttttctattttatacatttgttttcaattttaattatgtctgctcattattattttcttccttctactttctttgggtataatttatcattttttgcaacttttaaaaaatatatacttaaacatatatatcagtttccctttaagtacagcattacaaccctcatcttttgctatcttatttttattatttaattgaaaatactggctaatttgtttttatttttattcatgaattatttagaaatacagtgttactgtactttttggtgtttggggatttttctagttgcctttttatgattgacttttagcttaattccattgtagtcagaaaatattctctggatggatttcagtttttttgaaatttgtggtgacttattttatgatctaatacatggtctatgttggtaaaatatcccatgtaatcctatgaagaatacatgttctgtagtttgggagtatcttatttttatatatcagaagtctagtttgttaattgtgttcatatttgtatacatttgttgatattttatgtgcttctgttagttactaagacattttgttttcatttattactgaaggggggcatctttttacatctttctgttttttttttttttttgagatggagtctcgctctgtcgcccaggctggagtgcagtggcgggatctcggctcactgcaagctccgccacccgggttcacgccattctcctgcctcagcctcccaagtagctgggactacaggcgcccgccactacgcccggctaatttttttgtatttttagtagagatggggtttcaccgttttagccaggatggtctcaatctcctgacctcgtgatccgcccgcctcagcctcccaaagtgctgggattacaggcgtgagccaccgcgcccggccctttttacgtttttctatgacatttttcttttataaattacatttgtatcatttacctgtttttctgttgagttgtttgcctctctctcagactctattggagctctttattatggatatgaaattttataagtatattaaattataaatacattatgttacaaatattttctttaaaaattctattgcccgaggcgggcggatcacgaggtcaggagatcgagaccatcctggctaacacggtgaaaccccgtctctactaaaaatacaaaaaattagccgggcgttgtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaatctattgcaaaaatacaaatactttaatattaagtaattttcatattattatgccaatctgtctttatagctgctgggttttattatagaggttagttgtttcagattcttaactttctaattcctaatttttttatttttttattattattatttttgagacaggatcttactctgtcacctatgctggaatgcagtggcacaaatgtggctcactgaagccttaacctccaggctcaggcagtcctcctgcctcagactccagagtagctgaagactgtaggcatgtgccaccatgcccagctagtttttatttttttgtagaggtgagggtcttgccatgttgcccaggtttgtctcaaaatcctgggctcaagcaattcacacacatcagcctcccaaagtgctgcaattacaggcatgcaccaccatgcctggcccctaaattctcgtagtctgtttttatgtgtagatatttattccacacaaatttattatttttttctttttttgcagggtgtcaggtagggatgtaacctgaccatggaattaactaagttattttttaaaaattaaatcatttaaatatgcttgtctgaaaaacaattttatagcaccagtcatgagaataattgaggtgaaaataaaatatgtccctttctgaattcaattacaatgtattttgctttatgtttttcttttttaaattatttttattttaaaatgtttgatagaatttttttctaagacttttttgtacacaattttagagatgaggaaggaacagagcatttacgaaaacgaaatacgaagttgccttttatgtttaccactggagaagctgtgttgtatgaggcaaccaacccttttcctgccataatggatcccttaccactaaggactaaaaatggcactagtggaaaagactctgctaccacatccactctaagcaaggactctctcaatcctagttccctcctggctgccatgatgcaacaagatgagtctatttatctctatcctgcttcaagtacttcaagtactgcaccttttgaaaacaactttttcaacgaatctatgaatgaatgcagaaattggcaagataatactgcaccgatgggaaatgatactatcctgaaacatgagcaaattgaccagcctcaggatgtgaactcatttgctggaggtcacccagggctctttcaagatagtaaaaacagtgacttgtacagcataatgaaaaacctaggcattgattttgaagacatcagacacatgcagaatgaaaaatttttcagaaatgatttttctggtgaggttgacttcagagacattgacttaacggatgaaatcctgacgtatgtccaagattctttaagtaagtctcccttcataccttcagattatcaacagcaacagtccttggctctgaactcaagctgtatggtacaggaacacctacatctagaacagcaacagcaacatcaccaaaagcaagtagtagtggagccacagcaacagctgtgtcagaagatgaagcacatgcaagttaatggcatgtttgaaaattggaactctaaccaattcgtgcctttcaattgtccacagcaagacccacaacaatataatgtctttacagacttacatgggatcagtcaagagttcccctacaaatctgaaatggattctatgccttatacacagaactttatttcctgtaatcagcctgtattaccacaacattccaaatgtacagagctggactaccctatggggagttttgaaccatccccataccccactacttctagtttagaagattttgtcacttgtttacaacttcctgaaaaccaaaagcatggattaaatccacagtcagccataataactcctcagacatgttatgctggggccgtgtcgatgtatcagtgccagccagaacctcagcacacccacgtgggtcagatgcagtacaatccagtactgccaggccaacaggcatttttaaacaaggtaagggtgttatcaaactgaattaaatctttcagtgattctttttaccttatagacatgttacacattttttatgtcagctgattttaatcggttatctacagcattcatggagacagcattttttattatatctgtgactaccttttttttttttacaagcctgtactttttccagtatgttactaatatatcattcacctgattcatgagggagaaagatatcaaggaatgaagcaacatgaatgtgtccttaatataagatgacctaaattaccaggtgaaattagtttttagaagtattttgtttaaaatagataatttggtttattctttctggattctttaaaaattatatggctatttaaaaattcaagctttaaaaaacgtattgcaatttttaagaataatttcttcaaagctctatttttttatatatgtttgaaattgagttaaactcaaatttaagtgtgctttcttacaatgcctctgaaaccctagattaattatatccctggaattcagtcctaggcacctccatgttctctagctattcctccacattacctcccctatcctcatctcatcctttaaggtagaatggctttcttttctggctttcttaaaagaggtaactcagctggctttctgtcatggaaatcaagcttgatagtgcatttttgttgtttaaatgtgtcatcagaatccgtctggcagtgagcacactttaaatctaaaaatgatcacttaaaaaaaaaaagttaacataatgtcagtaactgctgtctctggccactcaggaatacatcatgtttgcatttatgctttttccattagaaaatatttagttcattttaaagtatatttaagtttagttttaaagaatatcttgactaaaattaagctccttgacccctgtttactctgtattagagtataataattaaaagtatgacctgaagctttatctttaattgtattttctggctttttcatacattttgcttgtctaatcgtatatgagctttttaatagaaaaggacagtgaaaatttttcttattccttacatccttcaattatattaaaattgaataatattcagttatagtccgtctcacaaaattgctatgaaagtatattttactcttgagtaaagcaaattgtacagaagttaattctgtaaaattacattttgaataaggagttgcatgagtaccacataagttctttgcagtgttttcccctcactacttttcactgcttctttgtaaataaccaaataattctatagacctacatatgttccagagtacatgttattgagctaagacactcaatattgagccactgagtagctgttcagcacttgaaatgtggctagtccaaattgagatgtgctgtgtgaagtacacattggatttttggattttgaagacttggcttgaaaaaagaatgtaaaatatctcatcttttattattgatttcatgttgaaatgattagttttttcagtagccgcattacaaaagtaaaaagaaacaggtaaaattaactttagaaatgtattttatttaatctactgtagccaaaaatttaagttctgtatgtggctcacattatatttctattggattacactgctgtgtacagtatgtttaaagagaatggtgacatgaattgtgttatgagtaatgagaaaaaatcattttaaactcaccattatctggccaggtaatactaactttttatttaaagaatttacagtgttgccattcattgaagatacaggttatatgatttttactatagattttatatatgttttggaggatctgtgaagtaattaacattttgattactgaagccagttatgtaggctttacaaaggtagattttatttgaagcaattaaggatatgattttaatctgttttaaagtttccagaacgtccactaactaaaatataaaggctctgtaatctataaaactctacagagctatacatcttcatgtccttcctttttatttttcttttttctactagcagttatagaaaccagattcattgcagtgtaagaaaacaagacagagcattttcttttggtaacttgattctgtaattagaactatgctttcagtagtattcattaattaaaagtatattatgaataagaaatcggttaagaaaatcatacctgtggtgtaatattttaggaatatttagaaacatttagaaaatcagaagctgccttttttggaaaagttttgcattccagtgtaagattcctgaatataatcaattcattgatacctataacttcaaacctggtcaaagcaaacctacttatgatagagaatatttaactgtaatttgtgacttgtgctgtttgacccatcaattcctacctgacccaaaggcaatctgaaatgagacagctgcatcactcatctgtaccatgttcaaaatttcctgtcaaattaatggaaaaacaggttataaatgcaactaatacataatatgtcctcagtatgtcttcactgtgtgaaagatttaaaatttagcaacagtaaaggaacttgaagtttacaactctcaggggtaagattttaaaaatacatgttaatgttatttactggcttaagatacttggaagatctattccaataagttgcatcaccatttttgttttcagtttcagaatggagttttaaatgaaacatatccagctgaattaaataacataaataacactcagactaccacacatcttcagccacttcatcatccgtcagaagccagaccttttcctgatttgacatccagtggattcctgtaattccaagcccaattttgaccctggtttttggattaaattagtttgtgaaggattatggaaaaataaaactgtcactgttggacgtcagcaagttcacatggaggcattgatgcatgctattcacaattattccaaaccaaattttaatttttgcttttagaaaagggagtttaaaaatggtatcaaaattacatatactacagtcaagatagaaagggtgctgccacggagtggtgaggtaccgtctacatttcacattattctgggcaccacaaaatatacaaaactttatcagggaaactaagattcttttaaattagaaaatattctctatttgaattatttctgtcacagtaaaaataaaatactttgagttttgagctactggattcttattagttccccaaatacaaagttagagaactaaactagtttttcctatcatgttaacctctgcttttatctcagatgttaaaataaatggtttggtgctttttataaaaagataatctcagtgctttcctccttcactgtttcatctaagtgcctcacatttttttctacctataacactctaggatgtatattttatataaagtattctttttcttttttaaattaatatctttctgcacacaaatattatttgtgtttcctaaatccaaccattttcattaattcaggcatattttaactccactgcttacctactttcttcaggtaaagggcaaataatgatcgaaaaaataattatttattacataatttagttgtttctagactataaatgttgctatgtgccttatgttgaaaaaatttaaaagtaaaatgtctttccaaattatttcttaattattataaaaatattaagacaatagcacttaaattcctcaacagtgttttcagaagaaataaatataccactctttacctttattgatatctccatgatgatagttgaatgttgcaatgtgaaaaatctgctgttaactgcaaccttgtttattaaattgcaagaagctttatttctagctttttaattaagcaaagcacccatttcaatgtgtataaattgtctttaaaaactgttttagacctataatccttgataatatattgtgttgactttataaatttcgcttcttagaacagtggaaactatgtgtttttctcatatttgaggagtgttaagattgcagatagcaaggtttggtgcaaagtattgtaatgagtgaattgaatggtgcattgtatagatataatgaacaaaattatttgtaagatatttgcagtttttcattttaaaaagtccataccttatatatgcacttaatttgttggggctttacatactttatcaatgtgtctttctaagaaatcaagtaatgaatccaactgcttaaagttggtattaataaaaagacaaccacatagttcgtttaccttcaaactttaggtttttttaatgatatactgatcttcattaccaataggcaaattaatcaccctaccaactttactgtcctaacatggtttaaaagaaaaaatgacaccatcttttattcttttttttttttttttttgagagagagtcttactctgccgcccaaactggagtgcagtggcacaatcttggctcactgcaacctctacctcctgggttcaagtgattctcttgcctcagcctcccgagttgctgggattacaggcatgtgccaccatgcccagctaatttttgtatttttagtagaaacgggtttcaccatgttggccagactggtctcaaactcctgacctcaggtgagcctcccaccttggcctcccaaagtgctgggattacaggcgtgagccactgcattcagctcttcttttctttagatatgagagctgaagagcttagacacattttgcatgtattatttgaaaatctgatggaatcccaaactgagatgtattaaaatacaatttttggccgggtgcagtggctcacgcctgtaatcccagcacttggggagggcgaggagggtggatcacgaggtcaagagatggagaccatcctgaccaacatggtgaaaccctgtctctactaaaaatacagaaattagctgggcatggtggcgtgagcctgtagtcctagctactcaggaggctgaggcaggagaatagcctgaacctgggaatcggaggttgcagagccaagatcgccccactgcactccagcctggcaatagaccgagactccgtctccaaaaaaaaaaaaaatacaatttttatttcttttactttttttagtaagttaatgtatataaaaatggcttcggacaaaatatctctgagttctgtgtattttcagtcaaaactttaaacctgtagaatcaatttaagtgttggaaaaaatttgtctgaaacatttcataatttgtttccagcatgaggtatctaaggatttagaccagaggtctagattaatactctatttttacatttaaaccttttattataagtcttacataaaccatttttgttactctcttccacatgttactggataaattgtttagtggaaaataggctttttaatcatgaatatgatgacaatcagttatacagttataaaattaaaagtttgaaaagcaatattgtatatttttatctatataaaataactaaaatgtatctaagaataataaaatcacgttaaaccaaatacacgtttgtctgtattgttaagtgccaaacaaaggatacttagtgcactgctacattgtgggatttatttctagatgatgtgcacatctaaggatatggatgtgtctaatttagtcttttcctgtaccaggtttttcttacaatacctgaagacttaccagtattctagtgtattatgaagctttcaacattactatgcacaaactagtgtttttcgatgttactaaattttaggtaaatgctttcatggcttttttcttcaaaatgttactgcttacatatatcatgcatagatttttgcttaaagtatgatttataatatcctcattatcaaagttgtatacaataatatataataaaataacaaatatgaataat

SEQ ID NO: 22 - DNA, 45676 bp, Homo sapiens
cccgggccaccgcctccgcccggctgcccgcccggactgtcgcggcccgcggtggcgacggcggccgctgcaaagtttccccggcggcggcggcccgggggcgcatcctcccgcaactgtcaagcgctggcggcggaaatgatgaggcgctggccattttccgagcccgggtttcctgcctgagccccgctcgagcgagccgcgagcgaggagccggcgggcgggagaggacgcgcccagggcgggggcccgcccgccccctcgggatttcgagggcccgggggcgcgcgacgccatgggccggccgggcccagagctcctgtctctcaggtgagcccgcgtttgcccgcgctcccctcctccggggccgggagggcttttgtggggaaagaggaggccccgtgtccagttctgcccgggctctgcgcgtcacccccagcccaaaatactgccctgcagccaactcggatggaggcgttccggggtgacggctcccagcgcctacgaccccccactcagccactacccccacccacacacgtgttagggagagcgttggggtaactgaggccgtgggatgggggagagggcatcgctgacgcccccagcccaccctacggtagcctcgccggttgggggcggccctcccctcaacttgtgcctggggaggagcggtcaggggtcacccattgctaccctgaaagtgaaagtagtcaaactgaggctgtgagcacagatgagcccttgggtgtgggggcagcgggggaggatgcgccggcccctccccactccccattccgggccatgtggctaagagggtagcgccctgattcaggaagcccctctccccagttcctgcactgccgaggtcgaccgggccaggcccccgcatgctgctgaattgggcggttcaccagggctaggcgtcccagagacaggtttctggtattgatcctctcacacctcttgcctggggttccctgggaatcccccgaccaaaaaacgtctggagtgggtgttggtcttctagatccgcacggtagggatggtatttcagaggtctctgatgcctggtgcagaaatccccttccctcccttccctccctccggccaggggagctttgttttgagggagggggactgcatttgaggaactggagaggagggtctgcctgcctgtcccgcttctttttttctttagtttcctcattctcctgtttctcttccccattcccagccacaatgatgtaggtgaggaggtcactgcctgcagctgggggcctccagggaggggcacggccaggctgggctgagtgggaggaagtgaaatctggtggggggttcagagcagaggccccggcaaggcaatgtcggcaaggccagctttggtggcaggagggaccgagatctgacccgcagcccctcgccggggagatgacagttccctaacaccggctagcagccaggcctcagggcccctgccagggcagtgggcctgtgggtctccagggatgcaggcgcaggttgtgtctgggtttcccatcactgacctggctgcctgagtggaaacagctctggaggctcctcgggacctgccctcgtggagggagagccgtcagaggccacctgggcaggctgggctcggaggatgagagccagggcctactgggggaagttggaagggtttttagtgcacttttcctgagccttttcccactaccccctctgggagggaaaatccttctgagccaaagggtgagcattcctagaacttttgtgagtccctgtgggccacagagcggccagctgggaaggggctggctcagagcatcagcagctgggggctcagggtctgcaggggctcagcaggaagtggaggaggatagtgaggagggtcctggagactgtgctttgctactcctattgccttcgttcagctgaaggcgtgggcaagtgactctgccctcagtttccctctgtgtaaaatgggggagtaacaggtttgcagtggagatttaatgagccagtgtgtaatgagcacctgctgcctggcaaagagaggttcagttgcaggtggtggtggtggtagtggtgattttatcattgccttcattagtgttactgttattggctatgaggtgttccttcagtagtctcccttgagcatctcttgcatacatagcactggaatttcctgggtctttgggagcagaattttgaagatctgggcacttccttagctataaggccttgcttaactgccccacctcagtttcctcatctgtgaaatgggtgtgctaatagtacctacttcctagagtcatgggaaccatgaatgagtgaattcacagtgtccagagtggcttggtcaggaggaagagaagaagtagccttggggccggaatgatcttttttgttgtgggtgttagctctgctggctttctttcctcatccccccacccccacccccatgcctctcaaacctgggcctgatcctccttctccgataaatagaacttttgagtacaaatcagatcccacaggactgctgggcccttggatttctggctcccgctgtgccacccccagacattctccatatcccaggtggagcagagctggaggcacttggagggacctcctcgctgccagtctcagtgccacctgtctgtggtgaggggagaccgggtgccctccccagccaaggtggggagggtcagtgaagggggtctcagggacatagtagggagctgtcccacagtctgggccaatacagctccttttgctggaaacacctttgggacccagtttacatgaccccacacccttctgagggctggtgttcatgagggtctcccattgctggggatccttgggttgatcacttaagctctctgaggcaattgagacaacatctgtgaaaggctttaacagctgtgcacttgctgtgacatggcacataaggcactgagcgctgagcctggcactcaattaacaatagtagatattattgctattaataaaacaagaggcttttcctgctaaattcccacttctccctggtagcagtatggactttggggccaaatctgggctctgccagcttccaggtgtgaggtcctaggcaggttacctggcttctctgatctggacctcgcctgcccgatggaagccaagagttccagcctccccagggttgggtgtgtgaagtacagtgtcaggactggaactgtgttggctgtacttgctctggctgtgtgatcttgggctggtcccttagcctctctgagccacaggtctttttgtctatgaaataggggggttgccaactccctgttagggtggttttgagaaaaggaggcatgcacagtgcccggcatgcaattggcactcaataagtgcaagccattgctgttcatgcccaataggggctggcacttaattgccaggtttcctgggaaaacggtccccaagggcccgcgggcgttcagcatctttgacgaggcagggcaggaagccgctgccgtctggcagaccggagacgcctgcctcggcgagtccagaggtgcagtgctagctgctcattccctgcaaggccccattcctcactacccacccttcccaaggaaactgaggcgggcaggctgcccaggggaagtgactcagccggggtcacttgctgagtcaggccttattggacccaggagtcctgcaccccacctccccagcttcccggggcttgagatcccttggagactgagcaggtcctgaggcctggcagcaggaccaggccaccactggacatctgctcaatggggccctgctgccggggtgacaagcatccctctaccagggctccagggttctgctggccaccactagccatccttcctcccagcaggggcagaggacacttccccaggggaacaggtcctcctggtgcccccccactccccgccccagccggaaacccggcagctgagcagttagatagaagatttttcctcccacgccaccctgtcaggggtcggccagcagggccgggccacaggcttccttgtgagccaggcctggagcccctgggggcccacccggatgtggctaccccccggctggccagtggggagccggctgctgcccaggacatgtgtccccagggaggcctgggaggaaggaagtctatgccctcctcacaggggctgactcacaggcgcccaatggggggccccagctgggagactgggaatgtgatcaatcagggaggtggacacacaaggggacctctgacatgggatctgccctttcagagctcacacactggtgtcatctcaaggaggagaggcccagagagggcagtgtatgctgcgagaggcgaggtaattgagggcttacgcatgtccctcctgatgtccccaagtagcacgtggggaggaggttcacccacaggctgccctcctagggtctcccctgtctgccctcaacccagaggcagtgaaaggaagaagccacggccttgggatggggggcgtgcgggattctgatggtgctgcctctccgcagggagatggttcttggctttccctacctcctgaaacctagctggggcggtggctgggatgtcaggctggcggtgtgggcagaggaagccagaggacatttcctgttctggcagactgtgctggcctgataaatgtccccggggcacaaggtgaccccagcaagggtgcatcagtgtccagccagctgccccgtggcctggggtagaacccacctctctgcctccccagggaccctgccttcgccccctgcttcccgtgggcatctctgcgtctggcaacttgaatgctgtgaaagggcagcaggccgcctgccaggcagtgtgtgtcagaatcccacaactctctgggcctccttaggccacagggtctggactctatgaacagttggtgctaggcatggggaagatagaaatgttccttgtggcctagattcaaccaggaggcctccctggaggaagtgtcccctctgaaggtgtgagacttcctgcctatatctgtgagctggggcaggggagagtggaggctctggactggcccaatttaacttcatttaccacactttaagaaatcttccccaggctgggagcagtggttcacacttgtaatcccagcactttggaaggtcaaggtgggaggatcacttgagcccaggagtttgaaaccagcctgggcagcatagtgagaccctgtctctaccaaaaaaaaaaaaaaaaaactgggtatggtggcacatggctgtggtcccagatacttggaaggactgcttaaacccacaaggtcatggttgcagtgagctgtgatcatgccactacactccagcctgggtgacagtcagacctgtctcaaagaaagaaaaaaaaaaaaaaatcctccccaagcctcagcaggggaattcctaccatggataaaggctttctctgcatgtgggcctgatcattaaaaggaaaatttcagcctcccgaattatactattatccagggtgattagatagaggagaggctgagaagccctgatctcagcggggcatggattaaggggaccccacagctcagccaggttcccagcgattcaaggtcctgcgatttctcaccgacttggtggctccagggtgcctggggctggctgtgcttcttggggagcaccaggtaccactgctgcttcgaggccctcaatagccctttggtggacccggttgtgttctgagcttggggtcctgagatgcagacaccccccagccccagctggtgggcgtgcagctagctgccttcatgtatttgctgcagggagggaattgggggtgggggacaaggatgcaccaggctgagttattccttctgcacatcatcctgcccctcacctggctgcaggatttggggaaaccctggagacccccatagctgggaggttcctgccccagaaggaagcctgagcaggtgaggggggtctcctgggtgctagggcccctctgtggggaaggtggtttatttctctggctgaccaggctcccccgaagctctgtgctgggatagaagtgtcccaaagacaccctgcccccaactcataccctacagtcgaagcctgagggccccaggtccccctggcgtgatcatcattgcttcctgtccacagcttgtcagcccctgatcccagggttgtcaccatctgatcaattgctttttctctgagcctggcacatagtaagcactcaataaacgccactgcctgggccagggtcagggagggatccaccttcctgtggcctggctgccctcctggccaaaaaacctccctggtacagtcccttccatagcttgggctacagctgactgctctgggctggcaacttcctgcccggagcagcctgagcagccgcctctcccctgagaagatcttttgttccagctccccatgacgccccgagcccagcccatgaggcaggcccccctgtggcttcccatggcgggcgagcctgactccgaggctggagctcttttctaacagtgatagtaagaaaatagcagcagccacttatgagccccatgccaagcacactgcagacatcagttctttttttcttatagagatatggtctgcttctgtcactcaggctggagtgcagtggtgtgatcacagctcactgcagcctcgacctcctgggctcaagcagtcttcccgcctcagcctcctgagtagctgggactacaggcccagccaccatgccaggctaatttttttgtatttatagtagacatagggtttcatcatgttgcccagcttggtctcgaactctggggctcaagcaatcctcccgccttggcctcccgaggtattgagattataggtgtgagccactgagcccagcctctcatttttgttttcttccttttgccctgtgacagttttcctgatcattggtgggtttgggacctggacagagctactgccgactcctgactccagctggagctgaatgagacttttatattctctcctctgggactcactcattcatccaataagcattcattgggaagtgagcccttcttgtgtgcatttaggcactgatcagacattttgggggtcccttctgtgtgggggtctctataatcactagctatggggccgctctgtttcagcttccccatctgtaaaatggaagtaatcgttggacctacctcctagggctgttaggaaccttggaagaggtaatattttgtaaaagtagttagaaaaatgcctgatatgatcttggcatggtgccatgagcctgtagttacagctactctggaggctgaggcaggaggatctcttgagcccaggagttcgaggcagcagtgagctatgattgtgccactgcactccagcctggaagacagagtgagaccctgtctctaaaaagagaaagaaaaatgtctgatgtgtagcagacgctcagtaaatgccattgttcttatgggaggactggtggaaggggcccaatgaggggggttcccagccgttgcctgggcagtgagggacctggagtgggcgcagctgttttgaggaaggctcctgccagggtttggccctgcagttgggcttcaagactctgacatacaggactgtgcggaaggtaggacggcccagggggcgggggaggttggttcatgctggttgccacgcatctgccgggagagcaagagtttgcaaatccaactgatctcttgcattcatatgttcattcattcaagaggcattcattgagctcctgctgtatgctaggagctggagatgaagcagggaatgactcatggaactcagtctgttggagagatagtcactaataaaagcatcctgtcaagtgataatgtgtgaccagtgtgctgaaggtcaagttcagttgggagctcagaagaggcttctcttatgtgcaattggaagcagacagggcagagggaatggctcatgccaacacccacaggcagctggcgtgactagcggcagatcgtgtaggtggggcaggagcctgggtctggaatcacagctctctgggcatcctccaagccacgagggcagcgcctgtctcacctgtgtcaaggctgggtgtggggaacagtaggattgaccaagaacaggtgcccttatgcaggggttagaccctccccccttcagggacaggaagaacatggtggccaagggcagatgtggcccctccctctcccttcccatctgcctgacagcttttagtgactacttactgtgtgcaaagtacctactgtgtgcgggtcccaaggaccctgtgatgtacaaaactgtcgactgtagcagggcacggtggctcatgcctgtaatcccagcactttgggaggccaaggtgggagggctgcttgagcccaggagtttgagaccagcctaggcaacatagtgagaccccatctctacaaaaaatacaaaaattaaccaggtgtcgtggtgtgtgcctgtagtcttagcactttgggaggccaagatgaggatagcttgagcctgggaggtctaggctgcggtgagccatgattatgccactgcactccagcctgggtgacagaacaagaccctgtctcaaaaaaaaaaaaaaacattgtcgactctgcatggcccccttccatcgagggcagaggcacctacttgtgctttttggggtttcttgtggagatcggctgtgggggttccaccatggcctcatgtgacaggggcaccctttgttgccactgccctgcagacatgcccagcgggacatggccacatgcagagacggtgcctgtggaggggtgtgtggcagagagaaccccatttgtaggttcgcatctccaccttgcattccctttcatgggttccttacaaggtggatgccatgctgccacctggttggcagatgaggaaatccaggttcagagaggttgagtcaggcatcaggagccacacagccaatacgtgtccccctttgtaatgtgagccctgggagagcagtgttcgtctgtgctgttcaccgtggtatctcagggcctagcctggtgtctggcacacagtaggccctcagtaacacctgaagagaggatgaaggagggcgtgattggaggcagcaaatgtacccaggcccctgtggctctgtagcttgtgttgtgcagccactacccctgcccagggagggagtcactggagtggcactgctgttcattcagggtgtgctgatatcagggatctggggcccagttttgtgcctgagtagggagagcatttcttctccatcttgaagagcgacatggatatgtggtctccagcaggggcccaggcccagcgagggtcaggaggaaggaaggatggtgcccacagaggtcccaactgttctgcaccgtggacagcagaggggtggggggtctcagtgccattctggtgatgttgtgcctaaggaaaggccctctggccatgggaggagaaggatgccccagttgagatgggcaggcaaaggtctgctagtcctgagggtcccctgtcagtgcaggaaatccctctggacccccgtgacttttgttaatttattcctttttattttttgttttatatttatttttgtttttgtttttgtgtgtgtgattttttttttttttttttccaggacagagtctcgctctgtcacccaggctggaatgcagtggcatgatctcagctcgttgcaacctctgcctcccaggttcaagtgattctcctgcctcaccctcccgagtagctggcattacaggcacacaccaccacacccagctaatttttgtatttttagtagaggcgaggtttcaccatgttggccaggctggtttcgaattcctgacctcaggtgatccacccacctaagcctcccagagtgctgggattacaggtatgagcagctgcccccggcccatttatttatgtatttatttatttatttatttatttagagacggagtctcgctctgttgcccaggctggagtccagtggcgtgatctcggctcactgcaagctccgcctcccgggttcatgccattctcctgcctcagcctccccagtagctgggactacaggtgcctgccaccaccccccgctaatttttttgtatttttactagagatggggtttcaccgtgttagccaggatagtcttgatctcctgacctcgtgatctgcctgcctcggcctcccaaagtgctgggattacaggcgtgagccaccgtgcccggccctatgtttattttttagacaccaataatatactctcaccgcaacatggaaataacaacaatgtcagccagtctttatgaagcattttctacgttctagggctttacagctatcaattcatgctcataatatgcctatgaagtaggcatgatcatggttcccctcttacagatggagaaactgaggtagagagtaatcatgtaagtcacttaaggtcccataactaataagttgttgctggttttttttttgtttttttgttttttgttttgagacggggtcttgctctatcacccaggctggagtgcagtggcaccatctcagctcactgcaacctccacctcctggattcaagcatttctcctgccccagcctcccaagtagctgggactacaggcgcccgccaccacagccggctaatttttgtatttttagtagagatggggtttcaccacgttggccaggctggtctctaactcctgacctcaagtaatccacctgcctcggcctcccagagtgctgggattacaggcatgagccaccgcgcgtggctggtgctgggatttgaacccacattccagaatctgtgttcttagccatccttgctagagataagcagtaacaggcttcttggttacttccccaccatccccacatcccaattctgattcctccccagaggtacccactgtttcaggatgagtttgcttctgccagacctttcttgcccaccatttacaaaatgtttgtgtacacataaacattagtagtttggttttatgtatgttcatgtaaatgggattgtctagggtgtcttgttctgccgtttgctttttcgcttagtgaatagccctggagctctcccagaggtggtggggtgggtcttcctcactctttccccctgatgcagtcttccagcctatagatgggccagattctttcactagtctccagctggtgggcatttaggttgtttctagtttttccccattataaacgttttagcaggaaatatgtagcagggagcaagtatttctcttgaatacttaaaagtgggattcctagagcctgattgctctggctaggactgtcagtactgtgttaaatagaagtgaccagagtgggcatccttttctcatccctggccttaaaggaaaagttgtcaacttttcaccattaagtacaatgttaactgtgtgcttgtcatatagggcctttattttatttatttatttatttatttatttattttttgagacagagtcattctgttgcctgggctggcgttcaatggctgatcatggcttactgcagccttgacttcctcaggctcccaagtagttaggactacaggtgcacgtgctgcaatgccaactcatttttgtgttggtttttttggagagatgggctttcaccatgttgcccaggctggtctcaaactcctgggctcaagtgatcctcctgcctcagcctcccaaagtgctgggattacaggcctgagcctccgacctcagcctttatatagggccttcagtgtgttgaggtacaacataccatctatgcttaatttgttgaaaatttttatcataaagatatgttgaattttgtcaaatactctctctgcatctatcgaaatgatcatatggtttttgtacttcaaaagcaggattccagagtctagaatcaattctgccaaattggccatgccccttacagaatgccacttggtaggggagtgcatgtttccctgcatcttcaccagcactgggtgttatggtcgcgttggatttctgctgtggtgcccggtgtgtaatggggcctacacctgcttgcccacctgcttactccttgtcgcccccccacccacgtgtctttcagcccggccgcaccacctgggtctccgccatgaacgggcctgccctgcagccctcctcgccctcttccgcgccctcagcctccccggcggcggccccgcggggctggagcgagttctgtgagttgcacgccgtagcggcggcccgggagctggcccgccagtactggctgttcgcccgggagcatccgcagcacgcgccgctgcgcgccgagctggtgtcgctgcagttcaccgacctcttccagcgctacttctgccgcgaggtgcgcgacggacgggcgccgggccgcgactaccgggacacaggccgtgggcccccagccaaggccgaggcgtccccggagccaggccccggccccgccgcccctggcctgcccaaggcccgcagctctgaggagctggccccgccgcggccgcccgggccctgctccttccagcactttcgccgcagcctccgccacatcttccgccgccgctcggccggggagctgccagcggcccacaccgctgccgcccccgggacccccggagaggctgctgagacccccgcccggcctggcctggccaagaagttcctgccctggagcctggcccgggagccgccacccgaggcgctgaaggaggcggtgctgcgctacagcctggccgacgaggcctccatggacagcggggcacgctggcagcgcgggaggctggcgctgcgccgggccccgggccccgatggccccgaccgcgtgctggagctcttcgacccacccaaggtaagtaagccctgcccgcggggttgcgcactgcactgcgcccttcgccttcaccctggggagagcgcgggctggggaggtgtgatggctttccagctggtggccacagagtgtccagagggaactaggccctcttaaaaaagaacttttaggccgagtgcaatggcttatgcctgtaatcccagcactttgggaggccgaggcaggaggatcgcttgagcccaggacttcgagaccagcttgggcaacttagcgagaccctatctctataaaaaattagccaggtgtggtggtacgtgcctgtggtcccagctgttcgggagactgagaagggaggatgacttgagcccaggaggtggaggctacagtgagccgtgatcatgccaacacactccaggcctgggtgacggagtaagaccctgtctctaaataaatacaataaaaaagaactttttattttagaattttagatttacaggccaggtgcagtgggtcacgcctgtaaccccagcactttgggaggccaaggtgggcagatcatttgaggccaggggttcaagaccagtctggccaacatagtgaaaccctgtctctactaaaaatataaaaaattagctgagcacagtggcgcacgcctgtaatcccggctacttgggaggctgagtcaggagaatcacttgaacctgggagggaaagttacagtgaactaagattgcaccactgtattccagccttggtgacagagtgagactctgtctcaaaaaaaaaaaaaatgtttagacttacaggaaagttgcaaagatagtagtgttcctttacacatggtacccagcttcaccgaaggttcacatcttacgctgctgtgtacattggttgtagctaaggaaccagcacgggtacatcactattagctaaacgccactgttcctttggctttgccagttttcctctaatgtcccttttctgttccagctcgccccccaggataccacatgaggtttagtcgttacgtctccttaggcgcctcttggctgtaacagtttccaaccctctattgctgatagccttggcagttttgaggagtaccagtcaaggattttgtacaatgtccagggacacctttttgccttcacatttgggtgtcatgtgggctggtagcaggtcactctgagatccttgaaaactctgactcagactggcattgacagattggtgttttatttccagagggctcaaaaccttcagagaatttctctgcacctcctgtgccttggtgatattttgcaagttaaaaagaatttgtcctttggcaaaaccccattgctacaaaaaataccaaaattagctgggcgtggtggtgtacgcctgtggtcccagctactctggaggctatggtgggaagatcgcttgagcccagaagaccaaggctgcagtaagccaagatcgcaccacagcactacagcctgggtgacagagcgagaccctgtctcaaaaaaaaaaaaaaaaaaaaaaaagttcttttccttcacagctggcttaattcctactgtgctgttgaggctgaaaggtcactgtgttctactcacctgccaggctccatctttgactcaggtatgcatgacaggaccatatggctttcatctaattgctctctctttgggatcctgttacgtggtagggccttactctagagcatgagcaaaaccgatgtgatactggccctgaggggctcacgatgtgggtggaggaaacctatggttactctttcattcattcgacagatatttatggagcacctactatgtgttaggcacatgctaggttctgggggaccaggcgggcctagtggaggctgttgaggaaataaacccacagatgagcatggaattaaaaatggtaaattgtgttctaaaactggtaaactgtgttctaaaggaaaagcaatggggtctctgggaggataactgaggggtcccggtttggcggtttgggagagtcatccaagaatcctttgaagtaacgttttcaaactgactgtggcgtaacatgcagacctggaagtgcgtgaagtatgagacacatctgaagctctgtgtttccgtgtgtgtgcaacgcaagtgaccagcaccccagagggctccttcatgcctcccccaggccagtgccctgtacccccatacataccacctcttatcaccacaggttcgatttctctgttcttgaacatcatgtgaatggaatcacacagtaggttgcctttgttttattttattattttattttgagacagggtctcactgtgtcacctaggctggagtgcagtggtgtgatcatggctcgctgtagcctcaacctcccagcctcaggttatttttgtgcctcagcctccccagtagctgggatcaccgtcacgtgccaccactcccagctaataaataggttgccttttatttctggctccttttgcccagcataatgttttagagattgatcacattgatcgtacatgtcaatagtgtcttccttttttttttttttttttttttttttgagacaaggtctcgctctgtcacccagctggagtgcagtggctcgatctcagcttactgcatcctctgcctcttgtcctcctgggctcaagcagtcctcccacctcagcctcccgagtaactgggaccacaggcacgtgccaccacacctggctaatttttgtattttttgtggagatgggatttcaccatgttgtcaaggctggtctcgaactcctgggctcaaatggtccccccacttcggcctcccaaagtgctatgattactggcatgagccaccacgcccagccgggtttgtttcttttgagtactgaatagattccattgcacagctgtctccatgtcccacaactggtttatcccatcctctgttgatgaatatttgggttgtttctattttgatttatgaataaggctgttatgaagtttgcatctaagtctaaatatggacacacatcctttcatttctctcaggtggattcctagaagtggcattgctcagtcatagagtaggtacatgtttactttagagaaattgccagacttttcccaaagcagctgcaccattttgtgtcccaccggcagtgggcgagagttccgggtggttccacttccccaccaacacttgagcacttggtgggtttttttgtgttttgttttgtttttgttttgagacggagtctcgctctgttgcccaggctggagtgcagaggcacaatctcggctcactgcaacctccgtctcctgggttcaagctgattctcctgccttagcctcccaaatagctgggattataggcgcgtgccaccacacccagctgatttttgtatttttagtagaaatggggtttcaccatgttagccaggcacttggtgttaagagtcaccggtggctgcgttcctggatctcaaggtggctttactttgcgtttccctgatgactcatggaggttaagcaccttttcatatgttgattggtcatcggaatttggtcgtttgtgaagtacatgtttttattgtctgtctagccttttttaaattgatttgtaaaagtcgttatatattttggacaatttctttgcgttttaaaagtcatttcctacctttatggcctggcttttcatgaaaagcagttttgttttttttttttcttgagatggagtctcactctgttgcccaggctggagtgcagtggcgcaatctcggctcactgcaacctctgcctcctgggttcaagtgattctcctgcctcagcctcccgagtagctgggattacaggcgcatgctaccacgcctggctaattttttgtatttttagtagagatgggttttcgccatgttggccaggctggtcttgaactcctgacctcgtgatccacctgcctcagcctcccaaagtactgggattacaggcgtgagctacctcacctggcctcaatttttgtatttttagtagagatggggtttcaccatgttggccaggctggtcttgaactcctgacctcaggtgatctgtccgcctcggcctcccaaagtgctgggattacaggtgcgagccaccacgcctggccaggaggtcacttactttcattctaagtaaatcactgggccttccaggcagagggaacagcatgggcagagaccctgagtggggaatgtgtttagcatgttttgaagaaaggacaagatgaagaggccggtgtggtgggagcagagagagggagatggggagcggggaagtacatgtgggtggaggacactgggaacgggaggacttagtgttttattccaagcacaatcagaagctgttgggaaaacgcaagcacagataaaaaacggttttctcatcatttgttaaaaatctcaaggctgtgacaccatgaaagacaaataacggccccaatacatgatcttatctgtgtgacactgtagggtttttatttttttattttttatttttttgagacagagtcttgctctgttgcccaggctggagtgcagtggcacggtgtcagctcactgccagctccgcctcccgggttcatgccattctcctgcctcagcctcccgggtagctgggactacaggtgcccgccaccacacctggctaatttttttgtatttttagtagagatggggtttcaccgtgttagcccggatggtttcgatctcctgacctcgttatctgcccgcctcagcctcccaaagtgccgggattacaggcgtgagccactgcgcccgtccagggttcttaactttgaaaaacaacctcagtggctgggcgcagtggctcatgccagtaatcccagcacttagggagaccaaggcagattacctgagattgggagttcgagaccagcctggccaacatggtgaaaccccatctctactaaaaaatatcttggtggccaagcgtgtggctcatgcctataatcccagcactttgggaggctgaggcaggtggatcacctgaggtcaggagctcgagaccagcctgaccaacatggtgaaaccccgtctctactgaaaatacaaaatcagcctggtgtggtggcacatgcctgtaatcccagctactacggaggctgaggcaggagaatcacttgaacccaggaggcggaggttgcagtgagctgagatcgtgctactgcactctagcctgggtgacaagagcgaaactccatctcaaaaaaaataagtgaatatataaaaaataaaaataatcttggaagaccaggtgttgggagccagctggccctgggtttgaataatgaataagctgctttctcagctctggggcaagtggtatcacctttctgagactcagtttccccctctgtacattggggagtactcatagttcctaccccatagggctgtgtgtcttggtggggagagcgcatgtgccacgtgcctggcgctgggagcactccatgagtgctgtgttgctgttgccacggttgttgtggtttgttatggttttgacttcgggcggagagcccaggtttatttccagtgcttgctgggccctcaggggaaggcatccccaagactcctgttctctgggagccctgatggtagggccccatcagattccctagggcttttcccaatccagggaaccagggaggtcccagagggctttgccgtagccctggctggctggggctctgtcacaggcttggtggttgctgcggggcctggggtgttgcctgcaaagcaagcagccctggggaggcttctcctagcccctcggagcatcaggtggaacagtccgggcggctcaggagcatcccctcagtgaatctgtgtccaggtgatgggccaagggtgctcagaccacaactcttgcatgtgcttgccaggtagtcaactgtgatccctgacctctgacccaggggttggctctgggaacagttttaaacaaaaggaaaagaaagttctgacaaccagtgggtggtggcgaagggcttaagctttggatctgcacttgcggtctgtcacttaaagggtgccctgcaggagagggagcagatgtaatcccagtgccagcctggtggaggccctgctgcccagggctggaagaggctttggagggagagagccaagagaaggcagctgcccagaagtctgaggccaggtgggctcagcccacaggtgagcctcacctgcagttgggccccagctctgcccccgacctgctgtgtgaccgcgggcctgtggagcagggtgatggtgtagcacctccaggggcactatgagggctgcgctgggagagagcactctgtgactgggggtgtgcaaggggaggccacagtctgccaggggtgggggtgtgggtggaggagatgtctgtgttttccaaggtcaggagaaggggagaaaagtcccagctttctctggtctcagtggccactgcaggtgggcagcgtgggcccttgggaggtgggagggggctgccttccctggggttggtcagcaaaggcctcttggcaggaggtgccggtcctgatagatctgggtgaaggagtcccaggctgaggaatggcaggttcacaagccctgcagtgagcgtgaaccaggcaagtctgtggaatagccaggcagcctggggttcgagggaacagggaaaggtgagactacaaaggtagtggagctggattgcagaggcccttgtgggccacagtggggactttaggttttacattaagagcagtgggaacaattggaggatttaggcctagagggcctgagacacttgctggggccacctgcgggtgggagtgggtgcaagggaccctccaggctacctgtggtctcccagctcaggcccgcgatgggagagtatgacatgatttcctctagagacctccttcccctgggcaccgctgagccccaggcagggcaaggacaggggagcagtgtgtttatgtgggggaaaggtgacatgcctttcatcacccatcatcgggctcacgaccgacactcttgcaacaagacaggtcagcaagagaaaagcatcacaaagttattgaatcaaagttttatgtgacatgtagctttcaggaatgaagaccgaaagaccccaggaaatggtctgtctggtgtttaggtttgatggagcagggatagctgcgtaaaaatgggattgggatgaaagggtgtgatctactgagctagacggaggcggggtcccagcaaggcctgcttgctcccattcctcttggcctctgcatggtgttccttcctcccagtgtagggcaggacctctctggaacttacaacctgcctccctcagacaaggtaggttggagaatttctctgtggatggctcctacccaggaaggtaggggaaggttagagttatatttctaggttttatggctggctttcagggagaggggttctagattctatgacccaccttgaggaagaattctggttccgtttttcttttatctttttttttttttttttgttaagagagatggggatttgctatgttgcccaggctggcctcaaactcctggccctaagtgatcctgctgcctcggcctcccagtgttgggattacaggcgtgagccactgtgcccagccgcctagtttctatgactcacttcaggggagaaggagggtgagagagacaggaaggcagaaggaggtgagagagactttgcttctgagggccccttctgagcccttctagtcttctttagttcaagtacgtagcatgccacagcacctttctttggggtgttgttttctgagccccaacattcatggccctgaatgtgtgcctctgtgtgccaggtgttgccctaaattcttggaccttcccatgagctggagttcactgacccatttgacagatgaggaaactgaggcactgggagaagcaaagagacttgcctgggatcatgaagtgaaggggatctgaaccctgagacaggaactcttgatcccaaggggcccctaaccccaagcccttggccccctgctgaactggttcaaaccagagagtgtataacccagctccaggctgggctgggcctgaggtgtgcgtctcacctggcagttacaagaaaaggaaattgcactggttgccacatttacaacctaaagtggagagatttcacagaatccagacttctggtttctcttatggaatccggggatcggccgggcacggtggctcatacctgtaatcccaggactttgggaggcgaaggcagatggatcacttgaagtcaggagttcaagagcagcctggccaacatagtgaaacccagtctctactaaaaatacaaaaattagctggccacagtgacgtgtgcctataatcccagctactcgggaggctgaggcaggagaatcacttgagcccaggaggtggaggttgcagtgagctgagattgcaccgttgcactccagcctgggcgacagagcaagactccatctcaggaaaaaaaaaaaaaaaaaaaaaaaaaaaaaatcagaggatctggcggtgccaggccggcattggagggggtcccagcctggcctgtgctgccactggttcagcagtagagcctgacggggaggcgacagcagtgggggccctggggaaggagaaagattccagaagcagctgctaggctggagtagctggccttgggccccagaccttcccggggaggtggggctcccatttcaaatggagcatgcccagcctgccgggcctcagtttccacatatgcccaggcatggattagctgagatggtcttgaagtcctgccagccctggtggttctggaggttgagcctcagtttccctcatctgtacagtggaggacatgatggagttctagctgttgataaatgccctttaaaccaccatttattgggctttccttatacagcaggcactttactgacctggcttcattttatcttcacagcagccctgtgagggtagctgcgccattaggcccatttcacagatggggagactaggtctgagagagcttaggtggctttcccaaggtctcacagcccctgtggctgagaggggcttggaacctgggcagttggctgcccctgtgagcagtgagtccttagaaggacttactgtcagtattattgatggcactgttaactagggccctgcatgggtgggggcacctgttttgggctcttggactaggaaaagctcctgtggccattttatccaatgccaccattttatagatgccaacaccgaggcctggagggggctggccagcgcccctcacctggcacagaggactctgcctggcacatcccacccatggcctctgatgcccagcttggagaagtggtagaagaggggctgggggatgcctcagggggacttagccctcagccctgagccccccgctgcatcacaggcctgagtgcccaggaggcagtggtctgggtaggagtggctgcgactcgggggtaaatgtgcttcagagccatccccactcatcccctttcgagccaggtgtccctggtgtgtgacttggccttgctgggcctcagttttctgtaaaatgggaagtgtcatggggggcaggctacggtaagcactgatagggcaggtgaggggcagcctcgggcccacccttctggcaggagacatttggggcacccagaagccctggaacaaggccactctgaccttgattttgggagcacaaagtccccccaagagatagcacaggagcccgggtgtctgatgagtgacccatcccttgggggcctcagttcctgggtgtgcctgggtctgcaggggaggcagaggcacaggtgggcagctccctctgggtgggctgtgaccctgcctgggcacaggagggccgtgcccgtccgtccttgccgtgattcttccaggtggcaccgccctcacacctcctttacagaggaggaaactgagacccggggaggccacaggagtcgggctctgggcccagagcctgggcggggatgcaggagccacccccatgcctccgggtgtgggaaaggggaagcagaaagaaagggacgtccgccggaggcctggcggtttcctctcggggcaggagtgcgaggaggaggaggaggaggaggaggaggaggaagaggaggaggaggaggaggagggacggcaggaagccgcctcgggctctgacaggcgcggccctggcctgcccctgcccttctgagcctgtgacccgggggaaggcactttgccctccctgtgcctcagtttactcaagtaaaaatagctgaatgatggctctggccccaggatcaactgagtgctgtctgggaagcacctagagctgtgcccaacacagccagtgttcgctggggcctgcaaggctttgctgctgcaggcatcactggcgttgtcatttttatttattatttatttatttagagacagagtctcgctctgtcgcccaggttagagcagagtggtgtgattttggctcactgcaaccaccgcctccagggttcaagcaattctcctgcctcagcctcccgagtagctgggattacaggcgcccaccaccacactccgctaatttttctgtattattagtagagacgcggtttcgccatgttggccaggctggtctcgaactcctgacctcaggctgtccatctgcctcggcctcccaaagtgctaggattacaggcgtgagctaccgcgcccggcccatttttattaattggttttcatgtgacaggcactgtgtccgccacatcccatggaatccttatcacaaaccccggaggtggggacagtgatgaagccatttcacagccaagaaaactaggcacagagaggtccagaaacttgcccgaggccccagagctggtaggtggccaaggcaggaaatgagagtccctggtgggtctgccaaggccggggggcaccaggggcagggggtttgtgtttaggccccagaacaggcagagagaaagcacccaccctccaccgtcagaacgagcctgtatcccggcaagcagggcagactgagccctggtcttggcaggcaggcagagctcaggtcctggcgggcacacaggcttaagaagccggttttttcctgggcccacgtctcctgggggaccagcggcagaggctcctggggtaggaacggaggtcccatcctcactctgtcctgggctgccatgtgacccaggtaggtcccagtcctctctgagctatgaagtgggtgcctgcagtggccttctgggagtgatggggaaggcggagggggttgggcagggctcctgcggccccagccacacacactgccgccccatctgcccccttattggggttccatgcaaaatcgtttttgaaaaagtgctttgccgcttagaagaaacccccgaccgcgctgaaaccacgggggcagatggtatcggggcgcttcctttcagctcaaattctgcgaatccacagcctagaaagtgccctgaatccaacaaggggggaggtaattaagttattatggtccatccccaccccgtcctgcttaccaggcctggctgctcttactggcttcttggaagttggtgtggtatttttttgttttgtttgttttcgaggctggtattgagatgagaccattgtggggtcagggctgtgggcttggaggttcagggtcagggtccaggggcgaagggtggacgctggagaagggcagatggacagggtcaggttcagatcttggctcttgtatccttgctgtgtggctctgggccaagaacttggcctctctgcgcctcagtggctcattacagaaaatgggatgccagcacttgccttagtgggttgttttgagccaactgcaggctcagggagtagctggcatgatgtgttcctaccctgaagggcagaaaaaggggaaggaggccaccagatcccacaggtgggtccccagggtccctccccgggatgggcagcagcctggctgctccccacagcaacccctggctggccccattggcgctgctggaaatcccccacccagttctttgtgaatggaaaaggaaactgattgcgctagaagaggtctccgagtgccggccgggggtgggtggcggggggggcttcctctcccagtgcaggaggggccacctcagcttcctttctggggccccataccccctttttcctatctcccctctgagttgaggaggaggctgtggcccccgcccactgtttggcctctggaaccactgggtgacagtgtacatcggggcgattaagggccacctggcctctggacctccctcgctgtggctgctgcctgggaccccccaccccatgcctgactcgaaccccaacctcagctctacacacaggggcaccactgtaggaggagagagaaacctctctctagggaaccacctggaagggggccctgccctgtcttcatctgggtaccccatgtaatctaggaaactgtctttaacttgccgagggcctccatgtctgagttcctaatgttttttattttgctttttcaattaataaagctcatggcagaaactatttaaagaaaaaaaaaaacagtaacagaaaaacctcccacaatctcactacccagagagaaccagcatttccattttggtagatgtcattattgtcatttttgttttgtttttgagactgggggacactgctctgtcccccagactagagagcagtggcgccatctcggctcattgcatcctcaacctcctgggctcaaggtgatcttcccaccttagcctcccaagtagctgggactatttaatgctgctattagcatagacattagcaggacaccatgccctgctaatttttaagtttttttgtagagacaaactctcactatgttgcctaggctggtcttaaactcctaggctcaacagtcctcccgcctaggtctcccaaagttctgggatttacaggcgtgagccaccaccacacccagcctcttctctgtctttaactttgcctccgcctccaaattccaattccttcagcaattgctgggaagggattactttttgcacaattctgtctggagccccttgcaggcaggagcttgtggacaaaagcttcagcctttgcgggcctggcattcctcatactatttggtcacctgcttttttcactttgtgtcctgaacatttttctgtattcttttttttttttttttccacctcctaggttcaagcgattctcgtgcctcaggctcccgagtagccaggattacaggcatgtgccaccacgcccggctaatttttgtatttttagtagagacggggtttcaccatgttggccaggctggtctcgaactcctgacctcaggtgagctgcctgcctcagcctcccaaagtgctgggattacagccaccgcgcccagcccatttttaacttttttgctggtatagatgaccctgggatgaacaccttatgtcagtcttggtgcccacttctgatgattttcttctgctcagttcctagaaggggagtgctgggtcaaggcttttatgagcacttctgatccgttcttgctctcaaagctttattgagatataatttacaaaccataaaattcgcttgtgtaaagcgtataattcattggtttttagtatatttacagagttgtgcaaccctcaccataatctaattttagaacattttcgtcatcaaaaaaggaaaccttgtacccatgaacagtcactccccatacctccctccccagcccctggcaaccactcatctcttttctgtctccagggatttacctattctggacatttcacatatgtggaatcagactatgcagccttttgtgtctggcttccttcacttagcatcatgttttccaggttcaccgaagttacagcatgtattttgttcctttttatggctgaaaaatactccattgtatggacttgccacattttgcttatttgtttatcacttaatagacatcaggttatctccactttctggctgttatgaataatgctgctatgaacattcgtggacaagtttttgtgtggatctgtgttttcatttctcttgggatacatctaggagtggaattgctggggcatacagtaattctatgtttaattacattgggctgggcacggtggctaacgccagtaatcccagcactttggggggctaaggcaggtggatcacttgatgtcaggagtttgagaccagcctggccaacatggtgaaaccctgtctctactaaaaatcaaaaattagctgggcgtggtggtggatacctttaatcccagttactcaggaggctgaggcagaagaatcacttgaacccaggaggtggaggctgcagcgagccaagatcatgccattgcactccagcctgggaaacagagagagactctgactcagaaaaaaaaatgactatatataaaataaatatgactgccaacctgttttctgaagcaactgtaccattttatattcccaccatcattgcgtgagaggactccagtttctccatgtccttgccaatatttgttattgtctgtcttcttgattgtagccatcccagccatcttaaagggtgtgaagtagtttctcagtgtggtgttttttttgtttgtttgttttaaacggggtcttgttctgtcacccagcctggagtgcagtgcagtggagtgcagaggcagctcactgcagcctcaaactcctgggctcaagtgatcctcccacctcagcctcttgagtagctaggattacaggcacacaccaccacactcagctaatttttgtttttcactacactacgtgatgtggttttttgttttgttttgttttgtttttgttttttgaagagctggggtttcaccatatttcccaggctggtctcgaacacctggattcaagcagtcctctcacctcagcctcccaaaatgctgggattacaggtgtgagctaccgggtttagcctcagtgtggttttgatatgcatttccccaatggaaaatggtgttgagcatcttttcatctgcctaatagccatttcttattcttttgatacattatttttgagtagccttccagaaaggtcatacgcatatatcttcttgtcaccagtggtgccagcactgggtattattttaaaaacctttgccaccttctggatgaaaagtggttatcttatcctaagtgtgaccatttctctttgttaaaagttgagatatgattcatataccataaaattcacttgtttaaattgtacaatgcattggcttttagtatatttaaacaattgtgcaatgatcaccactatctaatccagaacatttctatcaccctagaaaaaaaccttggagcagtcagtccccaaactccctgcctccagcccgtggcaaccactgatctttctgtccccgtggatttcgactgtttctcttctgttccttggtcatacacatttcttgtgatttatcaggctatttactcagcatgtttgtctcctgcactgcttgcctttttcttgatttgtgaacactacacggcacggatattagctctgtcccatgttgcaaatatttccctggtttccgatttgacttttcaattttgttcctgcatttcttgacctatgggggtttgcaagttccaggcagtaaaatccagtgatcttcgcctttatggttttattctttgcttttttaccttagaaaggcctttccccacccaggatcagataaataattatttaggttttattctagttctgtcttggtttcctttttcacttttaattatttaatccagctggcttttgttatgggtggctggtgtgatttcctgttgttttgtgatgtcagtgagaggctgtgtgtcaaggcctgtgacatggtaaacgttcagtaaacaggagctaaaatgatagtaatgagagtccgtgccgggtggagctcagaaggacatgggattcccggtgctgtgccgtggctggaggaggacctggcctggctgtgccagttggctgggagcccctccaccccagaagcccttgctctcacctcttcttctgaatcatcgttcttcgaaggcaggcagtagctaccgttgtctggggctttggggatcttggtggtgatgagtcccctctcctctggtgcactctccccacccgagacgggcgacagaggttttttgttgtttcttaaccacatctttttccacccacacccggtgcagagcagatgcttggccggggcttccccgagctggtcctggctttgtggcagggggatgggccgtcggtgccccattcaagcctggggacctggccccactggtgtgtgctacagtagacccagccctggggccccatgataccctcggtgatcgtcacagatgcggggcctggctacagtcctcactggtgttttttttgtttttgagagggcatcttgaagtagcccaagctggagtgcagtagcacgatctcggctcactgcaacctccgcctcccaggttcaggtgattctcgtgcctcagcctcctgagtagctgggactacacatgcaccaccacgcctggctaatttttgaatttttagtagaggcagggtttcaccatgttggccaggctggtctcgaactcctgacctcaagtgatccgcccatcttgacctcccaaagtgctcgttacaggcgtgaacaacccccacccactgcagggagccaggcttgccccctccctgccaagggagctgcttgctctcccttctgtctggaggccagcacagcctggcactcgtggtttggggcgtccatggccttgtggtggcgaggcgcagaaggcgtcacctgaaaaaggtgtggccaagggcaggggtgctgggcttagccaggctggccacagtgggggcaggaaggattcctatggggacagctgggagcaggatgtggtcgtgcgaggcgtgggctgacggcagctggcatgcagcaggggaggtgtgctgggaccaggcctggagtggggtctggtggccctgccgtgtatggagctgagtgccgcccagagatgccatcttgtcctcagaagtggctcaggcctgctggggacagggcaggtggatggaagctgctgcatgccctgtgccatgtgctggtccgctggctgtcactgcagcgcagctcccggtgcctgacagttgtttgtccttatttggcacttgagagcccagcggtttggccagaatggtgccctgtgcggagggcacagcttgagtgaaggtgtggagggaaaaggccctgggggtgtggctcccctccttccatcctgctccttgcaaggcccatcccaggacattcctcctagccaggtgggctggatggggcctggacttcctgtccatgttttgggatgagagcgctggggaaggtgcccactgcggggtagggagtgtagggctttccaggcatcatccctcataagccactgtccccaggccttgcgcatagccccaccatagccgactcgagagtagaagcgtcccaccatcctcacagcaggctgcaggggccatgtgtaactggcccctccacccttggccacctggtggagtcggcacccatatctgaggaggggaccatgttgctgctggggactctgggctggtctggaagcaggtgtggtgggggctgtctttccctgtgtgaaggtcttcctctgggtgtctgtcgctgtagggaggccactctagcatctgaaaggggctctgtgtccttgccatcatgattctgtcttgggggtggttgctgtctctctgagagggggctttgtgtctgtgtccctgtgattctctgacatgcaggaggaccatgatgggaactgaggggaattcactctgggtggggttttagacaggtccccaagtcctgtctgtcccagcatctctggctaatcacttcccccgcccccctcccgccacacacatacacgggaaaagcaggctggtgtgtgtcctgttgcccacaccacactcagtcactcagtgtgagctggggtgccacctgcctggaggggggatctgactctgaacagggattctgtatttgggggtgtcacactcaccatgtctgtctctgcctctaccatctgactgggagaattcctctcagggttggggtgggcaccaagccttgctcccagtaggggcccagccctgggttgggcccagaggaggtgcttggagggtttactgggtgacggatggatgagcacttgggtaggggcctgggacccccaaagccctcagatgtctgggaaatagggggatgttctggctggggaggtttctgtttctggcattggggggctcttgccaggaaagggcttttctggcagccatgtggcttttctggaagcttctcccttctctttcctcctcccctgtggggaaagggcacctgaggcagcagggtgcagaccaaggggggatctggcccataatgaacagggtggtgggggtctaggagccgctgccctgtggactgggcccactgggcattcagggcagcaggcaggctcacttgcagaagtccaaggagtgggttccattatcatccccattttacaggtgaggaaactgaagcacagcaggccaaagtaatttgctcagggccctgcatttgaagggtctgtgaaggaggggccaggcttggggtcctggtggggggcatagcagagaagcgccctgtggcctgtagttcccacactgattcagtcgctggaactggctcattgccttcagtttgccccccaaggagcttgggaaaagggggctgagatcacccatcacaccccatacctccctctgcttcccaggcacttgagagcttgggggtcccaggtacctacccaagtgctcatccatccatccacccggcaaaccctccaagtacctcctctgggcctgccccagggctgggcctcagctggcagcaaggtttggtgcccaccccaaccccaagaggaactctcccagtcagatggtggaggcagttgcgtcatcaggaaaccatggccggttcgataagctcagtgctgagggagaggcacaggaattgcactaatcatgactcagtggtcagagaaggctccctggaggagaggaggttttctctgctgggacatgcagtttgggtcaggcagggcatcacaaatgcaggggctgggtggcaggggaaggtgtgggcgggaaggcggctggagggagaccaggtgttgggggtgctggggctgggggtgggcagatgaggattggagccctggctcaacttcgctattccccatggctggggcactgtgtgattttgcctcccagagtctgtagcatgggggctcctgatggtgcccttgcggggaggtcagggggctgacacacctgagcctgcctttcagcctgccctcttccctggccagctggaggggccttgggaggcccaccccagccctgagagcactggcttgtagcatccctatcactcatggtcttcactggggtgggggatggcagtaggggcaatcatcccctatgccccccgttacccagcacaaaggatgcttagccagcaaaaggcttatgatgaatgttgagcacccaccacctagcaggcactggctcgcggccaaggacaccacgtgagcaaagccaagtcacggcagagacctgcatcaaggaatgcgcaagaccagctcataggacaacaggcagggaaggaagtgaaggggctttgcggctcaggatttagatactgtggtcagggagggcctccctgaggagatgacactttgaaaagcagtcgtatttgtgaagctgtggggggagctgcctctctgagggcacaggccccgagtgcacctggcacactgaagaagagtggaggccagtgtggctggatcagagcgagggagggaggcccaagaggctgaggaggtaacaagggtctctcagatgaggaccacaggggagtttttgtttttgtttgtttgtttgtttgagacagtcttgctctgttgcccaggctgcagtgcagtgcagtggcgcaatcttggcttactgcagcctctgcctcccgggttcaagtgattctcctgcgtcagcctcccaagtagctgggactacaggtacctgccaccatgcccgactaatttttgtatttttgtatttttttgtgtgagatggagtttcgcttttgttgcccaggctggagtgcagtggcacgatcttggctcaccacaacctctgcctcctgggttcaagcgattctcctgcctcagcctcccgagcagctgggattataggcatgtgtcatcacacctggctaattttgtatttttagtagagacagagtttctccatgttggtcaggctggtctctaactcctgacctcaggtgatctgccttgcctcggcctcccaacgtgctgggattacaggtgtgagccaccgcacctggcctaatttttgtatttttaatagagacagggtttcaccatattggccaggctggtcttgaactcctgaccttgtgatctgcccgcctcggcctcccaaagtgctgggattacaggcgtgagccactgcgcccagccgggagtttcttttgagatgagattttgctctgttgtcgaggctggagtgcagtggcaccatcatagttcacggcagcctcaacctccctgcttcagcctcctgtagctgcaggggagttttgagcagaggagggtaggctggagcagcatcgtgggggtggaaatagctaattctggacagacttgggcgtagtctgatgggacagatggcagtttttgatgctgcccaactcccagagggcccaggtgtcacactgttttccagcctggctcctctctcctcactgggggctccttgtgaatacccctttcccccagcagttgtctgggactggaatcctggctgctcagctcccagcatccaccactttctgcctttgagggatgggccgtgcctcccctcctgcacatcttctgtggctccctggcatctggggggaagatgggggacagaggcctccctgcccagctctcttgacctccctcagaccccagccagctcagacgccggtgggtttcagatccacccaggtcccatgttaaaatgcagattccaaactcccttccagatgtcctgtgccagaaggtcatagaagcaccctgtgctgtgttccctcccccttggaggtctcttgagccttctcatctgtgaagtaggtgaaggcacaggagggccacgtgaggctcacacacactgctccttgcacggagctggcccggggcagggtggtgaggcttctccttcataaatctcactgtccatgatgcccaggattgtcctttgctctggaaccatgacgtcctacttggtttcgtcgtgtccttgatggtgtgacgcccatttctgtgacttcctcttggaaaactttttgaaaaatgttgccggcctgtgaattccacaatctggaaactgctggtgggccacccaggctccaatgcctggcacacagcagatgctcactaaagggtggtgtggggtctgtgagcgtcatttccagccacagtcaagcccctgttgtcttctagcctggggctgtgccttctgccctctgggtgttggctcttgctattcccttaacctccagtgcctttttctccaccatccccacttcggagaatattcagcaaacgctactgctcctttctctgtgctgggccaagaccgaaagatggaaaaggaagagggaggccttcttgagcctctggtgcatgggagcaggggagtgacaactcaggagggctgcccagggaggggccttcttgctcccaagtctctcttaaggctgagctcctaaggccctgtgctcctccctgccttaccattcccctgagatccttccataccctctgggagctctgttgcacacactatgctcatattctcagggaagggtagtggcattattatcccaccttagaaaatgcagactcttgcttgagcccaggaggtcaaggctgcagtgagctagtttatactctatcctgggcaacagagagggaccctgcctcaaaaacaaaatacacacatacacacacgtggagactcattttgggaggccgaggcagaaggatcacttgagccaggagtttgagaccagcctggacaacatagcgagatcctatctctatctctacaaaaaaataatcagccacatgtggtggcatgtgtctgtaatcccagctacctgggaggccaaggcaggaggctcacttgagcccccaggagtttgaggctgcagtgagccagcctgggccacagagcaagaccctatctcttaaaaaaaaaaaaaaaaaattggaaactgaggctaggagaggtgagtcacttacctatggaagagaaggcagagttgggactcaagcacaggactaagtgtcccaaacctcagggtggtggagttgctgcatggttctgtagccctgagggatttctgggcaggatactggatggcttttagcaactaggctcagtcttgatgctgcccaccccagagaatggctgagacgcaccacgtttaaggagagtttgcgaggtgaggaccttcctcacctcaatcccatattaccttgggatggtaataaggtctatgaaataggtcaaagtctgggtaatgcatgggtttaataggtttctctgctgcaggacttctcagagtctttaatggagctaagcagcagcattctctttccacaaatgactgtcctctctcggagaatcctttgcattggctgccaggaagctgagacaaatctgtggcaagagggactcttgtctgtgttgtagcagctgtctcccagctctttctgggtgtctctaacctgactaaaatcatttttggaagaaggcaaggaatttttttttttttaagacagggtctcactctgtcgaccagtctagagtacagtggtgtgatctcagctcactgcaacctctgtctcccaggttcaagtgattctcctgcctcagcttcccaagtaggttaactttttgatactagacagtaagcttggattaagtattttccataataatgctcattcatccatcccggcagcatccacttcaggttctgggctggggtgagggatcagccatccaggacaggatctggaatttgctctgcgccttttcagaccccctccctctcctcctccagtaagaaggtcaacaggacctgcttacgggccccctattactgaaagcaaactgaacctccagttagtgtcctgcccaaggtttcaaaggtaagtaagtggtagagctagggctcttctaaccacctgcagagctaggcccaggctgccagatgcctccttgagggcataggtgtggcagaggcgtggctggtgctggagggtcgactgaggcctgtcccactgctgctgtacaccgatgaatgtgggctcaaaggctctggggaaggtaagaccaccagggagctagtgtggcctgggggttcccgggggtgggcggtctctccccaaggctggtctgggccaagcccttgctgctggggtgtgtcagatgctgccctgtgtcctgccctccagcgtcccaggaggagggggacagccagcaggcctgctcctgttaagagcttccccaccttagaaaaagctcaagctgattcccacagccttgaagccccatggtggcttctgggggctgctgtggcaggctcacctgtgcctcttcctgctctctcaccaggggaggccatgccatcagccttgctctccacgtaggtgtggtcacgccaccagagctctggcctttgactggtgtgtccagccctgccctgccccgacttgcctgtctctgctggatgcctgagggcaaagccagtaacagaccccactcaagctggttatgaggacctggcagggtggggtgtggcccagtacctcggaagactctagaaggaccatcctggccctataaatgtttgtgccaagacaggtcccctgtccctcagcacacagaaggggcagtaggtagcaagacagtttccaggtaacagcatgagggcaagtgaggcctagagaggaagggtctgttgcctccagcccgtcacattccctccaggcgggctgaacagccaggtgggataaagagggatcctcaggtaggcgggagtcagggagagctgcgtgaccccacgaggctggtgtttcatcttcacttctcagatgacgggactgtggctcagagacctttgcagggggactgatcacagcacagggccatttgaagggagcctggaactgcatttccatgtggaccctgggtggatctggaaccaggctggctttcctagctcccccgccctttggctcccttatgttcttagccttcttttcacaacaaactcagaagcagccaagtagttggttgtgttgtctacagttgcactgaagcacgttttatttcggctaagtgacagtcctttgttgtccctgtcttgtaattagttagggtagtatgttggttatattacgtggaatttattgcaggatggtaagggagcatgtgacaaaatggggcaatagttctgtaaggctgagagaagctgggggtgtacccagtggctaggctactcagaggggactgtcatgctgtcagtagtgtagtgggagccaagcaggctttactcaccggcctgttaacagctgtgcgagtcatgcctttcgcttgtgttagctcaccttgtgaaaaaatgagctgaattacagatgacaactaaggcatggagagccgaaccctttgtccagggttgtataggaagtggcagggtcagggctgaatcttaaaacctagagggtagggcccggcgtggtggctcatgcctaaaatcccagcactttgggaggccaaggcaggcggatcacctgatgtcagaagttcgagaccaacctggccaaccaacatggtgaaaccccagctctactaaaaatacaaaaattagctgggtgtggtggcaggtgcctgtaatcccagctactgcggaagctgaggcaggagaatcgcttgaactcaggaggtggaggttgcagtgagccgagattgcgccactgcactccagcatgggcaacagagtgagaatctgtctcaaaaaacaaaaacaaaaacaaaaaaccctagaggataaaagaactataacatctgggataaagttagctgttgaaataaatacaagtcctaccgttggggtagtgtcagagctggcctacgtctttgactttgagattcctagcagccagagcaaagaaagaaatagattattcccactggagccacagtggccataacaaacagaagccatctgtttagatgctgggaatggagaagacttgctccccagaaatagacgctgcaggatgtggtgggtgacagtctactgttcaaaccacaaatacctgaaatgatctggccactttgatcacccaaccacagtagagcctaagcaggtccagggagcagctgttgctggcctggcttgacttttgagggaggtggaatagcgggcatgtgagaggagggaacctaaagcccccacagctgctgacacagcccccaggtttgagcttgtctcccctctcacccatccccccatatagcttaatctgatgggcatctggtggtccttccctgcagtggccagagcctgagctgtccaggctgaggtttcgactgaagcaggggttgtcctgcgcccatgtgcccacacgctgcaacctggctggagcctctggcataggacacccataccaccaggtagctgtggcggaccccggctctgatacttggcaggcttgggttgagattgtggctctaggcctgggcggctctcaagggctgctgtggggagttggggaagaacccctgggttgtgctcagccagcaggcaggtggcagccaggctggctgtgggtgaggaggggaatcctcttttcaccactgcctgcctgctcagcagttcttcagctgctgggggtggagctggcttgcaggggccagagtcagagtgaaaccggcagctctgctggttaatctcagcactgacactggggactaggcctggagcggaactgaaagggagactaggcccgttcccctccaggcctggcctatatcctcccagtgatccttaaggcctgcagctagggggaagggtgggggccaggcagggccaactggaaaagtagcagaaggaagtggccactcgcaccttacccatcttgcagacaggaaatgaaggctctgcagggctggctggctgtaaaaatgggcaagagcgcagaggccccactccctgcacactgcgctgtgtccagtgcccgtgtggatgccatgtgaggcctcactctgcctgcctcatcctggctgctggacctctgggagtaggaggtgaaaatgccctggctggtgctggtcacttcaggctggaattttctagtcatgcagctttctagtgagaggccgcagtgagctgtggccagtgttggtcattcctgcagtcattactctcccccagcctgttgtgtccgccaaccagagcaggcccgggagctcccaggagacctgctggaagggcccaggtggccatcctgctcctgccaggctgccctcttacctcactgttcattgactctcctgcaggtggggaagggggataaaggaggtggccaggccactgggtcatgatccttccgacagaggtacctggcagctctgtgtgtaaagcaaggttaagcatctggttttgggggtcaattcaacagcactagcagctccagtccaaggtatgcccttttgggctgaggtggaggtcagatcacctgaagccacagttgctagaacatcctttgggctgcctctgctttccatccccctgctcccagtttgttaggggttgtagtattttccaggaacacctgaatggagggggcccagtggggccacttctgtccattccttatctcatctctttctccccttttcttctttcggccatcttttcagtcttatcacaagaggaaggggaggggcaaaccccctcctgagcagattcccaaccggaagcagctggctccttggctgcagagtgccccagcacaggaagcatctgcagggtcagttgagacctgggagcaggtttcctgctggcaaaggaacagtggctaactcctatccaccctgcagccctatgtgccgggtgtcctgggaaggtccagggccaagattttgtggttttacagtggctataacttaggctagccatgctaggctctgcccctttgggcttggtgtgatgagaccgtcagcagctgctatcacctggctgggcagggagctgtttgctcaccagtgggaagagaaacggatgtcagacacatggcagagtgggaatgtctccaagtccaggtccaatgggctgggagggctggccttctgccagcacatgcttcttgggatctcagtgtgaatggtgttcaggaggctaggggaaggcaccctggacctcactacaggctcagagcctgcccagcaacaccatggatactctctctaaaagggggactcctggggagactatagacaaactcaggcctggctggaagaaagagcatcaggaacaagccttgagtaccccaacttggtctcgtagagttcaaggcccaagctacaagcagcttgctccagcatccaggaggtccggtggtgcacacggcttgagatgcctgacaacctttacacctttgtgctgaaggtgagtgacaaggcttttcacaccctggggcaatacaaatacatacacatacagcagacccaaccctgttcccttcctccctgccaggtgaaggaccggacagacatcatctttgaggtgggagacgagcagcagctgaattcatggatggctgagctctcggagtgcacaggccgagggtgaggtcctgggccctcgtccctggcaccacctttgctgctaccacccctgacctgcccagatccttaacctcagcctcttctccagcaggctggagagcacagaagcagagatgcatattccctcagccctagagcctagcacgtccagctccccaaggggcagcacagattcccttaaccaaggtgggtaaaccaatagctaggccattgtcttctgggtacgctggaacccagactcagcccaggacataaggtagccccctgcgaccaccatcacccatcttatctaacaggtgcttctcctggggggctgctggacccggcctgccagaagacggaccatttcctgtcctgctacccctggttccacggccccatctccagagtgaaagcagctcagctggttcagctgcagggccctgatgctcatggagtgttcctggtgcggcagagcgagacgcggcgtggggaatacgtgctcactttcaactttcaggggatagccaaggtatggggtggggtggggtggggtggggcaggcaggaccgtgccacccctctccactggagttcagggtcctagagggacagcccgagcccaccatcctctcctcccacagcacctgcgcctgtcgctgacagagcggggccagtgccgtgtgcagcacctccactttccctcggtcgtggacatgctccaccacttccagcgctcgcccatcccactcgagtgcggcgccgcctgtgatgtccggctctccagctacgtggtagtcgtctcccaaccaccaggtctgaccctactgccctttgctgaagggggtggctgacactgggtagagggtagaggcttgctgcagacccaggggtacaggtatcttgttctgtgtcctgtcagcacttgcctcaagactgatggtgtggtctctcttggtcacttgtcctaggttcctgcaacacggtcctcttccctttctcccttcctcactgggattcagagtcccttcctcactggggttcagagttgggccttccccaccttagttcttctggctgtccccgggggctcagcccagagggtctcccagggcgatcctcaccccccgagcagatcttccacctggtgccttcgcccgaagaactggccaacagcctgcagcacctggagcatgagcctgtgaatcgagcccgggactcggactacgaaatggactcatcctcccggagccacctgcgggccatagacaatcagtacacacctctctgaccagtgaggaattccaggcctcaacagctgcccttgaggagcacaggcagaagtgtgaacttgtgaatgtaattgatctttccttccttccagagaaagatttaagggacactgttaactgctcgtgccagtttggaagtgacccttctattaggcctgttgaagggccctcctgtaggtttcatctatccacctggctttctccttattgtttacagatgtagttcttgttagaggatgccgctagctcctgcccggggtccctatgcccagtccccgttactcttagagaaaggagttggggtgagggccagagctggcagtggaaacttgttctctttttcactgacactgtcacagcggatgacagactttctacggggaggagggggggatcatcaggaagcccagaacactaacaagcggttctcccatctaccgtcagtccacatggcaggtctgctgtgtccacaccacagatgaccacatctaatcctgcttctactctcagctttaggacaaaagctctgtcagaggcacaagctgaaggtcaaaaatgatttaaaacattttacctcagactaatttctttaaaggattcaggttcaaaacttaaccactgcttatttcagtgcactgtttcaactaacacccatgctatttttgtagtcagaaacagctatgcaaaccctacctaatttacagtctgagccagcatgctggcttgtctactgcatcctcgggacagtcacctgccactgagtggccactgtccttcctaaatgtcaagaagtgaagtatgtcaccctttcagggaaattcaggcaattactgaaataggagggtggcaagaacagttctatcctggtgccttacgaataaaaaactggattctggtttacagcagctttacagtgatagttaaattaactggggctaggggaagagcaagcaaaaagggaagaaggactcctaggccctttctagtaaatccttcagcaacaaggctggcttggtgccctccaagcatctaatggcttattaaattatcccacaagtgggttttaggctccttttttgagccaaaatggaagctgggaatctggtgccataactaatgagaaactcctttaatagcccacaatcagtgttctgttctagctggctactgcttcactggattgagaatctatctatctccttgcacacatgggcacacacaatctccaccatccagggaggtcctgaagtcaaatctctatctatacaagtgatacaattcatagggggctggctcctcccagaacctgtctggaggctcagaaacgggggcagtgacagtggagtcagctgctcttgggtgccagcagagccattcagtacaacccccaggctcacagcagtggcttctaggaaactgggagtttagatcagctttacagatacatcgatcagaggctaaaatgaaacctcagcctaaaactcataggactgactgcctgggaggagggttaggtctgcttcttccacttatacttagtctctgtgctccaagaggtcaaatttttgcttctagaatttccttggggtctttcagagggtgggggaacaaacccctatgcacttttcttttttttttttttgagatggagtttctcttgtcaaccgggctggagtgcagtggtgcaatcttggctcactgcaacctccaccttcctggttcaagcgattctgcctcgacctctcaagtagctgggattacaagcaccagccaccatgcctggctaattttgtatttttagtagagacagggtttcaccatgttggccaggctggtctcgaatgtctgacctcaggtgatccacccgccttggcctcccaaagtgctgggattacaggcgcgagccaccgcgcccagcctacaccacttttagtaccaacactcttgggtgatttcatggaccctaaagcagacctgacactgatccagatttgcagtccatttttaaggacacctgtctttatttcctcaaagtcaagcagctttctctggaaaatgaatgctaattagtgtgaaccaaaagagtaagtaagagtctgaagtttttttaaaggagaaagcttattatggaaagtcactggtcctcccctccgcacaggaaaggtacccagtagataatgaaccaaattaagttccctccctccagccagaagttaaacatctgggatatgacgtcttcatgccaggggcactcatttcttagcagcctctctacatacatctctcaggtggtgccaagaggcacaccaggtagagcaaacttagcagctctgactaacaggctgcaaagtgcaagttcagattctgtggcagagatttggaaggcacccacctccagactgcttcccgtccaagttaccaggacagctcaaaaacatgctgacagaaaactcccatggctctaggaagaagtgacactaagccaacacctttctttatgtgggagcagaatcagctgatgaaggggtgggcagcagtgtggggcaggcaccccactggctgcagctagcccaccataggcacagcacatcccaccactctccttccagtcctgaccaggccccagccggcaacttctaccgagagccatggctcaacaccaaactggacagtagacatcatgatccctccagttagctctaattacagaccccaccagtacagcttgacagctcccggcaccatcccttccttcatctgacttattgaacttttacaaactaacagtcaccagcaccaaagaattaagtcaactaacctgccttgaattttagaccagcaatccatatggctttatctggtataaatcttctgcctttgatcatttctggaccgtaggaaaaaggaatagcaatcattaaaatcttgggccagagaacactatttttacataacagtttcttaacctaaagtcaaggccttggactcttccctgagggttgcctgagattccttcatgctttctattcaggactaagtcccttactgcaaatgtgttagctctaacatctcccacaagctagaggaacttgcgagtatattaacaaggacacatctgacatcctgtgtttggttagaatatacagcacattgtgataacataaagtggattcatcttgtatcattataggcagaaggtatttggcaaatttttatgtattgttttatgtactgtacaagtaacttattcttgaataatgcaaattttgctataatgtacaaattgctatatgtgaattaaaaagttttcagaatcttga

SEQ ID NO: 23 - DNA, 148136 bp, Homo sapiens
agctgctaactcgctctagtagctgtggtcggggctcgggaccggctgccatcttagtctagggactgaggagtcgccgccgccccgagtcccggtaaacccccgtgggcgaactcggggcagttctcgggagagggagggagtccggatgagaatgttcggggagaacaagggtagaggagtgggacgcgcggttcgggaccccagagccgcggctgctccttcttccagagccggtgtctgtcgggggcgcggggagctcgggctactggcccgggccgggcggtgggggtgggctgtctggacgttcggagggatcagtgtctggcacaggcttccggaggccttcgcctcgcgggctccaagttgtgaaagggctgggccacgacgggcgtgggtaccgtccggcccctcgcccctcagccccgcctggggccactctccagcggttcggcctacgcgcggggcccggcggcggggccggcgcgggcgacgtggccctgtgaggggcggcggccgggtcccccgccacccagctggccttctcgagacttttccgagcccccagtcgctagaagggcccagcacagcggctgcctccccttctctctcccactcgcctcctccttccattgtccccccggcgtggtggtcccggctccggcgccctccccgacgtccgccaccgtcgtggcggcgccgacttttgcagacgtggagctgtcactgcccttcctccggagagtgcccttttctttagcagagaaaagaaaaaagtagccgttgaacgtattctaagggtcgaagcatgatgacatgacttttaagtggagaactcaagttttccaagacagttcccgtccgcgacagaaaagagaatcgcagaatttgtgcgcccgaagtagacgctgggcctaaagttagttggacatgtaaccaggtgaacttgtaaagggccaggaattcattatccccaataacaacgcaaccttgttttcttggttgtattcgtatcctaatcttattacattttctgccattcttccacagctacagtatacttggtttggagtgggggatggggaattgtgggatttttgccttgaaaattagtaagaccctggagccagccacccacttaggtgggttctcctgcatcttttcctgatagaattcgatcttcaggaagtatgtaaaggcagtgtatttccattctcaaaatcggctaaggaatggtttatcagttgaggtgaagttctcatggcagatctgctgagagcttgctatgtgtatttacaggtttatttaagctacgatttatttttaaaattaagaaatgggccgggtgctgtgtgtggctcacgcctgtaatcctagcactttgggaggccgaggcgggcagatcatgaggtcaggagttcgagaccatcctgaccaacatggtgaaaccccgtctctactaaaaatacaaaaaaaaaaaaaaaaaaatcctgggcgtggtggcgcgcgcctgtagtcccagctactagggaggctgaggcaggagaatggcttgaacccgggaggtggaggctgcagtgagccgagatctcgctgctgcactccagcctgggcgacagagcgagactccgcctcaaaaaaaaaaaaaaaaaaaaagcaatgggatgggtgtggtggctcacgcctgtaatcccagcaatttgggaggccgaggtgggcggattacgtgagcccaggaattggagatcagcctgggcaacatcgtgaaaccccgtctctacaaaaaatacaaagatttgccgggtgtggtggcaggcgcctgtggtcccagatactcaggaggctgaggtgggggaatcacctgattccggggggtcgaggctgcggtgagctgttaccgttgccactgcattccagcctgggtgacagagtgagaccctgtctcaaaaaataaaaaaaaaaatttagaaatgaaggtggaaattctactgcttctttgaactgtgaatttaagttttgtgagtccaaaaggaattccattacattcagcatacaggtcaagtcaccaaggatatcatatgaaattaaaaaaatgagtacagactacttcttcagtgtattctagaatcaaacatttctacattctttagaaaaaaatacatctttttcgtttgtttgagacggaatctaactttgtcacccaggctggagtacagtggtgtgatctcggcttactgtaacctccgcctcccgggttccagcaattcttctgcctcagcctccgtagtagctgggactacaggcgcgtgccaccactcccggtaatttttttgtattttttagtagagatgggttttcaccatattggctaggctgctctcgaactcctgacctcgtgatccacctgccttggcctcccaaagtgctgggtttacaggcatgagccgccactcctggccctaaaaaatacatcttttcacttattgggagaggcagacaaaccagtagtccccaaatttgaccattagaataattaggagagcttagaaattccagggtcttaccttctgagatggatttagtaggtctgggttggcttcaaccaatttatttttaacacatgccttaggtaattctgatggtcttgggaaccactgagggcaaatggtactgtagacttaccaaggaaaggtcaaaaagctttgagacgaaagaggaagtggccaattctgtgtgtgtggctgggtggaggattggttagaagaggcaagatttgcccctgaacccacattgggtcttacatgaagagtaggagtgtgaaaagggcggggaaagagaaggacagggtaggctgagtgaatgccttaatgctgtgtgtaaattcagaggattatggaacaaaaagggttcagggagtgtggagtggaatggggaaggagagcattttgaaacgcaaggtcagaaaggttaaaggaagcaagtatatgaaaagtgattttttgttgttgagactgggtctcacccagtcacgcaggatggagtgcagtgacaggacctccagagctcaagtgatcttcccaccccagcctccctgagtagctgggacaacaggcatgcattagcatgcctggctaatttttaaaatttttttgtagagaaaggttctctttacgttgcccagtctggtcttaactcctgtgttcaagtggtcctcccaccttggcctcctaaagtgttgggattacaggtgtaagctactgtgctctggctaaaaattttaagtataatgctaaaaatttggaattttatgttggtagaaatagatacagggctgggctcagtgcctcacacctgtaatctcaacagtttgggaggctgaggcgggtgaatcgcttgagctcaggagtttgagaccagcctgggcaacatggtgatatcccgtctttaccaaaaggaaggaaaaaaaaaagaaatagatgcagatttttcgacagggaaatgatgtgagtacatatgtgttttagaaaattgctgtgtactgcaggacaaagataggtatcaaaaaacaaaaaaagaaagattgctctggtgatgagcgaaaggatgagggactagctattgcagaagtctagttgagaaatgacaaagcctgaccttggagagaggttgtggggatgatcaaaggattggatttagctttttactagtgcaatattgttgccatatttctagatgaggatttttttttagagctaatctgggtaaagggctccttttctgtcaaaagaacccattagcaagtctgaataatattgccaggttatatatgttatgtatagtatgagagtgaattgttcattagcatctttgagaaattagaaagaactatgtcaggtgtaatttggaagagaacttgaagtttgttattgttactatttttgagatggagtcttgctcggccatgcaggctggagtgcagtgatgtgatctcggctcactgcaacctccacctcccgggttcaagcgattcgtgtgcctcagcctcccaagtagctgcctcccaagtagctggaattagaggtgcatgccaccaagcctggctaatttagaacttggagtttggactgaaatttatgcctctaaaaagtattgatctaggcttgttgggagtgtttttctctcagcctgtagaaacttgctcattgagttttagagttgggagaagttacagagattatctagtccataatggcaaatatattttggtttattggccagctctaattggaagctgccagaagcactgtgtttggaaggattctggttgtaggtgctctgagaaattgtattatggttgatcaatgatgtctcctgtggacagttacctagtaggtagtagacatgtgccaaatatatgctattcttagataagtccaatctcttagttttctgttttgttgttgtttgtttgttttaaaggttaaataatttacctgaaactagttaagagtaagaactagaactagaactgggttttgcaactctgggactggtattctctggtttacaaagatcaatgtgcactactgaagagcttctggtattagactagaaaaagaaaagggagatttttagcctatgcgtcttgactgtttaggcaagagtttaacaaggaattttggcctagaagtaagttttgttatattcattctgtcttaagaaatttgtgttagggccgggcgcggtggctcatgcctgtaatcccagcattttgggaggctgaagcgggcagatcacgaggtcaggagatcgagatcatcctggccaacgtggtgaaacccccatctctactaaaatacaaaaaattagctgggtgtggtggcatgcacctgtagtaccagctacttgggaggctgaggcaggagaaccacttgaacctgggaggcggaggttgcagtgagctgagattgtgccactgcactccagcctggcaacaagaatgagactctgtctcaaaagaaaaaaaaaaaaaaattcgtgttagtactgataggaaatagtcatctgttttgttgttttgccactttctttcttgttgcctaagtctctgttttgggttttggacattggtttcctttataagaattaagacaaaagtatgtaaggtttttttttttgtatttatagtacttagccaaattctgggctcataataagcactcagtaaaggcatgttgacttttatggctgtaaatgatactggctttgagtcaatataaattagttagggggttcacagaattgaattgcagcattctctggttgggaggttggctttcagctaaaaacgttaagttggaaactatcctcacaactagaagcagttaaccagaagctgttcctgcctctatcctcctccagtctagcagtgtactgtccttgaaaaactgctgccagctcagcttttttgcttcttttcagaactgactatatccagatccttggtgtgcgttttcttttcctgtccctaggagtctccaagttgcagggactatcattatggtgacccttataggctggtgtatttatatggccatcctcattttgccagatagccgtcatactcagattggtgtcttcccataatacgtatgaatcattttccctcagttctttcttttgtcctgtctgctccattaaaaattgtctatcccatgaaatttttaaaagagactttgtgcttttagagtcgcaaggtcgttgcagatctgatgtgtttaactatagaggtcagctgggtgtggtggctcacgcctgtaatcccagcactttgggaggctgaggtgggtggatcacgaggtcaggagttcaagaccagcctgaccaatatggtgaaaccccatctctactaaaaatacgaaagttagccaggcgtgggggtgcacgcccgtagtgtagtcccagctactcaggaggctgaggcagaagaatcgcttgaacccaggaggcgaaggttggagtgagccaatatcatgccactgcactctagcctggcgacagagcaagactccatctcaaaaacaaacaaacaaaaaaatatagaagtaatgtgttaatggactctccttcctttctcaggtaccatgcatttcacggtggccttgtggagacaacgccttaacccaaggaagtgactcaaactgtgagaacttcaggtatggtgtcatcctgtgagagaaattgggggcaagagtgatgcagaggccaagctctggtcttatacctgctgtgaggagagagcagtgttgatgctagctgcatcagttggttgaacatataggcagcttcacttggttctgaaacccaaagctgcctctgcagggattagcctgtgagccacactgtgctcttaatttgggttttctgacttttctgtttttgtagatcccttatacatatgcatactttttctatttgtgttgttttatacacgtttatctctgtatactatctagacatgtgcatgtgtaggattaagaggtgggtaatgaaaaagaagacaacatggccaggtgcagtggctcatgcctgtaattccagcactttgtggggcgaaggtgggaggatcacttgaacccaagagtttgggaccagcctggacaacatggcgagaccctgactccacacaaaataaaaaaaaatcagctggtgtggtggcgtgtacctgtaatcctagctgattgggagggtgacatgggaggatcacttgagccaggaggttgaggctgcagtaagccatgatcacgccactgcactccagcctgggtgacagagtgagaccctgtctcggaaaaaaaaaaaaaaaaagacaacatggttcagtgcagaaagaatagaggacttggaaagaaaagatctgggttccgatttgattttgccactgacaagctgtgtgactttcggcgagttatttaatcttgccagacctcagtttatacatctgtaaaataggtaagtattgattcctgtctccatccctggcattgttgtgtggatcaaataaaataatacctattaaatctttagaaaaggaaatgaattcatgacttaaaatgttatcttggccaggcacggtggctcatacctgtaatcccagcactttgtttgggaggccaaggtgggcggatcacttgaggtcaggagtttgaaaccagtctggtcaacatggtgaaactccatctctactaagaatacaaaaattagcagggtgtgatgtcatgtgcctgtattcccagttacttgggtggctgaggcaggagaatcatttgaacccaggaggtggacactgcagtaagccaagatcgtgccactatactccagcctgggcaacagagtgagagtctgtctcaaaaaaaaaaaaaagaaaagaaatgttatctagatccacaaggtgagagaagttgagagtagtattgtgaaggaaaacatgaggacaagacagatcactttaaaaggtcaggagagaaggaagactactgggtaagaaaaaaaaagtagaacataaagttaagcagtaagagaatagggagtgaatgaagttgagcaggtgaattgttagataagaaagctgaggctgggcgcggtggctcacgcctgtaattccagcactttgggaggccgaggcgggtggatcacgaggtcaggagatcgagaccatcctggctaacaaggtgaaaccccgtctctactaaaagtacaaaaaattagccgggtgtggtggcgggcgcctgtagtcccagctactcaggaggctgaggcaggagaatggtgtgaacctgggaggcagagcttgcagtgagccgagatcacaccactgcactccagcctgggcaacagagccagactccgtctcaaaaaaaaaaaaaaaaaaactgaataggaagatactggtaaaatagaactaaaatatgatccagcattgccacttttgggtatatatccaaaggaaataaaatcacttcttctttttttttttttttttttttttgagatggagccttccccaggctagagtgcaatggcacgatcttggctcactgcagcttctgccccctgggttcaagtgattctcttgtctcagcctcctgagtagctgggactcgggaggccaggcgtgcacccaccacacctggctaattttgtattttcagtagagacggggtttcaccacattggccaggctgctcttgaactcctgacctcaggtgatctgcccgcctctgcctcccaaagtgctgggattataggcgtgagccactgagcccggtcctgttcttttttataatcaagagaaaggaaggagatggtggaatgatttgtgatagctactataaagcaatttaaaaaatatttaaaagtattattaaagtatttaaaatatctttaaaatatttctcagcatatccatttcatctcaagctcacaaagcagttctttgtggaaaggtatttgagccaacattcaggaaacctgcgttctaccattggtattactggtcacttaaattctgtgatcttgggcagctcacttaaatctccctgggcctggtttcctcacttatctattgaatgttttgaatatgatttctcaggttctttccagctcactctattatattctgtgctttatgtgctaactcactgatttttcatggctttgttgacatcacaatatatgactgatatcagaaatatcttataataaggcttcggccggccgcggtggctcatccctgtaatcctagcactttgggaggctgaggcgggcggatcacgaggtcagaagatcgagaccatcctgactaacatggtgaaaccccgtctctactaaaaatacaaaaattagctgggcgtggtggcgggcgcctgtagtcccagctactcgggaggctgaggcaggagaatggtgtgaacctgggaggcggagcttgcagtgagccgagttcacgccactgcactccagcctgggcgacagagtgacagagggatactttgtctcaaaaaaaaaaaaaaaaggcttcataatgtctgatgctttataaatgagaaaccatgttattttaagttctacttttttttttttttgagagggaatctaactctgtcacccaggctggagtgcagtggcgcaatctcagctcactgcaacctccgcctctcgggttcaagcaattctcctgcctcagcctcctgagtagctgggattacaggcacccgccaccatgcctggctaattttttgtatttttagtagagatggggtttcaccgtgctagccaggatggtctcgatctcctgacctcgtgatctgcctgcctcagcctctcaatgtgctgggattacaggcatgagccaccgtacccggccttttaagttctactttaaacattggcactgagagacctgtaggaaacgtaagtatagaaagtttaagtttgtaatatttgacaaggatctgttgagctgctattgtgctaggttccaaggggtactcaaatataaaactcctggtttaaggattttgtcaggaaaacagacttccttgccagtgattgattattataaaaaggaagttggtaaatgttacaagagtttcattgcattgcaatttaagaaattaagttcagattttttggttcctttaacatgattgaatgtattccttttttttttttttttgagacggagtttttctcttgttgccgaggctggagtgcaatggtgcaatctcagcttactgcagcctccacccactgggttcaagcgattctcctgcctgagtctcccaagtagctgggattataggcatgtgccaccacgcccagctaattttgtatttttactagagatagggtttcactgtgttggtgaggctgatcttgaactcctgacctcaaataatccacctgcctcgacctcccaaagttctggattacaggcataagccactgcacctggccaaatgtattttttttttttttttgaggcagagtcttgttctcgcccaggctggagtgcagtggtgtaatcttggctcactgcaacctccgtttcctgggttcaagccattctcctgcctcagcctcctgggtagctgggattacaggtccctgccaccatggccagctaatttttatatttttactagagatggggtttcaccatattggccaggctggtctcaaactcctgacttcaggtgatccgcctgccttggcctcccaaagtgctggaattataggcattcggcctaacataattgaatgtattttaagtgctgctgtatgctagacattgcagggacttatggtgaaaaagcaatctctgccctccagcaactcgcattttttttttccatgtctggaaattgcttaccctaatagatctatccttctgtcccagaaaagagaagaattttaatactatgtcgtcacttacaggttaagaattcatctacatattgagcttaactcactccagaaatatctactaacttgcttgctttctgagaaaatgtttattattttactggattctgcaaccttttctttgtcagttacaagagaacattttttttttttatttttttttaagatgcagtcttgctttgtcgccaggctggagtgcagtggcacagtcttggctgactgtaatctccgtctcccaggttaaagcgattcccctgcctcagcctcccatgtagctgggactacaggtgcccaccaccacgcctggctaattttttgtattttagtagagacgaggtttcaccatgttggccaggatggactttttattttttatttttttaagacaagataagctccagaaataaccatctgcatttaggtgaactttgatttgcttgttaggccttcattcgtgcaaagaaagagtaggttctacaattttgtcttgattatttttccaaataatcaaatttagttggtcacaaaggaaaggattttagtctaaaggattatattgtaggagttaacagtatttactggagttaggttgattttgaattttgactctactgtgtagtggtggtatgatcttggccaacttgcctaacttctttttgccttagttttctcatctgtaaattgggaatagtaatagcatatatacataggtattatgtgtttctgcttgaacaagacttggctcacagtgtgaagtgttagtcgttgttttcattatcatcattaccatcaaccttatctttcttaaagttatgatctgagttcataaatcattcagtaaacctatttaggctgagcattgtaaatttaaatgcctgcaagggctggatgaacatttagtaagtaatgacagatcagactgggactttaagtgaactagggaattcattcccctccaagggagcagctgttaaagcagctttggcttacaattttttttttttttttttgagacagagtttcacttttgttgcccaggctggagtgcagtggcatgatcttggctcactgcaacctcaacctctgcctcccgggttcaagcaattctcctgcctcagcctcccaagtagctggaattacaggtgctctccaccacgcctggctaattttttgtatttttagtagagatagggtttcatcattttggccaggctggtgttgaactcctgacctcaggtgatccacccgcctcggcctcccaaagtgctgggattacaggtgtcagccaccacgcctggcgtgatttttttttttttttgagacagaatctcgctcttgttgccccagctggagtgcaatggcgcaatcttggcttaccataacctccgctttccgggttcaagcaattctcctgcctcagcctcccaagtagctgggtttacaggtgtgtgccaccacgcctggctaattttgtatttttagcagagacgaggtttctccatgttggtcaggctggtctggaacttccgacctcaggtgatccgcctgcctcggcctcccaaaatgctgagattacaggcgtgagccaccgcacctggccttttttttttttttttttaatggaggtaaaagtaagtatgtgaggttatgtatatgttatttagctcgatttagccattccacaacatgtacatattttaaaacatgttatgggctggtcatggtggctcatgccataatcccagcactttgagaggctaaggcaggaggatctcttgaggccaggagtttgagaccagcctggataacatagtgagaccccatctttattttatttcaataaataaataaaacctgttttacatgataaatatataccttttttgtcaattaaaataatttaaaaatactacttaaggtcttgggaatacactcacaatgtattggtagttaaaatttttttttttttttttttttttttttttttttttaccagttgctgtggctcacttctataatcccagcactttgggaggccaaggtgataggattgcttgagcctcggagtttgagaccagcttgagcaacatagcgagatcccgtcattatatttttcaaaattaaaaaggaaagttttcagaaggttaaaaaaaaaattgaggcaaaattcactactttaaagtgtacaattcagtgttggcttactattgaccttggggagagtgcagaccatgtgcagactgaacatcttaatttttttctaaggagaagctagaaatcaagattttgtaagaaatttttattgttaaatgttggcaacttatgaattcatagtgtaaacactgtgggccaaacaaattaccactgtgggcagaatttgtcttgtggcttgctgctttacaacctctgatctggggcttgactttctgcagcataacccatgttttctgccctctgagaagaaagttcttacccacgtaaagatttaaccagcctcggcagcatggtgaaacactgtctctacaaaaaagcctcataaaaatcagccaggcatgatggtggtgtgcgcctattgtcccagctgaagtgcaagggtcgcttaagcccaggaggttgagactgcagtgagctcatgtcgctgcactgatatcatgtcactgatatgtcactgcactctagtctgggcaacagagtgtgaccgtgtctcaaaaataaatacataaataaataaataaataaaacagttcaaacaatacctcaaagcagtctagtactgaatgagaggtatgtagaataattattaaggcagatagaaggagggaagttagttagttattcagaatatgccaaggtgcagaggcaggaatggacttcagggggtggagtttaattttagaggatttgtgtgagcctggtaataggcaaaagttttgaaaatttgagaagagtcaagcctcttattttgaataaccttgagacctattggctgataggattaaatggtattttgtgaagatttatttggcaaaattgaggtctgtagtatttactatggttggaggcagggagactaggctatggaggtgtcagtgggaatgaaaataaagggggaaaagcttgtataagagacaaaaggttgcattaaagagggccaattttgaaagctttttctctattgacttttgtacaaactgggatatgggaagatactgtgtacaggctatattcctcttttcatgttgtgaatcttgctttatcccttactcagtcccaacagtgtccttgttgatggattacatacgttttcttacgatttcccagcttgcacaagtagaatagaaagaaaaaccaggccaggcatggtggctcacacctgtaatcccagcactttgcgaggctgaggtgggcggatcacttgaggctggagtttgagaccagcctggccaacagggcgaaacctcgtgtctactaaaaatataaaaaattagccgggtgtggtgttacactcctgtagtcctagctacttgggaggctgaggcatgagtattgctggaaacagggaggcggaggttgcaatgagccgagattgaactgctgcactccagcctgggcgacagagcgagaccctgtctcaaaaagaaaaaaccaccaaagtagtgtaattgctcatcacatttcttgtttggggtttttggttatctcggttaacctctggtatatttcattagaccttagtttatacattgaattttcatatttaccaaggtttggaaacaatgacaaatggcttgctcttagaaagatgtagaattttcttgcagaaacttgagataactgatatggctgggcgcggtggcttacacctgtaatcccagcactttgagagtccaaggtgggcagatcgcttgagcccaggtattcaagaccagcctgggcaacatggagaaaccctgtctctacgaaaaacacaaaaaattagttgggcatggtggcgtgtgcacctgtagtcccagctacccaggaggctgaggtgggaggattacctggtcccgggaggtcaagactgcagtgatttacctgtgattgtatcacagtactccagcctaggcaacagagtgagaccctgtctcaaaaaaaaaaaaaaattatgattcagaatgtctgaagaaaccaataaatgtagaaaggtagaatatgttcagcataacctgagaaagtgatttttaggatgttattttctaaataagatttagattttgttctgtttatacttctcatgaaacttttttttttttttaacatacctgggaagttattcctcattcttggccaaatacaaaatgtcattattttagagactatagtctttttcgcaagtaagctgaatagtgtgttaggtatagtctgagtattaagaatatctgagatctgtattaaaaacagatgtaggccgggcacagtggctcacgcctgtaatcccagcactttgggaggccaaggcgggaggatcacctgaggtcaggagttcgagagcagcctggccaacatggtgaaaccccatttgtactaaaaataccaaaaaattagccgggtggggtggtggcgcacctataatcccagatactcggtgggctgaggctggagaattcctgaacccaggaggcagaggttgcagtgagcagagattgcgccattgtactccagcctgggcgatgagcgaaactctgtctcaaaaaaaaaaaaaaaaaaaaaaagatgtaaaaaaaaaaaaagaatatctgagatatagcaaagtttttctcatggatgaataaaaatactgaactgggcctttaaaaaagtacagaaaattgcctgtcatagaaatgcagcagtagggtcaaatgagctaaatctaaatctcatttaatgaaaaaggcactttccttttatttttgaaggatggtctaacctagtttttggaattcatttgttctgcaattaaaagagatttttaggtggttcatttaattatagctgttttggaaatgggaaggaaaataatgtgagagagagattaactgcatttgcttctgtggaaagtcagtagtcaaaaggagagtctggattatttatttatttatctaaaaatatggaacacttggtgaattttaaccttatctttgctcagaggatgtactaatctctgtgttctaattttttagtatatgtgctgctgaagcaaacacacagattatttttttctgttcttaagttttttggaggaaagctatttactataggtcttaattaaaaactatattctgtatatattgaaatgtgcagaaccctgctgctgctgtttttcgttacccagaatgttgcttacatctctctactatcatcttctctccctttgttttagcccttggggtttatgtttttttattcctttatttcatttttttctgaggttttggaagggaatggagaatggggtgtgttcattctgcctggctggttgattttatatagaaattgatgttttcatctctaagggacaaagaaaattaggaaaatatcctagttttatttttattgtggtaaaatacacattacatacagtttaccattttaaatattgttaagtcttcagttcagtgacattaagtacatttaccttgttatgtgaccattaccactatcggtctctagaactttttcatcatccagaactgaaacttgtacctattaaacaataattatccatttcacctcctccctgctctggtaactactattctgttttctgtctctgtgaatttagctattctaggcacctcatcgtaaaagtggagtcatacaatatttgtcctttttttttttttttttttagacggagtctcgctctgtcacccaggctggagtgcagtagtgcgatcttgactcactgcaacctccgcctcctgggttcaccccattctcctgcctcagcctcccaagtagctgggagtacaggcacccgccaccactcccggctaatttctttttgtgtttttagtagagacagggtttcactgtgttagccaggatggtctcaatctcctgacctcgtgatctgcctgccttggcctcccaaagtgctgggattacaggcgtgaaccactgcacccggcccccccccccctttttttttgagacagagtctcgctctgtcaccaagctggaatgcagtggtgcgatgttggctcactgcaacctccgcctcccaggttcaagcaattctcctgcctcagtctcccgagtagctggaactacaggtgtgtgccaccacgcccagctaatttttgtattttttaatagagacagggtttcaccatgttgaccaggatcgtctcaatctcttgaccttcctcagctgggattacagccgtgagccactgcgcctggccaatatttgtccttttgtgtctggtgtatttcacttagcataatgtcttcaggtttcatgttgtaccatatgtcagaattttattcctttttaaatgctgagtaatattccattttatatattttagtaattttttgaaggactgaataggctcaagtacagcaatgacatattttttgttcatttccctgttatcttttttgctgtgaactgtgattttaaatcatgaaattaacattgaactatgctagagatactagttttgggccacatcttctgttctgaccttaggtaatccactttgcttgggttgtttgtcttactcagttgtaagagtttattgtaaattctggatacaactcctttatcagatatgtgttttgcagattttatctcctagcctattgcttgtctttccattttcttaatgatgtcttttttttttttttttttttttgcaaaaatagagatggggtcttgcattgttgcccaggctggtcaaactcctgggctcaggtgattttcctgcctttgcctcctaaggtgctatgattacaggcatgagccaccggccctgcccaagaagttgagtgcaatcaaaattagaaactttgacccttttttaatttatttttgagacagagtctcactctgtcacccaggctggagtgcagtggcacaatcgtggctcactgtaatctccacctcccgggttcaggagattctcctgcctcagcctcctgagtagctgggattacacgtatgtgtcactgcacctgggtaatttttgtatttttagtggagatggagtttcaccatgttggccaggctggtcttgaactcctggtctcaagtaatctacctgcctcagcctttcgaagtgctgggattaccggtgtgagccactgcgcctgtttgaaatttcaactctttagaagacatcattaagtccaggcatggtggcttgcgcctgtaatcccagcagtttgggaagctgaggcgagaggatcgcttgagcccaggagttggagaccagcctggccaacaaagtctacaaaacgaaacaaaacaaaaaaacaactatctggacgtggtggtgtgtgcctgttttttttgtttgtttgttttttgagatggagtcttgttctgttgcccaggctggagtgtggtggcgcaatctcagctcactgcagcctccacctcccaggatcaagtgattctcctgctgggactacaggtgtgtgccaccatgcctggctaatttttttttttttgagatggagtcttgctttgtcgcccaggctggagtgcaatggcgcgatctctgctcaccgcaacctccgcctcctgggttcaagtgattctcctgcctcagactcccgggtagctgggactacaggcgcacgccaccatgcttggctaattttttgtttttttagtagagacggggtttcaccatgttggccagactggtctcaaactccttgcctcgtgatccgccctccttggcctcccaaagtgctgagattacagacgtgagcgaccgcgcctggcgtgtgtgcctgtatgtagtcccagctatttgggggagaatgaggcagtaggatcccttgagcccaggagttccaggctatagtgagccatgatcacaccactgtaccctagctctgtcaacagagcaagtccctgtctctaaaaaaaaaaaaaaaaatggaaagaaatactcaaaacactcaatttctcaattgttttctttaatgaataacgtgtttggtgtcatatctacaaaaatctttgccttactggacaatcagtaaatctttcttagatttccatttgtttacctgtgagaacaaataagatgtatgtggtattttgccttctttggaatagtatttctattctgtttctttttgaatttaatcggaggggctaatcttgggatagaatgctaaggtgaaaatctacagactcaaattggaatgtagaattgactttgctttcataatcctagttgtttcctaataggtaagtggtaatttggttcctctccccttcctagtattgagggctcttggatgtaaactcttatttgtgtcattttattctaggtttttgacacaatctgaataacaatgctttttggctccttttaaggaaaatgcatgaattttgttaataaagtgcatatacatattactcagtgactcagtattgactttcagtttacttgataattcttttttcttttcttgagatggagtctcgccttgtcacccaggctggagtgcagtggcgtgatctcagcatactgcaacctctgcctcctgggttcaagtgattctctgtctcagcctctcaagtggctggaattacaggcgcctgccaccatgcccagctaatttttttgtatttttagtagagatggggtttcaccatgttggccaggctggtcttgaactcctgaccacaagtgatccacctgccttggcctcccaaagtgctgggattataggtgtgagccaccacgctcagccaataattctttttattattactattattattattattattattattattattttgagacagagtcttgccttgtagctcaggctggagtgcactggcatgatctctgttcactgaaacctctgcctcctggcttcaagcaattctcctgccttagcctcctgagtagctgggattacaggcacgcaccgccacgcctggctaatttttgtatttttagtagaaacggagtttcaccatgttggtcaggctggtctcaaactcctgaccttgtgatccgcctgcctcggcctcccaaaatgctgatattacagccatgagccaccgcgcccagtccttttttaaattttttttcagacagggtcctactttgttgctcagccgggaatgcagtggcatgatcatggctcactatagtcttgacctctcgggctctggcgatgctcctgtgtcagtctcctgagtagctgggaccacaggtgcataccaccatgcctgggtagtttatttttattttttgtagagatagtgtcttgctatgttgcccaggctggtgttgaactcctggtctcaagcgaccctcctaccttgacctcccaaggtattgggggtgggattacaggcgtgagccaccatgccagtcttgggcttgatagatttttttttttttttttttttgagacagagttttgctcttgttgctcaggctggagtgcaatggtgcaatcttggctcactgcaacctccgcctccttggttcaagcaattctcctgcctcagcctctcgagtagctgggattacaggtatgcgccaccacgcccagctaattttgtattatcagtagagacagggtttctccatgttagtcaggctggtctcgaactcctgacctcaggtgatctgcctgcctcggcttcccaaagtgctgggattacaggcatgagccactgtcactgtgcctggctgatatatatatgtgtgtatttattattattttttttaatagaaatggggtttcactgggttggccaggttggtctcggaactcctaggctcaagagatcctctcaccttagcctcccaaagtaccaggattacaggcatgagccaccaggactggtctgtaattcttttttttttttttttttttttttgaggtggagttttcactcttgttgtctaggctggagtgcagtggcacaatcttggctcgtcgcaacctctgcctcctgggttcaagctattcttctgtctcagcctcccgagtatctgagattacaggcatgcaccactgcacccggctaattttgtatttatagtagagacgggatttattcatgttggtcaggctgctcttgaactcctgacctcaggtgatccacctgcctcagcctcccaaagtgtttttggattacaggcatgagccactgtgtccggcctcttttttttttttttttttttaagtcaagtgaagcagtgggagtggagaaggaacaaagaaatctgtaactggttgtgatcagttagttgtaaataccagtgcagcagttggaccagccgaggcctgtaattcttttttttttttttttttttgagatagagtcttgctctgtcgcccaggctggagtgcagtggcgtgatcttgtctcactgcaacctccgccttccggcttcaagcaattctcttcctcagcctcctgagtaactgggattacaggcatgcgccaccatgtccagctcatttttgtatttttagtagagacggggtttgaccatcttgggcaggctggtcttgaactcctgaccttgtgatccacccgcctcagcttcccaaagtgctcagattataggcttgagccgccgtgcccagctgcctgtaattctcaaataatgtttagaaatgatctgaggaaagtttgctggaaggatgaggcatggtggatattagggtcttttttttttttttttttttgagacggagtctcgctctgtcgcccaggctggagtgcagtggcatgtcctcagctcactgcaagctctgcctcccgggttcttgccattctcctgcctcagcctcccaagtacctgggactacaggcgcccgccaccacacctggctaattttttttgtatttttagtagaggcggggtttcaccgtgttagccaggttggtcttgatctcctgacctcgtgatccacctgcctcggcctcctaaaatgttgggattacaggcgtgagccaccgcacctggctattagggtcttatattttgagaatgtatagatatttagagatggtcagaaacaagaaaattttttttttttaagaccgagtctcactctgtcgctcaggttggagtgcagtggtgtgatctcagctcactgtaacctctgcctctgagattcaagccattctcctgcctcagcctaccgaggagctgggaccacagtaggcacatgccaccacgtccagctaattttatgtgtgtgtgtgtgtgtgtgtgtgtgtgtgtggttttttttttttttttttttagcagagatagggtgtcactatgttggccaggctggtctcgaactcctgaccttaggtgatccgctcaccttggccttgcaaagtgctgggattacaggcatgaagcatcacctctggccagaaacaaggattccttagagccaggaaattcaacattatatataattagatgtataatttcacacaagcattgtttgcaagagaacaatacttatagtagtacaacaatatttatatttattgtggcaaatgctttaaactctagcagtagtcaaagctagaaacaacttgtaataatagttaattttgaaatgtttgtcagaaagctgtttgtcagaaaggggaagagatcaccttctggaatattaggtacagagttcaaaccagcaaatgaatgctaaaagttagatcctggtgccaatttccttaaattctgtgttcatactgttgaacctcgaaagttgtagcttcctctttttctctttgaacagtataattgtccttttttgcatatgctttagaatccatactgatgtatttggttggaaagtgggaaaggagcttttagtgttctgagatcattgtgtgatgttcagaaccaaagtgtcactgttacatggtagagaatggttcatattctacctccttgttaagtcaattaagtcaatttttcctagtaaatgggaaagcaaccgccttccctatcatgaggggttttgggtttcttcccacttgctttgctgagaaattaaattatggaaatgggtatttaccctctgctccaatctaaataaacttttattccttctaccaataccttcagtagcccatgcaccaaagtcggctcctttgtttttttttttgttttgttttgtttttttgaggcagagtcttgatctctcgtccaggctgtagtgcagtggtgtgatctcagctcatggcagtctctgcctcctgggatcaagcaattctcagcttccaagtagctgggatcacaggtgcctgccaccacacctggctaaggtcctttgtgattattagcagagaatgggatctaaaaatgttcctgtgttccttctagctagtctttgctactgtgcccctagataatgggatctagtttttcacttgagtagtgtttttgggaacatgacagagtagccctgtccaagggagacatactactcggtatgtatttctaatttaccacacatgcttctgtattttatagtaagattgtttgagagtttgacttcattagggtttagttctctgaataccactatatactttagctctttgaactctggactttctgtcacaagcagtttttctccttagtttttttgtgtcttctgaaatgtcattttggtggattcttttaagaaaatatagggctatattaaattacttaaaggttttctaagtcacaatagatgtggcctgtaaaaatatctgattatgttgggtgtgttatttctgaaaataaaccaagcaaatttcacaatatcctgtttaataatttctctataaataagatacatatttaaaataaggctataaatgtttgttgcttaagagacaagatattactgaaattatcttctgaagtatgtctatccctttccacatttttgtttccactgccttttcagtaagatgttaaggttaattttacctctggcagtgttttagcaccttctttctatattgggcattctatctgtcacatttttctagtgtagtagtaaacattactcaaaataaatgatttataggccgggtgtgatggctcatgcctgtaatctcagcactttgggaggccgaggcgggcagatcacctgaggtcaggcattcgagaccagcctggccaacatggcaaaaccccatctctactaaagatacaaaaaaaattagccagatgtcatgcatgcctgtaatcccagctactagggaggctgaggcaggagaattgcttgaacccaggaagcagaggttgcagcgaggggagattgcgcccctgcactccagcctgggcgacacagtgaatgtctgtctcaaaaaaaaaaaaaaaaatttataaagtgctaagtattgacccagctgggcacagtggcttatccttgtaatcccagcactttgggaggccaaggcaggaggacgacttacttgagcccaggagttcgagactagcccgggcaatatagtgagaccctgtctctatctccctgtaaaaaattagatgggcctgtggtatgtggctgttagtctcagttactcaggaggctgaagcaggaggattgtttcagcccaggtggttgaggctgcagtgagctcgatcactctagcataggcaacagagcgagaccctgtctcaaaaaaaaaaaaaaagaccctgttttgagtttctttgtaactgacagactaataggtaatagagttttaaaatattgttactagatttttttttttcttttttgagatggagtctcgctctgtcacccagactggagtgtagtggtatgatcttagctcactgtaacctccgcctcctgggttcaagcggttctcttgcctcagcctcccaagtagctgggattacaggtacctgccatcatgcctggctgatttttgaatttttagtggagatagggtttcaccatgttggacaggatggtcttaatctcttgaccttgtgatccacccgcctcggcctcccaaagtgctgggattacaggcgtgagccacagcacccagccatgatttagattcaaactgtatcttcattggttccagtcacaatggtttacagtgctaagtaaagctatagattgcatactgctgtacttgtgaaggctttttttttttttttgagacacggtctcactgtgttgcccaggctggaatgcagaggtgcagtcacggttcactgctgcctcaacttcctgagttcaagcggtcctcccagtttggcctcccatgtagctgggactataggcatgcaccactgcatctggctaatttttaaattttttgtagagatgggagtctcactgtattgcccaggctggtcttctgaaggcttttagtttgtttgtttgcttgtttgtttttaaatcttgtttgcttgcttttcttttctttcttttctttttttttgagatgaggtcttgctctgtcgcccaagatggattgcagtggccttatcacagctcactgcagcctctacctgctggtctctggcgatctttgtgcctcagcctccccgttaactgggacaacaggcacataccaccacatccagctaatttatttttattttctgtagagacaggattttgctctcacatatctgtccaaccaataaacaaaaaagttatgatactcagggcaaagttttctctaactcctcaaaaggacttgaatttaggacatctttcttactgagacatacaaaggcttaaaagtatgtattacaatccaaaacactcaccactgtctaacatttctcctccaaatgagagcactaatagcctattcaaaagagtctctaattatgaatgtgaagttacaaagaatgtttttttgttcttaggtctatgaagataacacaaaggcaacaagtgaaaacacaatgcataaagtttcactttagtccccttataacagaaaacaagagatgaagataagagcacttttgctgtcctagaacttaaaatgaaaatctaagagagacccataatataaatacaatcatcccttggtatatgtggggattgcttctaggaccaccacatataaccacgtcagtgcatgctctgaagtacgccctgtggaacctgcgtatgtgaaaagtccttcccatagacttgggtttcacatcccatgagtactgtattttcaattggtgttttttatttttattttttattattttttaaaaaattttaattaaatagaaatgaagtctcactatgttgcccacactgctttcaaccttctgggctcaagcaaccctccctgctcagccttccaaagtactgggattacggacgtgagccactgctcctggcctcaatctgtgtgtggttgaaaaaaggctgcatataagtggatctgtgcagttcaaaccagttgttggacaagagccaactgtaattagaattataaaaattgatgccatgctctctgtgtgagtaacaatgcagtaagaatatgcagattttagtcactaattttgacttcctgggaaaggaactgcagactagatgctacaaaagagaaacacttgagggtgaaaaagattaactgtaagaaaatgggaaagagactaagaaatccagtttcttagaaaaacatattcagtagaaacttaggaacagaagtgatgtcttggttggccatgagatagtagatccctacaaccactagaaagtgtccattagggacattatccctttagggtaatgtgcaggtggtcatgtttcagactttattactcaaagtcatgaccactggggaggttagataagcatctttttaaggggttatctatgttacaggcattgtttaaagactctgctgaagaacaccttggtatgcaggaatcaaacattggtcagggccaggcactttgggaggccaaggtgggtggatcatttgaggtcaggagtttgagaccagcctggccagcatgatgaaaccccatctctactaaaaatacaaaaattagctaggtgtggttgtgggtgcctgtagtcccagctactggggaggctaaggcaggagagtcgcttgaacctgggaagtggaggttgcagtgagctaagactgtgccactgcactcagccagggcaacagagtgagactctgtctcaaaaaaaaaaagaaaaaaattggttagcatgatggtttcacttcaagatggcatcactcttgcatgcaacaggcagttttcctacaactgtctttaattctgcagatactctgtcagtctttaaaatgaaactataaattatagattatctacatttatggatgaagaaagtgtggcttagagaaattcagtaatttgctcaagttcacagagtaacagttcaggaatgttgatctagatctgtctgacttcacagttggcatacataattaccatgctataatgtcttctgtaatacatggtatagaattgacaagaataatctttttttttttttttttttttttttgagatgaagtctagctctattgcccaggctggagtgcaatggcacgatgttggttcactgcaatctcagccttccaggttcaacgatcctcccgcctcagcctcccaagtagctgggattacaagcgtgtaccaccatacctggctaatgtttgtatttttagtagagacgaggtttcactatgttggccaggctggtctcgaactcctgacctcaggtgatccacctaccttagcctcccaaagtgctgggattacaggcttgagccactgtgcccagcctagatttaatcttttttttttcttttttgagatggagttttgctcctgttgcccaggctggagtgcaatggcgcaatcttggcttactgcctcccgggttcaagcgattctcctgcctcagacttccgagtagctggaattacaagcatccaccaccacgcccagctaattttttgtatttttaatacagacggggtttcaacgtgttggccaggctggtgttgaactcctgacctcaggtgattcacccgtcttggcctcccagagtgctgggattacaggagtgagtcaccacgcccggcctagatttaatcattttaaccatgtatgcagttgtaaataatagaactaagaatctggcgctcaggggagaggatgggacttgaagtgtggattatctatttttttctactctgttttatgctgtttttaaaattgtacaagtcaacagtgatacatgtcctatttttagaacttcaaacaatacatacaaaactacagttccctatgaccacgtacttgagtacacaggtgccctcctcctgaatggaatgtatactttttctgcgtttatttacaagtatttgtacccataggaatatatagtgttttagtttgcaaaaataatatcatactgtacatactgatggggtgatcattttgtgatctaccccaccccaactcatgcatgttgttctgcagctttaaagatgttcttgggatctctcaatttcagtcattaataagttacttattttgatacctgcatagtaatctgttctgtggtatgaatatgtgatttaatatatgaatatgccaattgataatatacattatttctaatttttttgatattatacaaatagtgatttttattcttttcaagaatttaaaatgcttggtgaattttagttatgtaaaactagtatgtatatgtacataaattattgaaattactaggatgtttttggcagagcacctcacaattctaccataatgattctctgtaattatctggagagtcattgttcttaaaaatgtttgcagtctgtgctttcattaatcttttctacatgaagttaattttagtttttaaaatatacttaatttttgtagagatttaaaattttttgcaactttgtaattaatgttaatttatatttagaataatttatactgtactattagggcattttcttgcaataaattattagtggagatgagcgactgttgatggagatgagtacctttatcatgcagaatatcagcagccagatattggacttttctcactgccccttaatttacttttttaaatattgtcgttctatggcaatcaggcttttggatattgaggaggaagtaataactacataatgtgataatgatgtctttgaggacccttgtccataacaagccatttgaatcagaataatatcttgtgcaatatggtgttaaggcttttaggttacatctcatttctgaatgtattctggatgacttctattgactttatgatgtttacctttcatatttaaaataagagggttcttttagcagattttggttataaaacatctgaaatagtgtaatttaaataaacaaggtaaaggaaaaaaatatatgtattatgccaagaaaatgagctagtccttatggttgagacgaaattttagctttgagcatttttgtatgtaagcacagaatgaagtaggttggattttatagggttcatctcagtaggaattattgttctatttcatttttgcagaattgagaagccatcataattgttttatatttaaacttttaaacagaaacctaaatttgcataaaaactcaagacagcgtttacagtttcaaggcattttgtttgccttattgtatttgtgtacttcttccatagtgcctggcaaacagtaagtgccaagtacatttagtttgattttattttttattgtatcttatttcttcttttttgaggtagggtcttcctctgttgcccaggctggagtacagtggtgtgatcatagctcactgcagacttgacctcctggttggggagtggtggtgtgcaccagtggtcccagctactcaggaggctgaagcagaaggacccccccagcccgggaggcgctccagaacaccccagcttgggtgacagagtgagaatctttctcaaaaaataaataagtaaataaagttgcttgggacatgttatatagatgctatttaaagctatctggataggtaatatcctccagtttataatcatctgtaccccattgcgtgatggggtgatcattttgtgatccaccccaccccaactcatgcatgctaacagttatgtatgccttttctccaatttatctgcctgcgttttgtcagacgattttcagcgaaccttcactgggcaaaggggaagtcttcttttggcccctgtattactaacaagcattgacttctaaatgggtaatttggtcaggtgtggtggctcacgctgtaatccagcactttgggaagtcagggtgggaggatcacttgagcccaggagtttgaaaccagcctgggcaatatagtgagatcctgtctctaccaaaaattaaaaaaattaggcgagcatggtggcacccacttatagtcttacctactggggactgggtaatatcctcaagggaatgagtgtagatagaatagggaaccaagaactaagatctaggatatgttagcgttaagataatggagaaaaggaatcatcagaggatactgagaaggaacaatctgtgtggtaggacacaaacctggagaatatatcttgggaactgaagggaagaaagtaaaacatggaggaggggatgaacagccatgtcagattttgccaataagttaggtaggataaagactagaattgaaaattgtgtttagtaatgtaaaggcctttgttacatggtcttcagtaataaatagccaggttagcactatttatatatgtttttaatgggaatttttgatgtttacagtgtctcctttgccatgttcaggttttccaacctattggtggtatgtctgacagtggatcacaacttggttcaatgggtagcctcaccatgaaatcacagcttcagatcactggtaagttttagaaacatcggctgctttactttatttttccccgtatgattaaaaaataatcctgagagggccaggcatggtggcccatgcctgtaatcccagcactttgggaggctgaggtgggcgggtcacttgaggccaggagtttgagaccagccaagccaacatggagaatacaaaattagttgggcatggtggtgcacacctgctgtcccagctactccggaggctgaggcgtgagaatcacttgactttgggaggtagaggttgcagtgagctgagtttgcgccactgcaccccagcctgggtgatagggtgagactgtctcaaaaaaaaaaaaaaaaaaaaaaacttaagagaacaaaatatttaatggctgttacatgtcaggtgctcaattaatatttgctgaatgaatgaatgaagatatatagatatgtaatttttattgtagaatattcttggatattttacagtttttttttccttcaacatgggtacctgggtagttgctaattatgtttgtttggtattgtattaacagctttattgagatataattcatatcccctacaggttacccatttatttatttacttataacaatgacacaatcatgcagcctcctgctgtggtgactacagtcgtgcaccaccatgcccagcaaattctgcagttttttgtagagatggggtctcactataatgcccaggctggtttcgaacacctgggcttaaatgatcctcccactagggcctgacaaaaggcagataaataggagaaaaggcgtacatcttattagcatgcatgggtaggggtgggatggatcacagaatgattaccccatcatgcaatggggtacaaaatggttatatactcttcttcataggggaaagggagatggggaagtgtggatgattttagagggatggtaaatgatttttaggggaattcagagggctcgaagaatgtacaatggcctgggacaaagtctgtttggcccacagaatagatagtggtttatgacaaaagtctctccaggtccaggtctgttaaaagacttcagtcagccaggcacggtggctcacacctgtaatcctagcactttgggaggtcgaggtgggcagatcacctgagattaggagtttgagaccagcctggccaacatggtgaaaccccgtctctattaaaaataaaaaaaattagctgtgcgtggcggtgcacacctgtaatcccagctacttgggaggctgaggcaggagaatcacttgaacctaggaggcggaggttgcagtgagccgagatcatgccattgcactccagcctgggcgacagagcgagactccatctcaaaaaaaaaaaaaaaagacttcagtttttatattcctgctatacaagtttagttattgaaaactcagggatgggaacaaaggtaattgtaattgttttcttctttggtggatctggacttaggcagataagagaacttcagaaaacaacttcatgctgtactttgagagaaagaagattgagggaggggagctgcaggggtcagagagaccttgagagacttacttagttcagcaagtcaaagcaccatattttgggatatcagtttctgaaccccaaaagtacattcacagagttcagcaaccatcaccacaatcaactttaaaataatttcgtccttccaaaaagaagccctgtactcattagcagtcactctccatttaacccatccatccacaaacccaggcaactcctgatctttctgtctctagatttgcctattctggatctcgcatataaatggaattgtacagtgtgtggccttttgtgtcttctttgggcagactgttttcaaagttcatccatgttgtagcatgtatcattacttaattcctttttttgctaaataatattttattgtatgagtatacgaaattttaaaaatccattcttcagttgagggatgtttgggttgtttccactttttggattttatgaaaaatgctataaacatttgtgtacaagtttttgtgagaatgtatgttttcagttttcttgggtatatacctaagagtgaaattcctaggttatatggaatccatgtttattagcattttgaggaattccaaattattattattattatttttttttttttttggagacagagttttgctttgttgcctaggttggagtgcagtggtgtgatctcggctcactgcaacctccgcctcccgggttcaagtgattctcatgcctcagtcttctgagtagctgggactaccggcacgcacaaccatgtgtagctaatttttgtatttttagtaaagacggggtttcaccatgttggccaggctggtctcgaactcctgacctcaggtgatccacctgcctcggcctaccaatgtgctgggattacaggcgtgagccactgcacgggcccaaattatttttaaaagcagctgcaccgttttgcattcccactagcaatatatgaggattccgatttctctacattcttgccagtgattatcttttttatgatagccattctagtgggtgtgaagtagtattttactctgtgtgtgtgcgcgcgtgcatatgttttatccatttaatttttttttttttgagaccaggtgacagtctgctctgtcacccaggctggagtgtagtggtgcagtcatggctgactgcaatctctaacttctaggctgaagtgatcgtgccacctcagccttctgagtagctgggactacaggcatgcaccaccctacccagctccctattgtggttttggcttgtactcctttgatggctaatgatattgtcttttcatgtgcttattggtcatttttgtatcttttttggagaaatgtctgttcacgtcctttgtttgctattgtaaattaatacctctgtcgctattgttaattaatatctctgtctttatttcttcctctaatcatttcatgtatcactttcagattaatgtttttaatatgccatccaatatgtttcattcctgctcaaaaaccttaggtacttctcttagactatggtatagagtttctttagtatgagctaagacccttcaccctctgtctacatatggtgtctttgtaggttttttgtttttttttgtttgtttttttgagacagagtctctgtcgcccaggctggagtgcagtggtgcaatcttggctcactgcaacctccgcctcccaggttcaagcagttctctgcctcagcctcccaagtagctggggttataggcgcccgccaccaagcctggctaatttttgcatttttaatagagacgaggtttcaccatcttgaccaggctggccttgaactcctgacctcaggatccacccacctcggcctcccaaagtacagggattacaggcgtgagccaccgcgtccggcctgtaagtctttatatgtgaaccatttgtactagctagaatgagtcactggattttcctcagatgtgtatgtatttctatcacacaatgaaaacctcagtctctctccctttaatttgatagcatttattgattatagaacaattatcatcccctgtattttagtttcaactatgcctggcttatagcttcttctggattatgaataggttaggtcacagatcatatcttagcattattattttcctcaaaacctaaaggtgattgaatataaaaataatactgtcttaaggtaaccacatcatagaattttttcggatttccttcactaactctggtatctttccttagcagtatctgtttcttaaagaaagatattactatttcttagttaaggccagattccagttttttttccccacccctcatcctcaatctagcttgagtagctgacttatatcagaccagaattcttatgacttctttttggcagcttaaagttgaaagcttttattagaacagtcctttaaaacattctgtactttttcagattacataagtaatatacattttattttttactgatttaaaaaaatagtaaattattctcagagacaatgattaatagtttttttctttgcataaagtgtcttacttgctatcatcgttattgaattttttttttttttttactgtgatgataaacactaattgcacctcaaaaagtcgtaatccatagccatctcagatttttttctttttgagatggggtctcaccctgtcgtccaggctggtgtgcagcagcgtgatcatggctcactgcaaccttgatctcctggggtcaggtgagcctcccacctcagcctcctgagtagctggaactacaagtgtgcgccaccacacccggctaatttttgtattttttatagagatggcgttttgctctgttgcccaggctggtctcaaactcctgggctcaggtgatccacccacctcagcctcctaaattgcttgtattagaggtgtgagccacctcactcagccacatctcagatttttaaaatcttgggtctaaaatattctttttctttttttttttgagacagtttccctttcgttgcccaggctggagtgcaatggcgtgatctcggctcaccgcaacctccgcttcccagattcaagcaattctcctgcctcagcctcccagatagctgggattacaggcacccgccactatgcctggctaatttttgtatttttagtaaaaacgggttttcaccatgttggtcaggctggtcttgaactcctgacctcaggtgatccacccgcctctgcctcccaaagtgctgggattacagcgtgagccaccgcgcctggccttaaatattctcttttttataaatttttttctttttctttttttaattcattgcttgctaattgggtctaaatattctgagaatgctcaagtgtagggcaaaaaacaaacacaccatgaataatggataatagttcagcaaacattattagatatgaaacagagaacataagataaaagacagaagtgccaagtttaatagaatcaaatgcccttcataattcattgagttacttctttagttttgatgctagaatgcttagaactaagtattttgcacaaatctccttaatgacatcgtgggttttaatctcttaatgggtcagttatagtatttgttttattgtaagctgcctcaatgcctttctagaatcaagtaaggaataaatatgcaagtgtaaaaaatgaatgtcaggctgggcgtggtggctctcacctgtaatcccagcactttgggaggccgaggtgggcggatcacttgaggttgggagttcgagaccagcctgagcaacatggagaaactctgtctctagtaaaaatacaaaattagccgggcatggtggcacatgcctgtaattccagctactcggggggctgaggcaggagaattgcttgaacccgggaggcggagatggcggtgagctgagatcgtgccattacactccaatctgggcaacaacagtgaaactgtctcaaaaaaataaataaataaaataaaaataaacaaatgtcaccatgtattgtaatggcaagaagggataactggtcaggaaaaagagatcagtatttgactagagtcactgggaaagaaggtggtgtttggattgctgctatttatttatttattttaagggacagggttggccgggcatggtggctcatgcctgtaatcccagcactttgggaggctgaggcgggcagatcatccgaggtcaggagttcaggatagcctggccaacatggtgaaaccctgtttctactaaaaaatacaaaaattagccaggtgtggtcatacgtgcctgtaatcccagctactcgggaggctgaggcaggagaatcacttgaacctgggagacagaggttgcagtaagccaagatcgcacccctacactccagtggcacagtcacggttcactgcagccttaacctcctgggctgaagcaatcctctctcctcatcctctatagtggctgggactacaggcgtgtaccacccaccacacttgactaattaaaaaaaattgtgtttttgttttttttttaagacggagtctcgctctgttgccaggctggagtgtggtggcgtgatcttggctcactgcaacctctgactccctggttcaagcaatttttctgcgtcagcctcctgagtagctgggattacagacatgcgccaccaagcccagctgatttttgtatttttagtagagatggaattttatcatgttggccaagatggtcttgatctcctgaccttgtgatctgcccacctcagcctcccaaagtgctggaattataggcgtgagccactgcgcccggccaaaaatttttttttgtagaaacaaggtcttgctatgttgcccaggatggtcttgaactcctggcctcaagtgatgctcccagcctttccataagtgttggaattataggcctgagccactttccctgacctaggattgctgctgtttaaatgcagaaaagtagaaaaggaccctttgaggagacagcatgagcaagacacatgaaagaagagagtacacaaaacttgccctaatacccatgaaagctcctataataggggagccctgagaatgggaatggctgtgtctttcaggtgtgccttaaaaaaaatttattgctaagggagatggagatgggaagtaatcagtgtttgtatcaaagggtggttagatttgtgttttagaaagttagaaagagaaaaaaatttttctttttttttttttttttttgagacagtttcgttctgtcgcccaggctggagtgcagtggcgcgatctcagttgactgcaatctctgcctaccatgcccacccccttacctacttatttatttatttattgaaacagagtctcgctctgtcacccaggctggagtgcagtgacttgatcttggctcactgcaacctccgcctcccaggttcaagcaattctcatgcctcagcctccccagtagctggtattgcaggcatgcaccactgtgcctggctcactttttgtatttttagtagagatgaggctttaccatgttgtccaggctggtctcaaaatcctgacctcaagtgattcacccacctcggcctcccaaagttctcgaattacaggcgtgagccattgttcccagccaatttttaaattttttataaagacacattctcattgtgttgcccaggctggtcttggatgttttgaatcagctcttcgtttcttcttatcatttagccagggtctttaccttggatgtaagtctgttttcttatgtccttccctgtctttatttacctgtttcccatttaattatgcctatatataacctcagctattatactgaactcatctgtgtcttctttttttcttttttttgaccgggaaatttttttttaaattacaagctcaacccatttgttctgtttcaaaggtgcagtacttttcttcatttattagtgaaagaagttaagaaattaactttccaaaaaaatcagcaaatggcaagcaaatgtccttgaaagtcacagtcacatatagtgtctcctagaaaagaggaggggtaagacagcttccacctactttcacaagtttcatcaaatactggatctactcaagggtggagtaaaaaggcaactaactttctaaaaggagtatgttattaaatgaggcatttactatactccttcctaaaagccccagatggggaacatgttttctcaactagacctaggaagtagactgtggaatcaatcggtcctcctcaccttaagggccatccactggttaatgaattaaaaaaaaaaaaaagactgaaaaacaaacctcccccctaaaaaagaggagggggaaaaaaaaccactaagatgtcccagagtggaatcagggagcagcttcaacaattccaattagtctgttacagagtcatccacaagcatgccttgctttaaaaatacatatatatatcttgaaataacaaaactgaaactagtactaatcacttttctgacaatatacaattactcaaaattaactagtactgggctagggatacgggggggcggcgcatagctatgggccttgtctcacatgagtacatgtgggtaggtgcagtgcgtttgtcattatggcaaaaacgaattttaatttttaagctttagtttgacttaaacattgcttttagtatgatgccaacaccagctatgcagaaagggctctggagagatgttcctagcagcacacatatgcagctcttcttcggttctggaggttccagggcagccaatattgcttcgtcaaatacattctttaggcctttctttgtgagtgcagaacactccacgtacttgacagtcttcaggtcatgggccagcttattcagcagtctctggagtgataggcttctgtttgttcttggcaagtttctcaatagtagaggggtcatctttgagatcaatttgggtcccaacaagcaacaaaggagtctttggacagtggcacccacttttctttcacattttcaaatgaaaatggagagatggagagaccactgaaaaatagactagaaatacatctgtttgtggataactcagcagtcataatccctcataatcctcttgctctgcagtatcaaaaagtccaagagtatatggttctctaccagtcataattgttattgcatagttgtcaaaaacagttggtacatattctgatggaaatttgttgtgttgttttaccaacagcaccatcacccacaacaacacacttaattgtctgcattgctgaaatagttttgtatccactttaaatatttcaaatctcatgttgacctcagcttctccaccgggttgttggctcatctgtgttttttatttgatgtttgttgtatgctagcattgggcaaaataaggaaactttggtatttgccagaaatttcttatattctcgtctttactccaaattaagaaagagatgtcatcttgattttagttattattaatcgtaattaaatattactgcatttcccaaatttattacagtgcaatatatttagctctgattgttcagggaccaaaaagatcccttatacttatgttttttttttattcctttaacagtccagcaaactaagaattttatacttgtgatttagagtttagttctccacaggcagggcagagttcttccaaagaggtagctgttgctcaactggattgatctcttacctggttcattattctgcttatttatttagttttggcactgttttttgttgtaccttgttctctgtagcttatccaaagtaggttggataagctacggatccattctttttttgtatgtgtattgtaaaatgtttatgttgtatcagagagatacactgaagaactgaaatcagttcaaagcactgaccttcagaacagaaggatcagaggaacattagtttaagccatgaaaactatttgcattagtatgtgggtatgtgtgagagagagagagcacatgcatgggagagtgagatagccagcaagtgattataataaagcagagagacagagactagaaagcagggaaacttttgttttctaccatgactaataacaatttggttcaacatgtatattaaatacctctcagattaaatcccagagtgctccagaaattgtaaagattggctatatctgtgcaatcaagaggctcacggggtgagttaaatgagtaaaaactagtaagacaaaatgaaaagctgacggagccatacgtaaatatggaaatactctggaagcatggaggtggaaataatttaacttcttaggattaaagaagcctttaatcaaagagaagggggatgtttatctctcaggaaatataattttaatgggtataaaagaaaaaaggatcattctcagtagagggagcagcatgtttagaggaatgaaagtacatggcccatgtgaggaacagcaaataactaagggtggctagaatatagaactttggtaagatttgttttagtaggaggctggaagggtaagttatagctgcatactaagcaaggtaaaatgccccataagagtttaaacttcatttttcaggcaataaagaatcatttaagtaatgacattaatgacatgatcacatctattctaggaaaataattctggtggctgtgaggggttggattggagaagtgagagtaaaaacagttgtttagaggcctctcatagtagcccaggtgtcagatgagactgacatttttctggtcctttttcttccggtttataatgtatagttttttatttttttgtttaatttactgtgggactttagttttcagaaagtagtacattgatttattttatatttgtaatattatgactataacacaaggttttgttcaatctagactttgagatttgaatactgaaataatttctcctgtccaggttttgcagtgtgacccttgggcaaatttttaaatttcacagagcttctgtctttttttttttttttttttttgagatggagtttcacccttgtcacccaggctggagtgcaatggcatgatctcagctcactacaacctctgcctcccgggttgcagtgattctcctgcctcagcctcctgagtagctgggattacaggcgcctgccaccatgtctggctaatttttgtatttttagtagagacagggtttcaccatgtttgtcaagctggctcctgacccccgttgatccttttgactgagacttccaaagtgctgggattacaggtgtgagccaccacgttcggccgcttctctttttatgtgtaaaaattaggacagtaatacctgtttcgcagaataggtattgatattgaaatgacatttacgtgcaggcacatggtaaatactaaaattggtgctcctccacccccatgtaaagtttgttaggccaggcctggtggctcatgcctgtaattccaccactttggtaggcctaggtaagtggatctcttgagcccaggagtacaagaccaacttgggcaacatgtcaaaagcttgtctctaccagaaaaaaaatacaaaaatttcctggatgtggtggcacgcacctgtagtcccagccacttgggaggctgagatgggaggatggcttgagcccaggaggtagaggtggttgcagtgacctgagattgtgccactattctccagcctgggtgacagaaccagaccctgtctcaaaaaaaaaaaaaaaaaaaaaaaggtttcagtggggtttggtaggattatgtagcaaaattgctttcacaaatttgaatccagctgggcacagtggctcacgcctgtaattccaacactttgaggggccgaggcaagtggatcacctgaggtcaggagtttgacaccagcctgaccaacatggtgaaaccccgtttctactaaaaatacaaacattagctgggtgtggcggcgcgtgcctataaccccagctgcttgggaggctgaggcaggagaatcgcttgaacccggcaggcggaggttgcaatgagctaagattgtgccattgcactctagcctgggcaacaagagcgaaactcctatctcaaaaaaaaaaaaaagtttgaatcctagaaagccccaacaggggcagatcatttccttcctgtcagccttgcgttgctctttgttctaggtttaatcagtttcagagtgccacaagaggccctgaaatataatgagtgtatgtataattgtttgggctatttcaccttttcttagaccagaaatgtattaaaagtagaaattttatgtttttctgtgtaatcctgtgattaaatttggctctatattaagcactagagctttgatttaatagtactttttttttattttttattttttaatagagatgaggtctcactgtgttgtccaggctggtcttaaactcctgggctcaagtgatcctcccgcctgggtctcccaaagtgctggtattacgtgcccggcctattacattttaatcagagatagctctggtccccacttgaatattttttattctgaaaaagactttctatttcatgtttccacttagatgtctcagcatctggcatctgaaacaatttttttttttttttaaaagacagttttgctctgttgcccaggctggattgcagtggtgcaatcacagctcactgcagccttgacctcccaggcttaagcgatcctcctgcctcagcctccccccagcagctgggactacaggtgcacgccatcatgcctggctaatttttaaaattatcttttgtagagacgggggtctcactatgttgaccaggctttgttcaaactcctgggcctcaagtgatcttcctgcctcagcctcccaaagtgctgggattacaagtgtgaaccaccatgcttgatcttgtctatttgtctagccttctcttgcaactcttctttttcttcagtcagtcattctgtggctagcttgaatgccagcaaaaaggcacataagcctaaaaggatggaagtatttgatatatgtaaagtcagagtttcagggccaccacctgggactatgcctgcttagatatccagactttggactatatattaagctgctgcttcttttttttttttttttcgagatggagtcttgctctgtcacccaggctggagtgcagtggcgcaatcgcggctcactgcaatctccgcctcccaggttcacaccattctcctgcctcagcctcccgagtagctgggactacaggcgctcgccaccacgcccggctaattttttgtatttttagtagagacagggtttcaccatgttagccaggatggtctcgatctcctgacctcgtgatccgcccgtctcagtctcccaaagtgctgggattacaggcgtgagccaccgcacccggctatattaagcttcttaagtagttactatataaactcctctgacaactccaagggttccttaactttgatgagcattagcattctctgaactttgctgctacagtagataacctgggtggtggagtagataacctgggtggtgggtacttccttggttgtagttacctcatctatgagggagcaatgacttgccacctatggaaatcatggggtgctttgctagatttctacttgttataccctgtaaggggcctggacaaagctattttatggcaaaggtaactaaaaggtgaaggctaaggagtttgtatctcaagatgcacaacataggaagctcttccttagataatatgagatgttaaaaaaaaaatcttagtttgtagttcttattcttccattggctttccaacacacagaataaaatctaaattatcattattattattatttttgatacgtaggctcactctgtcgcccaggctggagtgcagtggcataatctcggctcactgcaacctccacctcccgggttcaagcaattctcctgcctcagcctcctgagtagctgggattacaggcgcctgccaccacacctggctaagttttgaaattttttagtagagacgaggtttcgccatgttggccaggctggtcttaaactcatgacctcaggtgatccacccgcctcagtctcccaaagtgctgggattacagatgtgagccgctgcgcccagctgcagattctttatcttggtttataaagctcccatacaaagcttatctcccttcatttaccattccttcctgtgcttgctatactttccctcagatatgccatactcatttgcctctctctggaatgctgtcttattcccacctccagtcttcccatggctggctccttgtcatttggatggttgcttaaaagtcttctcaaaggccttccttactaccaagtcattttctgtcacatcagcctgtttttcatcataaatactttaattagaatataatctccttgaaagccagatccttttctttactgttaattgctttagggacagagtttagagcagtatctgacacatggtaggcattcagatgtttgttgaccatgtttgtcaatgcatacattttgttttgagatatggtctcgctgtgctgcccaggcaggtcttaaacacctaggctcaagcagtcctcctgcctcagccacctgagtagctgagattacgggtgcaagccaccctgcccagccatatgtgtactttttaaatttttcctcctgttgcccaagctgttgcacaaccatagctcactgaagccttgacctcctggactcaagcgattctcctacctcagcctcccaggtagctgggactggcacacgccactgtgcccagctaatttttttgatttttaggcgagatgaggtctcgctatgttttccagattggtcttcgactactgagcttaggcaatcctcttgcctcagcctcccaaagttctgggattagaggtgtaacccgctgcacctggcccatatgtgtactttttaattgatctgagttctggacttttgataccatggaattctactgatgggaatttgggttgatggtacggggaacctctatatttatttactaggatgggaggaagtacaaaggttatgagtgaataaaattggtaagtattttagagcaaaactgcttctctgagtaaatttagtaaatctgcctaattatgaagttaaattgatttcatgagtgaattaacatctcaactattgaaatgtttttcatttcttttacatagtcatctcagcaaaacttaaggaaaataagaagaattggtttggaccaagtccttacgtagaggtcacagtagatggacagtcaaagaagacagaaaaatgcaacaacacaaacagtcccaagtggaagcaaccccttacagtgtaagcttggaagtatttttctgtagtatgttttgtttagtagatgattggaaatacaatttaaaaaaatgtttctcactttggttttttgttgttgttgttcctcctttctctcttctttttttttttttctttttcctaggactccttctggtttgcttttttgtttctctttagatagctgtttttatagtcactccctttctttaccttctctagtttagttggtgatgatattgctcttgtcactgaacaatagctgaagagagcccatggtcacggacctaaaaagatttctggcagcactttttattaactttgggttgaattaagggattatgagtactccccccccccccggtttttaacttaatttttaaaaatagtgttttagttagttttttctttgttttattttgagatagagcctcaccctgtcacccaagctggagtatagtggcactatcatagctcacagcagccttgaactcctggcctcaagagatcctcctgcctcagcctcccaagtagctgggaccacaggcatgcatcactgtgcctggccagttttttagtattttaaattacggtgccagttatactatagttatttattttttgaatgagatggggtcttgctatgttgtccagagtgctctctctcaaccttcccggctcaagcagtcttcccatctcagcctttttctcaagtagctaggactactactatacattttattctacaactttctttttgtacttaacactaatatcatggtgatctctttacctgagtacatatggatcttgcatcccttatatagctttgtaatattccttaatatgggtggactatattttaatagttctgtattgattctaatgttgcgctattaggcagtattgcagatacatccatgtgcatatatctttgtatgttggtgtttatatctgtagtgtagattcatagaagtaggatatagctcagttttaacacaaagccctaaatgttggcattataatctaagatattgacagccattgaaggttaattttaatttttaaaattaaaatagaagaaaacacatggagccaggtgtggtggctcacacctgtaatctcaactctttgggagactgagccaggaggattgtgtgaggccaggagttcaaaggtgcagtttgatatgattgctcctgtgaattgctactgaactctagcctgggcaatgtagcgagaccctgtctctacaaaaagacaaacaaaaaaaaaaaagaagaaaaagaatgatttggccaggcatggtggctcactcctgtaatcccagcagtttgggaggccaaggtaggaggatcacctgaggtcaggagttcgagaccagcctgaccaacgtggtgaaaccccatctctactaaaaatacaaaaattagctgggtgtggtggtgggtgcctgtaatcccagctactcgggaggctgagacaggagaattggttgaacctggtaggcggaggttgcaggttgcagtgaactgagatcgtgccattgcgttccagccagggtgacaagtgaaactctgtctcaaaaaagagagaaaaaaaaaaaagaatttatggaagatcatcttgaggcccgtatcattcggatgttcctcatctgtgtttatttcaacataaatgtttcttaggctttctgtttttaattaatggctcattgtagattgagtcaaaatgaaacaatgtattaaaaaaatttcaagaaaacacatgaggctgggaatgactgcacatgtttgcaatcccagctacttgggaggataaggggggaggataaggtgctttagcccaggaggagttcgagattaaccagggcaaagtagcgagttcccatctcatatatattttttcctttaccaagtggtatctataaaaggtcgcagcctccatgattaccagagatgttcagttgaattagatggtcacctaatttcctccaactgctttgagtgcgtacttgatatgcacttggaggctcttaagctgttaatgtcatgaacttcttagtgatttaaaagttaagaaaaaaactctctttgtagttctatgtggtagtgctgttcttgattacaaacatttgggaaacttacaattcaagttattttcctgaatttttaagttgcagcaaattggcctggaattttatcacttcccattgtcgtgtagcaggctttccagactgcttttgagaattgaattgaaacaagactgatcatctttgacagtatactttatgtacctttcttccatattaaaaatagtaaagttaaatttatttgccttttatttcatccctattatgatttctaaattgactaatagagaatttcttttttccactctcacccaggctggagtgcagtggcgcgatctctgctcactgcaacctccacctcccgggttcaagcaagtctcatgcctcagcctcctgagtagctgggattacaggcatgcaccaccactctcaactaatttttttgtaattttagtagagacggggtttcaccatgttggccaggctggtctcaagctcctgacctcaaataatcctcccacctcggcctcccaaagtgctgggattacaggcgtgagccattgcgcctggccgagaaatattttcctaatacaatatataatatgagctctcttgaaagtagcatctgagaattttgaaaagaatgaatacattgtatcccctaagaagaggagcagtgtggaatttttagtttgttttatctacccattgccgttgaaatggtgctggttaataggtacaggtagacttcaaaggaggaaagtgtttctttgtcccctcaaacttgatcacattagagagaatgagatatagagaaattcaaacatttctggaatttgtctttatatcattgaaccataggactgtggtagattatatagtgctcctagctttctgatccttgactctaacattgattctacgtgtattgcaaattcaaatttcccagtttactttggaaataccattatagagagtattccttattggcatttagcaaggaatgagtgagtctatatttgatagttggactgggcaacttgcctctagcagtgtgaaacttgatgtatgtaatgtaatcttcaaatcatgtagctgacgtgaggaactttttaatataaagtttaatgtcaggattttattcacattacagttctgctacttactggctatttgaccttaggcaatttgctttatctctgaatgtcagtttctgtatctgaagaaggaagtgataattcccatctcatagggttactataaagataaaataacttcaagctctgtaaactactgacataaggatttaatttgtaaagttactcttctgtactactaaagtaaaactagttttccaatagtttgttgcaggataagcagttttacttttgttgacaatatgtgtatgaatttacttctgtagttcaactgcaagtgaaaatgaccatgtaagcatgtagcattcagataattttcataagattactgaacataggccaatattgattgtatagtaaactgtaaggggtgcagtgagaagactttagactttaaaactgaattgtaatttatttaggatctcattgtgatattcaataaaaataatattttttccctcttttttcacttttagtatcgttacccctgtgagtaaattacattttcgtgtgtggagtcaccagacactgaaatctgatgttttgttgggaactgctgcattagatatttatgaaacattaaagtcaaacaatatgaaacgtatgtatgtaagactaatagaaattgcacttagctgtttgtttttctggaagaaatttatatgtcaaacatgcataatgactcatgttctctcatacttttaagttttacttggttataggatgaacagatagaagaattttacactattgaaagtattctgagtaaccttggtttgaccattcattttggattcagtatttttagtaaatatttatttacttagtaaatagatattagtaaagtaatacatatttaattgtggaaaaactaaaatatagagcagagatgaaagaaaaaaataaaaatcccacttcagagacaaatcaaatgaatatttttacagatttccttttcagatgcttttttttttgggagggacatggggagacaaggactcactctgttttccaggctggagtacagtggctatttacaggtgcagttgcagcacagtgtagttttgaactcctggcctcaagtgattatcctgcctcagcctcctgatatatctgggactataggcaggtaccaccacatccagctttaaatgcttatttttaaaaatgtcctcattttatgaggttgtggttttatttgtatatgtatatttatataatgtagaaatatgtgtagcttgttttaattaatatatcattttaaaacacatttttttctacccctcgactatctaaaacatacttctaaaaattttctcatttaggacttattagatataatttatccttaaattatgtttttactcaatttaattcaggtatatattactaaataatccttattcttatgctagcccattttatactatagtcttacaaggatggaatgaacataataaacgatggagatactattttacatcatgaatatgaaacattagcatattttgtcctttttaaaagatacagtgttttggtgtcaaaagtgttcaaaccttgggtgaaacatagttaaacagtttatatgcacatatagttatattttctcggacatcacatttttaacagttaatttttaacagttaagatgccttaactgggaaaaaagtgaccattttttctgtaactttattttcttccagttgaagaagtagttgtgactttgcagcttggaggtgacaaagagccaacagagacaataggagacttgtcaatttgtcttgatgggctacagttagagtctgaagttgttaccaatggtgaaactacatgttcagaaagtaagtgactacctttttaaggtctttaatgattcttcttaaataaaatagccattgtagtatgctgtatgtaattattttgattttttttttcagattaataaaatgtttttttctggccggtgtggtggctcacgcctctaatcccagcactttgggaggctgaggcagaaagattgcttgagcttggaggttgaggctgctctgagccgtaatcacatcactgcggtccagcctaggtgacagagtgagaccttgtttctttaaaaaaaaaaaaaaagaaatgatttttttctttttaaaaaatagacattgatcactttaaacattttctttttagtgtattatttctgtgaataaaagcatgtatatttttacataagttgaaaaattagccaaatagaagttgaatcacatttgcctccatgagtaatgagttgatttatatagtatttattctgtaggccaactatggcagttaatatgggttagatgccacaccgccaactgtgccaatggcagacagagtttattgtctgtagcagtgtggggctgagtttaaatatgactttaaatccttttttttttttttttttttttttttaagacggagttttgctcttgttatccaggctggagtgcaatggcacgatttcggctcaccgcaacctctgcctcctgggttcaagcgattttcctgtctcagtctcctaagtagctgggattacaggcatgcgccaccacgcccagctaattttgtggggtttttttgtttgttttagtagagatggggtttttccatgttggtcaggctagtctcgaactcttgacctcaggtgatccgcccacctcagccttccaaagtgctgggattacaggcgtgagccactgcatccggcctctaaatccttcttaacactgtaccacccagccattacaagctgagatccatttaccattccccttctacttctgtaggctacacttacagagatatcttttgacaatactaattcattatatgtgtgttagtaaatcttacaatctctgatttactagtgtgtgttaaaggaaaataaactgtggtggcacctgagttagtggctacacagctagcatatttaatagaactttttcctactgttttcccatcagatatttccatcacttcctaatgtgttaagtgtattttcacttgaaacatagattttagaggcctgaagacaatgaaagagcagattaatggatttcttgatgagtttgacgaagatcttttggtttacttaggaactgcattatgaaacattttgcagctgtcaggtgctattctttttctactttgttgaaagacttcctacttcctctttgactcatgacttttaaaaatagcaggacaggctgggcacagtggctcacaccccttgatcccagcactttgggaggctgaggcaggagggttacttgagcccaggagttcgagaccagcctaggcaacataacaaaaccccatctttgcaaaaaataaaaaaatttgccatgtgtgtggcacgtgcctgtagtccaggtacttgggaggctgaggtgggaggatcacttgagcccaggagtcagaggttgcagtgagcgaagatcacaccactgcacttccagcctgggtgacagagtgagactctctctcaaaaaaaataaagtagcaggacctaatatgtcttccagattagttctctggggctagtgaggatggaaagaaatttctgaaatggttatactgatttggagttgacactttgattattgttcacaagaactctataatagtcacatttttctatacataaattggaagtctggaagataaaatacaagttacattgttaaactgccctaaacctcagtatttatagtgtcctaagcttcgcccatagaagatgaaactattactaatgtaaaatatttaataattgctggacgcggtggctcacgcctgtaatcccagcactttgggaggccgaggtgggctgatcacgaggtcaggagtttgagaccagcctggccaacatggtgaaaccccgtctctactaaaaatataaaaattagcctggcctggtggcgagcacccgtaagcccagctgctcgagaggctgaggcaggagaatctcttgaaaccagaaggtggaggttgcagtgagccaagatagtgccactgcactctatcctgggtgacagagcgagactctggccaggcgtggtggctcacgcctgtaatcccagcactctgggaggccgaggcaggcggatcacctaaggttgggagtttgagaccagcctgaccaacatggagaaacctcgtctctactaaaaatacaaaattagctgggcgtggtggcgcatgcctgtaatcccagctactcgggaggctgaggcaggagaatcacttgaacccgggagttagaggttgtggtaagctgagatcatgccattgcactccagcctgggcaacaagagcgaaactgggtctcaaaagaaagaaaagaaaacagttataacaatatactacaataaaagttttatgaatgtcatctctcagtttcactttcactccaaaaatacctcatttttttagctttgtttggacctttggggggaaaaataccttattgtactcacctacttttggaccacagttgactgtgggtaattaaaaccatagctaaggggaggcctactgtacttacagacacatttttggttctttctttttggtggataaattttctaaaaatggcagttatataaataaactttttccaagtgaaacaaagtgtacttgttttttctttttctttaagttgtattttaaaactagctggctttatattcccctactttcaactagtttaatatctcgctacaagtctcggttataatgttttctttacataatttgttatggtgttttccttcaatgaagtacagagttaatctgctttcatgagtctctaggaagaggtcttcctagacctcttcctagctttttttttttttttgagacggagtcttgctcttgtcacccaggctggagtgcagtggcactatctcagctcactgcaacctctgcctcctgggttcaagcgattctcctgcctcagcctccagagtagctaggattacaggcatctgccaccacacctggctaattttaatttgctatttaggtagacttttttttttttttagatggggtttcgctctgtttgcccaggctggagtgcaatagtgcgatctcggctcactgcaacctctgcctcccgggtttaagtgattctcctatctcagcctcccgagtagctgggtttacaggtatgcaccaccacgcctggctaattttgtattttttagtagagacagggtttctccgtgttggccaggctggctttgaactcccgacctcaggtgatccgcctgcctcgacctcccaaagttgctgggattacaggcgtgagccactgcgcccggctgacttttttttttttttttgagacggagtatcgctcttgttgcccaggctggagtgcaatggcacgatctcggctcactacaacctctgcctcccgggttcaagcagttctccttcctcagcctcccgagtagctgggatgacaggcatgcgccaccatgcttggctaagttttgtatttttagtacagatggggttttaccaagttggccagggtggtgtcgaactccggacctcagttgatccgcctgcctaggcctcccaaagtgctgggattataggcatgagccactgcgcctggcccagctttttttttttttttttttttttaaaggttgacttattcttatagcccacagatctgaagtaacaaatcaggtgtgggcagttcatatctgccagaggttctcattcatctttttttttcttttcgtgggatggtttttttggtgagttgtttagtaccagtcttcccatttatgacctctaacaagaaacacagaaaagtattattgccaaagactaatactagttttcctaaagagaaagaaaaatgcattttactttcaactttttttttttttttttgagacagggtatcgttcatttgccaagggtggagtgcaatggtgtcatcttggctcactgcaacctccgcctcctgggctcaagtgatccttccacctcagcctccccagtagctaggactacaggcgcactcagccacacccagctaatttttgtattttttgtagacaggttctcactctgttgcccagggtggtctcaaactcctgagctcaggcaatcctcctgcttcggcctgccaaagtgctgggtttataggcacgaaccaccactcccggccacttttaacttcctgattataaaataggaacatagtcttgtagagatctttactttttatcatgtatactctggatgtgtttaaaattaatttagggagagtttttggaatatgtgacttctggttcttattctatgcattccattccttttaaattttgatgctttgttatgttaggctctagtttttgtagttccgcttaacgttagggtaaaccattttcatatgtgaatatattgataatgtttcttactagaataataaattctaagaaagtagcctaaattttttgctttaccaaatattatttgaagttactagatctcaactggtctgggtaccaactaattttatagagataaactttgctgtaatgttagttttaaaaatttgacttacatttgaacatacgtctatgtatgctcagaatattaaataatttacttccccacaaaaggtagtaaaactcctttctttctttttctttttttcctttttttttttttttttttgtgagacggggtttcgctcttattgcccagactggagtgcaatggcgcgatctcggcttaccgcaacctccatctcctgggttcaagcaattctcctgcctcagcctcccaagtagctgggattacaggcaagcgccaccacgcccgactaattttgtatttttagtacaggtgaagtttctccatgttggtcaggctggtctcaaattcccagcctcaggtggtccatgtgccttggcctcccaaaaagtgctgggattacaggcatgagccaccgcgcccagctgtaaaactcctttcaacatgtatgttatgtttttgtcttactttatgagaggtactgtaaaaattgaagtattgaggctatgatatggtcattgtgaatttcagtgaagactactgtgttcttcatttttacaatttacaaagcccgttgttatatattctcatatttaatatgtgtaagatataaaataaaatgagcatccctgaaactatcacacagcttaagaattagagtattaacaatatcattgcatccacctgtatttttcttcctatattattcctttcctttcctttcgagataatcactattctgaactttgaatttgtcatttctgtggtctttaaaagttttatcctatgtgtgatatataagcattacttgtacgtttctcttgagctctatgaaaatatcagtgttgccttttttttttttttgagacggagtcttccactgttgccccggcttgagtgcagtggcacgatcttggctcactgcaacctccttccgggttcaggcaattctcctgcctcagcctcccgagtagttgggattacaggcgtccacgaccacactcagctaattttcagtgttgcctttttctttttttgagacggagtctcactctgtcgcccaggctggagggcagtggtactatctccgctcgctgcaagctccgccttgccggttcacgccattctcctgccttagcctcccgagtagctgggactacaggccttttaagatttagtcatataattgtgtccgtaggccattcacttttactgctgcatagtaatctgttgagtgaatgtaccgggttttaagatttttatatcaaaggacatttgcttagctttttttttttaaaccatgtgaacagtgatacattgaacatttttgcatatgtttattaataaacagatttaacgtttatgttgggtgtatgcttgagagtagaattgctggggtatgtttattttcagctttacaatattaattgcttcttcaaatatgctattaggatatttaaaactgagatgaaatttacataacatacaattaacggttatataaagtgtacaccttaatggtgtttagtgtattcacagtgatacacatccaccatctgtctctagtttcagatctttcttgtcaccttagaaaaaaaaccccatttctttcttctcccaagccgtgataactgctaatctgctctctgtctctatggatttgcctattcaggatatatcacataaaaggaatcatgtgactttttttgtttggattctttacttagcataatgaatttgaggttcatccaagttgtagaatgtgtcagtacttcctttcttcttatggctgtatgatagttctactgtgtatatttgtgccacaatttgtttatccattcattcattgatagacatttggcttatttccatttggctgttatgaattataaataatgctgctttaagcatttgtgtacaaggttctgtgtagaaatatgttttcatttcttttggatatatacctaggagtagaattgctgatgcttgtttttgatagtttctcatttcttccttgtaattttttttgatacataggaattttttttaaaacattctccatttgataattccagtgtctgaaatccttgggtatataaatggttttttgtctcttcctcattgaggtttgttttcatttgcgtttgttcttcgattgcaagtttatatttgatcttaatctgttggaatcttaagggcctaaattggggattctttgccccagagaacatttcttttgcttctgctagttgctagggtgtaccactgaactgggaacgcttcaactcctttcctacctgtgtatttgtaaaatggatcagacccttgccagcctaatgtggcaggctctggttcagcaccacaggtttgctgctggcccaaggctttctaatccatctgaggattgaaaatggccctctggataacccagtttttcacatttgctcactgcatacttgtgtctgttcactttgtttttttatttttgagacaggatctcgctgtgttgccagactagagctcagtggctattcacaggaacggtcatagcacactgcacctttgaactcctggcctcaagcagtcctcctgcctcagctttttgagtagctgggacttcaggtgctcaccaccatacctggcttcagttcacttttttgtttttgagattgctagacacccaagaaataggaacgatgtgttaaaacgctttatctaggagctagttattttgtggtgggagggcaggtcattttgacgagcttactgagtagaagagatctttgtagatcttccaaagtttcttgtgggtaaaacatgtgcatttagcaatctgacaaattttcttgaggaaatttggattgggaatacctttaaagggcaatacgtttgtaaaatgcagtggttaagaacaaccattgttctcttcttggacgttcattgtataagtgttagatttttgttgttgttgttgttgttgttgttaactattctctggttgtaagcagaggataagatctaaataatttatgccatacatttcatagttatggcttcactttatattggagtgaaataaaagagtgaatattccaggcatggtggctcacacctgtaatcccagcactctgggaagccaaggcgggaggatcacttgagtccgggagtttgagaccagcatggacaacatggtgagactccatgtattcaaaaaaatctttaaaaaatgtagctgattttgtggtggcatgcatccatgcatcctcccactttgggaggctgaggtgggaggattgcttgagcctgagaagttgaggctgcagtgagctatgaccatgccactgtactccagcctggacaatgcatcaagaccctttgtcaagaaaaaatatgtatattgaaaaatggcccttgtttagactttcctaatgagatgtaaattttgagaaataatagttattatttatggactgcttatatgtgctagcactgtgctaaatgtttacatgtttaattcttataaatactctatgaagtaggtgtcatccccattttatatatgcaggaaaagtacaggaagtcaagtgtacttttgtaaggtcacaaagtacatgataaagctaggatacaaacctaggcagtctgccctcagaactcatgtgattaaaatactgttctttgtcactgtagtgagaaggttttagaagtagcaaataatggtagacttaaatggattattcctaatgattttcttaattttgagaaaagtgaaatactaatagacacattttagtaacttatgctggatattttgttgtggttttctgatttcatttatgtcccattcataagccattttaggggactatattccatattaaaacattcttaaactgaaatttaccctttatagccaaaatgtatttcattgtaccattttttttttaccacattaacatctttattgagttgtattttatacaacataaaatttacccatttcaagtgtacagcttaatgaattttagtaacttaagtgagggattcaactatcactgtaatttaggtttaaaacatttttatcccctcagtaaaatccctcttgcctgtttacaggagcttcccttctcactgtcagccacaggcaaccaccactaatctgtatttttttttttttgagacggagtttcactctgtcgcccagggtggagtgcagtggtgtgatctctgctcactgcaacctctgcctcccaggttcaagtgattctcttgcctcagactcctgagtggttgggactacaggcgcccgccaccacactcagctaatttttgtatttttagtagagatgaagtttcgccatgttggccaggctggtctcaagttcctgacctcaggcaatctgcccaccttggcctcccaaagtgctgggattacaggtgtgagccaccacgcccggcctctattttttatctctaaaaattcgtctttttctggacatttgtataaatggaatcataaaatatctgttttttgtgtctggcttctttcacttagcataatgtttttgaggttcatccatgatatagcgtgtttcattagtttgttctcttttattaccaaatagtaataattccattgtatagacaacattttattcacatactatttaaagataatatatttctagactggatgagacatttaccaactttgctaagtatggtatagtatagtggtgactcagtattttttaaattaatccaccaattatgcttttttgtaatatggtaataatttccctgtttaaactatgattctatccagggcctattgttcctttactaaatggacctaggtggtcgaacttgagttcttgtttctacatgtgatcatttttctttctctttccctgtttaccatttgttttgcttctttaaatgtacctacctttagcaccctccctccaacacttttcttttttttgagacgggagttttgctcttgttgcccagactggagtgcaatggcgcgatcttggctcactgaaatttctgcctcccgggttctagcgattctcctgcctcagcctcccaagtagctgggattgcaggtgcctgccaccactcttggctaatttttgtatttttagtagagacaggttttaccatgtaggccaggctggtcttgaactcctgacctgaggtgattcgcccaccttggcctcccaaaatgctgggattacaggtgtaagctgttgcacctggctcctccaacactgttacagtaaaaagatctggactgaatcctaaggatattttccatcaataaagcaagaagaaaataagaatattgtggcattaatttttggcaacttggattcccatttgtggctttcctaatttttaagctgttttccaggcccctgcaataagagagacgttatgggtttttctgtttctggaatcgattttattctgctggcgatcagcacttttttatgaggtatttgggatacaaagatggttaaaacactaggtttaaattagagttagacatatttcaaaaaaaaggcaggaatagtagtgcatgagtaatatacctttagtaattaacatcccgtatccattaattattaagcatttgcacaaaattttggcagactttttttgcagtcatgaaaatcacatttttccttttaaagtcatgttggaatttaagttatagtatgttctttattacaaagatttaataaaaggctttgcttacatggcatgtttagaaaagaaaaaacatgaaaactactctcttgaaactcattttctgtttaaactttgatgttaaaggtgcttctcagaatgatgatggctccagatccaaggatgaaacaaggtaagcattcactgattgcttaccacagaatgcggagagatagatttcctaaatattcattttagtgtaaacttcaggttcatgatataagaataaatgagtgtcagaaagaattcagaactcaagaagggaaatatttgcaaaagattgaagactcttagcttatcggattagagctatctggagtgggctagggataaccaaattgttgatacttcttcagcttgtagaaaataaatatactggcaccttttcagaggcagggtgttctaaactccagagccatcattgggctgttaagtttcttttccagaaaaactgagataggagctttacacatatgtgaaggtcaccactagctatggtatcatctgggccttcattagaaggggcagagctttgtaccagctgtttgctgttagcccaggtttagcactttcatgtgcctatctgcgttcaagtttggacctaaatgtggggaaaagaaagaaagatcagtttgttactgtgtctatgtagaaaaggaagacgtaagaaactccattttgatctgtactaagaaaaattgttctgctttgagatgctgttaatctgtaactttagccccaaccctgtgctcacagaaacatgtgctgtattgattcaaggtttaatggatttaggtctgtgcagaatgtgccttgttaacaatatgtttgcaggcagtatgcctggtaaaagtcatcgccattctccattctcgattaaccacggacacaatgcactgcagaaagccgcagggacctctgcccaagaaagcctgggtattgtccaaggtttccccccactgagacagcctgagatatggcctcgtgggaaaggaaaaaccttacatcctccagcccaacacctgtgaagggtctgtgctgaggaggagtagtgaaagagggaggcctctttgcagttgagataagaggaaggtttctatctcctgctcgtccctgggaatggaatgtcggggtgtaaagccaaccattcgttctattctgagataggagaaaaccgccccgtggctggaggtgagatatgctggcagcaatactgctctgttactctttgctacagtgagatgtttgtgtaaagtgaaacataaatctagcctctgtgcacatccagacatagtacctttccttgaacgtattcatgatacagattcctttgctcacatttttccctgctgaccttctccccaccatcaccctgttgccctgccacactcccctcgccaagatagtaaaaatagtgatcaataaatactaagggaaatcagagaccagcgctggtgtgggtcctcacacgttgagcgccggtctcctgggcccactgttctttctctgtactttgtgtctgtgtcttatttcttttctcagtctctcgtctccacctgacgagaaatacccacaggtatggaggggcaggcccccttcatctaaaaggtgttgaaagccttggactatgtgaattaacttagactgcttattggttatcctggaaatcggtaacagcagataaaattaggaaaggagatgattctgcttttaaagtagtgagtggcatgtcttaaccacagagtgcagtgattataaaattccatttgattctttgtttttctcaaattgcataagcagtgagtaggaagaagatgatgaaccacaggaggagtagtcagaaggggagaagaacgagaaaagtaatgtcacagactgtgagggaaaattatccacaaagatgggatgttacagtgtcagatgctgcagaggaggataggtgaagaaagtagaacaaaatactgatggttgttgaagctgtgctgttaggtacatgggagtttattgtattagtctatctgcatttgtcatctatgggttgttttttttttgttttgtttttttgtttttttagatggagattcactctttcacccaggctgaagtgcagtggcgcgatctcagctcactgcaacctccatctcccgggttcaagcgattctcttgccttagcctcttgtgtagctgggactactcaagacaggcatacaccaccatgcccggctgtttgtatttttagtaagagatggggttttgccatattggccaggctggtcttgaactcctgatctcaagtgatctgcctgccttagcctcccaaagtgctgggattataggcataagctgccatgcctggccgatagatatatggaagttttttggtttttttttttgttttttgaggtgttgtctcactctgtcacccaggctggagtgcagtgacacaattttggctctctacaacctccacctcacaggttcaagcaattctcctggctcagcctcccgagtagctgggattacaggcacccgccacgatgcccagctaattttttgtatttttagtagagacggggtttcaccatgttggccactatggtctcattcttctgacttcgtgatctgcctgcctcagccttccaaagtgctgggattacaggcatgagccactgcacctggccaatatatgaaggttttcataataaaaagttaaagacaactcatcaagatctttaggaaaatcaaagtttataaagctgttgtgtcttacctaataggtatctgctttcacattcttatgatgaagaatcatctatactaatctcacgagatgtagaatattaaggagcagtaacttcttgcctagaaaatagcagatacttttattctttactgtgattaaaatcaatggggccggtcatggtagctcacacctgtaatcccagcattttgggaggccaagacaggaggatcacttgagcctaggagtttgagaccagcctgggcaattgagtgagacctcatctctacagaaaacaaaacaaaacagaaaaattaaaattaaaaaacggtagttaggcctggtggcacgtgcctgtagtcccagctgttggagtggggagggtgcccaaagtgggaggatcacttgagctggggagatcagtgagctgagatcgtgccactgtactcagagtgatgacagttagaccctgtgtcaaaaaaaaagaaaagcaataggacaggtttaaaaaattacataaatatctgtagactgaatactattatgtctcttttgtctttttttagtattattatatttagtagaagatttcttgaattcctgaaagttcccgcataaaatgttttagtatttcagtttgctttggtcatttacctcttcttttacacaataaacctgtatctttgtataataagtgggtatgtaaaaggttctgttatgccagtgtgttgttgcttgtttgttttctgcaagtctctttcttaacagtattttcatcaaaccacttgtactctggacatctttaaatttattataaaggcattgctggatcccctacttaatgctatttaatcaatgctttttatcttcctaagtgaatcattgtcataaataatttggggaacaatgtcttctgggtttctgagactgcctgacaaatctttgagtatgtcttttgtgttaagcataagttcctgtgcatcagcttttattccagagcacacacacctttgagatgacaggtttgaaaagcattttgtaaactgtcaaaagctgtttaaatgttactacacaataatgagtagtagtcatagctgtgactgccttttctcctttgcttttgtttgcagatttcatgactataaaactaaaaggcagggaagtatatatgtagtggacaggtgcttcgcatccttccctccatcctcatagttatcgtttaggaagtttactaatagccttaggttattttagcttttgatcttaattcaagcactgtctgtatcataaatgtgtttattgatctgttttccccacagcatgtctggatgttttatgtttctcagtgttgtcttctgtttatttgtttatttttatgaagtaccaagattattggtgcctgattttcaagattttgtcgttagatgtgcgttctttttttttgtttttaagtttattattatattttaaagacaggctctcgctctgttgcccaggctggtctcaaacttttcacctgaagcatttgccctatctcggcctcccgaagtgttgggattacagacatgagccaccacgcccagcacatgtgccttcttgattgttactctttgatttgaacatttatgacctgattgtactataatagcattttggttgtctgaggttctctatcctgtccataacttatgaacttatttataggtcagatgtatatgtatgttgaaattagcagatgtttgtaagattgtggaataggggaacaaagttatagtagcacataattagcagctaattgcttttctttttttacttttttgtttgagaccgtgtctcgttctgttgcccaggctggagtgcagtggcgtgatctcagctcactgcaacctccacctcccggattcaagtgattctcctgcttcagcctttcaagtagctgggattataggcatgcaccaccacgcccagctaattttttgtatttttagtagagatgaggtttcaccatgttggccaggttggtctcgaacttctgacctcaagtgatctgcccacctcggacctcccaaagtgctgggattataggtgtgagccaccatgcccagccagctaattgcttttcaaagccaattggtggtgttggttccactgacaattctgtatactgtatagatttccggattgattttcaaaatctatttttagttttagcctcacactttactaaaataacttaaacttattaaaataaataaaataaaccgccagtactattttagtatggccctgcttctcaaactttagagcacattagaatcacctggttcattagatttggggtgggactcaagatttgcatttctaacacatttccatgtgatgctgatgctgctagaccagggtctacattttgagaacacaatggttagacacacacgtttttgcaaattataactattctgggaaataataacagatagtaaaaacagacttcttatataatttttcattatataagaagtctgtttccccaaagtccgagcgtgcagaatttgctagaactgtcaatctgtcttaatttcatacccttttgagagctgctttgaatcttgttttttttgaactgttgtttttatgtttattattagaaaataatacgattgttttaaaagatgtaaattaaatagtgtgtaaagtaaaatgtataaagccttaatagaagaaataatatttgacctaagaccttaaaacagagctataaactgtatatgcatgtcttagaggaattggagggaagaaggaattctaaatggaaagaagaaaaacaaaggtggtgagaaacacaggttgttttgtaggaaatctttaatcataatatttggctaaaggtgcagtaatttcaagggagaaatggcatttggtgattagaaagtagaatcagtcaatatatatttatttgcctttatatgatagacactatgctcaacatgtctattcttccttcagttagtccttaaacgtttatgagttaggtactattgtctcatttttataggtgatgagaaaacaagcttagaaagggaaaggaacttgcttgagttaatagcgttaagtgggacagttgaacctggaacagccttaccttcagagcctgagctcgtaactatcatgctttactgccacctgaagttgggatgatatacaagtaagtgcttagccattaataaatgttatttttattgttagataggttgggaccagatggaggtttgcaagtaaagacttacggaattttttttttttaaaggaaaggaatgtcttttaaaataatttgattttgttctgttttgttttgttttaagactgagttttgctctgtcgcccagatgggaatgcagtgacgcgatctcggctcactgcaacctctgcctcctgggttcaagcagttttcctgcctcagcctccgagtagctgggattacatgcgtgcaccactagacttggctaattttttgtatttttagtagagatggggtttcaccatgttggccaggcttgtgtcgaactcctgacctcaggtgatcttcttggccttcttggcctcccaaagtgctgggattacaggcgtgagtcaccacgcccagcctaaaatatttgaacaggagattgtatgatcagaactctgtaggaaaaaatattttgacagtggtatatagtttggattatgtggtggtgagtgaaggttgaaagtcctatatggaaatttgttacaataatctattgagaggtaacgaaggctagaactgaggcaatgatggtggaagagtagagggagaatgtgaaagtgggatcaacaggacttagatactgagtgtcaagaggcacagggaggaaaaatgtaaaattgcctgggaaagacagcagtatcgttcatagaaccaaaacctaagaaggaaggaagaccataggaagagaatgtttagagggacgggaggacctggcagggcatggtggctaacacctataatcccagtgctttttgggaggcccaaggggaagattgcctcagcccaggagtttgagaccagcctgagggacaaagcgagaccccatctctacataaaataaaaaattagccaggtgaggtagcatgctcctgtagtcccagctactcaggaggctaaggcgggaggatcccttgagcccaggagttcaaagttgcagtgaactgtgattgcgccattgcactccagcttgagtgacagagcaagactcttgtctttaaaaaaaggaagaaagggagggccttgggatgggagagttattctagatctcctgcatttagttgataatttgatactagaggatgggagtgagataaagagtagagattgatttgggaatcattctctgagataaccttagaaatcatggaatttgagatcctagggaaaaatctgtggagaaagaaaaggagaacaagaacacaatcatagagacctatatttagtggatggtaggaagaggacaaaccaaaaaagggatgatgagaagtgggagtacaagattacaaaagcactgtcacagaaattaagagaaaaatgttctttaaaagatgggacatttggccggtggcggtggctcactcctgtaatcccagcactttgggaggccaaggtgggcggatcacttgaggttaggagttcaagaccagcctggccaacatggtgaaaccccatctctactaaaaatacagaaaaaagttagctgggtgtggtggcatgcgcctgtaattccagctactcaggaggctgaggcaggagaattgcttgaacccgggaggtggagattgcagtgagctgagatcatgccactgcactccagcctgggcgacagagcaagattagattctatgtcaaaaaaaaaaaaaaaaaaaagatgggacatttaacactatcaaacgctgcaaaagagaattaagtcttagattaataagctagggaaaactggctgtttggaaaaagaaaagaaaaatcaggttaacgccttagcttatcccaagatgagagtagattcgatgttgacttttaaattgattttaaaaaattaaactaaaacctctagaagaaaatatgaatagttaattcattgctgaataaatttagctatatagaaatataaaactcttataatttaaaaatgtattatcaaaagcaaaggaaatatttaaccaatatgagatgctcagtattctcaggttgtaaagatctcttaaaaataggcaaaattattcccttgtaatagataaatgtacaaaaaacatatatatagttcatgaaagaagaaactgtaatgacctgtaattatatgaaatattgttcatctttaattttaaaaagcattaacatttttgcttaccaaattggcaaagattttaaaactttatggtgaaatgggtgtttttattttgcggtacataatttgaaaatgtccagcaaatgggagaacaggtcaattattacctttaaaactattttttagggccaggcacagtggctcatgcctgtaatcccagtacctttgggaggccaaggcaggtggattgcttgaatccaggagttcgagaccagcctgggtgacatggcgaaaccctgtctctactaaaaatacaaaaaataagtccagaatggtagcctgcacctgtagtcccaactacacaaaaggctgagtcaggagaatctgtggaacctgggagatggaggttgcagtgagctgaaatcatgccactgcgttccaggctgggtgacagagcgagaatctgtctcagaaaccaaaaacaaacaaacaaaaaaacactattaaaaagaaacaatttaggccgggcgcagtggctcatgcctgtaatcccagcactttaggaggctgaggcaggtggatcacctgaagtcaggagtttgagaccagcctggccaacatggtgaaaccccgtctctactgaaaatacaaaaattggccgggcatggtggcaggtgcctgtattcccagctacttgggaggctgaggctggagaatcgcttgaacccgggaggcggaggttgcagtgagccgagatcgtgccattgcactccaccctgggggacaagagcgagacttcgtctcaaaaaaacaaacaaacaagcaaccagtatttttcagctttcaaaatatttgcatgttttgacttggttgttctattctgcagtctgtctaaaagaaataagaaatccaggcagggcatggtggctcacgcctgtaatcacactttgggaggccgaggtgggtggatcacttgagcccaggaatttgagaccaccctggtcaccatggataaaccctgtgtctacaaaaaatacaaaaattagccaggtgtggttgggtgtgcctgtggaataggaggatcacgtgagcctggggaggtcagggctgcagcgagcagtgattgtgcctatgcactccagcctgggcaacagagtgaaacaatgtctcaaaaaaaaaaaaaaaacccaaaaagaccaatgaaaagaaagcgattaaattgtagtatgtctatatataattttctgcagtcattcaaaatgattttcagtaatttgggggaaatgcccaatatgtattgagtaactaaccaaataaaaacccacccagaatatgaaaccattatacagtgtgtgattgcagtcacatccaatgagaaggactgacaagagatagtcctatgtcttggtggtaattatctccaggatgtaggatggcagataatttttattttataattagaagttacttttgttctttgaaagtctgttttaaaaattaaaatgggggctaggcgcagtggctcatgcctataatcacagcactttgggaggctgaagtgggcagatagcctgaaccctggagtttgaaactagcatgggcaacatggcaggaccttgtctctacaaaaacatacaaaaattagctgggtatggtggtgcatgcctgtattcccagctactcaggagactgaggcgggaggatcatatgagcctgggaggtgaggcgggaggatcatgtgagtctgggaggctgaggctgtggtaaaccatggttgtgccacggcactccagcctgggtgacagaggaaaaccctaagggaaaaataaaataaaatgcactaactatattaactgcattttaaaaaagagtatagtgaaggaaaggggaatgggaagacagcttgaggaacagataggattgaggaaacattttaggtttgggtgcttgattatatttgtagagtaagacaaaaggtctataagaaaggaatataatgaagatgcaagaaagcattaaaagatgttgggcagtcctagaggatatagtctgccatccaggacatgataggcaaatcatccttgaagaaggggagagagactttttcagacatgggaaacagatggagatggttagtggcacagagaaattttaaggtatgggagaaagttgagggatagcagataatcattatttttagggaagaggaagagtcagggatagctacagattggtattagcaatttgaggagatggaaaaggtttgcaaggggcactggggatgggtccaggagtggattagagatgtagaaactgattgatttctaaacaatatagataacccaattaaagttgttgaataatgtcagctgtagccaatgttggcttggtctctttgacataatccagttttaggtatttgaatactctataaatacaaatacatagactgaagcacaattttaatttttttctatcttttgcctaggtctgtatgattgtataggacttagagctttttgtcctaccaaattattaatttctttattcccattaattttaataccagcacactaatctcattaaacccatggttttactactaggttgtcaaagtcatccgtcatttataattccctagctcccaggagtcttgagctgtgattttttggtttgtttgtttttgggttttttttttttgagacagagtcttgctctgttgcccaggctggagtgcagtagcacaatctccgcttaccacaacctcagcctcttgggttcaagcgatcctcccagctcagcctcccgagtagctgggactataggcgcatgccaccatacccagctaatttttgtatttttagtagagatggggtttcgccatataagccaggctagtctcaaactcctgacctcaagtgatctgtccaccttggcctccttaagtgctgggattacaggcgtgagccactgcagccggccgatttcttctttttaataagaggggagaaaagagtttttgtctttaataattggtagtgaaaacctctgaccttttctttactgggcccagtggaaaatttattagaattgtttgtagagcagcctgccctttaatatgctactttacttagtttatttactccactaaaatacacctgacttctcaactggaacattctccagcattacttgttggtctgaggaatagtagtgtaaaaagtagatgaggggttagttgtggtggaagaatttctgggaggggaagcagttgggcaagggtgaaggaattgaaactactagggaaagtagcctatgatgaagcccaagtgaggcgtgaagtgagctgatgaactgtaaagattctggaaggagttgaaggttgtgatgagactgaataaaagatactgtaggaggcaggtagaataaataggaagttgtagttagagggattttagagttcaagatctaggaagaggaacaattctaagtatcaaggaaatttaaggagttgtacgggttatcagatgactccattgaaaaattgagatcaaaatgttataagggtgtacagcattgatgttaaaattccccaaattttaatccccagcaacaaatcatagcatttttggaatcctgtttctgctgtacctttatcatagtaggtttatctcatgcactgaatgttccttctccctcacatatccctaaaacttgtctagcactgaatgtgcaatttagtatgaattttctagctgaaagatttttaggcatcttgtagtcaagataatacattttaggatgcattcttgggtcacataactcatcagtagcagagtcagagctagaaaccttaatttcccaatttttggtctattctactcattgttatctgttatttaaaaagcagcaggaactcttctttcaaactagttaggaacgtcttaaaatagcatggagaggcaatacgcagtcttaaaatggttttggaaacctcacttagaagaagagaagcagtgtcaaagctgactcttcctgcattcagaccatgttctgcaggtagtcaacactaatcaggtgctgactgccaattactgaggtgttccctctattataagcatttggggaacccccaaagtagtgcttgtcataccttactgtccacatatataatctggggatcttgttaaaatgctagctctgatatagtaggtctagaatagttttgaagattgtgcatttctgacaagctcctgtatagtgctcgtccgtttggtttaaagaccacactttgaatagaaaggccctaaaggatttctgtggtacacagtttgaaaatgtcttttaaaatatgaggactggtacgcagtggctcgtgcctgtaattgtagcactttgggaggccaaggagggaggattgcttgaggtcaggagttcaagaccagcctgggcaacatggcgaaaccctgtctctacgaaatacacaaaagaattagctgcgcgtggtggcaggtgcctgtagtcctacctactcgggaggctgaggcatgagaactgctagaacctggtaggtgaaggttgcagtgagccgagatcacaccactgcactccagcctgggcgatggaatgagacgctgtctcaaaaaaaaaaggaaaaagaaaaaaactatgtgaggagagagagatgttaattagcttgactgtagtactttttttcactatgtatatcaaaacatcatgttatactgtttttattaaaaataaatcattgcaatacatgttttacagaaaaaaaatttaatacatggtactgcctctctgtttctttgtttgtttgagacagggtctcactctgtctcccaggttggagtgcagtggcgcaagctcagctcactgcaacctccacttcccagactcaaacaatcctcccacctcagcctcctgagtagcttcgactacaggcgtgccctgccatgcccagctactttttaaatttttttgtacagacagggccttgtcatgttgcccaggctgatctgtcactcctgcgcttaagtgatcctttcacctcagcttcccaaagtgctgcaattacaagcgtgagccacagcacccggcctctgttagttgaatctattagattttattttgttcaggcttattctgaaattcctaggagtatgggttagtactgtgtagtggtttggaggtagtacataccagagccttggctgagacatctgcagactggctacttatgcccttgtttctaatctatgactagagcccctctggaaggaaaaggaatcagctatatttagccaacagtagcagtagctttcagtctgacatttcctgttagtgttcttccctcctttctatccctccagttgatgtcattgctttgaagaaatgacagctcctttaaaaaaagtctgtggttacttctactctaaatcactagctttgaacattttgtgtaccattgtactgagctgggggatatcagggcctagtccctgggaaatacgtgcactttgttctcctagcatgggaaaaccagacctgcctcaaacttgaattaatagccagtggaaatcaagaaacaacaatggtaaagttatctgtgcttgtgataaaatagtgccctaaggattttgagaacttgactttgttcctgttcatatagggccttctgaaggcttatttgaaatatgaaccagcatacctaatagttctgaatggccacaagtgttcctatgcccttggacaacccatcactctatctctagatgaactctgggggttatataggattctacccaaaggctctttatgtatttaataaggctgaccagaaattaaaaagcttttttgaagctcttaaaaacttagaagttttcatcctcctgctgttttttttcttaaattcaaggagtattcattatttccctctcccccttccttttcccctcttcttacccagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccaggtttgtattccagctctcaatactcttggaaataatgtcctggttggcaattaatcctcaggtaagtgtcaggaaattcttttaggtgaattttctgcgtgtatttttacatttaagattttggaaaagaatgatctttctgtaggtaaccaaaagtgcctttttcctaagtttttgttcaaggtgacgttactgaggcagtgctctactgagaatgaaaatgacatgtgcgtaatataaagtataggcggtgcagataacatggcaataacattgtacctttcttaaatggtttgtaactataacataaatgtattaaatactcaaggatttggttgcttattcctagtatatcctttaaaaagttagtaaacgaaaacccaacaatgctaaatgacccagattttaaataaaaagtacacaacaaacaagcatattttaggtagtaaaaatgtgaacacctataagactatccacttctctgatagccagaacaaatctttaatcttaccttttattgtttctctctgctaatatgtgggcagggaatgcagtgtagttaaaccaaatattactagaaaagtacattagttctctgcaaataattttaagtgaatttctattcaattatacatcttctcctttttatttttttattttttgagacagggtctcactctgtcacccagactggaatgtggtgacatctcagctcactgccacctctacttcccaggctcaagtgattgtctagcctcagcctcccgagtagctggtaccacagtcatgagccaccaatgcctggctaaatatttatattttttgtagagatggagttttgccatgttgcgcaggctggtctggagctcctgagctgaaagtgatccttctgcctcggcctcccaaagtgtgggagttacgggcgtaagccactgcagccacatcttccccttttatagataatttgattaattgtatattctgagtctttaagatgtctttattataatcttggtgacttcctctagacattgttttgtccacaagtatttcttaaaggtgtaatgtatgactactaactatctgtaagaatgttatttcttatcagaccttccatattataaccaacatcaaaaattggtctgtaatctgcacggtttcacaggaaagcagttttaaggagctggaaaattaataaatgaacttaaaataccaaggaatattaataatttcctattctaaaagtttttatttccagtgacatcagaagatcagagaacctgtacatgcttcgtgattagtaatgggacataggggtagacaggaccgaaaaagtaaacaagcctattaactggcagcgttggttatttcccattacttatttctttaagtaattttcacctgtgcatctacatcttaagttttatatttatcatctgccttttattttttacagttaaaattctatcctgaattttagaaaatgaaagattcttttcctattttccccaaatcttttatagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctgggtgagtaactttttaaattaacatatgttgtctttaggattcttcataagcctacttattaataaggaaaataacagtaaacaacagtaaactccattgttttgatgactgttttttaatttcttttttttttgagatggagtgctctgtctcccagactggagtgcagtggtgtgatcttggctcactgcgacctccgccccctgggttcaagtaattctcctgccttagcctccctagtagctaggattacaggtgtgcactaccaagcccggctaatttttgtatttttagtaggggatttcaccatgttggccaggctggtctcgaactcctgacctcaagtgatccgaccacctcaacctctgaaagtgctgggattacaggcatgagcacccagccaactgtttttatgttattgtaactgattttgggggatctggagtcaaaatgaacaaggaaaacacaccatatttgaaatcttcagggcaaagtggaaaaatatattttttcatcaaaggaccctgaaagttaaccaaggccaattaataaaataatcacctaattccaattgcctttttctttaagggggaaaaaatccctttttaatatcaaactatttttttcatcagaagatcattataggctgggcatggtggctcacgcctgtaataccagcattttgggaggccgaggcgggctgatcacctgaggtcaggagttcgagaccagcctggccaacatgctgaaaccccatctctactaaaaatacaaaaaattagctgggtgtggtggcaggcgcctgtaatcccagctactcgggaggctgaggcaggagaattgcttgaacctgggaggcagaggttgcattgagccaagatcttgccactgcactccagcctgggcaacaagagcaaaactccatcttggggaaaaaagaaaaatcatagcaaagtgatagataattcagaaaactgagaaaatttgtgctcataatcccattttaatctctcctgattattttaagattattttcatagattgtttttaagatactcaggtgtaattttttcttgtttattttttgtttgtttgtttgagatgaaatcttgctctgttgcccaagctgtagtgcagtggtgggatcttagctctctgtaacctccgtcttccgggttcaagtgattctcttgccttagcatcctgagtagctgggattacaggtacctgccaccatgcccagctaattttttttttttgagatggagtctcactctattgtccaggctggagtgcaatgatgcaatcttggcttactgcaaccactgcctcccaggttaaagtgattctcctgcctcagcctcctgagtagttgggactacaggtgcccaccatcatgcccagctaatttttgaattttcagtagaaacggggtttcaccatgttggccaggctggtcttgaactcctgacctcaagtaatccacccgtctcagcctcccaaagtgttgggattacaggtgagagccactgcgcctggccatttctttccactcaaatcctgattataccctgattgagagtgacttcggtatcctaggtagacggacctcaagcctcttcaggaatatgttttctcctagagcagatacttgcatagaagaaattgaattcactgtgtgccactgcctgaagattacatttcttcactttattcctaaattgtatttgtatcatctttctaaactgatgtccttatttgccttagttcaataatggtcatttaatggaaatgcaaagacaggattaaaaagccaggtaactcatgcaagtattttaccagccttttaattttgtatttaagttgggagcagagagtggaccagcacgggcgagtttactatgtagatcatgttgagaaaagaacaacatgggatagaccagaacctctacctcctgggtaagtatctaaatttaaaaagaataattttatttagtcagaattgtggattacaactgtataatcttccctacatttctgttttactttttcttgtcagccctttctctgtccactttgttttgtggtaagtacaaattaaaaagaagagagctagacaactgattaattcagagcctgataaacaatttttgttttcacgcatatttctttcttcttttctttattttatagaaacaacatctcgctatgtcgcccaggctggtttttaactcctgagctcaagccatcctcccgcgttggcctcccaaagtgctgggattacaggcgtgagctatcatgcctggccctattttcactcatttctgacctaaagattccttaggtgggctgggcgcagtggctcacgcctgtaatcccagcactttgggagggcgaggtgggaggatcacgagctcaggagattgagaccatcctggctaacgtggtgaaaccccgtttctactaaaaatacaaaaaaaaaaaagccaggcatggtggcgggcacctatagtcccagctacttgggaggctgaggcaggagaatggcatgaacccaggaggcggagcttgcagtgagccgagatctcgccactgcactccagcctgggcgacagagcaagactctgtctcaaaaaaaaaaaaaaaatttccttaacttttcttgtctaagctgtttacctgtttactaatactgtagtttaagtattagtttcttcaagagtaattatttaaatgaataagaattagagcccagtcatatattaaaatgaagttcttattatatttaaaaaactataatagcaaaatcatttcaaattaattggggaagttaaggatatattaatacatggtgttaagcctttaacattaacattaacattacagtccaggcacggtggctcgcgcctgtaatcccagcaccttgggaggctgaggtggggggatcatgaggtcaagagatcgagaccatcctagccaacatgttgaaaccccgtctctactaaaaatacaaaaattagctgggtgttgtggcgcgcctgtagtcccagctactccggaggctgaggcaggagaatgacttgaacctgggaggtggaggtgcagtgagctgagattgtgccactgtactccagcctggcgacagatcaagactccgtcaaaaaaaaaaaatcatataaaataattgaagaaaaaatattttttaatccagaggaaagtcagtaacaaacatggaaaaatacttaaccttgttagtaatctgaaggaaatataccctgaaaaggtactgttttagttattaaattagcagataatatcaagtgttggtgaggaactttacgttgttagttgctgccggggctgggcacagtggctgatgcctgaaatcctagcactttggggggacaaggtaggatgggcgcttgaggccaggagtttgagaccagcctgggcagcatagtgagaccccatcactatagaaagaaaaagaaattagctgggcatggtggcatgtacctgtagtcctagttaatcaggagtctgaagtgggaggatctgttgagcccaggagttcaagattacagtgagctgtgatcatgccactgtactcctgggcaacagagccagcccctgtcatcctccctgcatctgcccaccataattatgtattaatacatacccttcataatcctctctgtgaaatttatcctaaagaaacaattcaaaggaagaaacagtttgctacattaagaatattcattattttattatttatataacatcagaatattggaaaccaccctaaataaccaagcaggtagaaatcgtgaagtcattcgattcatttatgaattatgttcataatcaacacaatcaattctttcaaatgttttatttagaacatttgtatcaatgtttacttacatattttgtacccatttaaaatgataggaagtgggctgggcgtggtggttcacgcctgtaatcccagcactttgggaggccgaggtgggcggatcacctgaggtcgggagttcgagggccgcctgaccaacatggagaaaccctgtctctactaaaaatacaaaattagccaagtgtgatgacgcatgcctgtaatcacagctactcaggaggctgaggcaggagaatcgcttgaacctgagaggcagaggttgcggtgagccaagatcgcgccattgcactccatccagtctgggcaacaagagggaaactctgtctccaaaaaaaaaagataggaagttagttgtgttcttgcgaaaaattgtgtgtgtgtgttttgttttgttttttcgagatggagactcactctgttgcccaggctggagtgtattggcatgatctcggctcactgcagcccccacctcataggttcaagcagttctcctgctcagcgtcccgcgtagctgggactacagatctgcgccaccacacctggctaattttttgtatttttagtagagatggggcttcaccctggtggccaggctggtcttgaactactgacctcaagtgatccgcctgctttggcctcccaaagtgctaggatcacaggtgtgtgagccactgcacctggccaaaaagtgctttttgatatctgtccaaagctatatatttaatttgtgtccctgttatgattgaaacagtaaaaacaacattgaaaagaaactcaaaacttaatgactgtttgagacttactcactaaatatgtattgcattttaagaacctgactaatgcatcttatgaactttcaagaaataaatttttagaaacatttctcttaagttcacttacaaactatttaaaaatacataaaagactccagataaatcaaaagtagtttctcaaggtaaaatattctatctgtttcacaagtatttgttgaattagcttgtttttttttttttttttctgatttagctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatggggtgagcagcctgttgtttaataaactaataagagtattttgtttctagccccttccagcatatgtcatggaaagcactaaaaagaaggtgcaagtagcatggcaacccagttgttgctgttagtaggtattatttttagaccttgtctggggtattctgtgtgtgatctagtttttcctttttgtaatttgcttaagtccaacttcctcttctttttaatgtcaaaaataagcaaaaaataagttaggacaacttaaaattcatgaacaaaagagtagtaaaaataaacccaagcagcttgaaaataattaaaacatttctaaatttactagtgccttgggtctaagagcatctgactctaaagtggttgtgcaaaactgatgataagagtttaaccactttttaccatttggagataaggcttacctaataaattatttggtactctgggagactgaagaaatttgatcctttataggagtatgcttactatattattctggctttaaatctttattatctttgatataaaatcatttcccttctagaaacaaatggtttaaattttacatattttcctgtagagcaatattgcattaatagtgtaagtactccaaaatcttcctttgctatgcagtctttggtgctaggaattttataatctctcaagtcatacagtatcattgatcttttgtctggaggaaaaggaaaataatccattgagaaatttgattctctatgccaaacaagatggatttgttatatagaacctaaacgaagaaaatcaagccttaaggagctcaggagaaaatgccccctccatcaccatctcttcccctccactgccattggtatcatccctgttagaattcatcagagtaaaacagcacctttcgtattcctttcctaactaggcatagttttatcccttttcttcatattcttgtctattccttatacctcatcatcataatagcactcactgctgtagacaaattcacctttatcagcagccttccctttgttccttggttatgactcaggtacttttctttcatataacaattttttcctgccactttctctaaacagttgctaactatatttcccctcccagtggttttaaagctgaaatgaattttgtctcttgtgtactttacccttcttttcatttttttttctatgctgttttacatcttccttcctttctaaattataatcacctgtggtatgcctggccaaatttcttctaggtttgaatacctagcttgcctgccttctttaaaactagtttccacatgggtgttcagtggaaagagcatgggctttggagttagacacatctgggttcgaatcctggttccacagttactttctccatatgactttgagtaagttacttaacctcatcaagacttatttcctttatctgtaaaataaatacagatataaaattgcctcagagtttttctgaggattctttaaaataaagttccaatatatttggcatgtactgaatgcttagaaatgttagttcccatctcttctctccttcatattcacctctgtatatgtaccctttatctcatttttgcattacacaatctttcaaagcattcttttaaaagagagatttagcagatttctggagcaccaacggtgtgttttgactactgtaatcctgaccgatttagtactcagagtctttggaatttttttttttttttttaagtacagcttctccttcatcatcctgtccccccgcccaatccaatataatctgaacaattttctgaaatttttactctttgtacttgatgtggagtggtttgaaattacatagaatctaaacagcctattttagttaacatagccaaagatataagctgaagtgaattaatttaacatccttaataacatttactttaaaaatgatggggtgttgctgagaaatagtctgtttattgagtatatgaagtggcctttgtccaccattgttgttgtgactcttactagcatacagaaactgtcatacacccagcattttaagtctcagagtggatatggttgacagtaaaacactgtgaaatggttcttcctggtccatggcttaattgtgcatgtccctagtttatccgtccagggagagccagtggggttggcttcagtcttaaaattgtcagtacagtatgtcacgtgccaaacttttcagacctcatctgtaacttgggctcagaccttttgtagtgtttcccgctggcctccctgttctcacatcttctcataatcatgtctgtcaggaggagcaaggcagttctcgacagcactaaatccaagagttattatatcagtagtgtaatgtagctcatatcctttaaaacctcatcaatcaaccaggtttctgtagtaggccggtgacatggggttgtaggggtgggtaaagaaacaagaagagatagcaggggagctgctctatattgagatttaagagaaacaaccaaatgtaacgtaaggactttgtttgggtcctgattttaacaaataagcaatcatgcatttaattacagattaggtattggatgataaaattaacattgattctttaccacatgtcataatgacattgtgattatgtaagaaaatgtcagcctggcgcggtggctcacacctgtactcccagcactttgggaggccgaggcgggtggatcacgaacaaggtcaggagtttgaggccagcctgaccaacatggtgaaacccggtctctactaaaaatacaaaaagttagctgggcgtggtggcgcgtgcctgtgatcccagctactcaggaggctgaggcaggagaatcacttgaacctgggagccggaggctacagtgagccgagatcgtgccactgtactccagcctgggcgacagagtgagactccatctcaaaaaaaaaaagaaaagaaaatgtccacaaattttagagagacatactaaaatatgtagtagaggtgaaataatgtttgattttcgcttcaacaaagaacaggagaaaagagataaagaaacttggcaaagtcttgataagtgttgaatcctggtgctgggtatgtgggaatgtgggattcattgccctgttttacttgttttttttttcttccccatagtgaaattatttttaatgcagttagactcttgtttatcttactgaagaagaaagggtttaaccaaatcttactgggtgaccatgtaattatctctacttaattatacatttaaatcaaaacagacttaatatctctacacagagtcggtgtgacttttgttttctttttccaaaggttgactcttactttgctttcatcttatctagccaaatctttctctccaccaatgtgatcattggaaacagaacttttaagaagtcttccatacagttctaggctcattaacactttccagctggaaacctttccttccactcatctggcagcccactcttcctgttcccccaactctcttctcataattctttgtatgtcacatgttctgtatttgatcttctgtttttgagttacacatataatatttttcatagtcatatgatgattctgggtactggtattgtaaatgacttttgggggggttatatatttttctaatttttatattattgatgtaaaatttgtagcacaaaggaaaaatggtcattttggggatgaggattacatatatgtgtagtattttaattttaaataattttaaatgttaggtaatgtttgcaccggttttttttaaaccaaaccaaagtatattggcatttatccataaccatgtgacacttctttaagatacaaactctctgtgttatacattgccagggaagggaaatcagaactcataagcttgctgtcagataagttgttaagttgttaatagtttcatcactttttggttctttttagaatcaagatttatttgctacatcacaaagtaaagaatttgatcctcttggtccattgccacctggatggggtaagtcacatgagtttcttcatggagttctgtcatttcatttttgttctcttccaattgtgtcattttaaaacagatatttatgtctgatactgtgctcttgcttgagtgaatgtctgggaagtttttttttaatatatatatcagtttaattcacaaataggcattaaattccccaaacattaagtggactctttataaacttttattgacgctcataattattttactcttctacagtttaaagatactgatcacccagcaataaaaaaggaatgggctattgatacaccagtaggtctgggtggacttcaagagtaatttgcttgatgaagaaagtcagtctcaaaagatcacaacctgtgtgattcaattcatataacattctcaaaatgacaaaactagagatggagaacatattagtacttgtcaggaaacagggtaggtgggaagtggattcaaatattaaaaggtaacataagggggttttacgtgatgatgatgattccatcatcttgattccatcaagatccacaagatgattctgtatcttgattttggtaataggtgattacattaataatgtatacatgagataatgcatagaactatatatttacagaaatgaattcagcttttaaacatggtgaaaatttaacaatgtttgtggttaacaataatgtaccaatgccacttctgtggttttgatgctgtacgtagttatataagatgtcaccatcggagaaaaatgaagtatatgtgagactttttgtgcaattttttacatcctgtgagtaataattaattcaaaataaaaagctttttaaaaaattgtaatcacattggttctcctaaaacaaggttcagagtttggttaagttggcattatctgatagaagagtggaaggagcacatgtgaaggtgtcaacttcatgccctacaccatggagctcaagagcatgtgctgttattgagtatatctgataccttagttattaaaatagaccttaatagactttaagctggacctaccctactaggttctattttatagtgttatgtaatactcacagcattatatagcaaggtttcagaataaagatggcagtatagaaagttttgcatagaagcaagataataatatattataatagaaagtggaaaacaacataaagcccattgtggaaccagagttttgtcttcaagagctgaagatacttcataatattgggataaattcagaaagtgatatttctagattgggaaaagttggtccccaagatacattgttctagcagtactgagttatggagaaaaaatgttggtgtgtcaagatcacatgagtctggttaataaatgctactgggggtataagcttttctttgagaaatctcattgctcttatttaaaggtatctattctcaaaacacttctgtgagtcaaaaaggtaaaaaatgtattaggctatacatataactgttactgttattagcttataacaacaggaggacatatgaaagttgtgggcccgatgcagtgacttacacttataatcctaacactttaggaggctgaggcaggtgaatcgcttcagttcaggagttcaagaccagcctgaccaacatggtgaaaccctgtctctactaaaaatacaaaaaaattaggtgggcttggtggcccgtgcctgtaatcccagctgctcaggaggctgaggcaggagagtcacttgaacccgggaggcggaggttgcagtgagccaagatcgcgccattgcactccagcataggcgacagagcgagactccgtctcaaaaaaaaaaaaaaaaagaaattagccaggcatggtggtgcttgcctatagtcccagctactcttgaggctgacgtgggaggatcgcttgagcttgggagtttgaggcttcagagagtcgagatcacactattgcactccagcccgggtgagagagcaagaccctgtctcaaaaaaaagaagaaagcaagttgtgaatttctgtctttaccaaagcttggcagcctggagttaaggagtcctctgtctctactgaagcctaagagtggtcagtgaatggttagtatgaagtactttagaagaggggatgttgcccttacaaggcatattaaacttaacctgtcagattgcattcccagacatggatggttttatttctagagtttcccagctttatttctagagttgtatgtcttatgttagagaaggagaaaaaaactcatgcactaaaatgatatatccaaaggatatgtatttatcctcttaattttattgcaggccaggcatggtggctcacacctgtcatctcagcgctttgggaggctgaagtgggaacattgcttgagcccgggagttggagatcagcctgggcaacatagcgaaaacctgtctctataaaaaaatacaaaaaataatctgggtgtggtggaacacctcctaatcccagtttcttgggaggctgaggtgggaggatcacctgagcctgggaagtcaaggctgcaagtaggccaagattttgccactgtgcttcagcctgggcaacagagtgagaccctgtctcaaaaaaaaaaaaaaaaaaaaaaagttgcagaaaaactatgacctgcttcatggttatggtcagaaacaaaactgttttactccttctctagggcaggtgtagaaaaaaatgcctcttgcttatagaaagctgaattgtgagctgcgtttcctttatcagaaaagtctttcctgcaagttggtccttgccttctgtattcatcattttctttatcatccagatctttctcatttttactttagccaacaacactttgctaaaatactgtgttaaagatccagcttagccaggttattagagtcttgttctgctattttacagtctgcttatttttaggtttcacattcctcatgttgaagtagtgctacagatgtttatgattatcctactcagactgagcactgctttatgacttgctgcctttggggcatataccttacttgtagaaccagtgacagcactggtagtatatcagatgaagcaacccacccaacataccttcaggattgacccaggttgatagaatcccatcttacagcttgcaccctttcagacttttatatgctgggactagacattcctacctcataggctcagtgaataattggttttttagagatgggggtctccctgtgttgcccaggctggggtgcagtggctgttctccagcatagtaattgcacactacagctttgaactcctggcctcaagcaatgcctcagcctcctgagtagctggaattacagttttgtgccacgatgcctagctaggcagtgaagaatttttaacacatgaacatactatagaggataaagaacaaagaatacttttggttcaccagtattatttaatagggctttgtataggtgcttcgccaatcaagtatgaagatattctctcattttcctagttactgtgaaagaatacatagaatgacaagaagtgtctgttttttactgttttattcatcaagtcaggaagtgttctaatctgcatttggatttgatgttcagtaatttcagtattgtttattatgtgatttttcaacaaagtcagaaatccgttctacagccacagcacttcctctcattaagtagatgagccttcagttcacaaatgtgtcaaggctgatctcagcagcatatacttagaatatatatgagaaaaatgtttgctattaattttctcttgcctcatgttatcttctgaaaggaaaatcttggcctgaaatctgaactttgctgacagataaaagatgaatggccattacagtggctcacgcctgtaatcccagtactttgggaggccaaggcttgtgtccaggagttcgataccaggctggggcaacatggtgaaaccccatctttacaaaaaatacaaaaattagttgggtgtggcatgcctgtagttccagctacttgggtggctgaagtgggagggttgcttgagccccggaggtcaaggctatagtgcgctgtgattgtatcactgcactccagcctggacaacagagtgatactctttcaaaaaaccaaaagagagagaaaaaagttgagtatgtgctcagctatatacttagggtattatttttaggttataaaaattatttggatatatatttttaaacaagaaagagaattctggctgggtacggtggctcatgcctgtaattccagcactttgggaggtcgaggcgggtgaatcacctgaggtcaggagttcgtgatcagcctggccaacatggtgaaatgctgtctccacaaaaaatacaaaaattagctgggagtggtggtggacgtctgtagtaccagctatttgggaggctgaggcaggagaattgcttaaacctgagaggtcgaggctgcagtgagctgagatcgcgccactgcactccagtccagcctgggtgacagagcaaggctccgtctcaaaaaagaagaagaagaacaaagagagaattctggattctgcaaataaatttttaaaatttaatgcgactgccaagacatgaacttatcttgctgttatgttaacaagagcaagctgtaacactgtgggtgattatgtttttcttttttttttttttgagacggagtctcgctctgtcgcccaggctggagtgcagtggcgcgatctcggctcactgcaacctccgcctcccgggttcacgccattctcctgcctcagcctcccgagtagctgggactacaggcgcccgctaccacgcccggctaattttttgtatttttagtagagacggggtttcattgtgttagccaggatggtctcgatctcctgacctcgtgatccgcccgcctcggcctcccaaagtgctgggattacaggtgtgagccaccgcgcccggcctatgtttttctttagagacagcctttgagttggtgataagcttatgcactgaagatgccttgaccagcaaaacctgtgtcatgagaggcagagtcccacttttctgtatattatgcacattggaattctattttcattttaagctaagctgacatagaaagtagctttaaatgtcagaagcaggtttgtgatactcactagttacatccattttaacctctgttatcactactacttgaaatcatggaattatattggaaataaaattaaatattaccaaatacgaacttgacaagactataatttggcctaaaactgatccagatgatctcaagtgttaatttgaaaaatagttaccaatttttcttttttcttttcctgtttttttttttttttttttttttttttttgagatggagttttgctcttgtcgcccaggctggagtgcaatggcatgatcttggctcactgcaacctctgcctcccgggttcaagcgattctcctgcctcagtctccctagtagctgggattacagatgcccaccaccatgcccagctaatttttgtatttttagtagagacggggtttcgacctgttggccacgctggtctcaaactcgtgacctcaggccatccgcctgccttataactcccagagtcctgggattacaggcgtgagccacagcgcaggaccaatagttaaccaatttttcaaaccacaggaaataaaaattagccatgtcaacatggattaaacttaaatttttgtttaaaatgctgatttttagaaactaagctttttaaaaagtttcagttcattctatgatctttaatacatcagttcattctatgatctttaatacatcagttattcattttgagacatgttgaacaaataaattaggatacagcaggaatcccatcttttgaagaattcattggtaatgggacagagaagtcagtaattcaaaaacagcagaactgttaaaatttcaagtctacttaatttgtttactaaaagagaaaatgatatccacattttttttttttttttgagacggagtcttgctctgtcgccaggctgtaatccatctcggcacactgtaacctctgcgtcccgggttcaagcagttctcctgcctcagcctctcgagttgctgggactacaggtgcacaccgccatgcctggctaattttttttttttttcttttttgtattttagtagagacggggtttcaccatgttgcccaggttggtcttgaactcctgagctcaggcagtccgcccgcctctgcctcccaacgtgctagaattacaggcatgagctactgcgcccggccaatatccacatcttctaaaccttaatggggagatttcaggaaagcaggtaaaattttgtagactgggaacacgaaaacagggagaatcctaaagcaagaggcagttggggaacaagcagcagctttagatgcagtaattagagaactgagttctgcttcctatcccttgtatagaaagttactgtagatatttaggattttggaactgggtatattaaggaacgagatatgttaagtgtattaagaaaggaagtattttttagttgaataatgcctagcatataccttatattatattccttattttgtatatatatgtatgtatatatacacactctcaaattacattatattccttattttatatatatttttatatattgtgtgtgtgtgtgtgtgtgtgtgtgtatatatatatatatatatatatatatatattttttttttttttttttttttttttttgagacagggcctcactttgtcacccaggctagagtgcagtggcatgatctcagctcactgcagtcttgacctcccaggttcaggtgaccctcctgccttagccccggaagttgcttctctgtcccattaccaattaattcttcaaaagatgggactcttctggtatcctaatttttttatttgttcaacatgtctcaaaatgaataactgatgtattaaagatcacagaatggattcaaactttttaaaaagcttagtttctaaaaatcaagttgtacaagttgtggggctaaggcaggaggatcacctgaacctgggaggtcaaggctgtagtgagctgagatcacgccactgcactccagccagggtgacaaagtgagaccttgtctccaaaaaaaaaaatatatatatatatatgtgtgtgtgtgtgtatttatatatacaaatgtatatttaatatatatcaaatatatatattagtgggctgccagcacggcccactaatttttttgtattttttgtagagatgagggttcaccatatggcccaggctggtctcaaactcctgagctcaagcaatctacttgccttggcctcccaaagtgctgggattacaggcctgagccaccccgcctggcaccttactatattttaatgaatgaaatgatgtagtatggaaaaaaaaaaaatccctaaatggagacctgagtttccatctcatctctgctacttaattctgaggtttcaggtagattatttaacctatctgagttttcttatcttaaggataaagatgaattcacctaccctatgggactattaagaataaaatgaaggtcctgtgtaaaaaactaaataactgttcattattcttgctgctgttggttggattgagtagaaaggtcctatggttaggacattataaagtacgtctacttagcttctaagatgtaatatttctataacttcagtaaggatgtgcatttaatagtcaataaattaaaccttttaaagctgtgatttaaaaactataaaataaacaatgaaatgagaaatgggcaaattcaaataattttattttaccttgagcaagaagactatgccaatgctaatgctttgctttcccctgcccctgcccttcccaaagagaagagaacagacagcaatggcagagtatatttcgtcaaccacaacacacgaattacacaatgggaagaccccagaagtcaagggtaagaatagttacttgtgtatatttaatctcttaaagattataccttatttctaacaactgaagaagatcaaagttcatattcttttgccaaagacctaaaacgttaatcacctacatactttggaaagagaaaattatactattattttcatctaatggaaagtttgaagatttcttcaaagtgcaggaaaaaactttaactgggcatggtggctcatgcccattatcccagcactttgggaggccaaggagggaggattgtttgaggccaggaagacctgcgtaatgcagtgagaccctgtctctacaaaaaaaatccccaaaattgggtgtgttggcatgtatacctgtagtcccagctactcgggagactgaggtgggaggatcacctgagcccaggaagtcgagactgcagtgaactatgattgtgccagtacactccaacctggaggacaggttgagggctgtttcaaaaaaaaagaaaacaatagaaaagcaaaaaaaattatctggcacatgagaatatacttattaacatatatattatttttaattactaagaacagattagtgattctaaatcagggtttcttaacctgagatcactggttaagaatctagggtatgtggtttcaaggaggaagaaaatacatactcagtttcattactctttactggaaatttagtatcttcttccattatgaatgtaggctactaccatgtaggattaagaatgcctaggagtttgccatcagtagaagtcaaagggtgttaaaaaatattctgttgttgttgcagatatctctaaatacttgtttacacacataattacaaggacttgctaatttattaataaagagatatatgtacattatcacaaaggtctccctttgttgcccaggctgttctcaaactcctgtctttaagcgatcctcctgccttagcctcctgagtagctgggattacagaaataatgttttgacaattatgtatatataatggtttatctttgtaacatgttataatagtatgcatattagtttgctagggcgccttaacaaagtaccacagaggaggttgcttaaaccatagaaatgtatgtctcagggttctggaggctagaagcttgagatcacgatgtcagcagggccatgctcactcagaagatactaggaagagatctgttccaggtctttctcctggcttcttgtagctccttggcttgtggcagcatgaatccagtcttcacatggcactcttcctgtctgtatgtctatctgtacccaaatttcctgtttataaggacgtcagtcctataggattagtggcccacagtacttcagtatgacctcatcttgatacatctgcagtaactctgtttccaaataaggtcacattctgagttattgagggccaggatataaacgtgcatttggagggaggacacagttcaacccataacagtctgtaatactatgcatttatcagactgccagagggttccattctgcaaggaatttggcagactgtacagtcggagggcacagtcctccacaaaactgctgccctcacttcagacatcagcgacaatattgggggtccacaggatgactccagaccaactgtctacaaatttgggggcccccaccaccactcttaggtatgataagttactagaagtactgagaactcattgaaacctcttatagtcacatttattctagagaaagaatgcaaaataaaatcagcagaaggaataggtgcacagggcagagtctgggaggctctgaacatccagcattattgtgggacagtacacattgaggtcgagtatcaccaaccagggaagtctctggtatctagggtttttattggggcttgatcacgtactgccctcttggctgaccttttgtctccagtccttactggagatttgagctaatgccctttagtctctagttcctccagaggtcggaactgatgctccatgtcccacagccttgactgtccagtgatcaaagcctccaggcaaacaaagatattcccaatacgcagtgcattccaagggcctaaacatcactaccaagttgctgagggcaaaggccagacctctcttttggtataattataatttgagaacagatattccatggctcaaaagttttaaaaacttttgttctgaaggtatggtttaggttgcactgagctgagatcataccactgcattccagcttgggcgacagagtgtgactctgcctcaaaaaactaaaactaaaaataaataaaaaaagatatgctttcaacttaaaatagttttttctgcctaccaacaaatgaccattttctttttaaatggagttactgcatttacttaaatttattcagtttccataaaggaagaatgaagaatttcatagataacattttcttcctctttagaattttctaagaatagaattgaagaagtcacctttctataatttgctgccatttttttgtttcctcttagatgtttaaagttttatagctaaagttagaattctgctttaacaacacagatgacctagttctatctaatttattttcaagacaacaacttgttactgagcctatcttgcaagtataacaattgagaatcctcttttttacatttttggtagtctgttttatctgacatttgtattcagttaagtgaatacaggtttgttgttgttgttgttgttgttgtttgagacaaggtctcagtctgtcacccaggctggagtgaagtgacatgatcatggcttgctgcagccttgacctcctgggctcaagcaatcctcctgcctcagcctcccaagtagcttggactataggcatgggccaccacacccagctttttttttttttttttttttgggtagagacggtgtctctatgttgcccaggctggtgggaagttgttgccaaacacacaaactgaaatacttgattaaaagatgtcctggcagtatgtaggaaatacttttggaagacctaaagagaacgatcaatctatagttgtagtaggagtaataacaattacaacttgggctcactgtcactttttacctgtctcatttgttcctcatctagtctcatagacattgttgtctctgttttatggacaaagaacctaaagtttagagagattaagcagcttgtctagggtcacaagagttttcaaggtataaagataggtattaaggaggtggcatggaatacttcataaataacatattaggccttgtgtagtgtctcaacatctgtaatcccagtgctttgagaggccaaaataggaggatcacttccggtcaggagttcaagaccagcctggggaacatagtgagaccagtctctacataaaattttataatttaaccgggtgtggtggtatacaactgtagtcctagctactagggaggctgaggtgggaggattgcttgaacccaggagtttgaggctgcagtgaactatgatcatgccactgcactccaccctgggcagcagagtgagattctttctcttaaaagaatctttttttgagttgccttagtagaaaaacagtagatagataatatgggacaaaaggaaaggcttcatttggaggtgtggcagatcatatcttatttaaggaagtgctgagtggcctacactgtgaaatcatgggattcatgaaagggagttagaggtttgggtgcaaagggaacattgaggccagcgtatagaggctgagtgaatgccatatgaagaactttagatcttaatcattggtaatggtggaacagtgatagtactttacagagataactaatttaaataagatctctgttttaggatgatactctgttagcagtatcagtataagagcaggcctagagtgagatcagataaggaaaacgagttaggtggctgttcaaaaaatgcagaagatggctgggcgcagtggctcacgcctgtaattccagcaccttggaaggctgaggtgggaggatcacaaggtcaggagttcaaacccagcctggccaacatggtgaaaccctgtctctactaaaaatacaaaaattagctgggcgtgggcgtggtggcaggcgcctgtagtcccagctgaggctgaggcaggagaatcgcttgaacctgggaggtggaggttgcagtgagccaagatatcgtgcccctgcactccagcctgggcgacagtgagactccatctataaaaaaaaaaaaaaaaaaaaaaaagcaggagagaaacaaagccagagaaaggatggtgttggggattggaaatagagatgaacgtgaagatttctaaggcagaatggttagaacttggttaacaaaatgtttaggatgagtcattagctaatggggaacataagccaagtagacggcagaagacttgagttgtgtttgatggactttgagtttaacctgtttgtaggatgtccacatagagatgtctagtatacaactgaattactgaactgaatggttttctaaaagatttctgctatgtttattgacatttgtagcttctgtacttggcaaacaagcaactctttaatatttttgtttatgttttttcctgtgtttcgtagtcaattaaatgaaaagcccttacctgaaggttgggaaatgagattcacagtggatggaattccatattttgtggaccacaatagaagaactaccacctatatagatccccgcacaggaaaatctgccctgtaagttttctaaacattgtagattaagagtaaaatactagtccttcagattttgatataaagatttgtattagcaaggagtggaagtcttagtatttcttgagttagcttgacaataggtttctgttcattaagtattttgcatttccatctccctcttggatattttatcctagagattttcatatttgttaccatttgttcacacagcactaaatgaaactatttagaatttataggtattgtactgcttaccaattagtctctgatttctgattttcagcttctttcattcccttaactttttgacgttaaaaaagctctcattacttatcccccaccccatttctgagaaagtaaccttttgatttgcttatgtgctttatgacttcttttgtgggaaaatatataacctaaagtttcttgttttatgtgtacagttcagtggcattaagcacattcatattgtggcataatcattaccaccatccatctccagaatgttttcatcattccacactgaaacactgtaccctttaaacaataactttagcagacctggtaaccagccttctactgtctttctctatgaatttgactagtcgaagtacctcatattattggagtcatacaatatttatgcttatgtttctggattatttcactaaggtcatatcttcaagatttatccgtgttgtaatatgtatcaacattttcattccttattgaagctgaataatattccagtgtatgtatatatcacattttgtttattcacagatatttgcattgtttctgttttttggggggtattgtaaataatgctgctctgaacattggtatataattttctgcttgaggccgggcgcagtggctcatgcctgtaatcccagcgctttgggaggccaaggcaggcggatcacctgatatcaggagttggagaccagtctggtcaacatggcaaaaccccatctctactaaaaatacaaaaattagctgggcatggtggcgcacacctgtaatcccggctactcgggagtgtgaggcaggagaatcgcttgaacccaggaggcagaggttgcagtgagtcaagatcatgccacagcactccagcctgggtgacagagtgagactccgtctcaaaaaaacaccaaaaaccaagtatctgtttgagttcctacttcgatgacttttgtatctgtacccaaaagttagggttagggttagggttaggatgacttttgtatctattgctggattaaatattaattctgtatttaattttttgaagaaccaccatactgtttttcagtgtctgtgccagtttacatttccaccctagtgcagaagaattccaatttctgtacgttctcatcaactcttctgttttttttttcttcatcatcatcatatttttttttaaatagtcatccttaatgtcgtatttcattgtggtttttctttgtatttttctaatggtttgtgatgttgagtatcttttcatatgcttcttggctatttgtgtatcttctttggaaaaatgtctcttcaagtcctttgcccatttgttgttgttttttttttcttttgcgattgagttttaggtgttctctatatattctgggtgttaatcccttatcaaatacatgatttgcaattattttctcccattctgtggtttcctttttactgtagatagtgccctttgatgtataaaacattttagattttgatgtctgatttgtcaattttttcttttcttttcttttttgagacagtctctctctctgtcacccaggctggagtgcagtggcacaatctcggctcactgcaagctatgcctcccgggttcacgccattctcctgcctcagcctccgaagtagctgggcctacaggcgcccgccaccatgcccagctaatttttttttttttttttgtattttgtttaatagagacggggtttcaccacattagccaggatggtcttgatctcctgacctcgtgatccacccacctcagtctcccaaagtgctgggattacaggcgtgagccaccgtgcccggcctgttttttctttttttttttttctttctttctttcttttttttttttgagacggagcttcactctttttgcccactctttttgcccaggagtgcaatggtgcgatctcggctcaccacaacttctgcctcctgggttcaagctattctcctgcttcagcctcccgagtcgctgggattataggcatgcaccaccacgctgagctaattttgtatttttagtagaagacagggtttctccttgttggtcaggctgatctcgaactcccgacctcaggtgatccgcccgcctcagcctcctgaagtgttgggattacagatgtgaagctccgcgcccagcctgtcagttttttctttgtcacctgtacctttggtgtcatatccaagaaatcatttccaaatccagtgtcataaagctttgccctaagttctttttttttttgtagatagagtctcactctgtcgcccaggctggagtgcagtggcaagatctcagctcactgcaacctctaccgcctgggttgaagcgattcttttgcctcagcctcctgagtagctggggttttagctacgcaccaacacgcctggctaattttcgtatttttagtagagacggggtttcaccatgttggtcaggctggtctcaaactcctgacctcgtgatccacaccccctcagcctcccaaagagtagggattacaggcatgagccaccgcgcctggcctgagagttctttaattttagctcttacatttagatcttttatccattttaagttaaattttgtatatggtgttgggtaagggtccaacttcatctttttgaatgtgaatgtccaattttcccaagcaccatttgttgaaaagactgtcctttccccattgcatgactgtggcaccattgtcaatagcattttaccatatatgcaagggattatttctgggctgtttattctgttggtctgtatgtctgatttgccaataccacgctatagctttgtagcaagttttcaaatcaggaaggtatgtgtcctccaactttgttctttttttcaagctgcttttgactattcagggtcctttgagattccatatgaattttaggatggatttttttatttctgaaaaaaagaagtcatttggattttgataggggattttattgaatgtacagaatcaataaaatccatattgatcaatacagattcaatatctgtatcaatctggggtagtgtggtcatcttaacaatattaagtcttccaatccatgaatgtggatatctttgtgttttctttgatttctttcagcagtgttttgtggttttcagtgtacgagtcttttgccttcttgattaattcctaagtattttttattctttgtgatgctattgtaaatggaattgttatcttaattttctttttatattgtttattcttagtatatagaaatggaactattttgtgttgactttgtatgctgctactttcctgaattcacttactctggcagggtttttcgttgtggaatatttagtgttttcttcgtaaaagatcttagcttctgaaaacagataagttttcttcttcccaatttgggtatcttttatttatttatttatttttcttgcttaattcctctggctagagcttctggatactatgttgactacaagtggtgaaaataggcattcttatattgttcctcatcttaaagaaaaaacttctaaccattgaatgtgatgtttgctgttagatttttatatgtgccttttattatattgaggtagtttccctctattcctagtgtttcctatccctgttgtggagtgttttatcatgaaagtgtattaaattttgtaaaatccttttttggtatcaattgagatgatcatgtgattttttccccccctcattctgttaatgtggtatattatgttgatttttcataggttgaaccttttttgcattccaggaataaatcctacttagtcaaaaggattaaagtgtgcttttaatatgctgctgagatttttttgctgatttttttttagtatttttccatcagtgttcataaggaatgttgctctgtagttttcttatagtgtggctttcttagagcaaagatggttccctattactttctaattttatacttctacacattaacaacttttatatttaaagcagaaactggaaaatcaggccaatttggtattaatgaaattacagaggtaatttagatatgggaataaattaccgataatattattctatcattcattttagttacaataagtttgggtgcatataatagaaaacactaaaataatagtggtttatgtaagatagaagtgtatttgtccccattatttaaaaatagtccagcaggacttacaccacatcattaccattgctcaaaaaagaagttacaagaaaaatcaggaaatattttattttgaataataattaaaacaccacatatcacaatttgtgggatttaactaaagtagtcttcagcgggaaacttttgttttaaagaagtagatgagaatataggaaagataaaatcagtgatctctgcttttacattcagatgctagaaaaaggcaagttattaaacctaaagtaagtacaagaaaggaaataaataaaagcagaactcagtgaagtggaaaagaaacaatggagcgcccaaagctagaagataaatttttggtaaacattaataatattaacaacccttagtaattaaacctatagagaaaaaaagagggacatcactgtatattttatagacattaaaaggataataaagggtatatttatgccaataactttaacattttagatcaaatggacaaattctttgaaagatatgtcaccaaaatggacacaagaacagatagtaagtctaaatatccctatagctgataaatcaaatgcataattaaataatttttaagggcaggcccagtggctcatgccagtaatctcagcactttgggaggccaaggtgggcagatcacttgagctcaagagttcaagaccagcctaggcaacatggtgaaacccatgtctacaaaaaatacaaaatattagctgggtgtggtggtgcatgcctgtagtcccagttacttaggagggtgagatgggaagattgcttgagcccaggaagctgtggtgaaccgagatcgcgccactgtacttgagcctgggtggcagagtgtgaccctgtctcaaaaaaaaaaaaaaagaaaaaaaagatttctcatgaagaaaattctggcctagatggtgtcactggtaaggttcatcaaacttataagttataaattaatcagtcctattcaaattcattcataaaatacaagtgatggaaatgttttctttttcattttttttttttttttttttttttgagacggagtttcactcttgttgcccaggctggagtgcaatagcccaatctcggctcaccgcaacctcggcctccctggttcaagcaattctcctgcctcagcctcctgattagctgggattataggcatgcgccaccatgcctggctaattttgtatttttagtagagatgaggtttcaggccactgcattccaccctgggtgacagagtgacattccatgtcaaaacaaacaaaattggttaaaggagaaaaatcatacgataatttcaggaaaagcttgtgacataatacaatgcaaatttatgagaaactctcagctaccttggaaacttttttttttttttttttgagacggagtctcactttgtcgcccaggctggagtgcagtggcacaatctcagctcactgcaagctctgcctcccgggttcacgccattctcttgccttagcctcctgagtagctgggactacagacgcccgccaccacacctggctaattttttgtatttttagtagagacggggtttcactgtgttagccaggatgttctcaatctcctgacctcatgatccacctgccttggcctcccaaagtgctgggattacaggcgtgagctgctgcgccctgccggaaacatttttattggaaaaaaaaaatgtaacaaaagcttacagccaacatcacaattaatgatgaaataccgtagaacatcaagagtaagacaaggatatctactgtcattacttcattcaacattgtgtcagtattcctacccagtgcagtgagatgagaaaaagaaatacaggccgggcatggtggctcacgcctgtaatcccagcactttgggaggcccaggcaggcagatcacttgaggccaggagtttgagaccagcctggccaacatggctaaaccccgtctctactaaaaatacaaaaattagtcagtcatggtggtgggcacctgtaatcccagctactcaggaggcggaggcaggagaatcgcttgaacctctgggtcagaggttacagtgagcagagattgcgccactgcactccagcctgggagacagagcaagactccatctcaaaaaaaaaaagaaagaaaagaaaagaaatataagatgtataaattggaaaggaagaagtaaaactgtctcttttcccctcacacaaataacgtttgtatacagaaaatcatgaagaatctagaaaaaattactagaactagtaagtgaatttaacagcgtaacagaatataaagtcaatgtacaaaatcagttgtatttctacactgatatacattgaatttttaagagggagataatgcagtggtattaagtccaaggctagctgagaaaccccatggtgtcatagtgatctaaaattcttctgtcttttctaccatcctagagtatggtctcattcttcaaagtgacctcatggtctgtgacagctgctgaaacttaagctatcagggtcacactccaggctggaatttggaggaagggcaaattataaacacacacacacacacacacacacacactcacacactttttagctattttagctgtcttttagaaaatccttcctttttttttttagacggagtctagctttgtcacccttgctggagtgcagtggtgcaatctcagctcactgcagccttgacctctgaggttcaagtgattctcctgcctcagcctcccaagtagatgggattagaggcacctgccaccacacccggctaatttttataatttttagtagagacagagtttcaacatgttggtcaggctggtctcaaactcctgacctcagatgatccacccgcctcagcctcccaaagtgctgggatttataggtgtgaaccactgcacctggccaaaaagatccttcttgaacgtttcatatgccactttcacttagagacttaagtcacatggccatatagagaaatgtagtctttattatatgtagcattctacttagttaaatagcaagggttttacttaaagataaaggagggaattgatgttgggatagacagcattctcagatagaaacccctttacttagccctcttagatttggcagcatagaaaagaaaatcccacaacagtagtttcaacaaaatcaaagttttttgtttgttttttctgttacttaaaagtattcttggggaaatatctctcaaaaagcaaagaatggttttgttaggaatatgctatatttttgaatttggttagtaagtgctgagagagggtgggatattttgctttccttcagcattatcttttacaagcagctatatatgtcctgttaacccttgttcttcctcagagacaatggacctcagatagcctatgttcgggacttcaaagcaaaggttcagtatttccggttctggtgtcaggttagtattggaactgtatctctgtactgccttaactttgttgtgttgctttatattacacagtgattgtgaaaatacttaactcacagaaaaacaggtaaaatgggaagcattagaatctctttcatctatttttagagcaaataaatagatttatcaattagatcatttgtgactttggttattcagaaaattaaacctcatgtaatcccagcacttggggaggccgaggtgggcagatcacttgagctcaggagttcaggaccagtctgggcaacatattgaggccttacctctgtaaatatatatatatatatatttcttttttttaaagaagaaaattaaacttgaaaccatctgaataagcaaacattgtcactttttcctcagtagccattactttcagattgcaggaatcttcaaatgtaagtaaaagcattttctgatataattttaaccagctactttatttttatgtgttattttttctgagacaggatctcactctgtcacccaggctggagtgcagtggtgccatcatggcttactgcagccttgacctcctgggctcaagtgatcctcttgcctcagccttctcgctagctgacactacagatgcacttcaccacacctggctaatttttagtagagacagggtccccttatgttgcccaggctggttttgaactcctggattcaaatgatcctcctgcctcatcttccccaagtgctgggattacaggcataaaccagcatgcccggacaccagccatttttatatctatcgtttgatataactgactttgatagataaatacatgttcatacatacatgaacatgtccatacatagatgttatattcagtgtttttttcagatgtgatcaactatttcttatgtatagatatttactggaattaatgattttttatgggttctaaatcaaatgaatgccctagtcaaagttactgtgatgaatatttttacacttgaaactcttgtaattaactggacccatggaagcaagaagaatctggattaactgttggaaaataagattaagtaaagatttatttgacacattattaagtaagcagtgtgcatatgagatcaatcttgtggttcttctgtttgcatatattaatctgtaattagcatgaatactcacagatgcattggtaaacattattgaatataaatttatattcatagtctacttgtttctgtaaggaaagataaaagtaaataaaactttccccttttgatttttttgataggctattttaatgatgagagttgacttaagtcattcttctatttcagcaactggccatgccacagcacataaagattacagtgacaagaaaaacattgtttgaggattcctttcaacaggtactgtttcattctctcaataatttcccccctggctgctagcttaggacccgcttttggtgctgtcagtttaatctgaatggtttggaacttcttgagttgaagtccttgaatttttcaagtgcgatctcattgccagccaccttagggcttaaggggtgtgtgtgtgtgtgtgtgtgtgtgtgtgtgtgtgtgtgtgtttcaggtttccatttctaaactggtatgatttgaaataaatgcataggtaagataataggtttgtgtgtgtgtgtgtgtgtttcaggtttccatttctaaactggtatgatttgaaataaatgcataggtaagataataggtttttggtagccaaaggtctggaaatacccatatgaacattatcagattgtagaaaggagaggggaagacttgttttaacacttcagtattacttggatttttacaataagcgtatgttactcttaacaggaaaaaaagtggtcattggtgacagtatctaaaaaaagatatatatgagttttcagaagtagtcagcagattttttcacttgtcttcagaacttgagttaatttactcgctgggcacggtggctcatgcctgtaatcccagcactttgggaggctgaggcgggtacatcacctgaggtcaggagtttgagaccagcctggccaagatggtgaaaccttgtctctattaaaaatataaaaattagctgggcctggtggctgatgcctgtaatcctgtaaagctactcaggagagtcacctgaacctgggaggtggaggttgcagtgacccgagatcgtgccattgcattccagcctgggcaataagagcaaaactccatctcaatttaaaaaaaaaaaaaaaaaaaaaaaaacttgagttagtctactattcgcctttatcttgcagatattactaataacctactgcaactaaatgttaaaatgattgtgttatgtagaagattaaaataactagtgaaaaaatgtcttaaaatgaaaacatctggttagtccttgttttatagccagatgtggaagtgattattttcaagcaccagtggtattgtagcaactacagcttcaagaagtacggaattgaatgcaaagggaaaagaggttgagacagttggtggtagctggctacttgctttcagaatcagtttttttcatttcctaatggaacgtcagttattgacattaaaggaaaaagccagttggcatcctgctatgtaggagcagaaaactttttagtgggaagtggagctttactgaggccatttagagaattatgagtcatccccttgatctgatgtaagtgtcacaggccattccccaccctagtccttgcccatttctaattttatgttcataaccacccttttatcactctgttggacgagtttcttgaagcaacttgcttacctcttaccagtcctggaaagatgcaaaacatgattaaactgttcctttatcttttgactttgaaacctaaagtcttttgcaaaataactgactagatggttctaaaacagtttttaaagctgtcaccctagatttgacatccatcaaataactggttaataaaaatataagagaacaatttgattgcttaaaggtaacaaaactaattgcaaaattagcatcagattttctttggatagttgttggataaaattgctctgggatagtgtttcttctgttaacacagcacaactggttatgggggaaagtacaggagaaaacaattttttttccttcatattagacatttgttaaaaggaatttttttttaacttttatggatttatttatagtaaagataaggatacaaatgtcttccacacatatgtggtaatctgtggtgctcgccactccctccctcctgggactgacacactgcagcccacgctgctcaccctggagccccatccttgcatgagagaatggcgccacctggggctgtgcgacctgtcagcctgctcattcccttctccagctcaggacagagaaacacaattcattcataaggtttccattctgttataggcagagatttagaaatcagcattgtttttcaaagagtacaaagttaaaccattctgcttagaaaagaataaacccatccaatcatttttcctctagttgagtcaaaacagagatcaaacttctttgatagtgttatgtctgtgttcagacttagacttcagaatcaaaattttgaaatatttatgaggttaataactatctttgaagttaagcagtttactgagcaattaaatatttttgtccatacacattcagatttaactactgctataaattgtcgtaatttctccagtttcagtctgaacataatagcttatagtaataagacacaggttctagtacagattttccaattagtggtatggccctaaacaagccaggttagatctctgccttcacctacatcaaacaggaataataatatatttccttaacctatcttctaggtaaacagatgagattatatgaaaacattttgctaaagtcgctaaacaaacataagaaaaaatatgtaaggtttttctatcacattaactattttttaatatctgactgacatttttgtaatataaccaaaataaaaattgcatactttcttttttttttcttttattgatcattcttgggtgtttctcgcagagggggatttggcagggtcataggacaatagtggagggaaggtcagcagataaacaagtgaacaaaggtccctagttctcctaggcagaggaccctgcggccttccgcagtgtttgtgtccctgggtacttgagattagggagtggtgatgactcttaacgagcatgctgtcttcaagcatctgtttaacaaagcacatcttgcaccgcccttaatccatttaaccatgagtggacgcagcacatgtttcagagagcacagggttgggggtaaggtcacagatcaacaggatcccaaggcagaagaattattcttagtacagaacaaaatgaaaagtctcccatgtatacttctttctgcacagacacggcaaccatccgatttctcaatcctttacccacccttccccgctttctattccacaaaaccgccattgtcatcatggcccgttctcaatgagctgttgggtacacctcccagacggggtggtggcctggcagaggggctcctcacttcccagtaggggcggccgggcagaggcaccccgcacctccaggacagggcggctggccgggcgggggggctgacccccccacctccctcccggacagggtggctgccgggtggagacgctcctcacttcccagacggggcggctgccgggcggaggggctcctcacttctcagacggggcggttgccaggcagagggtctcctcacttctcagacggggtggctgggcagagatgctcctcacctcccagacggggtcgcggctgggcagaggcgctcctcacatcccagacggggcggcggggcagaggcgctccccacatcccagaccatgggcggccgggcagagacgctcctcacttcctagatgtgatggcggccgggaagaggcgctcctcactcctagatgggatggcggccgggcagagacgctcctcactttccagactgggcagccaggcagaggggctcctcacatcccagacgatgggcggccaggcagagatgctcctcacttcccagacgtggtggcggccgggcagaggctgcaatctcggcactttgggaggccaaggcaggcggctgggaggtggaggttgtagctagccgagatcacgccactgcactccagcctgggcaccattgagcactgagtgaacgagactccgtctgcaatcccggcacctcgggaggccaaggctggcggatcactcgcggttaggagctggagaccggcccggccaacacatcgaaatcccgtctccaccaaaaaaatacgaaaaccagtcaggcgtggcggcgcgtgcctgcaatcgtaggcactgggcaggctgaggcaggagaatcaggcagggaggttgcagtgagccgagatggcagcagtacagtccagcttcggctcggcatcagagggagaccgtggaaagagagggagagcgagaccgtggggagagggagacgggagaggcagaggcaggggcaggggcaggggcaggggcaggggcaggcagaggcaggcagaggcagaggcagagctgcatactttcaagaatgacaaaatttagtagttatacaaggtgaattaaaatagtaaagctagtcataatacagaatccactcatccatgtactcattttatcacttaaataataattataaaaataggtaaaattccatgaaaatattcctatttgttctagttcaggaggtcttgcccacatcaatggattgtctggcatcagccactgtttcaggcactcgaacagtcatattgtccatagattttgtcaaacagatttggcagcccaactgtgatctgttagtccatatgccagatcgagcatgtgattctcctcatcagtgattgcatctaacttctcatatatgtgatcttcaaagatgagatgacaggttaaacaagccagagttccctcacatgcaccaaagccatcaatatctggattattttcaaccacaacgtctagcagagaatcaccaacttttcctttggttgttaatgtttcaccatcacagtttataaagtggactgtcactttatcttctgagctgctccgcgcccctgccgacacgctcagcggccggctcgcctccgcgctccggcccggtccccggctcctcagcaggccgctggctccagcgcgggacccggcgtggtgcagccaccggccggccgggtcgccgaggaccgcagaagcggcgcgcagcagccgggcgcccccagcggcagccatcgcccgcggtcgggaactcaaaaggaattatttaaagatagttttaaccttgtgacatttctcatttgcgttgctgtatacctagcctatactatttgtagaatacttggcataaacgtacatatgtcataagcattgaagttttttgttacatctgggattttctggcttgagtgggatgtgaattcataaatgtgatggttttgtgggggaactgagagaacttggcataaacaggtatacttactgatcctctctggatatttaagtgtggtaattcattcttatcactcaataaagtataggcattatatcaacttctccctttccatgttcttctgtatttttttgtaagagtaattgatatatgcagtgctatacacacacaattttggtcaaataatttgttttacgtggcaggaaaccaaatgacctattgattgtagctcagtaatgtactgtccttcaaatatgaagatgatcctattaaccacctatagtgggacacaaaatttgagtagtttttgtaggtagttatcggttgccgctgagaatgagtttccaaagttaactgggtctctctgcagctactggttttcatttctcggttagccagttagctgttacgtgtacctttggctatgagtcaaaaccaaacctaataaccaaaccacaaaaaccccacccaacctctgaccatgcccatctatgttctaagtaattgatacaggcaacattataagtcaatagtagcatcattaaaaataaaatcacggccgggcatgatggctcacgtctgtaatcccagcactttgggaggccaaggccggtggatcacctgaggtcaggagtttgagaccagcctggccaacatggcaaaaccccgtctctactaaaaatacaaaattagccgggcgtggtggcgcacacctgtagttccagctacttgggaggctaaggcaagagaatcgcttgaacctgggaggtggaggtgtggtaagccgagattgtgccattgcactccagcctgggcaacgagggagagactgtctaaaagtaaataaataaataaaatcacgtgtataaggtatataaaaatatagatgtatataataaaatattattaaacgttactataatgtattttctgtatgtgtggttttgactagaccatgaacacctttctgtgtcatgggagatttcagttacctataacgaaggagatgtcaaacctagaagtggtagacagtgtttcaatagcttttttcaccaattattttactttatttttttttagataatgagcttcagtccccaagatctgcgaagacgtttgtgggtgatttttccaggagaagaaggtttagattatggaggtgtagcaaggtagtgataatatgaatactcagaacttagttcctttcctccaaaggtaccattgcacttcgcttgcaagtattattttctcaatcttatattttaggaaaatgaaatatgtcaaattattatacctttattatgctgtagctacagaatttcaaaaaacatttaattcagcactttctatgtcatgtacagctgattgatttcacccacatcactcatttaatcttcagggtaaccttgtgatacagaattttataggatgatgaagaggcttagagactcacctaaggcacataataaattgtagagcctggatgcaagctttgtatcatttaaccagtagccagtgttaagtctaccacctcttatatctgttccacagaaaacacaaggccttatagtgatcaggtcattttccccaccacagattactgatacaggtaatagttactcagtttccaactgagattagtatatttgtcatgaaaatatagaaggttgtacaaaataggagttgaaagtcttaattctgctttcttcatattggagcttaatattggaccctagaagatagtctctaatgagaattgctttgcttattccatgataatatatcacaccaggttaaaaatttgcgtagagctggcattgtgcaaagacaaggactttggggtcaagacgggatttgaatctttttctgctctcaatagtgtgatgttgggcaaatattttcatctgtaaaataagaatattaatatctacatttctaggattattgtgaagagtagaaatattgtttattaatgacctagttcagagcctgtgtctttgtgtgcacgctaattggtggctacattttagtagttcttatttatattatgtttctgagtttaaataaaattatttcaaaatatatgatataatcatagataaataacaaatatgagtagaaacatgtaattttgtataagttaaaggcaattgaagaggagagcaatcattcattcctaataagaaaataaactagcaatattaaagtgtgtcatgagatacacttatcacaagaggccatgaactaacaggaaaagagatccagattacaggcttatgccacatgtattaccccagatcacccatgttacgtagttttagtaaccataatcttatgttaatattggttcatattttcacaatatttgacttagagtctcagcatttttctaatatgtgagtggctggaattaagaactttactgcctctgtgaaattggaagaattttcatattgatttaacgtgtagggagtagtggcacttgatggggattattatgggacttagtaatagatggaatattagattgatttcagccactttactcacatgggtaaatagtgaattttaaatttgcctatgaatagtatttcattgattctaagactaatattttcattttataatatctctgaaattacgatgtatctcaggtaaaattggaaaatacaatttagtcgttcatcaaataaaggtgcaatgttgtcttagattttttgaaatgtaataatgtatatattaccatctaccaaagatggtgtatactaccaactctgtgaaacagatgtgagtttgaacagaagcaataaaaatcaacatgagattagaaaactatcactagctgaggggtatagatccctgagaactttatagcactgttctttgatttcttggtaacctacaagatttttcatcgtaaattttcttttgatttcagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggtaagtgcaggtcaagaattatgtttactttgcttattcagcaatactaaattttctcttaggtgccataacagatcatatgagagaaagggaagtggttttttgttttgttttgttttttatttttgagacagagtcttgctctgtcacccaggctggagtacagtgacacgatcttggctcactgcaacctctgcttcctggactcagtcagttctcccgcctcagcctcccaagtagctaggattacaggtgtgcgccaccatgcctggctaatttttgtatttttagtagagaaggggttttgctatgttggccaggctggtcttgaactcctgacctcaggtaatccacctgcctcagccttccaaagtgctgggattacaggtataagtcaccgtgcccagccagggaagtgtttttttgagacaaagtttcacttttgttgcccaggctagagtgcaatggcgtgatctcggcccaccacaacctctgcctcccaggttcgagcaattctcctgcctcagcctcccgagtagctgggattacaaacaagcatgtgccaccatgtccggctaatttttgtgtttttagtagagatggggtttctccatgttggtcagtctggtctcgaactcctgacctcaggtgatccacccacctcggcctcccagattgctgggattacaggcgtgagccaccatgcctgacccagggaagtgtttttaaatgaagagtacatctaatatattgaaataaaatgattattgtacagttattttaagcggtcttgtttccttttttcataggctctgttccatgggaaattcatagacacgggtttttctttaccattctataagcgtatcttgaacaaaccagttggactcaaggatttagaatctattgatccagaattttacaattctctcatctgggttaagtaagtttctttttccatgtaatttattaaaatatagttatatgcattccgtgtagtattgataacttatccaaactgtaggcacataattggtttattgtatttaaatgatctatatatcaaacctgtctttttattcaatatgataaattttacaaaaatacacccaagatcttcggcaataatatgaccttagaacagaatcttatttcttcttattttgatgttatagcttaatattaattatttaagaacatgggcctttcgttaaaaacttataaattatgattgaattggtggatataacaaatagtgctaatttcatttgtgcagggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcaggtgagagtgctccttttcacatttcactttttgtttgactacagcacattgataattgcttactaatactaatggagagtatctccaaaaatagtatttgtctctgtactaatcaatatcctatgtaggcagtcatttttcatttctaaaaagtttttttttaagtgtttttaaaagagttttaaggataaaagtcatgcaggatcactcttttaaacatcaactctgtaatcactatgctgtataacatgtcattaaaaagaatctcaaatatatatgcagatattttcatggggaatattgtaaggtgtattaatccactttcctgctgctgataaagacatatccaagactgggcaatttataaaaaaaagaggtttaatggacttacagttccacgagggtggggaggcctcaccctcatggtggaaggcaaggaggggcaagtcacatcttacatggatggcagcaggcaaagagagagcttgtacagggaaaactcctgtttttaaaacctcatcatcggccaggcatggtggctcatgcctgtaatcctagcactttggaaggccaaggcaggcagatcacaaggtcaggagattgagaccatcctggccaacatggtgaaaccccatctctactaaaaatacaaaaattagctgggtgtggtggcatgtgcctgtaatcccagctactagggaggctgaggcaggagaatggcttgaacctgggaggcggggattgcagtgagccaggatagcaccactgcactccagcctggtaacagagcaagactctgtctcaaaacaaacaaacgaatgaacaaaacacttcatcatatcttgtgagacatattcactgtcatgcaaacagcaggggaaagacccacccccgtgatcccatgattcagttatctcactgattctctcttacaacacatgggaattatgggagctacaaggtgagatttgggtgaggacacagagccaaaccatatcattctgcccctagcccctcccaaatctcatgtcttcacatttcaaaaccaatcatgccttctcaacagtccccaaagtcttaacttatttcagcattaactcaaaagtccacagtccaaagtctcatctgggacaagggaagtcccttccgcctatgagcctgtaaaattaaaagcaacttagttacttcctacatacactcagggatacaggcattgggtaaatacagccattcaagatgggagaaattggccaaaacaaaggagctacaggccccatgcaagtttgaaatccagcagggcagtcaaatcttcaagttccaaaatgatctcttttgactccatgtctcatatgcaagtcaccctgatgtaagaggtggcctcccatggccttgggcaacactgcccttggggctttgcagggtatagccccccaccggctgctttcatgggctggcgttgagtgtctatggcttttccagtctcatgctgcaagctgttggtggatctaccattctggggtctagaggacggttgccctcttctcacagctccactaggcagtgccccagtagggactctgtatgggcctccaacttcacatttcccctccacactgacctagcagaggttctctgtgagtgccctgcccctgcaagcagacttttgcctgggcatctaggcatttccatacgtcttctgaaatctagagggaggttcccaagcctcaatttttgccttctgtgcaccctcaggctcaacagcacgtggaagctgccaactcctggggcttccaccttctgaagcaacaacccgggctgtaccttggccacttttagtcatgctggagcagctaggaaacagggcaccaagtccctagagtgcacacagcgtggggaccctgggcccggcccatgaaaccatttttagctcctaggcctgcctacaggcttgtgataggaggggcttccatgaagacctctgacgtgccctgtagacattttccacattgtcttggggattaacattcgggtcctcattacttatgcaaatttctgtagccagcttgaatttctcctcagaaaatgagattttcttgtctattgcattgttgggctgcaaattttccaaacttttatgctctgcttcccttacaaaactgaatgcctttaacagcacgcaagtctcctcttgaatgctttgctacgtagaaatttattccaccagataccctaaatcatctctctcaagtacaatcgtctctctcaagtacaaatccaccagtctctagcacaggggcaaaatgctgccagtctctttgctaaaacatacaaagagtcacctttgctgcatttcccaaaaagtttctcatttccatctgagaacacctcagcctggactttattgtccatattgctgtcagcattttggtcaaagccattcaacaagtctataggaagttccaaactttcccacattttcctgtcttcttctgagccctccaaactgttccaacgtctgcctgttacccagttctaaagttgcatccacattttcagttatcttcagcagcaccccactcctggtaacaatttactgttttcacactgctgctaagacatacttgagagtggacaatttacaaaagaaagaggtttaatggacttacagttccacatggctggggaggcctcacaatcatggtggaaggcaaggaggagcaaggcatgtcttacatgaatggcagccggcaaagagagagcttgtgctgggaaactcccattttaaaaccatcagatcttgtgagacttattcactataatgagaacaacatgagaaaggcccgcccccatgattcagttatctcccactggttctctcccataacacataggaattatgggagctacaagatgtgatttgggtggggacacagagccaaactatatcataaggttgggtaaaatttataactaaaatgatgttttatagtatatcctttctgattatttccattggttcatgctactagtctcaagagctaatagttcattcataccttctcaatagcaattctttccagccagggttaaattttgcatatttacttcttaagttccaggttctgcatttcttgcctggtttttccctccataattgaaagtatactatttaacacagtgagctgtcattatttcttggacaacaatctatccttttaaaagtttattttgaaatctgcttttaaaatgtagtcaattcgtttttgtcaattctccagtttttttgttatgatatgtctagattttcttatgacatatatagcattccagagaggtaatgtttcgagattccaagagatgtactacgtttctcaatagttttgttgctggtaagataacattttaagataatgggcagctaaaaaggtttttagtatctacttcaataatggcttgcttaaaatttcagagtttggggggtttttagtattattttaagaagtataatttccttagaataaaatgcactgatcttaagtgtagaagttgaaatttgacaaatatatatatcagcccaaatgtgagcattttatggagagaagttgtgctagatcactcatttaattaccaattctgtaaacactgttccatatagcatcatgtccatgtaaatatgacccaaattgtggaacttttttttctattctggaccccataaggggccaatgcacacctcccttctcccagccccagaggtaggtaaccatttctgtttagttctatttctataactgtttctgatcttaaacttcctaaaatgaaatcatagagtatgtactcttatatctagcttttagtgtctgattttttcttttatttggtgtttgtgagattcagccatattgtttatatcagtattttattcctttttattgctgagaaatattccatgctgtgtatatctcaaaatttgtttatccattctcttggtgataaatgttttggttgtttctcagtctggggcttttatgagtaaagctgctataaacattcttgtgatatctttttgtggacatatttttcttgagtaaatacctaggagtgaaatcgctgggaaggtgtttgactttattagaaactgccagtttcccaaagtgtttttgccattttgcagtcctactagtagtatttgagagttgcttcacgttcttgccaattcagcattgtcagtaatatctcgtggttttaatttccatttcttccttgactaatggtgtcaagtacgtttttctatgcatattgatcatttgtagctcttcttttgtgatgtgtgtatccaaatcttttatccattctaaaattgggttgtctttttatttgtgattgttggagttcttaaatatttcagatcattgattttagaaactcttctctttcccttcttccctccctcttcttaccttttcttacgatacgtctagatttcgttcttctttgacggagtcttgctctgttgccgaggctgaaatacagtggcacgatcagagctcactgcagtcttaaactcctgggatcaaaggatccccctgcctcatccttctgagtagctgggactgtaggcacaagccactgtgcccagcaaattttttggttgtttttatttttgtagagatgtggtcttgctattttgcacaggctggtctcagactccttgccttaagctatcctcccacattctgttttctaatataaggatttaaagctgcatatttccaactaagtatttctttagctatatcccacaattttttgttgttttttttgagatagagtctcactctgaggttgcccaggttggagcgtagtggtagagtcatggttcactgtagcctcaacctcctgagctccagatagcctcccacctcagccttccaggtagctggaaccacaggcatgtactaccatgcctggcttacttatttatttatttattgtaggagatgggtcttactatgttacccaggctggtctccaaactcctggatccaaacgatactcccactttggtctcccaaagtgctggaattataggcatgagccgccgcatccagtccacacctttttttttttttcccccgagacagagtcttgctctgtcgcccaggctggagtgcagcggtacaatcttggctcactgcaacttccatctcctgggttcaagtgattcttctgcctcagcctcccgagcagctgggattataggcacccgccaccacgcctggctaatttttgtatttttagtagagacggggttttgccatgttggccgggcttgtcttgaactcttgacctcaggtgatccgcctgcctcagcgtcccaaagtgcctggattacaggcatgagccaccacgcccagccacaaattttggaatgttgtgtttttcactattttatttattttatttatttatttatttattatgttttatttttattattaatatttttgagacagggtctcactttgtcacccaggctggagtgcagtggtgaaatcttgcctcacggcagcgtccaattccagaagttcaagcgatcctctagcctcagccaccaagcagctgggactacaggcatgtgccaccacacccacatgacgagatggggtttcaccgtgttgcccaggctggtcttgaactcctgtgctcaagggatccacccacctcagcctcccaaagtgctgggattacagatgtcagccaccacatccagccactctcattgtatttaaaagtatttgttaggtttttttttttttttttttgagacgtagtctctctgtcacccaggctggagtgcagtggtgccatcttggctcactctaagctccgctccctgggttcacgtcattctcctgcctcagcctccggagtagctgggactacaggcgcctgccaccacacctggctaatttttttgtatttttagtagagacagggtttcaccgtgttagccaggaaagtctcaatctcctgacctcgtgatcccctcacctcagcctcccgaagtgctgggattacaggcatgagccgccgtgcccagccaggtcttatttttatcctgataatctctactttataattggagtgttcagtacatttatgtttaattttattactgatttggttggacttttgtctaaccattgctatttgttttgctatttgtctcgccttgctatttgtttcttatttttcccaactttctttgttctttcttgccttctttgaataaagcaatttttactttcttttttagctcttctgttggttttttagctatacctctttgtttaactttagtggcaactctaatgattatgataatatgcatctttaacatccctgttgacccaaacactacttcttgtacaatataagaagctcataggccaggtgcggtggctcacacctgtaattccagcactttgggaggctgaggtgggcagattgcctgaggtcaggagttcgagaccagcctggccaacatgatgaaaccccgtctctactaaaaatacaaaaatcaactaggcatggtggcaggcacctgtaatcccagctacttgggaggctgaggcatgagaatcgcttgaacccaggtggcggaggttgcagtaagtcgagatcacgccattgcgcttcagcctaggggacaagagcgagacttcgtctcaaaaaaaaagaaaagaagctcataataatataattccatttacctcactccagctctttgtggtgttgttgtcatatattatttctacgtgtcacaaatataatactttgttactgttattcttgctttaatttttaactattttcaaaaataatcttatatttactcacattactatttctggttctccttatttctttcctcagattcatttttttttttaaatctaagataatttccctttggctttaaaaaaaaaaacctttctttaccatttcacttagtgtgcatctcttggtgatgcattttctcagcttttatatgaaaacatctttatttcaccttcattttttgaaggatattgtcactagatttagaattctaggtttataaaataatgatgcggtcatgtttttatgatttatgttttaaaaaataatcctcatgtcgtagagacaaatactgaagtatttgttcaaaatgaaattatctgggctgggcgtggtggttcgtgcctgtaatcccagcactttgggagaaggcaggcagaacacttaaggtcaggagttcaagaccagcctggccaacatgatgaaacccccgtctctactaaaaacacaaaaatcagttgggtgtggtggcgcacgcctgtaatgccagctactcgggaggctgatgcacaagaactgactgaacctggaaggtggaggctgcagtgagcccagattgtatcattgcactccagcctgagtgacagagcgcgaccctgtctcaaaaaacaaaaacaaaataaaagaaaaaaaaagaaattatatggcttggcaatggtggcacacgcttgtaatcgtagcactttggatggcggaggcaggaagatcacttgaggccagatatttaaaaccagccttggcatcacagagagacaccatctctacatacaaaaccaaaaatcagccaggcgtggtgttgcacacctatattcccagctactcgggaggccgaggtaggaggattgcttgagcccaggaggtcgagactgcagtgagccaagattgtgccactgtgcttcagctcaggtgacagagtgagacctcatctcaaataaataaagaaatgaaattatatgatatctagggtatgagtaggggcatagatgaaatagatgggcattgtgatgaagcttggacatgtgtacagggggttcattatgttctatttttgaatatattttaagttttttataattttttaaaatttatttttatactcagaatttaaagataagatctaaacttcctgataggttttttcatatttgtttgccagaatggtagctgagtggaggttgtctcgaggtgttgaagaacagacacaagctttctttgaaggctttaatgaaattcttccccagcaatatttgcaatactttgatgcaaaggaattagaggtaatgaattttccttcattcccctgtaccatgctagtaaataatgccttaagttgtctgcttttaactcactttcccatctttcctgttactgtaatatttttatactggggcaaaatgttttgaaacatccccaatggttaaagaaaatcataatttatccaaagataaaacaatagtattaccccctccaaagtacaaatattagagcttctctacaattgagtgacttacttccctgaatatgtatatgtatgacaccgtgcatggaaccttgccttcatgagcaatgtagatataattcttgatgtatagttgataagtcaaagcctactgattgttattctaaagctttgcttaatcttagtctttcctatgtccagctgtttttgtacagttaccttaggaaacaatttgtctttttcatccctaaaggtccttttatgtggaatgcaagagattgatttgaatgactggcaaagacatgccatctaccgtcattatgcaaggaccagcaaacaaatcatgtggttttggcaggtttgtttttaaatttatttctattagttgacacagcataattttttagaatttgcttaccacatatggaaatgagtcacaggtcaaaatgtaccagtgtatatagaccccttagtccatgcattataataaatgaatacagattataacagcatccttttaagactattgccaagtaagccacacataccttgcatctttgtccaaagtagagtgtaagacaattcgagatgataaaatttacctacatttgatttaaatggttttggggttttataggactgtctgtagtatgttttttatcatttatacttcattaattatataatgttcagaaggcaaaatttaagagacttggtttagaatcataacggtatctctctaaaaatgtctgccttaagcatggtatatgatgttcaacatcattagtcattagagaaatgcaaatcaaaaccacagtgaggctgggtgcggtggctcacacctgtaatcccagcactttgggaggctgaggcgggtggatcacgacatcaggagatcaagaccgtcctggctaacacggtgaaattagccgggcatggtggcacatgcctgtaatcccagctacttgggaggctgaggcaggagaatcacttgaacctggaaggcagaggttgcagtaagccaagttcgcactactgcactccagcctgggtgacagagcaagactctgtctcaaaaacaaaaacaacacaatgagataccacatcacacttagtattatttttgaagtggagaagaacaaccattgccaagggcgcattgctggtgatagtgtgaaataccacaccactgtggaaatagtttggtggttcctcaaaatttaagcatagaattaacataagatccaggaattccaatccttggtatacagcccaaaaaattgaaagcagagacttaaacagatacttgtacaccagtattcatcattcatttaccgacacaaaatgtggtatatgcatatacagatgctcctggacttggagttttatcctttatatccatcataagtcaaaaatatcttgagttgaaaatgcatttaatacccttatcataacttacccattgtaaagttacattgtaaagtcacaacttacccattgtaaagttgaaaaatcataaatcgaaccatcataagtctaggtgctcctcaacttatgatggagtaataaacccaataaacccatcataaagtcaaaaatttgtaagccagggactgtctgtgttggaatattattcagtgctaaaaagtaatgagatttttggtatgtgctacaacatgaacaaaccttaaaaacattatgaattttaagataagcgaagtaagccagacacaaacggacaaatactgtataattccacttacatgaggttactagaatgggcagcttcatagaggcaaaagtagaatatgggttaccagaagttgcagggagcaaaaatgggaagttgtgtttaatgtgtacataggttttacttgggatgatgaaaaagttggaaatagtggcgatggttgcacaacagtgtgaatgtacttaatgtcattgaattacactcaaaatggttaaaatcgagaatttatgttgtgtatattttacaaaaatttaaaaaattaaaaaaatctggtatacatgtaaaaaaattcatatgagaaacttaaactgagcttctataacaagtaggattacaattgtaggataattaataatactacaataggataaaaattattccttataaaaaagtttttggctgaggcaaaacagttatactacaggctagaaacattctctagaaacctattgttttgagtcagaattgtatgaaataaaaaccacataattctagctatgtgtagctataccaaaacctaaagaacattgagaagaggaggttattgggatatatcctgcatcagaattctgtgagtttgattaaagttgtttattaaaccaaaaagtctgtactgactcaaggtctgtatcctgtgtagccacatgaatatcctataggacgggcaaatctggggctggggggtggtggtgggatggggagtcacaggaagggctgccctagaagagctaatcttaaaactgcgtgagtacggagctcttggtctcagtccatcttactttttatatatccctttaagtatattgtcctaagatttatataaggaaaggttgatggggctttgataaacatggaaatggccctccatccatagtgttaagtaattatctaatgcagtggacaccagagtttcctttaagatttgaaatgagctagatacagtggcttgctcctatagtcccagctactctggaggccgaggcaggaggactgcctgctgcccaggagttagaggctgcaatcagctatgattgcatcactgcaccccagcctgggccacagagcctgggacacctcacctctaaaaaaaataaaagatttgaaatcattatttttctttcttgtttagtgtgctgaatgtaacatatgacatatatatctctttaaatatacttttaacagtttgttaaagaaattgataatgagaagagaatgagacttctgcagtttgttactggaacctgccgattgccagtaggaggatttgctgatctcatgggtatgtatacaggatcacttttcctatacagaaaattgtttatcatgctgctttacgtaaatgtgtatacagcctattagaaacaacaaagacagggatagccattttcttaactattcccagttagcatggatttgtataacctttaaaatagtttttagtacttacctttgtagaaatttttaaccctcaagctagaacccacagctaactcattaggacagcagtcttctgtatgtaaatctgttcatttagtcatttgccaaacatgtgtgtcttaagagtcaatcactattggttgtattatttaacgatgtgagaagatcattatataatacaatagtggattgtagttttagtattgtgtttaaaaccactcttccctgggagactgggagttctttgagaaatggattcatatcttcatctttttatttcttgcccctggcaaagtgcctgttacacagcaatctgctgcttaaaatgaatattaagatgaatgactatggcaatattcactttgttactaaacagtaaattatttaagtaaattcaagtatttccttaataccaacattatcccaagtatcataggaggatacacagagaagttaagatcccaaagcctgcttcagactgaagcagagagtcaaaattagaactaagcaggtaattttgaatacagaacatggaaccagtttagaaagtactttaaaagccagaaaaagaatttggatattaccgtgagggtattggaagctgtggaactttttgaagagaggaaaacttgctcaagctgcatttaggaagatttgataggaagatacagtgtggattaaaagatggtgaggacctagagataagaagaaatcattgcagcaatgatgagaacctgatctcaggagatgaaagtagaaatagaaaagagggggtagatactaggtaaagttccaaaggagaaacaggtagagagcttaatagaaaatgaagtataagagaggggtatcagcaataattgtcatgttatggaacatggagcaatggagaagttaggaagtataaagagtatgcatcgttgtagatgttgggggaggatgctgagcagcttagaacctgctgaatttaaggtaactgctggacaaccaagtagaaaaccccagggaagcagctgtagaggtaacaatatatgagttagagttaagaggaaacagaagaagttgaatcatcagagcagtgacatttgatgctgtgagaatgtgtggtcttaggcagaaatagaatacagagaggagtagaaggctaatgcctagaaccatctgcatgtagggataagacagaaggagatagcctaggccaggcacagcctcacgcctataattccagcactttaggaggctgagctgggtggatcgcttgagctcaggagtttgagaacagctgggacgacatggtgagaccccgtctctactaaaaatacaaaaattagccgggcgtgttgacgcacacttgtagccctggctactgggaaggctgaggcagggagaatcgcttgaacccgggaggcagaggttgcagtgagctgagatcgtgccactgcactccagcctgggaaacagagtgagactccatctcaaaggaaaagaaaagatggaattagtctggaagccaatttaaaggagagaccagagagtgaaaagaagaaagaaatagtatactgtctgagaagcaattagaaggaatttttttcttcagtgctacagagaggtctcagaagggtgatttctaggaaagtttttgttaggagacagaaacagtgagattgtggtttggagaattttagaggctatctagtttattttgtttgttttagtttcaaatctttattcttttttactttttaaattattcttaattgacataattgtacacatttaggggtacagtttttattttgatacatgtatacaatatataatgatcaaattggggtaattaggatatccatcacctcaaacatttatcaattctttgtgttgggaacattcaaaactcacttttctaactatttataaacatacagtccaggtgcagtggttcacacctgtaatcccagcactttgggaggctgaggcaggaggaacacacgagcccaggggtttgagacaagcctgggcaatatagggaaaccctgtctctacagaaaatttagaagttagctgagcgtggtggtacatgcctgtagtccctgctattcaggaggttgaggtgggaggatcacttgagccttggaggtgaaggttgcagtgagccaaggtcatgccactgcactacagtctcagtgacagagtgagaccctatcttaaaaatacatacactgggcgcggtggctcacgcctgtaatcccagcacgttgggaggccgaggcaggcggatcacctgaggtcaggagttcgagaccagcctggccatcgtggtaaatccccttctctacgaaaaatacaaaaaaaaaaaaaaaaaattagccaggtgaggtggtgggagcctgtaatcccagctacttgggaggctgaggcaggagaatcacttgaacctgggaggcagaggttgcagtgagtcgagattatgccactacattccagcctgggcgacaagaacaaaactccaactcaaaaataaataaataaataaataaataaaatatatatatatatatatatatacacacgcacacacacacacggtaaattgttgttgattatagtcattgtatagtgctatagaacactagaacttattcttcctgccgatctgtacttgtgcatctattaaccaacctttggctctcccctcattcctgccacccttctgtagtaaccactgttgcactttgtacttctatgagatcggctttcttagctttcacgagtgacagcatgtagtatttatctttttgtgcttggcttatgtcacttaaaacgtcctccatgttcatctatgttgtcataaatgacagaattttgcttttttaaaaaatgtctgaagtattccagtttgtttatacaccacattttctttatccgtttacctgattatggacacataagttgattccctatcttggcccttgtgaatggtgctgcagtagacatgggagtgcagatgtctgttcgacatactgatttccttttctttagatgaatgccccatagtgggattgctggatcatatggttgttgggcggctgagatgggtggatctcttaaggccaggagtttgagatcagcctggtcaacatagtgaaaccctgtctctactgaaaatactaaaaaaaaaaaaaaaaaaaaaaaaaaattagctgagagtggtggtacacgcctgtaatcccagctgcttgggaggctgaggcacaacaattgcttgaaactgggaggcagaggttgcactctagcctgggtgacagagcaatactctgtctcaagaagttaagaagaagaatttacatccctgccaacagtatataagagttcctctttttccacatccttgccagcatttgttattttttaatctgtaacagttattctaactgaggtgagatgatagttcattgtggttttgatttgcatctccctgatgattagttatgtttaacattgttattttgagagatatctatttagctcatatcccattttttaatgcattgtttttcttcttttttgctgttgagttcttgtatattctggatattctttgttggatgaattgtttgcatatactttctcccattctgcagattgtctctacactgtgttgattgtttcctttgctatgccaaagctttttagtttcatataacccaatttgtctatttttatttttgttgcctgtgctttttatgtcctctttataaaatcttggctcagggccaggtgtggtggctcgtgcctgtaggcccagcactttgggaggccgagacgggtggatcacctgaggtcgggagttcaagaccagcctgaccatggagaaaccctgtctctactaaaaatacaaaaattagctgggcgtggtggcgcgtgcctgtcatcccaactactcaggaggctgaggcaggagaatcacttgaacccaggaggcggaggttgaggtgagccgagatcgtgccattgcactccagcctgggcaagaagagcaacactccatctcaaaaaaaaaaaaaaaaaagaaaagagaaaagaaaaatctttgctcagacctgtgccctgaagcatttttcctgtttaatccattttgagttgatttttgtacgttatgagagaaaggggtctactttcatttttagcatatagatttctgttattccagtaccatttattgaagagagtgtcctttcccccaatgattgttctttttttttgagacagagtctcactccgttgcccaggctggagtacagtggcgagatctcggctccccacaacctccgcctcccaggatcaagcaattctcttgccttagcctcctgagtagctgggattgcaggcacgcaccaccacacctggctagtttttttatttttaatagagatagggtttctctatgttggtcaggctggtcttgaactcctaaccttgtgatctgcctgcctcagcctcccaaagtgctgggattacaggcatgagccactgtgcccagccaatgattgttcttagtgtctttattgaaaatcagttggatgtaaatatgtgggtttgtttctgggttctctcttctgttccactggtctatgtgtctgctattatgccagtaccatgctgtattggttactatagctttgttgtatatcttgaagtccagtagtgtgatgcctccagctttttgttcagaatggctttggctattcaggatcctttgtagttcagtatgagttttaggattgttttttctatttctgtgaagaatgtcattggttgtttttgtttttgtttttgttttttgagatggaatgtatcactctgtcacccaggctggagtgcagtggcacattctcagttcactgcaacctctgcctcccgggttcaagtgattcttgtgcctcagcctcccaagtagctgggattacaggcatatgccaccacacccagccactttctgtatttttagtagagatggcatttcgccatgtgactggtctggtcttgcactcctgaactccggtgatccgcccaccttggcctcccaaagtgatgggattacaggcatgagccactacacccagccgtcattggtatttctgtagggattgcattgaatctgtagaaatttttaatagtacggttattagtggagtattaatttttcaacccatgaacatgggatgtctttacattttttgtgtgttttcttcagtttcttttgtcagtgttttatagtttttgctgtagagatcgttcacctccttggttaaatttatttctaggtttttgttgtttgtagctattgtaagtaggattgctttcttgatttctttttcttctagtttgttgttttatagaaacgctgctgatttttgtatgttgattttgtatcctacaactttacttaatgtgtttatcagttccaataattttttattggtggagtttttaaggttttctgtatataaggtcatgttgtctgcgaacaggtatagttcaacttcttcctaattctgatgccctttatttctttctcttatctaattgctttggctgggacttccagcagtttgttgaataaatgtggtgaaagtgggcacccttgtcttgttccatatcttggaggaaaagcttttagttttctctgttcagtatgatgttggctgtggatttgtaatatatagcctttattatgttcaggtacattccttctgtgcctaacttgttgagagtttttcttatgaagtgatgttggattttattaaatattttttctgcatctattgagaagatcatatggtttagcccatcattctgttgatatactttgttatgttcattgatttgtatatgttgaaccatccttgcaacctttggatctttttaatgtgttgctgaatttgatttgcttgtatttcattgaggatttttgcatctgtgttcatcaaggatattggcctatagttttcttttcttttcttttcctcctttttttttgtttttgtttcttgtatccttgtctagttttggtatcagggtaacgctggccttgtagaatgagtttcaaaaaattctctcttctattttctggaagagcttgaaaagaattggtattagttcttctttaaatgtttgttagaattccattggttcctagacttttgttttctttgatgggagactttttttttttttcttgagacagagtcttgctctttcacccaggctggaacgcagtggcacgatctcggctcactgcaacctccgcctcacaggttcacaccattctcctgcctcagcctcccgagtaactgggactacaggcgcctgccaccatgctcggctaattttttgtctttttagtagagacggggtttcaccgcgttagccaggatggtctcgatgtcctgaccttgtgatccgcccgccttggactcccaaagtgctgggattacaggcgtgagccactgtgcccagcttttttttttttttttttttttttttttttttttgagacagtcttgctctgacacccaggctggagtgcaaatgggagactttttattacagattcagtcatgttacttataattgatcttttcagcttttctgctttttcttggttcagtcttggtaggttgtatgtgtccaggaatgtatcaatttccataggttttctaagttggtgtgtagttcataataatttctaaaggtcctttgtatttctgtggtgtcagttgtaatgtctcccttttcgtttctgatttcatttatttgggtcttttcttggttagtttagctaatggtttgtcaattttgtttatcctttcagaaaaaaaaaacaacttttcatttcatctatcttttgtcttcatttttagcctcatttttgtttatttctgttttgatctttattatttctttccttctagtaattttgggtttgctttgttcttttctggtttctcaaagtgtatcattaggtcatttgtttaaaatctttccacttttttgatgtaggcatttattgctataacattaccttttaatattggttttgctatattttataattttcaatatattgtgtttccatttgcatttgtttcatggaatttttaaatttccttttacatttccccattggtcattctggagcacgtttaattaccatgtatttatacaattttgaaagttcctctttttattgatttctagtcttactccattgtgttcagaaaatatatttgacattatgacttcagttcttttaaatttgttgagacttgttttgggtcttaatatgtggtttccccaggagaatgttccatgtgctgatgacaagaatgtgtattctgaagctgttggatgaaatgttctatgaatgactgttaggttcatttggtctgaagtacagtttaaatccaatgtttctttgttgattttctgtctagatgatctatccagtgctaagagtggatattgaagtccccaactagtattggagtctgtctttcccttttaatataataatatttcctttttatatctgggttctttggtgttgggtgcatatataactctcatatcctcttgttgaattggtccctttatcattatagaatgccctttgtctctttttatagtttttgatttaaagtctgtttgcctgatatatgtatagctactcctgctcactttggatttccatttccatgaaatatctttttcatcccttccttttcagtctgtatgtgtctttacgggtaaagcaagtttcttggaggcagcatatgatagttgggtcatttttctttttaaatctattcagccagtctatatctttttagtagggaatttatccacttatattcaaggttattactgatagatcaggacttactcctgtcatttttaaaattactttctggttgctttatatatccgtcgttcctttctttctctcattgtttatcattatggtttttgtggttttctatagtgctaaggtttggttcttttttcccttagtgtatctgctctaccagagtgttatattaatactttttgtgtattttcatgagagtgattatcatcttttcacttccagatgtgggagtccgttgagcatttttcataaggtgatgaattccttctatttccccatgtctgggaaatactgtatttctcctgtatgtctgaaggatggttttgctgggtatagtaatcttggctgccaatttttttcttgcagtactttgaatattatctcattctctcctggtctgtaaggtttctgcagaggcagccatctagtttaaacttccacccagcacaggaagagaaagttttagaggtggtgtatgtaattctttaatgggatgcttcttgaccttggggatggatccaacatcccacttttattaatatatattcataatatcccctttaatacactgatttatggatgaaataacctattgacaatccctagaaaatacttatacatagccttaactgtaatataagggattaaattttttaggtaatttataatgaaataatatgtgttttaatacatgtctatttggggacaactatgttagaagatatacctaagaagtcaaatatttgttcctacttataagaaaaggtttaagacaagatcaaaagccattctctataagaagtactggaagggctgagtgcggtggctcacgcctgtaatcccagcactttgggaggctgaggcgggcggatcacctgaggttgggagttcgagaccagcctgaccaacatggagaaaccgtatctctactgaaaatacaaaattagccaggcgtggtggcacatgcctgtaatcccagctactcaggaggctgaggcaggagaatcgcttgaacctgggaggcagaggttgcagtgagccaagatcgtaccattgcactccagcctgggcaacaagagcgaaactccatctctcaaaaaaaaaaaaaaaaagacaagtagtactggaaaataaaagaacagatgagaaagataaataatagacttaagtatatgtgataacaaaaggagggaagtaacctaaataaagtatgaataatacacctgcaaaaataaattatagatggtcaaggtggagaaaaatgaggtagatcttaaaagcatagtatcagaatatatctcctgtcatgaaattctactataggccaaggctctaacatgtaaaaaagcctcggagaccataacatttgaaaggtggctgaaaataggaacataggtacaaaagtctacacagaagttgtatggtttagggacagtgtcagagtttttaatatttgattttaaatctattaaaagcaattcagaatcctttctgttacatactaatgtgcattattaaatgaaactcctttccatctatgtagttaaatatgctgctaggtatggtgggcacacatctgtagtcccagttatttggaggctgaggcaggagaattgctggagcccaagatttcaagcctgtagtgcactataactgcttctgtgaacagccactgcacttaagcatgggcaacatagtgataccctgtgtcttaaaaaaaaaaaaaatttataaaaaatgaaatgtgaccaggtacagtgactcacacctgtaatcccagcactttgggaggccgagacggttgggtcacttgaggtcaggagttcaagaccagcctggccaacatagtgaaaccccatctctactaaaaatacaaaattagccgggtgtggtagtgggcacctgtaatcccagctactcgggaggctgaggcaggagaatcacttgaacctgggaggcggaggttgtggtgagccaagattgctccactgcactccagcctgggagactccgcctcaaaaaaaaaaagaaaatgctataaaatattgagtagagggtggacatgatggctcatgcctgtaatcccaacactttgggaggccaaggtgggttgatcacttgaggccaggagttcaagaccagcctggccaacatggtaaaaccccatctttactaaaaatacaaaaattagttgggtgtggtggtgcacatctgtaatcacagctactctaaggcatgagaattgcttgaacccaggaggcggaggttgcattgagccaagattgtgccactgccatccagccagggtgacagagtgagactctgtctcaaaaaatatatatattgagtagagagaacatgcagagtgagttaattttaatttatgtgttgaacaccacagaataattttaactgtgtttttttataacgtttcctatattactggactttttacataactacttgtgaaaacaattcagtgttattaaatacaaaagcactaacaatttatattgggtatacttagtatacagggtttagtcaggtgggacatgagaaagttctttgtgtctgacagtgtcacacattacagaacagttagcgttcctgcccccttctaattaaaatatctagtagcattaccagtcattgagaccaacaataaatgcttcctagggaaaagtaatgatcttttaaaaaactggtgtttttatgatcacttttcacagctatctccagcagcaggtaaatgaggggcaagtattttatgtttatacttgtatctgtttttgtaagggtatgccctacaggacctgtaagttccagcattaataatatggaattcactaggatatttacatttcacaaagttttattaattctgtatttaaatcaagttttattgtagtttcgttattgtctttcaaactactaaaattaagctttttgtgtgttcttgtgtaagcttttattaatcataacaatgtgttattccggctaaagaatgctagatgaaactgcattttctcctttgggacttatattaacaaactatagtcttcaaacttttacacttaccagaatcacccggaagcttgttaaagcacatattgctgagcctgacccctagagtttcttttttgtttgttggttgcttttgggttttttgggtttttttttttttttggtttttttttttttggttttttttttttttgagatagggtctcgttctgtcacccgggcgagagtgtagtggcgcaatcttgagtcactgcaactttgctgcacagattcaagcaattctcctgcctcagtctcctgagtagctgggattacaggcatacgccaccacacttggctagtttttgtatttttagtagagatcaagattcaccatattggccaggctgatcttgaactcctgacctcaagtgagctgcccacctttgcctctcaaagtactgggataacagacatgagccaccacacctaacccagagtttctgaattagtaagatgatgtgtggggcctgaggatttatggttctaacaagttccctcatgatgctgatgcttgttggattggggaacacagtttgagatccactatactaacaaactgtttatgatttcattatgttttagggagcaatggaccacagaaattctgcattgaaaaagttgggaaagaaaattggctacccagaagtcatacctggtaagtacaatcagaatggatagagaaaacagcttttgtccagttatctgtagctatttaaagccagctagaaattaaaaatgatatttagtagatttacagaatagcacagccatcatagcaatccagtttaagagcattttgttcacccctgtatgattccttacaactatttgcaatcaattccttttttcacatctgaattttttttctaatgtgggatttagagttattttaacttttctctgagggcagcaaaagaaagttaggagacctactttctaacatcatgaccacaaatattctcatttgaaatctaattacatttctaaaaatgtagaaaactaaatctaatcctaatctgacagcaaatatattaatattttggaattcaacctaatgtcaacaatatttgaaccttattttgtgggatatattttagtggtcacatatggtccttcctggatatctgacctctttactgttttactaatgtctattcctttttgtttttatacataatgttttgtacatgcatgtagtgtttaagtaacaattttattgatatataatttacagattataaaactcaccttcttaaattatgcagttcggttcagtggggtttttttgtttgtttgtttgtttgtttgtttttgagacagggtctcactttgtagaccaggctggagtgcagtggtttgatctcagctcactgcaacctccgcctcccaggttcaagcaattctcttgcctcagcctcccaagcagctgggattacaggcgcccgtgaccacgcccagctaatttttgtatttttagtacagatggggtttcgccatgttggtcaggccggtctcaaactcctgatctcaggtgatccacccacctcggcctcccaaagtgttgggattacaggtgtgagctgccgcatctggcctgttcagtgattttagcatatttatgaggttatgtaagcatcattactgtctaattccagaattttttcaccctcccaaaaatccatgtacccattagcagtcactccctttttaccccgtgtagcccttggtaaccactaatctactttctgtctctatgaattttcctgttctggacatttcatataaaatgaatcatacgataaatatataacttttttttttttggagacagtctctctctgtcacccaggctggagtgcagtggcgtgatctcagctcattgcaacctctgcctcctgggttcaagtgattctcctgcctcagcctcccgagtagctgggactataggcagcatgcaccaccacgcatggctaatttttgtatttttagtagagacggggtttcaccatgttcgccaggctgttcttgaactcctgacctcgtgatccacccactttggcctcccaaagtcctgggattacaggcgtgagccactgcccccagccaaaatatacaaccttttgtgtgtagcttcttttacttagcatgttttcaaggttcatccatggtctagcatatacagtatttcattcctttttaaaattaattgttgggtttaatatacataacatagaatttaccatttttaagtgtgtacagttcagtggcattaagtgcattcatatcattatgcagtcatcaccatcacctatctccaaaaattgtttcttttgttgagacatgtctcagtctgtcacccaggctggagtgctgtagctcaatctccactcatgcaccctccacctcccgggctcaaactgtcctcctaccttagcctcccaagtagctgggacgacaggcatgtgctaccacacctggccaatttctaaactttttgtagagacagagtcttactttgttgctcaggctggtctcgacctcttgagctcaagcaatactcctgctttgacctcccaaagtgctggaattacaggccatcatgccagacctaaaacatttttcatcttgtaaaattgaaatggtacccattaaagcaggggtccccaactcctggtccccagcctgttaggaactgggctgcacagcaggaggtaagtggagggtgaatgagcattacctgctgagctccaccacttgtcagatcagcagaggcattagattctcataggagcaataactattgtgaactgtgcatgtgagggatctagattgtgcattccttacgagaatctaacctgaggtggaacagtttcatcctgaaaccatccccctctccactcctgtccatggaaaaattgtcttccacaaaaccattgcctggtgccaaaaaggctggagactgctgctttaaacaatgactccccatgactccctttccccaggtcctggcaaacatcattctactactgtttctatgattctttagtacttcgtaagtgtagaatcgtaccttatttgtcctttcctggctggcttatttcacttagaataatatctttaattttcatccatgttgtagcatgtgtcagcatttcctcctttttaatgctgaataatatttgatagtctggatatactacattttgtttatacattcatctgtccatggacacttagattatttccaccttttggctgtttatgctgctataaacataggtatactgtggtagaatatagatatatctgtccatggccctgctttcagttgttttgggtatatacccagaggtggaattccttgatcatgtagtaattctatttttaattttttgggggaatctcgccatactgttttccatagtgactacactgcgttatattcctacctgtagtgcagaggggctccaatttctccacatcctcacatccttttcaactcttgttttcttctgttttttttttttttttttttttttttggttgttgttgttgttgttttggtgttttaaataatagccatcccaacaggtgtgaagtggtatctcattgtggtttttatttgcatggtaaaggatgttgagcatttttttaatgtgcttattggtcatttgtatgtctttggagaaatctctatcctttgttcatttttaaatattgttgagttttaagttttttatatattttggataccagtcctttatcagatatatgatttacaaatattttttcccatttttgggttatttcactttcttgatggtatcctttgaagcacagtagtatatttttgtgcttctatagtagtgtataaagtagtatactacactactcatttgattctttgcatatttccttttctcaaactaacaattcttgcttttagttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttagatttatctcccagtgatttctactcagcgtttccagaaatcaggtctgcaaatgactagtcagaaccttgcttaacatgagattttaacacaacaatgaaatttgccttgtcttattccactagtttattcctttaacaacaatattttatgtgtgtcaaaagtctcacttgggagtagtgtttttttcttttagacattctgcagacatgcagggaagtcctttggtaactgcaatatacaagattttcctattaagcctcttggtaagaggcatttgttaaaagtgcaagcttactcctgcttctggggatgtgagcaaaattcgggcttgtgttctccctctcattttagtctgacttgactattgtttttcctttctggcgcatgaatccatacatcattcctggaagtgaggcaagactcttgcatctctacaaagtagttttgtcaatttgaattcagggaaaagttggtcacagcctgcaaatgacttcatttggaagtctgattgtttcagttgcctgacaaatactacactttacaaacaatgttaacactgtgattccttcattgttttaagaagttaacctagggccgggcatggtggctcatacctgtaatcctagcactctgggaggccgaggcaggaggatccctttagcccaggagttaaagaccagcctgggcaacatagggagaccctgtcttttttttgggcagcgtggtgggggataaataaataaaaggaaaaaaaacttagcctagaattagaattaatttaattgaattcatctaaagatgtctctggtgatttttatatgttccgctatataattgatgctttatagttttatcataatccaacaacttcagttatatttaattattgttaaggagtttaagactagaaagactagagtgctttctagtccaaatagaggtcagtgaaacagcttttgacatcagattttcatttgagagggagagctgtggtactggctaaaaagaaaggaagataacatccagtaaccacaggaatatattctctgtgaattaaaagtcttcaaagttatcatttctctgacatatgttggagtagtcatttccattctttacattgtcatgaactggattgataaccctcatctgcaatattttcacccctaaaatttttaacagggtttcctttttttctcacgactatttaagtttagattgctccattattaactgattaatgcactttgaagttctctggaattaattattttaacttggcctagcttcgactgtcaaggtggctgttataaatttgacttcattggcagtggatgaagcctaagccagctgagtctctatcatagctgaaccctgaggacagcctcatagctcatgtatcagggacttttgccacatttcagaggcatagcatgaacaagtaatattaagccaagaataagcagcagaaccctgttccatatggaaaaaagaaaaacaattttttgtccctaatgttcttccttttacatcctggaacaacaataaaaacatttttttaaacttgtctactgtaagatactgccatcataaagcagagacttacatgagtgaaagggttgcctcatcaagcagctcagtgtaaatggggaggctaggctctccccagccctatggtttttttatttcatgtaccccaggaaatactgtgtggtttctaaaagccctggttgttaaaagtagggactctgcctttttgttggtagggagaaaaaacgctattgctttgtcttacagagcgaatgtctgccaactacccgttcattatataagtctgaacttggtaatatatggctaatgaagattaagccctctataaagacttcctgttgaggtgaattctcatactgaaatgtagttacctacaatatttactagagatttatgaaattaaattaagagataatgtaagaaaatacatttttttggttctatataatgcttcatgattcatttagggacctagaaatattgtgtgaaaatatataaatatcacccaaaaggctttctgccctatatttttaaaatacagaatagttatatttgaagtagccctggccctagttctatagggcttggctatttaatatttttatggaagaagtgtttagttctggaaaaggtaaatgcttgtatatatttttgcagcctgggatctccctactccattttttcctttaatttaagtggccacatgtatatgtcttccctgctgtgttaggaaaatgggggctggatatcccaagaatcagaggttatataaaaatactgcaaatagaccgcagacataaatatctaccaaatgctatcttaaattttggtccaaactgaacatatggaaatagatttattgtaagtatttacttagagctttttcttaaatctgaactaacttgcttttagaagtctttttctttgtaagcattgtaaatgctaataaatcctgttaatttttttttttttttttttttttttttactgcatgttacattgaaataaaaacaaagtaaaatgagcaattgccttattgttctgctcttttgcagggggtgggggttggggaagacagcattccacagcctggtcaggagggaaggacctcacttgtttcatcaaatggagtggtttaaaactgatcaagcagctcataataatgaattcctgctctgtcttttattatcttatgtcagagtgcagcaaaatctcatttcataagacttacaataactctatttaaaattcaggtattgtactcgggaggctgaggtgggaggatcccaggagttcaaatcctgcctgggcaacatagcaagaccctgtctcttaaaaaaacaaaaagacatattcaggtactgatttttcatcaagagattgacataccaatgatcagaaattttagtgaaacttgactcaactatacacaaagttctcatcctaaatgaactatttcagtcaagcccactctaccactttttacttatctggttaataatccgaaatgttaccggggttgactcacggaattaaatttttcctctatttttatatcctttcctgttgtggatgtaacttacctttctaaactcctttagagttaacgacttctatttagtattgttggtatctccagcccccctcctttttttgtttttcttttagttatttactttgtttgtataatttgctttcattaactaaaaatggaattggcagcactggattgtatattactgtattaagagtacctttgttttcttgatctgttacttttaatactcaggtaatgctacaaatattacagggagtcaaatgcttttataaagttccttcagtggttgcctcacttgcgttttcctacaagatgtaagactgtttataattaaacttttttggtccttc

SEQ ID NO: 24-99 - skipped (76 entries)

SEQ ID NO: 100 - RNA, 19 nt, Artificial Sequence
  source: /note=“Description of Artificial Sequence: Synthetic oligonucleotide”
gcauagagaccgacuuaau

SEQ ID NO: 101 - RNA, 19 nt, Artificial Sequence
  source: /note=“Description of Artificial Sequence: Synthetic oligonucleotide”
ccgaagaccgagcugaauu

SEQ ID NO: 102 - RNA, 19 nt, Artificial Sequence
  source: /note=“Description of Artificial Sequence: Synthetic oligonucleotide”
cagacaguagucuguuaua

SEQ ID NO: 103 - RNA, 19 nt, Artificial Sequence
  source: /note=“Description of Artificial Sequence: Synthetic oligonucleotide”
ggaaagauggaucaauacu

SEQ ID NO: 104 - RNA, 19 nt, Artificial Sequence
  source: /note=“Description of Artificial Sequence: Synthetic oligonucleotide”
cuucacaccuauugguugu

SEQ ID NO: 105 - RNA, 19 nt, Artificial Sequence
  source: /note=“Description of Artificial Sequence: Synthetic oligonucleotide”
cacgagaggcucagguuau

SEQ ID NO: 106 - RNA, 19 nt, Artificial Sequence
  source: /note=“Description of Artificial Sequence: Synthetic oligonucleotide”
cgagaggcucagguuauca

SEQ ID NO: 107 - RNA, 19 nt, Artificial Sequence
  source: /note=“Description of Artificial Sequence: Synthetic oligonucleotide”
gccgagucccauauccgaa

SEQ ID NO: 108 - RNA, 19 nt, Artificial Sequence
  source: /note=“Description of Artificial Sequence: Synthetic oligonucleotide”
ccgagucccauauccgaau

SEQ ID NO: 109 - RNA, 19 nt, Artificial Sequence
  source: /note=“Description of Artificial Sequence: Synthetic oligonucleotide”
cccauauccgaaugauuaa

SEQ ID NO: 110 - RNA, 19 nt, Artificial Sequence
  source: /note=“Description of Artificial Sequence: Synthetic oligonucleotide”
gaaaguggcaugauaguuu

SEQ ID NO: 111 - RNA, 19 nt, Artificial Sequence
  source: /note=“Description of Artificial Sequence: Synthetic oligonucleotide”
cauuuacgaaaacgaaaua

SEQ ID NO: 112 - RNA, 19 nt, Artificial Sequence
  source: /note=“Description of Artificial Sequence: Synthetic oligonucleotide”
gacucucucaauccuaguu

SEQ ID NO: 113 - RNA, 19 nt, Artificial Sequence
  source: /note=“Description of Artificial Sequence: Synthetic oligonucleotide”
caacaagaugagucuauuu

SEQ ID NO: 114 - RNA, 19 nt, Artificial Sequence
  source: /note=“Description of Artificial Sequence: Synthetic oligonucleotide”
cuauccugcuucaaguacu

SEQ ID NO: 115 - RNA, 19 nt, Artificial Sequence
  source: /note=“Description of Artificial Sequence: Synthetic oligonucleotide”
caacuuuuucaacgaaucu

SEQ ID NO: 116 - RNA, 19 nt, Artificial Sequence
  source: /note=“Description of Artificial Sequence: Synthetic oligonucleotide”
ccgaugggaaaugauacua

SEQ ID NO: 117 - RNA, 19 nt, Artificial Sequence
  source: /note=“Description of Artificial Sequence: Synthetic oligonucleotide”
gggcucuuucaagauagua

SEQ ID NO: 118 - RNA, 19 nt, Artificial Sequence
  source: /note=“Description of Artificial Sequence: Synthetic oligonucleotide”
cugacguauguccaagauu

SEQ ID NO: 119 - RNA, 19 nt, Artificial Sequence
  source: /note=“Description of Artificial Sequence: Synthetic oligonucleotide”
gacguauguccaagauucu

SEQ ID NO: 120 - RNA, 19 nt, Artificial Sequence
  source: /note=“Description of Artificial Sequence: Synthetic oligonucleotide”
caauucgugccuuucaauu

SEQ ID NO: 121 - RNA, 19 nt, Artificial Sequence
  source: /note=“Description of Artificial Sequence: Synthetic oligonucleotide”
ccacaacaauauaaugucu

SEQ ID NO: 122 - RNA, 19 nt, Artificial Sequence
  source: /note=“Description of Artificial Sequence: Synthetic oligonucleotide”
cuuacaugggaucagucaa

SEQ ID NO: 123 - RNA, 19 nt, Artificial Sequence
  source: /note=“Description of Artificial Sequence: Synthetic oligonucleotide”
gggccgugucgauguauca

SEQ ID NO: 124 - RNA, 19 nt, Artificial Sequence
  source: /note=“Description of Artificial Sequence: Synthetic oligonucleotide”
gccgugucgauguaucagu

SEQ ID NO: 125 - RNA, 19 nt, Artificial Sequence
  source: /note=“Description of Artificial Sequence: Synthetic oligonucleotide”
ccaguggauuccuguaauu

SEQ ID NO: 126 - RNA, 19 nt, Artificial Sequence
  source: /note=“Description of Artificial Sequence: Synthetic oligonucleotide”
gugagguaccgucuacauu

SEQ ID NO: 127 - RNA, 19 nt, Artificial Sequence
  source: /note=“Description of Artificial Sequence: Synthetic oligonucleotide”
cacucuaggauguauauuu

SEQ ID NO: 128 - RNA, 19 nt, Artificial Sequence
  source: /note=“Description of Artificial Sequence: Synthetic oligonucleotide”
cauuaauucaggcauauuu

SEQ ID NO: 129 - RNA, 19 nt, Artificial Sequence
  source: /note=“Description of Artificial Sequence: Synthetic oligonucleotide”
gcuaugugccuuauguuga

SEQ ID NO: 130 - RNA, 19 nt, Artificial Sequence
  source: /note=“Description of Artificial Sequence: Synthetic oligonucleotide”
gacuuuauaaauuucgcuu

SEQ ID NO: 131 - RNA, 19 nt, Artificial Sequence
  source: /note=“Description of Artificial Sequence: Synthetic oligonucleotide”
guuggggcuuuacauacuu

SEQ ID NO: 132 - RNA, 19 nt, Artificial Sequence
  source: /note=“Description of Artificial Sequence: Synthetic oligonucleotide”
gaauccaacugcuuaaagu

SEQ ID NO: 133 - RNA, 19 nt, Artificial Sequence
  source: /note=“Description of Artificial Sequence: Synthetic oligonucleotide”
caacugcuuaaaguuggua

SEQ ID NO: 134 - RNA, 19 nt, Artificial Sequence
  source: /note=“Description of Artificial Sequence: Synthetic oligonucleotide”
gcuuaaaguugguauuaau

SEQ ID NO: 135 - RNA, 19 nt, Artificial Sequence
  source: /note=“Description of Artificial Sequence: Synthetic oligonucleotide”
caaccacauaguucguuua

SEQ ID NO: 136 - RNA, 19 nt, Artificial Sequence
  source: /note=“Description of Artificial Sequence: Synthetic oligonucleotide”
cacauaguucguuuaccuu

SEQ ID NO: 137 - RNA, 19 nt, Artificial Sequence
  source: /note=“Description of Artificial Sequence: Synthetic oligonucleotide”
gcuuagacacauuuugcau

SEQ ID NO: 138 - RNA, 19 nt, Artificial Sequence
  source: /note=“Description of Artificial Sequence: Synthetic oligonucleotide”
ggcuucggacaaaauaucu

SEQ ID NO: 139 - RNA, 19 nt, Artificial Sequence
  source: /note=“Description of Artificial Sequence: Synthetic oligonucleotide”
gaccagaggucuagauuaa

SEQ ID NO: 140 - RNA, 19 nt, Artificial Sequence
  source: /note=“Description of Artificial Sequence: Synthetic oligonucleotide”
ccagaggucuagauuaaua

SEQ ID NO: 141 - RNA, 19 nt, Artificial Sequence
  source: /note=“Description of Artificial Sequence: Synthetic oligonucleotide”
gacaaucaguuauacaguu

SEQ ID NO: 142 - RNA, 19 nt, Artificial Sequence
  source: /note=“Description of Artificial Sequence: Synthetic oligonucleotide”
cguuaaaccaaauacacgu

SEQ ID NO: 143 - RNA, 19 nt, Artificial Sequence
  source: /note=“Description of Artificial Sequence: Synthetic oligonucleotide”
gccaaacaaaggauacuua

SEQ ID NO: 144 - RNA, 19 nt, Artificial Sequence
  source: /note=“Description of Artificial Sequence: Synthetic oligonucleotide”
gaugugucuaauuuagucu

SEQ ID NO: 145 - RNA, 19 nt, Artificial Sequence
  source: /note=“Description of Artificial Sequence: Synthetic oligonucleotide”
cuaguguuuuucgauguua

SEQ ID NO: 146 - RNA, 19 nt, Artificial Sequence
  source: /note=“Description of Artificial Sequence: Synthetic oligonucleotide”
guuuuucgauguuacuaaa

SEQ ID NO: 147-149 - skipped (3 entries)

SEQ ID NO: 150 - RNA, 19 nt, Artificial Sequence
  source: /note=“Description of Artificial Sequence: Synthetic oligonucleotide”
auuaagucggucucuaugc

SEQ ID NO: 151 - RNA, 19 nt, Artificial Sequence
  source: /note=“Description of Artificial Sequence: Synthetic oligonucleotide”
aauucagcucggucuucgg

SEQ ID NO: 152 - RNA, 19 nt, Artificial Sequence
  source: /note=“Description of Artificial Sequence: Synthetic oligonucleotide”
uauaacagacuacugucug

SEQ ID NO: 153 - RNA, 19 nt, Artificial Sequence
  source: /note=“Description of Artificial Sequence: Synthetic oligonucleotide”
aguauugauccaucuuucc

SEQ ID NO: 154 - RNA, 19 nt, Artificial Sequence
  source: /note=“Description of Artificial Sequence: Synthetic oligonucleotide”
acaaccaauaggugugaag

SEQ ID NO: 155 - RNA, 19 nt, Artificial Sequence
  source: /note=“Description of Artificial Sequence: Synthetic oligonucleotide”
auaaccugagccucucgug

SEQ ID NO: 156 - RNA, 19 nt, Artificial Sequence
  source: /note=“Description of Artificial Sequence: Synthetic oligonucleotide”
ugauaaccugagccucucg

SEQ ID NO: 157 - RNA, 19 nt, Artificial Sequence
  source: /note=“Description of Artificial Sequence: Synthetic oligonucleotide”
uucggauaugggacucggc

SEQ ID NO: 158 - RNA, 19 nt, Artificial Sequence
  source: /note=“Description of Artificial Sequence: Synthetic oligonucleotide”
auucggauaugggacucgg

SEQ ID NO: 159 - RNA, 19 nt, Artificial Sequence
  source: /note=“Description of Artificial Sequence: Synthetic oligonucleotide”
uuaaucauucggauauggg

SEQ ID NO: 160 - RNA, 19 nt, Artificial Sequence
  source: /note=“Description of Artificial Sequence: Synthetic oligonucleotide”
aaacuaucaugccacuuuc

SEQ ID NO: 161 - RNA, 19 nt, Artificial Sequence
  source: /note=“Description of Artificial Sequence: Synthetic oligonucleotide”
uauuucguuuucguaaaug

SEQ ID NO: 162 - RNA, 19 nt, Artificial Sequence
  source: /note=“Description of Artificial Sequence: Synthetic oligonucleotide”
aacuaggauugagagaguc

SEQ ID NO: 163 - RNA, 19 nt, Artificial Sequence
  source: /note=“Description of Artificial Sequence: Synthetic oligonucleotide”
aaauagacucaucuuguug

SEQ ID NO: 164 - RNA, 19 nt, Artificial Sequence
  source: /note=“Description of Artificial Sequence: Synthetic oligonucleotide”
aguacuugaagcaggauag

SEQ ID NO: 165 - RNA, 19 nt, Artificial Sequence
  source: /note=“Description of Artificial Sequence: Synthetic oligonucleotide”
agauucguugaaaaaguug

SEQ ID NO: 166 - RNA, 19 nt, Artificial Sequence
  source: /note=“Description of Artificial Sequence: Synthetic oligonucleotide”
uaguaucauuucccaucgg

SEQ ID NO: 167 - RNA, 19 nt, Artificial Sequence
  source: /note=“Description of Artificial Sequence: Synthetic oligonucleotide”
uacuaucuugaaagagccc

SEQ ID NO: 168 - RNA, 19 nt, Artificial Sequence
  source: /note=“Description of Artificial Sequence: Synthetic oligonucleotide”
aaucuuggacauacgucag

SEQ ID NO: 169 - RNA, 19 nt, Artificial Sequence
  source: /note=“Description of Artificial Sequence: Synthetic oligonucleotide”
agaaucuuggacauacguc

SEQ ID NO: 170 - RNA, 19 nt, Artificial Sequence
  source: /note=“Description of Artificial Sequence: Synthetic oligonucleotide”
aauugaaaggcacgaauug

SEQ ID NO: 171 - RNA, 19 nt, Artificial Sequence
  source: /note=“Description of Artificial Sequence: Synthetic oligonucleotide”
agacauuauauuguugugg

SEQ ID NO: 172 - RNA, 19 nt, Artificial Sequence
  source: /note=“Description of Artificial Sequence: Synthetic oligonucleotide”
uugacugaucccauguaag

SEQ ID NO: 173 - RNA, 19 nt, Artificial Sequence
  source: /note=“Description of Artificial Sequence: Synthetic oligonucleotide”
ugauacaucgacacggccc

SEQ ID NO: 174 - RNA, 19 nt, Artificial Sequence
  source: /note=“Description of Artificial Sequence: Synthetic oligonucleotide”
acugauacaucgacacggc

SEQ ID NO: 175 - RNA, 19 nt, Artificial Sequence
  source: /note=“Description of Artificial Sequence: Synthetic oligonucleotide”
aauuacaggaauccacugg

SEQ ID NO: 176 - RNA, 19 nt, Artificial Sequence
  source: /note=“Description of Artificial Sequence: Synthetic oligonucleotide”
aauguagacgguaccucac

SEQ ID NO: 177 - RNA, 19 nt, Artificial Sequence
  source: /note=“Description of Artificial Sequence: Synthetic oligonucleotide”
aaauauacauccuagagug

SEQ ID NO: 178 - RNA, 19 nt, Artificial Sequence
  source: /note=“Description of Artificial Sequence: Synthetic oligonucleotide”
aaauaugccugaauuaaug

SEQ ID NO: 179 - RNA, 19 nt, Artificial Sequence
  source: /note=“Description of Artificial Sequence: Synthetic oligonucleotide”
ucaacauaaggcacauagc

SEQ ID NO: 180 - RNA, 19 nt, Artificial Sequence
  source: /note=“Description of Artificial Sequence: Synthetic oligonucleotide”
aagcgaaauuuauaaaguc

SEQ ID NO: 181 - RNA, 19 nt, Artificial Sequence
  source: /note=“Description of Artificial Sequence: Synthetic oligonucleotide”
aaguauguaaagccccaac

SEQ ID NO: 182 - RNA, 19 nt, Artificial Sequence
  source: /note=“Description of Artificial Sequence: Synthetic oligonucleotide”
acuuuaagcaguuggauuc

SEQ ID NO: 183 - RNA, 19 nt, Artificial Sequence
  source: /note=“Description of Artificial Sequence: Synthetic oligonucleotide”
uaccaacuuuaagcaguug

SEQ ID NO: 184 - RNA, 19 nt, Artificial Sequence
  source: /note=“Description of Artificial Sequence: Synthetic oligonucleotide”
auuaauaccaacuuuaagc

SEQ ID NO: 185 - RNA, 19 nt, Artificial Sequence
  source: /note=“Description of Artificial Sequence: Synthetic oligonucleotide”
uaaacgaacuaugugguug

SEQ ID NO: 186 - RNA, 19 nt, Artificial Sequence
  source: /note=“Description of Artificial Sequence: Synthetic oligonucleotide”
aagguaaacgaacuaugug

SEQ ID NO: 187 - RNA, 19 nt, Artificial Sequence
  source: /note=“Description of Artificial Sequence: Synthetic oligonucleotide”
augcaaaaugugucuaagc

SEQ ID NO: 188 - RNA, 19 nt, Artificial Sequence
  source: /note=“Description of Artificial Sequence: Synthetic oligonucleotide”
agauauuuuguccgaagcc

SEQ ID NO: 189 - RNA, 19 nt, Artificial Sequence
  source: /note=“Description of Artificial Sequence: Synthetic oligonucleotide”
uuaaucuagaccucugguc

SEQ ID NO: 190 - RNA, 19 nt, Artificial Sequence
  source: /note=“Description of Artificial Sequence: Synthetic oligonucleotide”
uauuaaucuagaccucugg

SEQ ID NO: 191 - RNA, 19 nt, Artificial Sequence
  source: /note=“Description of Artificial Sequence: Synthetic oligonucleotide”
aacuguauaacugauuguc

SEQ ID NO: 192 - RNA, 19 nt, Artificial Sequence
  source: /note=“Description of Artificial Sequence: Synthetic oligonucleotide”
acguguauuugguuuaacg

SEQ ID NO: 193 - RNA, 19 nt, Artificial Sequence
  source: /note=“Description of Artificial Sequence: Synthetic oligonucleotide”
uaaguauccuuuguuuggc

SEQ ID NO: 194 - RNA, 19 nt, Artificial Sequence
  source: /note=“Description of Artificial Sequence: Synthetic oligonucleotide”
agacuaaauuagacacauc

SEQ ID NO: 195 - RNA, 19 nt, Artificial Sequence
  source: /note=“Description of Artificial Sequence: Synthetic oligonucleotide”
uaacaucgaaaaacacuag

SEQ ID NO: 196 - RNA, 19 nt, Artificial Sequence
  source: /note=“Description of Artificial Sequence: Synthetic oligonucleotide”
uuuaguaacaucgaaaaac

SEQ ID NO: 197-199 - skipped (3 entries)

SEQ ID NO: 200 - RNA, 19 nt, Artificial Sequence
  source: /note=“Description of Artificial Sequence: Synthetic oligonucleotide”
gagccucccauuacucaga

SEQ ID NO: 201 - RNA, 19 nt, Artificial Sequence
  source: /note=“Description of Artificial Sequence: Synthetic oligonucleotide”
cgcuccucccaguuccuaa

SEQ ID NO: 202 - RNA, 19 nt, Artificial Sequence
  source: /note=“Description of Artificial Sequence: Synthetic oligonucleotide”
gcgcccggguugagaauau

SEQ ID NO: 203 - RNA, 19 nt, Artificial Sequence
  source: /note=“Description of Artificial Sequence: Synthetic oligonucleotide”
cgcccggguugagaauaua

SEQ ID NO: 204 - RNA, 19 nt, Artificial Sequence
  source: /note=“Description of Artificial Sequence: Synthetic oligonucleotide”
ccggguugagaauauaauu

SEQ ID NO: 205 - RNA, 19 nt, Artificial Sequence
  source: /note=“Description of Artificial Sequence: Synthetic oligonucleotide”
cagcacugauucggaaaau

SEQ ID NO: 206 - RNA, 19 nt, Artificial Sequence
  source: /note=“Description of Artificial Sequence: Synthetic oligonucleotide”
cugucggaaggucagauaa

SEQ ID NO: 207 - RNA, 19 nt, Artificial Sequence
  source: /note=“Description of Artificial Sequence: Synthetic oligonucleotide”
caaccggaaggcaaacauu

SEQ ID NO: 208 - RNA, 19 nt, Artificial Sequence
  source: /note=“Description of Artificial Sequence: Synthetic oligonucleotide”
ccuugauggccuauccauu

SEQ ID NO: 209 - RNA, 19 nt, Artificial Sequence
  source: /note=“Description of Artificial Sequence: Synthetic oligonucleotide”
gcggcgaucuucaagauau

SEQ ID NO: 210 - RNA, 19 nt, Artificial Sequence
  source: /note=“Description of Artificial Sequence: Synthetic oligonucleotide”
gauugucacccaaucacuu

SEQ ID NO: 211 - RNA, 19 nt, Artificial Sequence
  source: /note=“Description of Artificial Sequence: Synthetic oligonucleotide”
gugguuuagcaauuuccgu

SEQ ID NO: 212 - RNA, 19 nt, Artificial Sequence
  source: /note=“Description of Artificial Sequence: Synthetic oligonucleotide”
gggucaccaguacugaaga

SEQ ID NO: 213 - RNA, 19 nt, Artificial Sequence
  source: /note=“Description of Artificial Sequence: Synthetic oligonucleotide”
gaagagcugucuauaacca

SEQ ID NO: 214 - RNA, 19 nt, Artificial Sequence
  source: /note=“Description of Artificial Sequence: Synthetic oligonucleotide”
cugcaagcuggauagugaa

SEQ ID NO: 215 - RNA, 19 nt, Artificial Sequence
  source: /note=“Description of Artificial Sequence: Synthetic oligonucleotide”
gcaagcuggauagugaagu

SEQ ID NO: 216 - RNA, 19 nt, Artificial Sequence
  source: /note=“Description of Artificial Sequence: Synthetic oligonucleotide”
gagcugcuucaugaguaga

SEQ ID NO: 217 - RNA, 19 nt, Artificial Sequence
  source: /note=“Description of Artificial Sequence: Synthetic oligonucleotide”
guaugggaggcauggauau

SEQ ID NO: 218 - RNA, 19 nt, Artificial Sequence
  source: /note=“Description of Artificial Sequence: Synthetic oligonucleotide”
gcccaaggcccuuaacauu

SEQ ID NO: 219 - RNA, 19 nt, Artificial Sequence
  source: /note=“Description of Artificial Sequence: Synthetic oligonucleotide”
gacacuuaaaauaggguua

SEQ ID NO: 220 - RNA, 19 nt, Artificial Sequence
  source: /note=“Description of Artificial Sequence: Synthetic oligonucleotide”
gacacagcacaacucuaca

SEQ ID NO: 221 - RNA, 19 nt, Artificial Sequence
  source: /note=“Description of Artificial Sequence: Synthetic oligonucleotide”
ccacuucauacauuuaagu

SEQ ID NO: 222 - RNA, 19 nt, Artificial Sequence
  source: /note=“Description of Artificial Sequence: Synthetic oligonucleotide”
cacuucauacauuuaagua

SEQ ID NO: 223-249 - skipped (27 entries)

SEQ ID NO: 250 - RNA, 19 nt, Artificial Sequence
  source: /note=“Description of Artificial Sequence: Synthetic oligonucleotide”
ucugaguaaugggaggcuc

SEQ ID NO: 251 - RNA, 19 nt, Artificial Sequence
  source: /note=“Description of Artificial Sequence: Synthetic oligonucleotide”
uuaggaacugggaggagcg

SEQ ID NO: 252 - RNA, 19 nt, Artificial Sequence
  source: /note=“Description of Artificial Sequence: Synthetic oligonucleotide”
auauucucaacccgggcgc

SEQ ID NO: 253 - RNA, 19 nt, Artificial Sequence
  source: /note=“Description of Artificial Sequence: Synthetic oligonucleotide”
uauauucucaacccgggcg

SEQ ID NO: 254 - RNA, 19 nt, Artificial Sequence
  source: /note=“Description of Artificial Sequence: Synthetic oligonucleotide”
aauuauauucucaacccgg

SEQ ID NO: 255 - RNA, 19 nt, Artificial Sequence
  source: /note=“Description of Artificial Sequence: Synthetic oligonucleotide”
auuuuccgaaucagugcug

SEQ ID NO: 256 - RNA, 19 nt, Artificial Sequence
  source: /note=“Description of Artificial Sequence: Synthetic oligonucleotide”
uuaucugaccuuccgacag

SEQ ID NO: 257 - RNA, 19 nt, Artificial Sequence
  source: /note=“Description of Artificial Sequence: Synthetic oligonucleotide”
aauguuugccuuccgguug

SEQ ID NO: 258 - RNA, 19 nt, Artificial Sequence
  source: /note=“Description of Artificial Sequence: Synthetic oligonucleotide”
aauggauaggccaucaagg

SEQ ID NO: 259 - RNA, 19 nt, Artificial Sequence
  source: /note=“Description of Artificial Sequence: Synthetic oligonucleotide”
auaucuugaagaucgccgc

SEQ ID NO: 260 - RNA, 19 nt, Artificial Sequence
  source: /note=“Description of Artificial Sequence: Synthetic oligonucleotide”
aagugauugggugacaauc

SEQ ID NO: 261 - RNA, 19 nt, Artificial Sequence
  source: /note=“Description of Artificial Sequence: Synthetic oligonucleotide”
acggaaauugcuaaaccac

SEQ ID NO: 262 - RNA, 19 nt, Artificial Sequence
  source: /note=“Description of Artificial Sequence: Synthetic oligonucleotide”
ucuucaguacuggugaccc

SEQ ID NO: 263 - RNA, 19 nt, Artificial Sequence
  source: /note=“Description of Artificial Sequence: Synthetic oligonucleotide”
ugguuauagacagcucuuc

SEQ ID NO: 264 - RNA, 19 nt, Artificial Sequence
  source: /note=“Description of Artificial Sequence: Synthetic oligonucleotide”
uucacuauccagcuugcag

SEQ ID NO: 265 - RNA, 19 nt, Artificial Sequence
  source: /note=“Description of Artificial Sequence: Synthetic oligonucleotide”
acuucacuauccagcuugc

SEQ ID NO: 266 - RNA, 19 nt, Artificial Sequence
  source: /note=“Description of Artificial Sequence: Synthetic oligonucleotide”
ucuacucaugaagcagcuc

SEQ ID NO: 267 - RNA, 19 nt, Artificial Sequence
  source: /note=“Description of Artificial Sequence: Synthetic oligonucleotide”
auauccaugccucccauac

SEQ ID NO: 268 - RNA, 19 nt, Artificial Sequence
  source: /note=“Description of Artificial Sequence: Synthetic oligonucleotide”
aauguuaagggccuugggc

SEQ ID NO: 269 - RNA, 19 nt, Artificial Sequence
  source: /note=“Description of Artificial Sequence: Synthetic oligonucleotide”
uaacccuauuuuaaguguc

SEQ ID NO: 270 - RNA, 19 nt, Artificial Sequence
  source: /note=“Description of Artificial Sequence: Synthetic oligonucleotide”
uguagaguugugcuguguc

SEQ ID NO: 271 - RNA, 19 nt, Artificial Sequence
  source: /note=“Description of Artificial Sequence: Synthetic oligonucleotide”
acuuaaauguaugaagugg

SEQ ID NO: 272 - RNA, 19 nt, Artificial Sequence
  source: /note=“Description of Artificial Sequence: Synthetic oligonucleotide”
uacuuaaauguaugaagug

SEQ ID NO: 273-299 - skipped (27 entries)

SEQ ID NO: 300 - RNA, 19 nt, Artificial Sequence
  source: /note=“Description of Artificial Sequence: Synthetic oligonucleotide”
ugcccagcugacgagcuuu

SEQ ID NO: 301 - RNA, 19 nt, Artificial Sequence
  source: /note=“Description of Artificial Sequence: Synthetic oligonucleotide”
gcccagcugacgagcuuuu

SEQ ID NO: 302 - RNA, 19 nt, Artificial Sequence
  source: /note=“Description of Artificial Sequence: Synthetic oligonucleotide”
ccagcugacgagcuuuuga

SEQ ID NO: 303 - RNA, 19 nt, Artificial Sequence
  source: /note=“Description of Artificial Sequence: Synthetic oligonucleotide”
cagcugacgagcuuuugaa

SEQ ID NO: 304 - RNA, 19 nt, Artificial Sequence
  source: /note=“Description of Artificial Sequence: Synthetic oligonucleotide”
gcugacgagcuuuugaaga

SEQ ID NO: 305 - RNA, 19 nt, Artificial Sequence
  source: /note=“Description of Artificial Sequence: Synthetic oligonucleotide”
cugacgagcuuuugaagau

SEQ ID NO: 306 - RNA, 19 nt, Artificial Sequence
  source: /note=“Description of Artificial Sequence: Synthetic oligonucleotide”
ucuuaagccggcaaaccaa

SEQ ID NO: 307 - RNA, 19 nt, Artificial Sequence
  source: /note=“Description of Artificial Sequence: Synthetic oligonucleotide”
uagggacagcaucugcauu

SEQ ID NO: 308 - RNA, 19 nt, Artificial Sequence
  source: /note=“Description of Artificial Sequence: Synthetic oligonucleotide”
caggcuccgaaguacauca

SEQ ID NO: 309 - RNA, 19 nt, Artificial Sequence
  source: /note=“Description of Artificial Sequence: Synthetic oligonucleotide”
ugucuuuccccuuuuggca

SEQ ID NO: 310 - RNA, 19 nt, Artificial Sequence
  source: /note=“Description of Artificial Sequence: Synthetic oligonucleotide”
cuacuaugagccaguuuga

SEQ ID NO: 311 - RNA, 19 nt, Artificial Sequence
  source: /note=“Description of Artificial Sequence: Synthetic oligonucleotide”
gaugagcaccugagagcaa

SEQ ID NO: 312 - RNA, 19 nt, Artificial Sequence
  source: /note=“Description of Artificial Sequence: Synthetic oligonucleotide”
augagcaccugagagcaaa

SEQ ID NO: 313 - RNA, 19 nt, Artificial Sequence
  source: /note=“Description of Artificial Sequence: Synthetic oligonucleotide”
auggccccgcagucuguga

SEQ ID NO: 314 - RNA, 19 nt, Artificial Sequence
  source: /note=“Description of Artificial Sequence: Synthetic oligonucleotide”
aguccccgagaaaguuaca

SEQ ID NO: 315 - RNA, 19 nt, Artificial Sequence
  source: /note=“Description of Artificial Sequence: Synthetic oligonucleotide”
uccccgagaaaguuacaga

SEQ ID NO: 316 - RNA, 19 nt, Artificial Sequence
  source: /note=“Description of Artificial Sequence: Synthetic oligonucleotide”
uacagagaaaacaaacgca

SEQ ID NO: 317 - RNA, 19 nt, Artificial Sequence
  source: /note=“Description of Artificial Sequence: Synthetic oligonucleotide”
acagagaaaacaaacgcaa

SEQ ID NO: 318 - RNA, 19 nt, Artificial Sequence
  source: /note=“Description of Artificial Sequence: Synthetic oligonucleotide”
ugguuucagcccgaaaaga

SEQ ID NO: 319 - RNA, 19 nt, Artificial Sequence
  source: /note=“Description of Artificial Sequence: Synthetic oligonucleotide”
gguuucagcccgaaaagaa

SEQ ID NO: 320 - RNA, 19 nt, Artificial Sequence
  source: /note=“Description of Artificial Sequence: Synthetic oligonucleotide”
ucacccaaucacuugaaaa

SEQ ID NO: 321 - RNA, 19 nt, Artificial Sequence
  source: /note=“Description of Artificial Sequence: Synthetic oligonucleotide”
agcaauuuccgugaguuuu

SEQ ID NO: 322 - RNA, 19 nt, Artificial Sequence
  source: /note=“Description of Artificial Sequence: Synthetic oligonucleotide”
aauuuccgugaguuuuacu

SEQ ID NO: 323-349 - skipped (27 entries)

SEQ ID NO: 350 - RNA, 19 nt, Artificial Sequence
  source: /note=“Description of Artificial Sequence: Synthetic oligonucleotide”
aaagcucgucagcugggca

SEQ ID NO: 351 - RNA, 19 nt, Artificial Sequence
  source: /note=“Description of Artificial Sequence: Synthetic oligonucleotide”
aaaagcucgucagcugggc

SEQ ID NO: 352 - RNA, 19 nt, Artificial Sequence
  source: /note=“Description of Artificial Sequence: Synthetic oligonucleotide”
ucaaaagcucgucagcugg

SEQ ID NO: 353 - RNA, 19 nt, Artificial Sequence
  source: /note=“Description of Artificial Sequence: Synthetic oligonucleotide”
uucaaaagcucgucagcug

SEQ ID NO: 354 - RNA, 19 nt, Artificial Sequence
  source: /note=“Description of Artificial Sequence: Synthetic oligonucleotide”
ucuucaaaagcucgucagc

SEQ ID NO: 355 - RNA, 19 nt, Artificial Sequence
  source: /note=“Description of Artificial Sequence: Synthetic oligonucleotide”
aucuucaaaagcucgucag

SEQ ID NO: 356 - RNA, 19 nt, Artificial Sequence
  source: /note=“Description of Artificial Sequence: Synthetic oligonucleotide”
uugguuugccggcuuaaga

SEQ ID NO: 357 - RNA, 19 nt, Artificial Sequence
  source: /note=“Description of Artificial Sequence: Synthetic oligonucleotide”
aaugcagaugcugucccua

SEQ ID NO: 358 - RNA, 19 nt, Artificial Sequence
  source: /note=“Description of Artificial Sequence: Synthetic oligonucleotide”
ugauguacuucggagccug

SEQ ID NO: 359 - RNA, 19 nt, Artificial Sequence
  source: /note=“Description of Artificial Sequence: Synthetic oligonucleotide”
ugccaaaaggggaaagaca

SEQ ID NO: 360 - RNA, 19 nt, Artificial Sequence
  source: /note=“Description of Artificial Sequence: Synthetic oligonucleotide”
ucaaacuggcucauaguag

SEQ ID NO: 361 - RNA, 19 nt, Artificial Sequence
  source: /note=“Description of Artificial Sequence: Synthetic oligonucleotide”
uugcucucaggugcucauc

SEQ ID NO: 362 - RNA, 19 nt, Artificial Sequence
  source: /note=“Description of Artificial Sequence: Synthetic oligonucleotide”
uuugcucucaggugcucau

SEQ ID NO: 363 - RNA, 19 nt, Artificial Sequence
  source: /note=“Description of Artificial Sequence: Synthetic oligonucleotide”
ucacagacugcggggccau

SEQ ID NO: 364 - RNA, 19 nt, Artificial Sequence
  source: /note=“Description of Artificial Sequence: Synthetic oligonucleotide”
uguaacuuucucggggacu

SEQ ID NO: 365 - RNA, 19 nt, Artificial Sequence
  source: /note=“Description of Artificial Sequence: Synthetic oligonucleotide”
ucuguaacuuucucgggga

SEQ ID NO: 366 - RNA, 19 nt, Artificial Sequence
  source: /note=“Description of Artificial Sequence: Synthetic oligonucleotide”
ugcguuuguuuucucugua

SEQ ID NO: 367 - RNA, 19 nt, Artificial Sequence
  source: /note=“Description of Artificial Sequence: Synthetic oligonucleotide”
uugcguuuguuuucucugu

SEQ ID NO: 368 - RNA, 19 nt, Artificial Sequence
  source: /note=“Description of Artificial Sequence: Synthetic oligonucleotide”
ucuuuucgggcugaaacca

SEQ ID NO: 369 - RNA, 19 nt, Artificial Sequence
  source: /note=“Description of Artificial Sequence: Synthetic oligonucleotide”
uucuuuucgggcugaaacc

SEQ ID NO: 370 - RNA, 19 nt, Artificial Sequence
  source: /note=“Description of Artificial Sequence: Synthetic oligonucleotide”
uuuucaagugauuggguga

SEQ ID NO: 371 - RNA, 19 nt, Artificial Sequence
  source: /note=“Description of Artificial Sequence: Synthetic oligonucleotide”
aaaacucacggaaauugcu

SEQ ID NO: 372 - RNA, 19 nt, Artificial Sequence
  source: /note=“Description of Artificial Sequence: Synthetic oligonucleotide”
aguaaaacucacggaaauu

SEQ ID NO: 373-399 - skipped (27 entries)

SEQ ID NO: 400 - RNA, 19 nt, Artificial Sequence
  source: /note=“Description of Artificial Sequence: Synthetic oligonucleotide”
gacccacccaagaguucaa

SEQ ID NO: 401 - RNA, 19 nt, Artificial Sequence
  source: /note=“Description of Artificial Sequence: Synthetic oligonucleotide”
ccugacaaccuuuacaccu

SEQ ID NO: 402 - RNA, 19 nt, Artificial Sequence
  source: /note=“Description of Artificial Sequence: Synthetic oligonucleotide”
gcagagaugcauauucccu

SEQ ID NO: 403 - RNA, 19 nt, Artificial Sequence
  source: /note=“Description of Artificial Sequence: Synthetic oligonucleotide”
gcacagauucccuuaacca

SEQ ID NO: 404 - RNA, 19 nt, Artificial Sequence
  source: /note=“Description of Artificial Sequence: Synthetic oligonucleotide”
gccagaagacggaccauuu

SEQ ID NO: 405 - RNA, 19 nt, Artificial Sequence
  source: /note=“Description of Artificial Sequence: Synthetic oligonucleotide”
cgggacucggacuacgaaa

SEQ ID NO: 406 - RNA, 19 nt, Artificial Sequence
  source: /note=“Description of Artificial Sequence: Synthetic oligonucleotide”
gggacucggacuacgaaau

SEQ ID NO: 407 - RNA, 19 nt, Artificial Sequence
  source: /note=“Description of Artificial Sequence: Synthetic oligonucleotide”
ggacuacgaaauggacuca

SEQ ID NO: 408 - RNA, 19 nt, Artificial Sequence
  source: /note=“Description of Artificial Sequence: Synthetic oligonucleotide”
cugcucgugccaguuugga

SEQ ID NO: 409 - RNA, 19 nt, Artificial Sequence
  source: /note=“Description of Artificial Sequence: Synthetic oligonucleotide”
cccguuacucuuagagaaa

SEQ ID NO: 410 - RNA, 19 nt, Artificial Sequence
  source: /note=“Description of Artificial Sequence: Synthetic oligonucleotide”
cgguucucccaucuaccgu

SEQ ID NO: 411 - RNA, 19 nt, Artificial Sequence
  source: /note=“Description of Artificial Sequence: Synthetic oligonucleotide”
gaccacaucuaauccugcu

SEQ ID NO: 412 - RNA, 19 nt, Artificial Sequence
  source: /note=“Description of Artificial Sequence: Synthetic oligonucleotide”
cagugcacuguuucaacua

SEQ ID NO: 413 - RNA, 19 nt, Artificial Sequence
  source: /note=“Description of Artificial Sequence: Synthetic oligonucleotide”
ggguggcaagaacaguucu

SEQ ID NO: 414 - RNA, 19 nt, Artificial Sequence
  source: /note=“Description of Artificial Sequence: Synthetic oligonucleotide”
ccuggugccuuacgaauaa

SEQ ID NO: 415 - RNA, 19 nt, Artificial Sequence
  source: /note=“Description of Artificial Sequence: Synthetic oligonucleotide”
gcuuuacagugauaguuaa

SEQ ID NO: 416 - RNA, 19 nt, Artificial Sequence
  source: /note=“Description of Artificial Sequence: Synthetic oligonucleotide”
ccuaggcccuuucuaguaa

SEQ ID NO: 417 - RNA, 19 nt, Artificial Sequence
  source: /note=“Description of Artificial Sequence: Synthetic oligonucleotide”
cccuuucuaguaaauccuu

SEQ ID NO: 418 - RNA, 19 nt, Artificial Sequence
  source: /note=“Description of Artificial Sequence: Synthetic oligonucleotide”
caagcaucuaauggcuuau

SEQ ID NO: 419 - RNA, 19 nt, Artificial Sequence
  source: /note=“Description of Artificial Sequence: Synthetic oligonucleotide”
ggcuuauuaaauuauccca

SEQ ID NO: 420 - RNA, 19 nt, Artificial Sequence
  source: /note=“Description of Artificial Sequence: Synthetic oligonucleotide”
cacaaguggguuuuaggcu

SEQ ID NO: 421 - RNA, 19 nt, Artificial Sequence
  source: /note=“Description of Artificial Sequence: Synthetic oligonucleotide”
ccacaaucaguguucuguu

SEQ ID NO: 422 - RNA, 19 nt, Artificial Sequence
  source: /note=“Description of Artificial Sequence: Synthetic oligonucleotide”
gagaaucuaucuaucuccu

SEQ ID NO: 423 - RNA, 19 nt, Artificial Sequence
  source: /note=“Description of Artificial Sequence: Synthetic oligonucleotide”
gccuaaaacucauaggacu

SEQ ID NO: 424 - RNA, 19 nt, Artificial Sequence
  source: /note=“Description of Artificial Sequence: Synthetic oligonucleotide”
gaggguuaggucugcuucu

SEQ ID NO: 425 - RNA, 19 nt, Artificial Sequence
  source: /note=“Description of Artificial Sequence: Synthetic oligonucleotide”
gcuucuuccacuuauacuu

SEQ ID NO: 426 - RNA, 19 nt, Artificial Sequence
  source: /note=“Description of Artificial Sequence: Synthetic oligonucleotide”
gcuaauuagugugaaccaa

SEQ ID NO: 427 - RNA, 19 nt, Artificial Sequence
  source: /note=“Description of Artificial Sequence: Synthetic oligonucleotide”
gugugaaccaaaagaguaa

SEQ ID NO: 428 - RNA, 19 nt, Artificial Sequence
  source: /note=“Description of Artificial Sequence: Synthetic oligonucleotide”
gaaccaaaagaguaaguaa

SEQ ID NO: 429 - RNA, 19 nt, Artificial Sequence
  source: /note=“Description of Artificial Sequence: Synthetic oligonucleotide”
cugggauaugacgucuuca

SEQ ID NO: 430 - RNA, 19 nt, Artificial Sequence
  source: /note=“Description of Artificial Sequence: Synthetic oligonucleotide”
caugccaggggcacucauu

SEQ ID NO: 431 - RNA, 19 nt, Artificial Sequence
  source: /note=“Description of Artificial Sequence: Synthetic oligonucleotide”
gggcacucauuucuuagca

SEQ ID NO: 432 - RNA, 19 nt, Artificial Sequence
  source: /note=“Description of Artificial Sequence: Synthetic oligonucleotide”
ccucucuacauacaucucu

SEQ ID NO: 433 - RNA, 19 nt, Artificial Sequence
  source: /note=“Description of Artificial Sequence: Synthetic oligonucleotide”
gaaaacucccauggcucua

SEQ ID NO: 434 - RNA, 19 nt, Artificial Sequence
  source: /note=“Description of Artificial Sequence: Synthetic oligonucleotide”
caccaaacuggacaguaga

SEQ ID NO: 435 - RNA, 19 nt, Artificial Sequence
  source: /note=“Description of Artificial Sequence: Synthetic oligonucleotide”
ccauauggcuuuaucuggu

SEQ ID NO: 436 - RNA, 19 nt, Artificial Sequence
  source: /note=“Description of Artificial Sequence: Synthetic oligonucleotide”
cauauggcuuuaucuggua

SEQ ID NO: 437 - RNA, 19 nt, Artificial Sequence
  source: /note=“Description of Artificial Sequence: Synthetic oligonucleotide”
gacuaagucccuuacugca

SEQ ID NO: 438 - RNA, 19 nt, Artificial Sequence
  source: /note=“Description of Artificial Sequence: Synthetic oligonucleotide”
gaggaacuugcgaguauau

SEQ ID NO: 439 - RNA, 19 nt, Artificial Sequence
  source: /note=“Description of Artificial Sequence: Synthetic oligonucleotide”
cauuauaggcagaagguau

SEQ ID NO: 440 - RNA, 19 nt, Artificial Sequence
  source: /note=“Description of Artificial Sequence: Synthetic oligonucleotide”
cuuauucuugaauaaugca

SEQ ID NO: 441-449 - skipped (9 entries)

SEQ ID NO: 450 - RNA, 19 nt, Artificial Sequence
  source: /note=“Description of Artificial Sequence: Synthetic oligonucleotide”
uugaacucuuggguggguc

SEQ ID NO: 451 - RNA, 19 nt, Artificial Sequence
  source: /note=“Description of Artificial Sequence: Synthetic oligonucleotide”
agguguaaagguugucagg

SEQ ID NO: 452 - RNA, 19 nt, Artificial Sequence
  source: /note=“Description of Artificial Sequence: Synthetic oligonucleotide”
agggaauaugcaucucugc

SEQ ID NO: 453 - RNA, 19 nt, Artificial Sequence
  source: /note=“Description of Artificial Sequence: Synthetic oligonucleotide”
ugguuaagggaaucugugc

SEQ ID NO: 454 - RNA, 19 nt, Artificial Sequence
  source: /note=“Description of Artificial Sequence: Synthetic oligonucleotide”
aaaugguccgucuucuggc

SEQ ID NO: 455 - RNA, 19 nt, Artificial Sequence
  source: /note=“Description of Artificial Sequence: Synthetic oligonucleotide”
uuucguaguccgagucccg

SEQ ID NO: 456 - RNA, 19 nt, Artificial Sequence
  source: /note=“Description of Artificial Sequence: Synthetic oligonucleotide”
auuucguaguccgaguccc

SEQ ID NO: 457 - RNA, 19 nt, Artificial Sequence
  source: /note=“Description of Artificial Sequence: Synthetic oligonucleotide”
ugaguccauuucguagucc

SEQ ID NO: 458 - RNA, 19 nt, Artificial Sequence
  source: /note=“Description of Artificial Sequence: Synthetic oligonucleotide”
uccaaacuggcacgagcag

SEQ ID NO: 459 - RNA, 19 nt, Artificial Sequence
  source: /note=“Description of Artificial Sequence: Synthetic oligonucleotide”
uuucucuaagaguaacggg

SEQ ID NO: 460 - RNA, 19 nt, Artificial Sequence
  source: /note=“Description of Artificial Sequence: Synthetic oligonucleotide”
acgguagaugggagaaccg

SEQ ID NO: 461 - RNA, 19 nt, Artificial Sequence
  source: /note=“Description of Artificial Sequence: Synthetic oligonucleotide”
agcaggauuagaugugguc

SEQ ID NO: 462 - RNA, 19 nt, Artificial Sequence
  source: /note=“Description of Artificial Sequence: Synthetic oligonucleotide”
uaguugaaacagugcacug

SEQ ID NO: 463 - RNA, 19 nt, Artificial Sequence
  source: /note=“Description of Artificial Sequence: Synthetic oligonucleotide”
agaacuguucuugccaccc

SEQ ID NO: 464 - RNA, 19 nt, Artificial Sequence
  source: /note=“Description of Artificial Sequence: Synthetic oligonucleotide”
uuauucguaaggcaccagg

SEQ ID NO: 465 - RNA, 19 nt, Artificial Sequence
  source: /note=“Description of Artificial Sequence: Synthetic oligonucleotide”
uuaacuaucacuguaaagc

SEQ ID NO: 466 - RNA, 19 nt, Artificial Sequence
  source: /note=“Description of Artificial Sequence: Synthetic oligonucleotide”
uuacuagaaagggccuagg

SEQ ID NO: 467 - RNA, 19 nt, Artificial Sequence
  source: /note=“Description of Artificial Sequence: Synthetic oligonucleotide”
aaggauuuacuagaaaggg

SEQ ID NO: 468 - RNA, 19 nt, Artificial Sequence
  source: /note=“Description of Artificial Sequence: Synthetic oligonucleotide”
auaagccauuagaugcuug

SEQ ID NO: 469 - RNA, 19 nt, Artificial Sequence
  source: /note=“Description of Artificial Sequence: Synthetic oligonucleotide”
ugggauaauuuaauaagcc

SEQ ID NO: 470 - RNA, 19 nt, Artificial Sequence
  source: /note=“Description of Artificial Sequence: Synthetic oligonucleotide”
agccuaaaacccacuugug

SEQ ID NO: 471 - RNA, 19 nt, Artificial Sequence
  source: /note=“Description of Artificial Sequence: Synthetic oligonucleotide”
aacagaacacugauugugg

SEQ ID NO: 472 - RNA, 19 nt, Artificial Sequence
  source: /note=“Description of Artificial Sequence: Synthetic oligonucleotide”
aggagauagauagauucuc

SEQ ID NO: 473 - RNA, 19 nt, Artificial Sequence
  source: /note=“Description of Artificial Sequence: Synthetic oligonucleotide”
aguccuaugaguuuuaggc

SEQ ID NO: 474 - RNA, 19 nt, Artificial Sequence
  source: /note=“Description of Artificial Sequence: Synthetic oligonucleotide”
agaagcagaccuaacccuc

SEQ ID NO: 475 - RNA, 19 nt, Artificial Sequence
  source: /note=“Description of Artificial Sequence: Synthetic oligonucleotide”
aaguauaaguggaagaagc

SEQ ID NO: 476 - RNA, 19 nt, Artificial Sequence
  source: /note=“Description of Artificial Sequence: Synthetic oligonucleotide”
uugguucacacuaauuagc

SEQ ID NO: 477 - RNA, 19 nt, Artificial Sequence
  source: /note=“Description of Artificial Sequence: Synthetic oligonucleotide”
uuacucuuuugguucacac

SEQ ID NO: 478 - RNA, 19 nt, Artificial Sequence
  source: /note=“Description of Artificial Sequence: Synthetic oligonucleotide”
uuacuuacucuuuugguuc

SEQ ID NO: 479 - RNA, 19 nt, Artificial Sequence
  source: /note=“Description of Artificial Sequence: Synthetic oligonucleotide”
ugaagacgucauaucccag

SEQ ID NO: 480 - RNA, 19 nt, Artificial Sequence
  source: /note=“Description of Artificial Sequence: Synthetic oligonucleotide”
aaugagugccccuggcaug

SEQ ID NO: 481 - RNA, 19 nt, Artificial Sequence
  source: /note=“Description of Artificial Sequence: Synthetic oligonucleotide”
ugcuaagaaaugagugccc

SEQ ID NO: 482 - RNA, 19 nt, Artificial Sequence
  source: /note=“Description of Artificial Sequence: Synthetic oligonucleotide”
agagauguauguagagagg

SEQ ID NO: 483 - RNA, 19 nt, Artificial Sequence
  source: /note=“Description of Artificial Sequence: Synthetic oligonucleotide”
uagagccaugggaguuuuc

SEQ ID NO: 484 - RNA, 19 nt, Artificial Sequence
  source: /note=“Description of Artificial Sequence: Synthetic oligonucleotide”
ucuacuguccaguuuggug

SEQ ID NO: 485 - RNA, 19 nt, Artificial Sequence
  source: /note=“Description of Artificial Sequence: Synthetic oligonucleotide”
accagauaaagccauaugg

SEQ ID NO: 486 - RNA, 19 nt, Artificial Sequence
  source: /note=“Description of Artificial Sequence: Synthetic oligonucleotide”
uaccagauaaagccauaug

SEQ ID NO: 487 - RNA, 19 nt, Artificial Sequence
  source: /note=“Description of Artificial Sequence: Synthetic oligonucleotide”
ugcaguaagggacuuaguc

SEQ ID NO: 488 - RNA, 19 nt, Artificial Sequence
  source: /note=“Description of Artificial Sequence: Synthetic oligonucleotide”
auauacucgcaaguuccuc

SEQ ID NO: 489 - RNA, 19 nt, Artificial Sequence
  source: /note=“Description of Artificial Sequence: Synthetic oligonucleotide”
auaccuucugccuauaaug

SEQ ID NO: 490 - RNA, 19 nt, Artificial Sequence
  source: /note=“Description of Artificial Sequence: Synthetic oligonucleotide”
ugcauuauucaagaauaag

SEQ ID NO: 491-499 - skipped (9 entries)

SEQ ID NO: 500 - RNA, 19 nt, Artificial Sequence
  source: /note=“Description of Artificial Sequence: Synthetic oligonucleotide”
cagacaucaucuuugaggu

SEQ ID NO: 501 - RNA, 19 nt, Artificial Sequence
  source: /note=“Description of Artificial Sequence: Synthetic oligonucleotide”
ucccuuccucacugggauu

SEQ ID NO: 502 - RNA, 19 nt, Artificial Sequence
  source: /note=“Description of Artificial Sequence: Synthetic oligonucleotide”
gaucuuccaccuggugccu

SEQ ID NO: 503 - RNA, 19 nt, Artificial Sequence
  source: /note=“Description of Artificial Sequence: Synthetic oligonucleotide”
aucuuccaccuggugccuu

SEQ ID NO: 504 - RNA, 19 nt, Artificial Sequence
  source: /note=“Description of Artificial Sequence: Synthetic oligonucleotide”
uguuuacagauguaguucu

SEQ ID NO: 505 - RNA, 19 nt, Artificial Sequence
  source: /note=“Description of Artificial Sequence: Synthetic oligonucleotide”
guuuacagauguaguucuu

SEQ ID NO: 506 - RNA, 19 nt, Artificial Sequence
  source: /note=“Description of Artificial Sequence: Synthetic oligonucleotide”
uuacagauguaguucuugu

SEQ ID NO: 507-549 - skipped (43 entries)

SEQ ID NO: 550 - RNA, 19 nt, Artificial Sequence
  source: /note=“Description of Artificial Sequence: Synthetic oligonucleotide”
accucaaagaugaugucug

SEQ ID NO: 551 - RNA, 19 nt, Artificial Sequence
  source: /note=“Description of Artificial Sequence: Synthetic oligonucleotide”
aaucccagugaggaaggga

SEQ ID NO: 552 - RNA, 19 nt, Artificial Sequence
  source: /note=“Description of Artificial Sequence: Synthetic oligonucleotide”
aggcaccagguggaagauc

SEQ ID NO: 553 - RNA, 19 nt, Artificial Sequence
  source: /note=“Description of Artificial Sequence: Synthetic oligonucleotide”
aaggcaccagguggaagau

SEQ ID NO: 554 - RNA, 19 nt, Artificial Sequence
  source: /note=“Description of Artificial Sequence: Synthetic oligonucleotide”
agaacuacaucuguaaaca

SEQ ID NO: 555 - RNA, 19 nt, Artificial Sequence
  source: /note=“Description of Artificial Sequence: Synthetic oligonucleotide”
aagaacuacaucuguaaac

SEQ ID NO: 556 - RNA, 19 nt, Artificial Sequence
  source: /note=“Description of Artificial Sequence: Synthetic oligonucleotide”
acaagaacuacaucuguaa

SEQ ID NO: 557-599 - skipped (43 entries)

SEQ ID NO: 600 - RNA, 19 nt, Artificial Sequence
  source: /note=“Description of Artificial Sequence: Synthetic oligonucleotide”
gucccgguaccaugcauuu

SEQ ID NO: 601 - RNA, 19 nt, Artificial Sequence
  source: /note=“Description of Artificial Sequence: Synthetic oligonucleotide”
cccaaggaagugacucaaa

SEQ ID NO: 602 - RNA, 19 nt, Artificial Sequence
  source: /note=“Description of Artificial Sequence: Synthetic oligonucleotide”
cuauuggugguaugucuga

SEQ ID NO: 603 - RNA, 19 nt, Artificial Sequence
  source: /note=“Description of Artificial Sequence: Synthetic oligonucleotide”
gccucaccaugaaaucaca

SEQ ID NO: 604 - RNA, 19 nt, Artificial Sequence
  source: /note=“Description of Artificial Sequence: Synthetic oligonucleotide”
cugucaucucagcaaaacu

SEQ ID NO: 605 - RNA, 19 nt, Artificial Sequence
  source: /note=“Description of Artificial Sequence: Synthetic oligonucleotide”
ccccugugaguaaauuaca

SEQ ID NO: 606 - RNA, 19 nt, Artificial Sequence
  source: /note=“Description of Artificial Sequence: Synthetic oligonucleotide”
cccugugaguaaauuacau

SEQ ID NO: 607 - RNA, 19 nt, Artificial Sequence
  source: /note=“Description of Artificial Sequence: Synthetic oligonucleotide”
guaaauuacauuuucgugu

SEQ ID NO: 608 - RNA, 19 nt, Artificial Sequence
  source: /note=“Description of Artificial Sequence: Synthetic oligonucleotide”
gggaacugcugcauuagau

SEQ ID NO: 609 - RNA, 19 nt, Artificial Sequence
  source: /note=“Description of Artificial Sequence: Synthetic oligonucleotide”
ggaacugcugcauuagaua

SEQ ID NO: 610 - RNA, 19 nt, Artificial Sequence
  source: /note=“Description of Artificial Sequence: Synthetic oligonucleotide”
cugcugcauuagauauuua

SEQ ID NO: 611 - RNA, 19 nt, Artificial Sequence
  source: /note=“Description of Artificial Sequence: Synthetic oligonucleotide”
cauuaaagucaaacaauau

SEQ ID NO: 612 - RNA, 19 nt, Artificial Sequence
  source: /note=“Description of Artificial Sequence: Synthetic oligonucleotide”
cuccagauccaaggaugaa

SEQ ID NO: 613 - RNA, 19 nt, Artificial Sequence
  source: /note=“Description of Artificial Sequence: Synthetic oligonucleotide”
cguagaccagcaucuguca

SEQ ID NO: 614 - RNA, 19 nt, Artificial Sequence
  source: /note=“Description of Artificial Sequence: Synthetic oligonucleotide”
gcaacaucuggauuaauaa

SEQ ID NO: 615 - RNA, 19 nt, Artificial Sequence
  source: /note=“Description of Artificial Sequence: Synthetic oligonucleotide”
cauuaaauccuguaacuca

SEQ ID NO: 616 - RNA, 19 nt, Artificial Sequence
  source: /note=“Description of Artificial Sequence: Synthetic oligonucleotide”
caacaugggauagaccaga

SEQ ID NO: 617 - RNA, 19 nt, Artificial Sequence
  source: /note=“Description of Artificial Sequence: Synthetic oligonucleotide”
gacaacaugggacguauuu

SEQ ID NO: 618 - RNA, 19 nt, Artificial Sequence
  source: /note=“Description of Artificial Sequence: Synthetic oligonucleotide”
gcuacagcguagucagcuu

SEQ ID NO: 619 - RNA, 19 nt, Artificial Sequence
  source: /note=“Description of Artificial Sequence: Synthetic oligonucleotide”
cagagauucauuuauggga

SEQ ID NO: 620 - RNA, 19 nt, Artificial Sequence
  source: /note=“Description of Artificial Sequence: Synthetic oligonucleotide”
ggcagaguauauuucguca

SEQ ID NO: 621 - RNA, 19 nt, Artificial Sequence
  source: /note=“Description of Artificial Sequence: Synthetic oligonucleotide”
gcagaguauauuucgucaa

SEQ ID NO: 622 - RNA, 19 nt, Artificial Sequence
  source: /note=“Description of Artificial Sequence: Synthetic oligonucleotide”
ccagaagucaaggucaauu

SEQ ID NO: 623 - RNA, 19 nt, Artificial Sequence
  source: /note=“Description of Artificial Sequence: Synthetic oligonucleotide”
gaacuaccaccuauauaga

SEQ ID NO: 624 - RNA, 19 nt, Artificial Sequence
  source: /note=“Description of Artificial Sequence: Synthetic oligonucleotide”
gccacagcacauaaagauu

SEQ ID NO: 625 - RNA, 19 nt, Artificial Sequence
  source: /note=“Description of Artificial Sequence: Synthetic oligonucleotide”
gauuauggagguguagcaa

SEQ ID NO: 626 - RNA, 19 nt, Artificial Sequence
  source: /note=“Description of Artificial Sequence: Synthetic oligonucleotide”
ggaaggauaacuacugcuu

SEQ ID NO: 627 - RNA, 19 nt, Artificial Sequence
  source: /note=“Description of Artificial Sequence: Synthetic oligonucleotide”
cgcuucuuacaucaaucca

SEQ ID NO: 628 - RNA, 19 nt, Artificial Sequence
  source: /note=“Description of Artificial Sequence: Synthetic oligonucleotide”
caccugaaauauuuucguu

SEQ ID NO: 629 - RNA, 19 nt, Artificial Sequence
  source: /note=“Description of Artificial Sequence: Synthetic oligonucleotide”
ccauucuauaagcguaucu

SEQ ID NO: 630 - RNA, 19 nt, Artificial Sequence
  source: /note=“Description of Artificial Sequence: Synthetic oligonucleotide”
guacuucuccguugacaaa

SEQ ID NO: 631 - RNA, 19 nt, Artificial Sequence
  source: /note=“Description of Artificial Sequence: Synthetic oligonucleotide”
gagucaugaucugaaaccu

SEQ ID NO: 632 - RNA, 19 nt, Artificial Sequence
  source: /note=“Description of Artificial Sequence: Synthetic oligonucleotide”
cuaaugguggcaauauucu

SEQ ID NO: 633 - RNA, 19 nt, Artificial Sequence
  source: /note=“Description of Artificial Sequence: Synthetic oligonucleotide”
gguugucucgagguguuga

SEQ ID NO: 634 - RNA, 19 nt, Artificial Sequence
  source: /note=“Description of Artificial Sequence: Synthetic oligonucleotide”
caugccaucuaccgucauu

SEQ ID NO: 635 - RNA, 19 nt, Artificial Sequence
  source: /note=“Description of Artificial Sequence: Synthetic oligonucleotide”
gauuuggacaagaguaacu

SEQ ID NO: 636 - RNA, 19 nt, Artificial Sequence
  source: /note=“Description of Artificial Sequence: Synthetic oligonucleotide”
gggcaagaacuuauuugca

SEQ ID NO: 637 - RNA, 19 nt, Artificial Sequence
  source: /note=“Description of Artificial Sequence: Synthetic oligonucleotide”
cacaucuuguaaaauugga

SEQ ID NO: 638 - RNA, 19 nt, Artificial Sequence
  source: /note=“Description of Artificial Sequence: Synthetic oligonucleotide”
gaguuaucugaguguaagu

SEQ ID NO: 639 - RNA, 19 nt, Artificial Sequence
  source: /note=“Description of Artificial Sequence: Synthetic oligonucleotide”
guuaucugaguguaaguaa

SEQ ID NO: 640 - RNA, 19 nt, Artificial Sequence
  source: /note=“Description of Artificial Sequence: Synthetic oligonucleotide”
cauuuagauuuaucuccca

SEQ ID NO: 641 - RNA, 19 nt, Artificial Sequence
  source: /note=“Description of Artificial Sequence: Synthetic oligonucleotide”
gucugcaaaugacuaguca

SEQ ID NO: 642 - RNA, 19 nt, Artificial Sequence
  source: /note=“Description of Artificial Sequence: Synthetic oligonucleotide”
cccucucauuuuagucuga

SEQ ID NO: 643 - RNA, 19 nt, Artificial Sequence
  source: /note=“Description of Artificial Sequence: Synthetic oligonucleotide”
gucugacuugacuauuguu

SEQ ID NO: 644 - RNA, 19 nt, Artificial Sequence
  source: /note=“Description of Artificial Sequence: Synthetic oligonucleotide”
gaauccauacaucauuccu

SEQ ID NO: 645-649 - skipped (5 entries)

SEQ ID NO: 650 - RNA, 19 nt, Artificial Sequence
  source: /note=“Description of Artificial Sequence: Synthetic oligonucleotide”
aaaugcaugguaccgggac

SEQ ID NO: 651 - RNA, 19 nt, Artificial Sequence
  source: /note=“Description of Artificial Sequence: Synthetic oligonucleotide”
uuugagucacuuccuuggg

SEQ ID NO: 652 - RNA, 19 nt, Artificial Sequence
  source: /note=“Description of Artificial Sequence: Synthetic oligonucleotide”
ucagacauaccaccaauag

SEQ ID NO: 653 - RNA, 19 nt, Artificial Sequence
  source: /note=“Description of Artificial Sequence: Synthetic oligonucleotide”
ugugauuucauggugaggc

SEQ ID NO: 654 - RNA, 19 nt, Artificial Sequence
  source: /note=“Description of Artificial Sequence: Synthetic oligonucleotide”
aguuuugcugagaugacag

SEQ ID NO: 655 - RNA, 19 nt, Artificial Sequence
  source: /note=“Description of Artificial Sequence: Synthetic oligonucleotide”
uguaauuuacucacagggg

SEQ ID NO: 656 - RNA, 19 nt, Artificial Sequence
  source: /note=“Description of Artificial Sequence: Synthetic oligonucleotide”
auguaauuuacucacaggg

SEQ ID NO: 657 - RNA, 19 nt, Artificial Sequence
  source: /note=“Description of Artificial Sequence: Synthetic oligonucleotide”
acacgaaaauguaauuuac

SEQ ID NO: 658 - RNA, 19 nt, Artificial Sequence
  source: /note=“Description of Artificial Sequence: Synthetic oligonucleotide”
aucuaaugcagcaguuccc

SEQ ID NO: 659 - RNA, 19 nt, Artificial Sequence
  source: /note=“Description of Artificial Sequence: Synthetic oligonucleotide”
uaucuaaugcagcaguucc

SEQ ID NO: 660 - RNA, 19 nt, Artificial Sequence
  source: /note=“Description of Artificial Sequence: Synthetic oligonucleotide”
uaaauaucuaaugcagcag

SEQ ID NO: 661 - RNA, 19 nt, Artificial Sequence
  source: /note=“Description of Artificial Sequence: Synthetic oligonucleotide”
auauuguuugacuuuaaug

SEQ ID NO: 662 - RNA, 19 nt, Artificial Sequence
  source: /note=“Description of Artificial Sequence: Synthetic oligonucleotide”
uucauccuuggaucuggag

SEQ ID NO: 663 - RNA, 19 nt, Artificial Sequence
  source: /note=“Description of Artificial Sequence: Synthetic oligonucleotide”
ugacagaugcuggucuacg

SEQ ID NO: 664 - RNA, 19 nt, Artificial Sequence
  source: /note=“Description of Artificial Sequence: Synthetic oligonucleotide”
uuauuaauccagauguugc

SEQ ID NO: 665 - RNA, 19 nt, Artificial Sequence
  source: /note=“Description of Artificial Sequence: Synthetic oligonucleotide”
ugaguuacaggauuuaaug

SEQ ID NO: 666 - RNA, 19 nt, Artificial Sequence
  source: /note=“Description of Artificial Sequence: Synthetic oligonucleotide”
ucuggucuaucccauguug

SEQ ID NO: 667 - RNA, 19 nt, Artificial Sequence
  source: /note=“Description of Artificial Sequence: Synthetic oligonucleotide”
aaauacgucccauguuguc

SEQ ID NO: 668 - RNA, 19 nt, Artificial Sequence
  source: /note=“Description of Artificial Sequence: Synthetic oligonucleotide”
aagcugacuacgcuguagc

SEQ ID NO: 669 - RNA, 19 nt, Artificial Sequence
  source: /note=“Description of Artificial Sequence: Synthetic oligonucleotide”
ucccauaaaugaaucucug

SEQ ID NO: 670 - RNA, 19 nt, Artificial Sequence
  source: /note=“Description of Artificial Sequence: Synthetic oligonucleotide”
ugacgaaauauacucugcc

SEQ ID NO: 671 - RNA, 19 nt, Artificial Sequence
  source: /note=“Description of Artificial Sequence: Synthetic oligonucleotide”
uugacgaaauauacucugc

SEQ ID NO: 672 - RNA, 19 nt, Artificial Sequence
  source: /note=“Description of Artificial Sequence: Synthetic oligonucleotide”
aauugaccuugacuucugg

SEQ ID NO: 673 - RNA, 19 nt, Artificial Sequence
  source: /note=“Description of Artificial Sequence: Synthetic oligonucleotide”
ucuauauaggugguaguuc

SEQ ID NO: 674 - RNA, 19 nt, Artificial Sequence
  source: /note=“Description of Artificial Sequence: Synthetic oligonucleotide”
aaucuuuaugugcuguggc

SEQ ID NO: 675 - RNA, 19 nt, Artificial Sequence
  source: /note=“Description of Artificial Sequence: Synthetic oligonucleotide”
uugcuacaccuccauaauc

SEQ ID NO: 676 - RNA, 19 nt, Artificial Sequence
  source: /note=“Description of Artificial Sequence: Synthetic oligonucleotide”
aagcaguaguuauccuucc

SEQ ID NO: 677 - RNA, 19 nt, Artificial Sequence
  source: /note=“Description of Artificial Sequence: Synthetic oligonucleotide”
uggauugauguaagaagcg

SEQ ID NO: 678 - RNA, 19 nt, Artificial Sequence
  source: /note=“Description of Artificial Sequence: Synthetic oligonucleotide”
aacgaaaauauuucaggug

SEQ ID NO: 679 - RNA, 19 nt, Artificial Sequence
  source: /note=“Description of Artificial Sequence: Synthetic oligonucleotide”
agauacgcuuauagaaugg

SEQ ID NO: 680 - RNA, 19 nt, Artificial Sequence
  source: /note=“Description of Artificial Sequence: Synthetic oligonucleotide”
uuugucaacggagaaguac

SEQ ID NO: 681 - RNA, 19 nt, Artificial Sequence
  source: /note=“Description of Artificial Sequence: Synthetic oligonucleotide”
agguuucagaucaugacuc

SEQ ID NO: 682 - RNA, 19 nt, Artificial Sequence
  source: /note=“Description of Artificial Sequence: Synthetic oligonucleotide”
agaauauugccaccauuag

SEQ ID NO: 683 - RNA, 19 nt, Artificial Sequence
  source: /note=“Description of Artificial Sequence: Synthetic oligonucleotide”
ucaacaccucgagacaacc

SEQ ID NO: 684 - RNA, 19 nt, Artificial Sequence
  source: /note=“Description of Artificial Sequence: Synthetic oligonucleotide”
aaugacgguagauggcaug

SEQ ID NO: 685 - RNA, 19 nt, Artificial Sequence
  source: /note=“Description of Artificial Sequence: Synthetic oligonucleotide”
aguuacucuuguccaaauc

SEQ ID NO: 686 - RNA, 19 nt, Artificial Sequence
  source: /note=“Description of Artificial Sequence: Synthetic oligonucleotide”
ugcaaauaaguucuugccc

SEQ ID NO: 687 - RNA, 19 nt, Artificial Sequence
  source: /note=“Description of Artificial Sequence: Synthetic oligonucleotide”
uccaauuuuacaagaugug

SEQ ID NO: 688 - RNA, 19 nt, Artificial Sequence
  source: /note=“Description of Artificial Sequence: Synthetic oligonucleotide”
acuuacacucagauaacuc

SEQ ID NO: 689 - RNA, 19 nt, Artificial Sequence
  source: /note=“Description of Artificial Sequence: Synthetic oligonucleotide”
uuacuuacacucagauaac

SEQ ID NO: 690 - RNA, 19 nt, Artificial Sequence
  source: /note=“Description of Artificial Sequence: Synthetic oligonucleotide”
ugggagauaaaucuaaaug

SEQ ID NO: 691 - RNA, 19 nt, Artificial Sequence
  source: /note=“Description of Artificial Sequence: Synthetic oligonucleotide”
ugacuagucauuugcagac

SEQ ID NO: 692 - RNA, 19 nt, Artificial Sequence
  source: /note=“Description of Artificial Sequence: Synthetic oligonucleotide”
ucagacuaaaaugagaggg

SEQ ID NO: 693 - RNA, 19 nt, Artificial Sequence
  source: /note=“Description of Artificial Sequence: Synthetic oligonucleotide”
aacaauagucaagucagac

SEQ ID NO: 694 - RNA, 19 nt, Artificial Sequence
  source: /note=“Description of Artificial Sequence: Synthetic oligonucleotide”
aggaaugauguauggauuc

(19) United States
(12) Patent Application Publication
(10) Pub. No.: US 2014/0328811 A1
(43) Pub. Date: Nov. 6, 2014

(54) METHOD FOR IMPROVING THE SUCCESS RATE OF HEMATOPOIETIC STEM CELL TRANSPLANTS

(71) Applicants: Jamie Wong, Cambridge, MA (US); Brian Bettencourt, Cambridge, MA (US)

(72) Inventors: Jamie Wong, Cambridge, MA (US); Brian Bettencourt, Cambridge, MA (US)

(73) Assignee: ALNYLAM PHARMACEUTICALS, INC., Cambridge, MA (US)

(21) Appl. No.: 14/236,493

(22) PCT Filed: Aug. 1, 2012
(86) PCT No.: PCT/US2012/049156

§ 371 Date: Jun. 10, 2014

Related U.S. Application Data
(60) Provisional application No. 61/513,820, filed on Aug. 1, 2011.

(51) Int. Cl. C12N 15/113; A61K 35/28

(52) U.S. Cl. CPC C12N 15/1136; A61K 35/28; C12N 2310/14; C12N 2320/00; C12N 2310/3515; C12N 2320/30
USPC 424/93.21; 536/24.5; 435/320.1; 435/377; 514/44 A; 424/93.7

(57) ABSTRACT

The technology described herein relates to double-stranded ribonucleic acid (dsRNA) compositions targeting the genes encoding negative regulators of MHC expansion (e.g. AhR, Itch, Prox1 and/or SH2B3), and methods of using such dsRNA compositions to inhibit expression of negative regulators of MHC expansion. The use of such compositions to provide, for example, enhanced quantitites and/or qualities of MHCs and/or hematopoietic progenitor cells for transplantation and/or to enhance engraftment of transplanted MHCs hematopoietic progenitor cells is described.

### CROSS-REFERENCE TO RELATED APPLICATIONS

This application claims benefit under 35 U.S.C. §119(e) of U.S. Provisional Application No. 61/513,820 filed Aug. 1, 2011, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Aug. 1, 2012, is named 515871PCT.txt and is 539,778 bytes in size.

### TECHNICAL FIELD

The technology described herein relates to methods of treating stem cells and/or progenitor cells and/or umbilical cord blood with siRNA in order to increase the availability of transplant materials and the success rate of their use.

### BACKGROUND

A number of conditions and diseases are responsive to treatment with hematopoietic cells. Such conditions include hematological malignancies, bone marrow failure, hemoglobinopathies, and inborn errors of metabolism (e.g. auto-immune diseases, leukemia, AML, ALL, CML, Hodgkins' disease, neutropenia, myelodysplastic syndrome, Fanconi's anemia, Blackfan Diamond anemia, severe aplastic anemia, severe combined immunodeficiency, Wiskott-Aldrich syndrome, osteopetrosis, Hurler syndrome, Hunter's syndrome, Lesch Nyhan syndrome, adrenoleukodystrophy, globoid cell leukodystrophy, X-linked lymphoproliferative syndrome sickle-cell anemia, HIV, Ewing's sarcoma, diabetes, system lupus erythromatosis, rheumatoid arthritis, Gaucher's disease, thalassemia, chemotherapy rescue of the immune system). Transplantation of hematopoietic stem cells (HSCs) is a common treatment in such cases. Clinically, human HSCs are obtained from three different sources: bone marrow, adult peripheral blood after mobilization, and cord blood obtained from umbilical cords after delivery.

Although there are more than 5 million unrelated bone marrow volunteer donors registered worldwide, finding a fully human leukocyte antigen (HLA)-matched unrelated donor remains a problem for many patients because of HLA polymorphism. Compared with bone marrow and adult peripheral blood, cord blood has several potential advantages, in particular the wide and rapid availability of cells and less stringent requirements for HLA identity between donor and recipient because of the lower risk of acute and chronic graft-versus-host disease (GVHD) (Rocha, V, et. al., Comparison of outcomes of unrelated bone marrow and umbilical cord blood transplants in children with acute leukemia. Blood. 97:2962-71.2001). Potential advantages of transplantation using cord blood HSCs rather than HSCs from bone marrow or adult peripheral blood include: (1) a large potential donor pool; (2) rapid availability, since the cord blood has been prescreened and tested; (3) greater racial diversity can be attained in the banks by focusing collection efforts on hospitals where children of under represented ethnic backgrounds are born; (4) reduced risk or discomfort for the donor; (5) rare contamination by viruses; and (6) lower risk of graft-versus-host disease (wherein the donor's cells attack the patient's organs and tissues), even for recipients with a less-than-perfect tissue match. Additionally, HSCs obtained from cord blood, as compared to adult HSCs are able to form colonies in culture, have a higher cell-cycle rate, display autocrine production of growth factors, and have longer telomeres, all of which favor transplantation success. Finally, as opposed to bone marrow, collection of cord blood poses no risk to the donor. Thus, cord blood-derived HSCs have been increasingly used for bone marrow transplantation in recent years.

However, a single umbilical cord typically yields enough graft material to suffice for only one pediatric bone marrow transplant procedure. Furthermore, the concentration of viable cells provided to the recipient is a key determinant of transplantation success (Gluckman, E. et al., NEJM 1997 337:373-381). In some instances, bone marrow transplants are used instead of cord blood transplants specifically because of concerns about graft cell dose (Barker, J. N. et al. BB&MT 2002 8:257-260) and only 12% of the current UCB inventory contains a graft cell dose sufficient for the treatment of a 60 kg patient. If the total nucleated cell count is below 2.5×10⁷ cells/kg² or the CD34+ count is below 1.7×10⁵ cells/kg², then poor engraftment, high nonrelapse mortality, and poor survival is much more likely to be observed. The transplantation benchmarks of neutrophil and platelet recovery are believed to be tied to graft cell dose.

HSCs and progenitor cells found in UCB can be expanded to increase the amount of graft material available for therapeutic use (Liao, Y. et al. Experimental Hematology 2011 39:393-412). Successful expansion of in vitro expansion of the stem cells can increase the possible uses for a single cord blood collection. Stem cell expansion can allow greater accessibility to this form of treatment, increase its rate of success in both pediatric and adult patients by facilitating a higher graft cell dose and allow for the development of cord blood stem cells for gene therapy.

### SUMMARY

Described herein are methods of ex vivo expansion of multipotent hematopoietic cells (MHC) collected from UCB. As used herein, the term “multipotent hematopoietic cells” refers to both hematopoietic stem cells and hematopoietic progenitor cells. A population of MHCs can be comprised of HSCs or hematopoietic progenitor cells or a mixture of both cell types. Application of these methods can enhance graft capacity and thus the success rate and recovery rate associated with UCB HSC transplantation. In one aspect of the methods described herein, hematopoietic cells from UCB are expanded for transplantation by introducing into the cells iRNA constructs that reduce or knock-down expression of gene targets that, for example, negatively regulate expansion cytokines. Following expansion, a therapeutically effective amount of MHCs can be administered to the patient. Successfully transplanted MHCs will engraft and then proliferate and differentiate, replacing the patient's damaged or absent hematopoietic cell populations. The methods described herein lead to the preparation of both a greater number of MHCs available for engraftment as well as MHCs with an increased ability to engraft, proliferate, and/or differentiate.

Expansion of MHCs treated according to the methods described herein can be measured by cell counts of the total population. In some embodiments, the treated population of MHC can be divided and expansion of the subpopulations can be measured. Methods for assessment of engraftment are discussed below.

Described herein are compositions and methods that reduce expression of genes which encode negative regulators of MHC expansion, e.g. in a cell or in a mammal. A negative regulator of MHC expansion can be a factor that inhibits the progress of the cell cycle, a factor that inhibits MHC division, a factor that inhibits MHC growth, or a factor that inhibits MHC proliferation. In certain embodiments a negative regulator of MHC expansion can be a factor that promotes differentiation. Also described are compositions and methods for promoting MHC expansion ex vivo, thus permitting an increase in the amount of transplant material available to patients as well as increasing the efficacy and/or potential of the same transplant material.

Also described herein are methods for increasing expansion or engraftment of transplanted MHCs involving administration of an iRNA that targets a negative regulator of MHC expansion with the transplanted cells. Also contemplated is administration of such iRNA compositions to enhance MHC expansion in vivo.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein inhibits the expression of a negative regulator of MHC expansion in a cell or mammal. In further embodiments, the iRNA inhibits, for example, the expression of one or more of Itch, SH2B3/Lnk, PROX1, or Ahr in a cell or a mammal.

The iRNAs included in the compositions featured herein encompass a dsRNA having an RNA strand (the antisense strand) having a region that is 30 nucleotides or less, generally 19-24 nucleotides in length, that is substantially complementary to at least part of an mRNA transcript of a gene encoding a negative regulator of MHC expansion. In further embodiments, the mRNA transcript encodes Itch, SH2B3/Lnk, PROX1, or Ahr. In certain embodiments, the dsRNA comprises a region of at least 15 contiguous nucleotides.

In one embodiment, an iRNA for inhibiting expression of a gene encoding a negative regulator of MHC expansion includes at least two sequences that are complementary to each other. The iRNA includes a sense strand having a first sequence and an antisense strand having a second sequence. The antisense strand includes a nucleotide sequence that is substantially complementary to at least part of an mRNA encoding a negative regulator of MHC expansion, and the region of complementarity is 30 nucleotides or less, and at least 15 nucleotides in length. Generally, the iRNA is 19 to 24, e.g., 19 to 21 nucleotides in length. In some embodiments the iRNA is from about 15 to about 25 nucleotides in length, and in other embodiments the iRNA is from about 25 to about 30 nucleotides in length. The iRNA, upon contacting with a cell expressing a negative regulator of MHC expansion, inhibits the expression of a negative regulator of MHC expansion by at least 10%, at least 20%, at least 25%, at least 30%, at least 35% or at least 40% or more, such as when assayed by a method as described herein. In one embodiment, the iRNA is formulated in a stable nucleic acid lipid particle (SNALP).

In one embodiment, an iRNA featured herein includes a first sequence of a dsRNA that is selected from the group consisting of the sense sequences of Tables 2-7, and a second sequence that is selected from the group consisting of the corresponding antisense sequences of Tables 2-7. The iRNA molecules featured herein can include naturally occurring nucleotides or can include at least one modified nucleotide, including, but not limited to a 2′-O-methyl modified nucleotide, a nucleotide having a 5′-phosphorothioate group, and a terminal nucleotide linked to a cholesteryl derivative. Alternatively, the modified nucleotide can be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. Generally, such a modified sequence will be based on a first sequence of iRNA selected from the group consisting of the sense sequences of Tables 2-7, and a second sequence selected from the group consisting of the corresponding antisense sequences of Tables 2-7.

In one embodiment, administration of the dsRNA increases the number of MHCs ex vivo by at least 10%, e.g., by at least 25%, by at least 50%, by at least 100%, at least 200%, at least 300%, at least 400%, at least 500%, or at least 600%, or more as compared to a population not treated with the dsRNA.

In one embodiment, administration of the dsRNA increases the number of CD34+MHCs ex vivo by at least 10%, e.g., by at least 25%, by at least 50%, by at least 100%, at least 200%, at least 300%, at least 400%, at least 500%, or at least 600%, or more as compared to a population not treated with the dsRNA.

In one embodiment, administration of the dsRNA decreases the necessary cell dose for a successful graft by at least 10%, e.g., by at least 25%, at least 50%, at least 75%, at least 90%, at least 95%, at least 99% or more compared to a population not treated with the dsRNA.

In one embodiment, administration of the dsRNA increases the number of MHCs of a given unit dose of cells which survive after administration to the transplant patient by at least 10%, e.g., by at least 25%, by at least 50%, by at least 100%, at least 200%, at least 300%, at least 400%, at least 500%, or at least 600%, or more as compared to a population not treated with the dsRNA at, for example, 4 weeks, 8 weeks, 12 weeks or 16 weeks after transplantation.

In one embodiment, administration of the dsRNA decreases the time required for platelet and/or neutrophil recovery in the transplant patient by at least 5%, e.g., by at least 10%, at least 15%, at least 20%, at least 25%, at least 30%, at least 40%, at least 50%, at least 60%, or more as compared to a patient receiving MHCs not treated with the dsRNA.

In one embodiment, administration of the dsRNA decreases the time required for lymphocyte recovery in the transplant patient by at least 5%, e.g., by at least 10%, at least 15%, at least 20%, at least 25%, at least 30%, at least 40%, at least 50%, at least 60%, or more as compared to a patient receiving MHCs not treated with the dsRNA.

In one embodiment, administration of the dsRNA increases the rate of patient survival by at least 5%, e.g., by at least 10%, at least 15%, at least 20%, at least 25%, at least 30%, at least 40%, at least 50%, at least 60%, or more as compared to patients receiving MHCs not treated with the dsRNA, at 6 months, 1 year, or 2 years after transplantation.

In one embodiment, an iRNA as described herein targets a wildtype negative regulator of MHC expansion RNA transcript, and in another embodiment, the iRNA targets a mutant transcript (e.g., a negative regulator RNA carrying an allelic variant). For example, an iRNA can target a polymorphic variant, such as a single nucleotide polymorphism (SNP), of a negative regulator of MHC expansion. In another embodiment, the iRNA targets both a wildtype and a mutant negative regulator of MHC expansion transcript. In yet another embodiment, the iRNA targets a transcript variant of a negative regulator of MHC expansion.

In one embodiment, an iRNA as described herein targets a non-coding region of a negative regulator of MHC expansion RNA transcript, such as the 5′ or 3′ untranslated region.

In one aspect, embodiments of the technology described herein provide a cell containing at least one of the iRNAs featured herein. The cell is generally a mammalian cell, such as a human cell.

In another aspect, described herein is a composition for inhibiting the expression of a negative regulator of MHC expansion gene in a cell, generally human MHC obtained from UCB. The composition typically includes one or more of the iRNAs described herein and an acceptable carrier or delivery vehicle.

In another embodiment, a composition containing an iRNA described herein, e.g., a dsRNA targeting a negative regulator of MHC expansion, is administered to a MHC cell with a non-iRNA expansion agent, such as a cytokine or a stromal cell.

In another embodiment, a composition containing an iRNA described herein, e.g., a dsRNA targeting a negative regulator of MHC expansion, is administered to a MHC cell with one or more additional iRNAs targeting a negative regulator of MHC expansion.

In another aspect, embodiments of the technology described herein provide a pharmaceutical composition for inhibiting the expression of a negative regulator of MHC expansion in an organism, preferably a human subject. The composition typically includes one or more of the iRNAs described herein and a pharmaceutically acceptable carrier or delivery vehicle. In one embodiment, the composition is used for treating a malignant or non-malignant hematopoietic disorder.

In another aspect, described herein is a method for inhibiting the expression of a gene encoding a negative regulator of MHC expansion in a cell by performing the following steps:
(a) introducing into the cell a double-stranded ribonucleic acid (dsRNA), wherein the dsRNA includes at least two sequences that are complementary to each other. The dsRNA has a sense strand having a first sequence and an antisense strand having a second sequence; the antisense strand has a region of complementarity that is substantially complementary to at least a part of an mRNA encoding a negative regulator of MHC expansion, and where the region of complementarity is 30 nucleotides or less, i.e., 15-30 nucleotides in length, and generally 19-24 nucleotides in length, and where the dsRNA, upon contact with a cell expressing the negative regulator of MHC expansion, inhibits expression of the gene encoding a negative regulator of MHC expansion by at least 10%, preferably at least 20%, at least 30%, at least 40%, at least 50%, at least 60%, at least 70%, at least 80%, at least 90%, at least 95%, at least 99% or more; and
(b) maintaining the cell produced in step (a) for a time and under conditions sufficient to obtain degradation of the mRNA transcript of the gene encoding a negative regulator of MHC expansion, thereby inhibiting expression of a gene encoding a negative regulator of MHC expansion in the cell.

In some embodiments, the method described above inhibits the expression of a gene encoding a negative regulator of MHC expansion in a cell. In some embodiments, the method described above reduces the expression of a gene encoding a negative regulator of MHC expansion in a cell.

In one embodiment, the method is for inhibiting gene expression in a human MHC cell or a human hematopoietic progenitor cell. In another embodiment, the method is for inhibiting gene expression in a human MHC cell or a human hematopoietic progenitor cell obtained from UCB.

In one aspect, the technology described herein provides a vector for inhibiting the expression of a gene encoding a negative regulator of MHC expansion in a cell. In one embodiment, the vector includes at least one regulatory sequence operably linked to a nucleotide sequence that encodes at least one strand of an iRNA as described herein.

In another aspect, the technology described herein provides a cell containing a vector for inhibiting the expression of a gene encoding a negative regulator of MHC expansion in a cell in a cell. The vector includes a regulatory sequence operably linked to a nucleotide sequence that encodes at least one strand of one of the iRNAs as described herein.

The details of various embodiments of the technology described herein are set forth in the description below. Other features, objects, and advantages of the technology described herein will be apparent from the description and the drawings, and from the claims.

### BRIEF DESCRIPTION OF THE FIGURES

FIGS. 1A-1C depict graphs of in vitro tests of SH2B3 silencing by iRNA duplexes. iRNAs were administered to Hep3b cells at 10 nM (darker bars) or 0.1 nM (lighter bars). Relative expression of SH2B3 24 hours after treatment is shown on the y-axis and the identity of the duplex is shown on the x-axis.

FIGS. 2A-2C depict graphs of in vitro tests of SH2B3 silencing by iRNA duplexes. iRNAs were administered to RKO cells at 10 nM (darker bars) or 0.1 nM (lighter bars). Relative expression of SH2B3 24 hours after treatment is shown on the y-axis and the identity of the duplex is shown on the x-axis.

FIGS. 3A-3B depict graphs of dose response in vitro tests of SH2B3 silencing by iRNA duplexes. FIG. 3A shows experiments performed in RKO cells. Expression was measured 24 hours after treatment. IC50 and IC80 are listed in nM. FIG. 3A shows experiments performed in Hep3b cells. Expression was measured 24 hours after treatment. IC50 is listed in nM.

### DETAILED DESCRIPTION

Described herein are iRNAs and methods of using them for inhibiting the expression of a gene encoding a negative regulator of MHC expansion in a cell or a mammal where the iRNA targets a gene encoding a negative regulator of MHC expansion in a cell. The subject iRNAs enhance the expansion of MHCs for transplantation, thereby increasing opportunities for successful engraftment of transplanted cells. Therapies based on the expansion of MHCs, using iRNAs as described herein, include, for example, the administration of expanded MHCs to a subject in need thereof. Also contemplated is, for example, the co-administration of MHCs and iRNAs as described herein, to enhance engraftment of transplanted MHCs. Autologous and non-autologous MHC transplants are contemplated.

iRNA directs the sequence-specific degradation of mRNA through a process known as RNA interference (RNAi). The iRNAs of the compositions featured herein comprise an RNA strand (the antisense strand) having a region which is 30 nucleotides or less in length, i.e., 15-30 nucleotides in length, generally 19-24 nucleotides in length, which region is substantially complementary to at least part of an mRNA transcript of a gene encoding a negative regulator of MHC expansion in a cell. The use of these iRNAs permits the targeted degradation of mRNAs of genes that limit expansion of MHCs in vitro and in vivo. iRNAs can specifically and efficiently mediate RNAi, resulting in significant inhibition of expression of a gene encoding a negative regulator of MHC expansion in a cell. Using cell-based assays, the present inventors have demonstrated that iRNAs targeting a gene encoding a negative regulator of MHC expansion can specifically and efficiently mediate RNAi, resulting in significant inhibition of target gene expression and increases in MHC expansion in vitro and in vivo. Thus, methods and compositions including these iRNAs are useful for increasing the amount of graft material and graft success in cases where a patient is in need of a MHC transplant (e.g. leukemia, Fanconi's anemia, etc). The following detailed description discloses how to make and use compositions containing iRNAs to inhibit the expression of a gene encoding a negative regulator of MHC expansion in a cell.

Embodiments of compositions featured herein include an iRNA having an antisense strand comprising a region which is 30 nucleotides or less in length, generally 19-24 nucleotides in length, which region is substantially complementary to at least part of an RNA transcript of a gene encoding a negative regulator of MHC expansion, together with an acceptable carrier.

Embodiments of the pharmaceutical compositions described herein comprise an iRNA having an antisense strand having a region of complementarity which is 30 nucleotides or less in length, generally 19-24 nucleotides in length, and is substantially complementary to at least part of an RNA transcript of a gene encoding a negative regulator of MHC expansion.

Accordingly, in some aspects, pharmaceutical compositions containing an iRNA targeting a gene encoding a negative regulator of MHC expansion and an acceptable carrier, methods of using the compositions to inhibit expression of a gene encoding a negative regulator of MHC expansion, and methods of administering the treated with the composition to a patient in need of a MHC transplant are encompassed.

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured herein by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods described herein.

As used herein, the term “multipotent hematopoietic cells” or “MHC” refers to both hematopoietic stem cells and hematopoietic progenitor cells. A population of MHCs can be comprised of HSCs or hematopoietic progenitor cells or a mixture of both cell types.

The term “hematopoietic stem cells” or “HSC” as used herein refers to pluripotent stem cells or multipotential stem cells or lymphoid or myeloid (derived from bone marrow) stem cells that, upon exposure to an appropriate cytokine or plurality of cytokines, can either differentiate into a progenitor cell of a lymphoid, erythroid or myeloid cell lineage or proliferate as a stem cell population without further differentiation having been initiated. HSCs can be isolated from bone marrow, peripheral blood, umbilical cord blood, or embryonic stem cells. HSCs can form cells such as erythrocytes (red blood cells), platelets, granulocytes (such as neutrophils, basophils, and eosinophils), macrophages, B-lymphocytes, T-lymphocytes, and Natural killer cells. HSC are capable of self-renewal or remaining a stem cell after cell division. HSCs are also capable of differentiation or starting a path to becoming a mature hematopoietic cell. HSCs can also be regulated in their mobility or migration or can be regulated by apoptosis or programmed cell death.

The terms “progenitor” and “progenitor cell” as used herein refer to primitive hematopoietic cells that have differentiated to a developmental stage that, when the cells are further exposed to an appropriate cytokine or a group of cytokines, they will differentiate further along the hematopoietic cell lineage. In contrast to HSCs, progenitors are only capable of limited self-renewal. “Progenitors” and “progenitor cells” as used herein also include “precursor” cells that are derived from differentiation of progenitor cells and are the immediate precursors of mature differentiated hematopoietic cells. The terms “progenitor”, and “progenitor cell” as used herein include, but are not limited to, granulocyte-macrophage colony-forming cell (GM-CFC), megakaryocyte colony-forming cell (Mk-CFC), burst-forming unit erythroid (BFU-E), B cell colony-forming cell (B-CFC) and T cell colony-forming cell (T-CFC). “Precursor cells” include, but are not limited to, colony-forming unit-erythroid (CFU-E), granulocyte colony forming cell (G-CFC), colony-forming cell-basophil (CFC-Bas), colonyforming cell-eosinophil (CFC-Eo) and macrophage colonyforming cell (M-CFC) cells.

The term “cytokine” as used herein refers to any cytokine, growth factor, or combination of cytokines and growth factors that can induce the differentiation of a lympho-hematopoietic stem cell to a lympho-hematopoietic progenitor or precursor cell and/or induce the proliferation thereof. Suitable cytokines for use in the embodiments described herein include, but are not limited to, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia 5 inhibitory factor, insulin-like growth factor, and insulin. The term “cytokine” as used herein further refers to any natural cytokine or growth factor as isolated from an animal or human tissue, and any fragment or derivative thereof that retains biological activity of the original parent cytokine. The cytokine or growth factor can further be a recombinant cytokine or a growth factor such as, for example, recombinant insulin. The term “cytokine” as used herein further includes species specific cytokines that while belonging to a structurally and functionally related group of cytokines, will have biological activity restricted to one animal species or group of taxonomically related species, or have reduced biological effect in other species.

As used herein, the terms “expanding” and “expansion” refer to substantially differentiation-less cell growth, i.e., increase of a cell population without differentiation accompanying such increase.

As used herein, the term “negative regulator of MHC expansion” refers to any peptide, gene, or transcript which when expression thereof is reduced a detectable amount, results in an increase in MHC population growth as compared to a population of MHCs maintained under the same conditions, except that the peptide, gene, or transcript's expression is not reduced. The increase in MHC population growth can be at least 10%, at least 20%, at least 50%, at least 70%, at least 90%, at least 100%, at least 200%, at least 500% or more. In exemplary embodiments a negative regulator of MHC expansion can include Itch, PROX-1, SH2B3, and/or AhR.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein inhibits expression of a gene encoding a negative regulator of MHC expansion.

As used herein, “target sequence” refers to a contiguous portion of the nucleotide sequence of an messenger RNA (mRNA) molecule formed during the transcription of a gene, including mRNA that is a product of RNA processing of a primary transcription product. The target portion of the sequence will be at least long enough to serve as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a negative regulator of MHC expansion). For example, a polynucleotide is complementary to at least a part of a mRNA if the sequence is substantially complementary to a non-interrupted portion of the mRNA.

The term “double-stranded RNA” or “dsRNA,” as used herein, refers to an iRNA that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. dsRNAs generated in the cell by processing with Dicer and similar enzymes are generally in the range of 19-22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA as described above.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure or in the ribose-phosphate backbone structure, e.g., as described herein below. However, the molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an RNA molecule can comprise at least two modified ribonucleosides, at least 3, at least 4, at least 5, at least 6, at least 7, at least 8, at least 9, at least 10, at least 15, at least 20 or more, up to the entire length of the dsRNA molecule. The modifications need not be the same for each of such a plurality of modified ribonucleosides in an RNA molecule. In one embodiment, modified RNAs contemplated for use in methods and compositions described herein are peptide nucleic acids (PNAs) that have the ability to form the required duplex structure and that permit or mediate the specific degradation of a target RNA via a RISC pathway.

In one aspect, a modified ribonucleoside includes a deoxyribonucleoside. In such an instance, an iRNA agent can comprise one or more deoxynucleosides, including, for example, a deoxynucleoside overhang(s), or one or more deoxynucleosides within the double stranded portion of a dsRNA. However, it is self evident that under no circumstances is a double stranded DNA molecule encompassed by the term “iRNA.”

In one aspect, an RNA interference agent includes a single stranded RNA that interacts with a target RNA sequence to direct the cleavage of the target RNA. Without wishing to be bound by theory, long double stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect the technology described herein relates to a single stranded RNA that promotes the formation of a RISC complex to effect silencing of the target gene.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′ end, 3′ end or both ends of either an antisense or sense strand of a dsRNA.

In one embodiment, the antisense strand of a dsRNA has a 1-10 nucleotide overhang at the 3′ end and/or the 5′ end. In one embodiment, the sense strand of a dsRNA has a 1-10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

The term “antisense strand” or “guide strand” refers to the strand of an iRNA, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand,” or “passenger strand” as used herein, refers to the strand of an iRNA that includes a region that is substantially complementary to a region of the antisense strand as that term is defined herein.

As used herein, in one embodiment, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety. Examples of “SNALP” formulations are described elsewhere herein.

“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA can also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art such as electroporation and lipofection. Further approaches are described herein below or are known in the art.

As used herein, the phrase “inhibit the expression of,” refers to at an least partial reduction of gene expression of a gene encoding a negative regulator of MHC expansion in a cell treated with an iRNA composition as described herein compared to the expression of the gene encoding a negative regulator of MHC expansion in an untreated cell.

The terms “silence,” “inhibit the expression of,” “down-regulate the expression of,” “suppress the expression of,” and the like, in so far as they refer to a gene encoding a negative regulator of MHC expansion, herein refer to the at least partial suppression of the expression of a gene encoding a negative regulator of MHC expansion, as manifested by a reduction of the amount of mRNA encoding a negative regulator of MHC expansion which can be isolated from or detected in a first cell or group of cells in which that gene is transcribed and which has or have been treated such that the expression of a gene encoding a negative regulator of MHC expansion is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition can be given in terms of a reduction of a parameter that is functionally linked to gene expression, e.g., the amount of protein encoded by a gene, or the number of cells displaying a certain phenotype. In principle, gene silencing can be determined in any cell expressing, either constitutively or by genomic engineering, and by any appropriate assay. However, when a reference is needed in order to determine whether a given iRNA inhibits the expression of the gene encoding a negative regulator of MHC expansion by a certain degree and therefore is encompassed by the technology described herein, the assays provided in the Examples below shall serve as such reference.

For example, in certain instances, expression of a gene encoding a negative regulator of MHC expansion is suppressed by at least about 10%, 15%, 20%, 25%, 30%, 35%, 40%, 45%, or 50% by administration of an iRNA featured herein. In some embodiments, a gene encoding a negative regulator of MHC expansion in a cell is suppressed by at least about 60%, 70%, or 80% by administration of an iRNA featured herein. In some embodiments, a gene encoding a negative regulator of MHC expansion in a cell is suppressed by at least about 85%, 90%, 95%, 98%, 99% or more by administration of an iRNA as described herein.

As used herein in the context of expression, the terms “treat,” “treatment,” and the like, refer to actions that leads to an increase in MHC expansion or an increase in traits which enhance the outcome of a MHC transplant (e.g. ability to engraft, ability to replace neutrophils, etc. as described herein). In the context of the technology described herein insofar as it relates to any of the other conditions recited herein below (other than phenotypes controlled by negative regulators of MHC expansion), the terms “treat,” “treatment,” and the like mean to relieve or alleviate at least one symptom associated with such condition, or to slow or reverse the progression or anticipated progression of such condition, such as slowing the progression of a hematopoietic disorder, such as leukemia.

As used herein, the phrases “therapeutically effective amount” and “prophylactically effective amount” refer to an amount that provides a therapeutic benefit in the treatment, prevention, or management of pathological processes resulting from a hematopoietic cell deficit or abnormality. The specific amount that is therapeutically effective can be readily determined by an ordinary medical practitioner, and can vary depending on factors known in the art, such as, for example, the type of pathological processes, the patient's history and age, the stage of pathological processes, and the administration of other agents that inhibit pathological processes.

As used herein, a “pharmaceutical composition” comprises a pharmacologically effective amount of an iRNA and a pharmaceutically acceptable carrier. As used herein, “pharmacologically effective amount,” “therapeutically effective amount” or simply “effective amount” refers to that amount of an iRNA effective to produce the intended pharmacological, therapeutic or preventive result. For example, if a given clinical treatment is considered effective when there is at least a 10% reduction in a measurable parameter associated with a disease or disorder, a therapeutically effective amount of a drug for the treatment of that disease or disorder is the amount necessary to effect at least a 10% reduction in that parameter. For example, a therapeutically effective amount of an iRNA targeting a gene encoding a negative regulator of MHC expansion can reduce protein levels of a negative regulator of MHC expansion by at least 10%.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents can include starch and gelatin, while the lubricating agent, if present, will generally be magnesium stearate, stearic acid or talc. If desired, the tablets can be coated with a material such as glyceryl monostearate or glyceryl distearate, to delay absorption in the gastrointestinal tract. Agents included in drug formulations are described further herein below.

As used herein, a “subject” is a mammal, e.g. a dog, horse, cat, or a non-human primate. In a preferred embodiment, a subject is a human.

As used herein, the term “LNPXX”, wherein the “XX” are numerals, is also referred to as “AFXX” herein. For example, LNP09 is also referred to AF09 and LNP12 is also known as or referred to as AF12.

As used herein, the term “comprising” or “comprises” is used in reference to compositions, methods, and respective component(s) thereof, that are essential to the technology described herein, yet open to the inclusion of unspecified elements, whether essential or not.

As used herein, the term “consisting essentially of” refers to those elements required for a given embodiment. The term permits the presence of elements that do not materially affect the basic and novel or functional characteristic(s) of that embodiment of the technology described herein.

While hematopoietic stem cells (HSC) are sometimes defined as primitive cells capable of differentiating to generate all cells of the hematopoietic lineage, HSC, as the term is used herein, refers more generally to immature blood cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and/or monocytes (e.g., monocytes, macrophages). During development, the site of hematopoiesis translocates from the fetal liver to the bone marrow, which then remains the site of hematopoiesis throughout adulthood. It is known in the art that HSCs can or can not include CD34+ cells. CD34+ cells are immature cells that express the CD34 cell surface marker. CD34+ cells are believed to include a subpopulation of cells with the stem cell properties defined above. HSCs include pluripotent stem cells, multipotent stem cells (e.g., a lymphoid stem cell), and/or stem cells committed to specific hematopoietic lineages. The stem cells committed to specific hematopoietic lineages can be of T cell lineage, B cell lineage, dendritic cell lineage, Langerhans cell lineage and/or lymphoid tissue-specific macrophage cell lineage. In addition, HSCs also refer to long term HSC (LT-HSC) and short term HSC (ST-HSC). A long term stem cell typically includes the long term (more than three months) contribution to multilineage engraftment after transplantation. A short term stem cell is typically anything that lasts shorter than three months, and/or that is not multilineage. LT-HSC and ST-HSC are distinguished, for example, based on their cell surface marker expression. LT-HSC are CD34−, SCA-1+, Thyl.1+/lo, C-kit+, Un−, CD135−, Slamfl/CD150+, whereas ST-HSC are CD34+, SCA-1+, Thyl.1+/lo, C-kit+, lin−, CD135−, Slamfl/CD150+, Mac-1 (CD11b)lo (Handbook of Stem Cells. Lanza, R. P. et al. (Eds.) Elsevier Academic Press Bulington, Mass. (2004)). In addition, ST-HSC are less quiescent (i.e., more active) and more proliferative than LT-HSC. LT-HSC have unlimited self renewal (i.e., they survive throughout adulthood), whereas ST-HSC have limited self renewal (i.e., they survive for only a limited period of time). Any of these HSCs can be used advantageously in any of the methods described herein.

HSC are optionally obtained from blood products. A blood product includes a product obtained from the body or an organ of the body containing cells of hematopoietic origin. Such sources include unfractionated bone marrow, umbilical cord, peripheral blood, liver, thymus, lymph and spleen. All of the aforementioned crude or unfractionated blood products can be enriched for cells having hematopoietic stem cell characteristics in a number of ways. For example, the more mature, differentiated cells are selected against, via cell surface molecules they express. Optionally, the blood product is fractionated by selecting for CD34+ cells. CD34+ cells include a subpopulation of cells capable of self-renewal and pluripotentiality. Such selection is accomplished using, for example, commercially available magnetic anti-CD34 beads (Dynal, Lake Success, N.Y.). Unfractionated blood products are optionally obtained directly from a donor or retrieved from cryopreservative storage.

Sources for HSC expansion also include aorta-gonad-mesonephros (AGM) derived cells, embryonic stem cells (ESC) and induced pluripotent stem cells (iPSC). ESC are well-known in the art, and can be obtained from commercial or academic sources (Thomson et al., 282 Sci. 1145-47 (1998)). iPSC are a type of pluripotent stem cell artificially derived from a non-pluripotent cell, typically an adult somatic cell, by inducing a “forced” expression of certain genes (Baker, Nature Rep. Stem Cells (Dec. 6, 2007); Vogel & Holden, 23 Sci. 1224-25 (2007)). ESC, AGM, and iPSC can be derived from animal or human sources. The AGM stem cell is a cell that is born inside the aorta, and colonizes the fetal liver.

Hematopoietic progenitor cells, as the term is used herein, are capable of differentiation into one or more mature cell types of the hematopoietic lineage, but are not capable of long-term self-renewal. Thus, hematopoietic progenitor cells can restore and sustain hematopoiesis for three to four months (Marshak, D. R., et al. (2001). Stem cell biology, Cold Spring Harbor, N.Y.: Cold Spring Harbor Laboratory Press) and are important for recovery in the period immediately following a hematopoietic progenitor cell transplant in an individual. Hematopoietic progenitor cells useful for transplantation can be obtained from a variety of sources including, for example, bone marrow, peripheral blood, and umbilical cord blood.

Bone marrow can be obtained by puncturing bone with a needle and removing bone marrow cells with a syringe (herein called “bone marrow aspirate”). Hematopoietic progenitor cells can be isolated from the bone marrow aspirate prior to transplantation by using surface markers specific for hematopoietic progenitor cells, or alternatively whole bone marrow can be transplanted into an individual to be treated with the methods described herein.

Hematopoietic progenitor cells can also be obtained from peripheral blood of a progenitor cell donor. Prior to harvest of the cells from peripheral blood, the donor can be treated with a cytokine, such as granulocyte-colony stimulating factor, to promote cell migration from the bone marrow to the blood compartment. Cells can be collected via an intravenous tube and filtered to isolate white blood cells for transplantation. The white blood cell population obtained (i.e., a mixture of stem cells, progenitors and white blood cells of various degrees of maturity) can be transplanted as a heterogeneous mixture or hematopoietic progenitor cells can further be isolated using cell surface markers known to those of skill in the art.

Hematopoietic progenitor cells and/or a heterogeneous hematopoietic progenitor cell population can also be isolated from human umbilical cord and/or placental blood.

A MHC population can be obtained from a biopsy removed from a donor employing techniques known by persons skilled in the art, including the removal of stem cells from the bone marrow of a donor from large bone masses utilizing a large needle intended for bone marrow harvesting. Alternatively, MHCs can be collected by apheresis, a process in which a donor's peripheral blood is withdrawn through a sterile needle and passed through a device that removes white blood cells, and that returns the red blood cells to the donor. The peripheral stem cell yield can be increased with daily subcutaneous injections of granulocyte-colony stimulating factor. The MHCs are preferably obtained from human donors; however, non-human donors are also contemplated, including non-human primates, pigs, cows, horses, cats, and dogs. A purified population of MHCs can be obtained by utilizing various methods known by persons skilled in the art and/or as described in U.S. Pat. No. 5,677,136; and U.S. Patent Publication No. 2006/0040389, which are incorporated by reference in their entirety.

In one embodiment described herein, MHCs are isolated prior to transplantation. Hematopoietic cell samples (e.g., cord blood, peripheral blood, bone marrow) can first be purified to isolate and obtain artificially high concentrations of e.g., MHCs by detecting expression of specific cell surface proteins or receptors, cell surface protein markers, or other markers. Highly purified MHCs and HSCs are increasingly being used clinically, in a variety of applications, such as for autologous transplants into patients after high-dose chemotherapy. In this setting it is advantageous to isolate MHCs or HSCs with the maximum degree of purity so as to minimize contamination by immune effector cells (such as lymphocytes) or cancer cells. In murine studies, the highest enrichment of HSC activity yet reported describes combinations of markers, such as those used to isolate Thy-1.11oSca-1+lineage-Mac-1-CD4-c-kit+ cells, from which about one out of every five intravenously injected cells are able to home to bone marrow and engraft. Such results are described in, for example, Uchida et al.; Morrison et al., 1994 and Morrison et al. 1997).

Stem cells as well as committed progenitor cells destined to become neutrophils, erythrocytes, platelets, etc., can be distinguished from most other cells by the presence or absence of particular progenitor marker antigens, such as CD34, that are present on the surface of these cells and/or by morphological characteristics. The phenotype for a highly enriched human stem cell fraction is reported as CD34+, Thy-1+ and lin−, but it is to be understood that the present technology described herein is not limited to the expansion of this stem cell population.

The CD34+ enriched human stem cell fraction can be separated by a number of reported methods, including affinity columns or beads, magnetic beads or flow cytometry using antibodies directed to surface antigens such as CD34. Further, physical separation methods such as counterflow elutriation can be used to enrich hematopoietic progenitors. The CD34+ progenitors are heterogeneous, and can be divided into several subpopulations characterized by the presence or absence of coexpression of different lineage associated cell surface associated molecules. The most immature progenitor cells do not express any known lineage-associated markers, such as HLA-DR or CD38, but they can express CD90 (thy-1). Other surface antigens such as CD33, CD38, CD41, CD71, HLA-DR or c-kit can also be used to selectively isolate hematopoietic progenitors. The separated cells can be incubated in selected medium in a culture flask, sterile bag or in hollow fibers. Various hematopoietic growth factors can be utilized in order to selectively expand cells. Representative factors that have been utilized for ex vivo expansion of bone marrow include, c-kit ligand, IL-3, G-CSF, GM-CSF, IL-1, IL-6, IL-11, flt-3 ligand or combinations thereof. The proliferation of stem cells can be monitored by enumerating the number of stem cells and other cells, by standard techniques (e.g., hemacytometer, CFU, LTCIC) or by flow cytometry prior and subsequent to incubation.

Any method suitable for identifying surface proteins, whether known or to be discovered, could be employed to isolate hematopoietic stem cells from a homogeneous population such as e.g., cord blood. For example, MHCs for use with the methods described herein can be identified using fluorescence activated cell sorting analysis (FACS) which typically uses antibodies conjugated to fluorochromes to directly or indirectly assess the level of expression of a given surface protein on individual cells within a heterogenous (or homogenous) cell preparation of hematopoietic tissue.

MHCs can be physically separated from other cells within a cellular preparation of hematopoietic tissue using any previously developed or as yet undeveloped technique whereby cells are directly or indirectly distinguished according to their expression or lack of expression of particular surface proteins. Common methods used to physically separate specific cells from within a heterogenous population of cells within a hematopoietic cell preparation include but are not limited to flow-cytometry using a cytometer which can have varying degrees of complexity and or detection specifications, magnetic separation, using antibody or protein coated beads, affinity chromatography, or solid-support affinity separation where cells are retained on a substrate according to their expression or lack of expression of a specific protein or type of protein. Such separation techniques need not, but can, completely purify or nearly completely purify (e.g. 99.9% are perfectly separated) MHCs or populations enriched in MHCs.

Stem cells for expansion are harvested, for example, from a bone marrow sample of a subject or from a culture. Harvesting MHCs is defined as the dislodging or separation of cells. This is accomplished using any of a number of methods, such as enzymatic, non-enzymatic, centrifugal, electrical, or size-based methods, or preferably, by flushing the cells using culture media (e.g., media in which cells are incubated) or buffered solution. The cells are optionally collected, separated, and further expanded generating even larger populations of MHC and differentiated progeny.

In general, cells as described herein can be maintained and expanded in culture medium that is available to and well-known in the art. Such media include, but are not limited to, Dulbecco's Modified Eagle's, F-12K®, Eagle's Minimum Essential Medium® (DMEM), DMEM F12 Medium®, and Serum-Free®, RPMI-1640 Medium®, Iscove's Modified Dulbecco's Medium®. Many media for culture and expansion of hematopoietic cells are also available as low-glucose formulations, with or without sodium pyruvate.

Also contemplated herein is supplementation of cell culture medium with mammalian sera. Sera often contain cellular factors and components that are necessary for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements and bovine embryonic fluid. It is understood that sera can be heat-inactivated at 55-65° C. if deemed necessary to inactivate components of the complement cascade.

Additional supplements also can be used advantageously to supply the cells with the necessary trace elements for optimal growth and expansion. Such supplements include, for example, insulin, transferrin, sodium selenium and combinations thereof. These components can be included in a salt solution including, but not limited to, (HBSS), Earle's Salt®, Hanks' Balanced Salt Solution, antioxidant supplements, MCDB-201® Solution saline (PBS), ascorbic acid and ascorbic acid-2-phosphate, as well as additional amino acids. Many cell culture media already contain amino acids, however, some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Hormones also can be advantageously used in the cell cultures described herein and include, but are not limited to, D-aldosterone, diethylstilbestrol (DES), dexamethasone, .beta.-estradiol, hydrocortisone, insulin, prolactin, progesterone, somatostatin/human growth hormone (HGH), thyrotropin, thyroxine and L-thyronine.

Lipids and lipid carriers also can be used to supplement cell culture media, depending on the type of cell and the fate of the differentiated cell. Such lipids and carriers can include, but are not limited to, cholesterol, linoleic acid conjugated to albumin, cyclodextrin, linoleic acid and oleic acid conjugated to albumin, unconjugated linoleic acid, linoleic-oleic-arachidonic acid conjugated to albumin and oleic acid unconjugated and conjugated to albumin, among others.

Also contemplated for the methods described herein is the use of feeder cell layers. Feeder cells are used to support the growth of fastidious cultured cells, such as stem cells. Feeder cells are normal cells that have been γ-irradiated to suppress cell division yet permit active metabolism. In culture, the feeder layer serves as a basal inactivated layer for other cells and supplies cellular factors without further growth or division of their own (Lim, J. W. and Bodnar, A., 2002). Examples of typical feeder layer cells include human diploid lung cells, mouse embryonic fibroblasts and Swiss mouse embryonic fibroblasts, but can be any post-mitotic cell that is capable of supplying cellular components and factors that are advantageous in allowing optimal growth, viability and expansion of MHCs. In many cases, feeder cell layers are not necessary to keep MHCs in an undifferentiated, proliferative state, as leukemia inhibitory factor (LIF) has anti-differentiation properties. Therefore, supplementation with LIF can be used to maintain cells in an undifferentiated state.

Cells can be cultured in low-serum or serum-free culture medium. Serum-free medium used to culture cells is described in, for example, U.S. Pat. No. 7,015,037. Many cells have been grown in serum-free or low-serum medium. For example, the medium can be supplemented with one or more growth factors. Commonly used growth factors include, but are not limited to, bone morphogenic protein, basic fibroblast growth factor, platelet-derived growth factor and epidermal growth factor, Stem cell factor, thrombopoietin, Flt3Ligand and 1′-3. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein for teaching growing cells in serum-free medium.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. MHCs can also be cultured in low attachment flasks such as but not limited to Corning Low attachment plates.

In one embodiment, hematopoietic stem and/or progenitor cells are treated ex vivo prior to transplantation to an individual in need thereof by contacting a population of hematopoetic cells with compositions comprising at least one of the iRNAs described herein. Contacting can be performed in vitro by adding the composition comprising the iRNA directly to suitable cell culture medium for hematopoietic cells. The effective concentration of the iRNA can be determined by those of skill in the art, for example by performing serial dilutions and testing efficacy in the Zebrafish competitive transplant model, or other suitable system. Example concentration ranges for the treatment of the hematopoietic stem and/or progenitor cells include, but are not limited to, about 1 nanomolar to about 10 millimolar; about 1 mM to about 5 mM; about 1 nM to about 500 nM; about 500 nM to about 1,000 nM; about 1 nM to about 1,000 nM; about 1 uM to about 1,000 uM; 1 uM to about 500 uM; about 1 uM to about 100 uM; about 1 uM to about 10 uM. In one embodiment, the range is about 5 uM to about 500 uM.

Cells can be treated for various times. Suitable times can be determined by those of skill in the art. For example, cells can be treated for minutes, e.g. 5 minutes, 10 minutes, 15 minutes, 30 minutes etc, or treated for hours e.g., 1 hour, 2 hours, 3 hours, 4 hours, up to 24 hours or even days. In one embodiment the cells are treated for 2 hours prior to changing to medium without a composition comprising at least one of the iRNAs described herein.

Once established in culture, cells treated as described herein to enhance MHC or progenitor cell populations, and/or untreated cells can be used fresh or frozen and stored as frozen stocks, using, for example, DMEM with 40% FCS and 10% DMSO. Other methods for preparing frozen stocks for cultured cells also are available to those skilled in the art.

In addition, the cells obtained from harvesting MHCs, expanded via the methods described herein, can be cryopreserved using techniques known in the art for stem cell cryopreservation. Accordingly, using cryopreservation, the cells can be maintained such that once it is determined that a subject is in need of MHC transplantation, the MHCs can be thawed and transplanted into the subject.

More specifically, an embodiment of the technology described herein provides for the enhancement of MHCs collected from cord blood or an equivalent neonatal or fetal stem cell source, which can be cryopreserved, for the therapeutic use of such stem cells upon thawing. Such blood can be collected by several methods known in the art. For example, because umbilical cord blood is a rich source of MHCs (see Nakahata & Ogawa, 70 J. Clin. Invest. 1324-28 (1982); Prindull et al., 67 Acta. Paediatr. Scand. 413-16 (1978); Tchernia et al., 97(3) J. Lab. Clin. Med. 322-31 (1981)), an excellent source for neonatal blood is the umbilical cord and placenta. Prior to cryopreservation, the neonatal blood can be obtained by direct drainage from the cord and/or by needle aspiration from the delivered placenta at the root and at distended veins. See, e.g., U.S. Pat. No. 7,160,714; No. 5,114,672; No. 5,004,681; U.S. patent application Ser. No. 10/076,180, Pub. No. 20030032179. Indeed, umbilical cord blood stem cells have been used to reconstitute hematopoiesis in children with malignant and nonmalignant diseases after treatment with myeloablative doses of chemo-radiotherapy. Sirchia & Rebulla, 84 Haematologica 738-47 (1999). See also Laughlin 27 Bone Marrow Transplant. 1-6 (2001); U.S. Pat. No. 6,852,534. Additionally, it has been reported that stem and progenitor cells in cord blood appear to have a greater proliferative capacity in culture than those in adult bone marrow. Salahuddin et al., 58 Blood 931-38 (1981); Cappellini et al., 57 Brit. J. Haematol. 61-70 (1984).

Alternatively, fetal blood can be taken from the fetal circulation at the placental root with the use of a needle guided by ultrasound (Daffos et al., 153 Am. J. Obstet. Gynecol. 655-60 (1985); Daffos et al., 146 Am. J. Obstet. Gynecol. 985-87 (1983), by placentocentesis (Valenti, 115 Am. J. Obstet. Gynecol. 851-53 (1973); Cao et al., 19 J. Med. Genet. 81-87 (1982)), by fetoscopy (Rodeck, in Prenatal Diagnosis, (Rodeck & Nicolaides, eds., Royal College of Obstetricians & Gynaecologists, London, 1984)) and cryopreserved. Indeed, the chorionic villus and amniotic fluid, in addition to cord blood and placenta, are sources of pluripotent fetal stem cells (see WO 2003 042405).

Various kits and collection devices are known for the collection, processing, and storage of cord blood. See, e.g., U.S. Pat. No. 7,147,626; No. 7,131,958. Collections should be made under sterile conditions, and the blood can be treated with an anticoagulant. Such anticoagulants include, for example, citrate-phosphate-dextrose, acid citrate-dextrose, Alsever's solution (Alsever & Ainslie, 41 N.Y. St. J. Med. 126-35 (1941), DeGowin's Solution (DeGowin et al., 114 JAMA 850-55 (1940)), Edglugate-Mg (Smith et al., 38 J. Thorac. Cardiovasc. Surg. 573-85 (1959)), Rous-Turner Solution (Rous & Turner, 23 J. Exp. Med. 219-37 (1916)), other glucose mixtures, heparin, or ethyl biscoumacetate. See Hum, Storage of Blood 26-160 (Acad. Press, NY, 1968).

Various procedures are known in the art or described herein and can be used to enrich collected cord blood for MHCs. These include but are not limited to equilibrium density centrifugation, velocity sedimentation at unit gravity, immune rosetting and immune adherence, counterflow centrifugal elutriation, T-lymphocyte depletion, and fluorescence-activated cell sorting, alone or in combination. See, e.g., U.S. Pat. No. 5,004,681.

Typically, collected blood is prepared for cryogenic storage by addition of cryoprotective agents such as DMSO (Lovelock & Bishop, 183 Nature 1394-95 (1959); Ashwood-Smith 190 Nature 1204-05 (1961)), glycerol, polyvinylpyrrolidine (Rinfret, 85 Ann. N.Y. Acad. Sci. 576-94 (1960)), polyethylene glycol (Sloviter & Ravdin, 196 Nature 899-900 (1962)), albumin, dextran, sucrose, ethylene glycol, i-erythritol, D-ribitol, D-mannitol (Rowe, 3(1) Cryobiology 12-18 (1966)), D-sorbitol, i-inositol, D-lactose, choline chloride (Bender et al., 15J. Appl. Physiol. 520-24 (1960)), amino acids (Phan & Bender, 20 Exp. Cell Res. 651-54 (1960)), methanol, acetamide, glycerol monoacetate (Lovelock, 56 Biochem. J. 265-70 (1954)), and inorganic salts (Phan & Bender, 104 Proc. Soc. Exp. Biol. Med. (1960)). Addition of plasma (e.g., to a concentration of 20%-25%) can augment the protective effect of DMSO.

Collected blood should be cooled at a controlled rate for cryogenic storage. Different cryoprotective agents and different cell types have different optimal cooling rates. See e.g., Rapatz, 5 Cryobiology 18-25 (1968), Rowe & Rinfret, 20 Blood 636-37 (1962); Rowe, 3 Cryobiology 12-18 (1966); Lewis et al., 7 Transfusion 17-32 (1967); Mazur, 168 Science 939-49 (1970). Considerations and procedures for the manipulation, cryopreservation, and long-term storage of MHC sources are known in the art. See e.g., U.S. Pat. No. 4,199,022; U.S. Pat. No. 3,753,357; U.S. Pat. No. 4,559,298; U.S. Pat. No. 5,004,681. There are also various devices with associated protocols for the storage of blood. U.S. Pat. No. 6,226,997; U.S. Pat. No. 7,179,643

Considerations in the thawing and reconstitution of MHC sources are also known in the art. U.S. Pat. No. 7,179,643; U.S. Pat. No. 5,004,681. The MHC source blood can also be treated to prevent clumping (see Spitzer, 45 Cancer 3075-85 (1980); Stiff et al., 20 Cryobiology 17-24 (1983), and to remove toxic cryoprotective agents (U.S. Pat. No. 5,004,681). Further, there are various approaches to determining an engrafting cell dose of MHC transplant units. See U.S. Pat. No. 6,852,534; Kuchler, in Biochem. Meths. Cell Culture & Virology 18-19 (Dowden, Hutchinson & Ross, Strodsburg, Pa., 1964); 10 Meths. Med. Res. 39-47 (Eisen, et al., eds., Year Book Med. Pub., Inc., Chicago, Ill., 1964).

Non-limiting examples of negative regulators of MHC expansion include Itch, SH2BE/Lnk, Ahr, and Prox1. These are described below.

Itch (NCBI Gene ID: 83737) is a E3 ubiquitin ligase belonging to the HECT family which has one known mRNA transcript (NCBI Accession No: NM_{—}031483; SEQ ID NO: 11). Itch has been shown to be important for proper function of T cells as well as hematopoiesis. Itch negatively regulates the development and function of HSCs.

Inhibitors of Itch expression as described herein can effect the expansion of MHCs.

Sh2b3 (also known as Lnk; NCBI Gene ID: 10019) is a member of intracellular adaptor protein family consisting of SH2-B/Sh2b1 and APS/Sh2b2 which has one known mRNA transcript (NCBI Accession No: NM_{—}005475; SEQ ID NO: 1). Sh2b3 is expressed primarily in lymphocytes and hematopoietic precursor cells and regulates early lymphohematopoiesis. Sh2b3-deficient mice show overproduction of B cells and megakaryocytes, which is due to hypersensitivity to cytokines including stem cell factor (SCF) and thrombopoietin. Lymphoid precursors overexpressing Sh2b3 resulted in a reduction of B and T cells.

Additionally, Sh2b3 functions in responses controlled by cell adhesion and in crosstalk between integrin- and cytokine-mediated signaling. Sh2b3-deficient mice overproduced platelets and megakaryocytes in response to thrombopoietin. The Sh2b3-deficient mice also displayed inceased Erk1/2 signaling in response to thrombopoietin, while Stat3, Stat5, and Akt response were normal. Sh2b3-deficient mice are also insensitive to perturbation of signaling by VCAM-1.

Inhibitors of SH2B3 expression as described herein can effect the expansion of MHCs.

PROX1 (NCBI Gene ID: 5629; also called PROX-1, prospero-related homeobox 1, and homeodomain protein) is a homeoprotein, a class of proteins known to play essential roles in cell fate determination and body plan establishment. Prox-1 is known to encode one mRNA transcript (NCBI Accession No: NM_{—}002763; SEQ ID NO: 2) and expressed in several human tissues including lens, heart, brain, lung, kidney, and liver, with the highest expression found in lens. Gene association studies have suggested a role for polymorphisms of PROX-1 in eye pathologies.

The biological function of prox-1 has been studied by generating prox-1 null mice. From these studies it was determined that prox-1 is required for hepatocyte migration during liver development, development of the lens and the lymphatic system, but not the vascular system and has been used as a marker to distinguish lymphatic vessels from blood vessels in vivo (Sosa-Pineda et al, Nat. Genet., 2000, 25, 254-255; Wigle et al., Nat. Genet., 1999, 21, 318-322; Wigle and Oliver, Cell, 1999, 98, 769-778.). Prox-1 function is also required for the expression of the cell-cycle inhibitors Cdknlb and Cdknlc (Wigle et al, Nat. Genet., 1999, 21, 318-322.). Several other functions for prox-1 as a transcription factor have been described. Prox-1 activates the SIX3 promoter, a human transcription factor essential for eye development (Lengler and Graw, Biochem. Biophys. Res. Commun., 2001, 287, 372-376). Prox-1 regulates differentiation of neurons and glia in neural progenitors (Yamamoto et al, /. Neurosci.; 2001, 21, 9814-9823.) and prox-1 also stimulates the Crygf promoter, a gene which has been reported to have mutations that result in a variety of lens opacities (Lengler et al., Nucleic Acids Res., 2001, 29, 515-526).

Prox1 is the vertebrate homolog of Prospero, a divergent homeodomain protein important for neuroblast fate determination and photoreceptor development in Drosophila (C. Q. Doe et al. (1991) “The Prospero gene specifies cell fates in the Drosophila central nervous system”, Cell 65: 451-464; B. Hassan et al. (1997) “Prospero is a panneural transcription factor that modulates homeodomain protein activity”, Proc. Natl. Acad. Sci. USA 94: 10991-10996; T. Cook et al. (2003) “Distinction between color photoreceptor cell fates is controlled by Prospero in Drosophila”, Dev. Cell 4: 853-864). Human Prox1 is a 736 amino acid protein containing an N-terminal nuclear localization signal, three nuclear receptor boxes, a nuclear export signal and a highly conserved C-terminus containing the divergent homeodomain and the novel Prospero domain (T. R. Burglin (1994) “A Caehorhabditis elegans homologue defines a novel domain”, Trends Biochem. Sci. 19: 70-71; S. I. Tomarev et al. (1998) “Characterization of the mouse Prox1 gene”, Biochem. Biophys. Res. Commun. 248: 684-689; J. Qin et al. (2004) “Prospero-related homeobox (Proxy) is a corepressor of human liver receptor homolog-1 and suppresses the transcription of the cholesterol 7-alpha-hydroxylase gene”, Mol. Endocrinol. 18: 2424-2439). Prox1 is highly expressed in lens fiber cells (M. K. Duncan et al. (2002) “Prox1 is differentially localized during lens development”, Mech. Dev. 112: 195-198) and Prox1 null mice are defective in lens fiber cell elongation (J. T. Wigle et al. (1999) “Prox1 function is crucial for mouse lens-fibre elongation”, Nat. Genet. 21: 318-322) and the differentiation of retinal horizontal cells (M. A. Dyer et al. (2003) “Prox1 function controls progenitor cell proliferation and horizontal cell genesis in the mammalian retina”, Nat. Genet. 34: 53-58). In this context, Prox1 functions as a transcription factor and has been shown to transactivate both the chicken βB1- and mouse γF-crystallin promoters (J. Lengler et al. (2001) “Antagonistic action of six3 and prox1 at the gamma-crystallin promoter”, Nucleic Acids Res. 29: 515-526; W. Cui et al. (2004) “Mafs, Prox1 and Pax6 can regulate chicken beta B1-crystallin gene expression”, J. Biol. Chem. 279: 11088-11095).

While Prox1 is critical for eye development, the correct dosage of this protein is essential for embryogenesis since heterozygous Prox 1 null mice die shortly after birth on most genetic backgrounds while homozygous Prox1 nulls die at 14.5 dpc (J. T. Wigle et al. (1999) “Prox1 function is crucial for mouse lens-fibre elongation”, Nat. Genet. 21: 318-322; J. T. Wigle et al. (1999) “Prox1 function is required for the development of the murine lymphatic system”, Cell 98: 769-778). Analysis of these animals has shown that Prox1 is essential for the delamination of hepatocytes from the liver bud into the surrounding mesenchyme which is necessary for normal liver development (B. Sosa-Pineda et al. (2000) “Hepatocyte migration during liver development requires Prox1”, Nat. Genet. 25: 254-255). Expression studies have shown Prox1 to be one of the earliest molecular markers of liver/pancreatic fated ventral foregut endoderm (Z. Burke et al. (2002) “Prox1 is an early specific marker for the developing liver and pancreas in the mammalian foregut endoderm”, Mech. Dev. 118: 147-155). Prox1 expression is maintained in hepatoblasts and hepatocytes throughout development and is highly upregulated in transformed hepatoma cell lines (J. Dudas et al. (2004) “The homeobox transcription factor Prox1 is highly conserved in embryonic hepatoblasts and in adult and transformed hepatocytes, but is absent from bile duct epithelium”, Anat. Embryol. (Berl) 208: 359-366). Prox1 interacts with liver receptor homolog-1 (LRH-1), a transcription factor essential for the expression of enzymes important for bile acid synthesis, repressing LRH-1 transcriptional activity by impairing its binding to DNA (J. Qin et al. (2004) “Prospero-related homeobox (Prox1) is a corepressor of human liver receptor homolog-1 and suppresses the transcription of the cholesterol 7-alpha-hydroxylase gene”, Mol. Endocrinol. 18: 2424-2439).

Prox 1 is also a key player in the formation of the lymphatic system (Y. K. Hong et al. (2002) “Prox1 is a master control gene in the program specifying lymphatic endothelial cell fate”, Dev. Dyn. 225: 351-357). Expression of Prox1 in a subpopulation of venous endothelial cells is one of the first indications that lymphangiogenesis has been initiated and cells biased to a lymphatic phenotype (J. T. Wigle et al. (2002) “An essential role for Prox1 in the induction of the lymphatic endothelial cell phenotype”, Embo. J 21: 1505-1513). Prox1 null mice do not develop lymphatics due to arrested endothelial budding from the primary vascular network (J. T. Wigle et al. (1999) “Prox1 function is required for the development of the murine lymphatic system”, Cell 98: 769-778). Overexpression of Prox1 can reprogram blood vascular endothelial cells into lymphatic endothelial cells confirming the central role of Prox1 in lymphatic specification (T. V. Petrova et al. (2002) “Lymphatic endothelial reprogramming of vascular endothelial cells by the Prox-1 homeobox transcription factor”, Embo J. 21: 4593-4599). Immunostaining of lymphatic tissues from healthy human adults and lymphedema patients with Prox1 polyclonal antibodies showed that Prox1 is a reliable and highly specific marker for lymphatic endothelial cells in normal and pathologic human tissues (J. Wilting et al. (2002) “The transcription factor Prox1 is a marker for lymphatic endothelial cells in normal and diseased human tissues”, Faseb J. 16: 1271-1273; J. S. Reis-Filho et al. (2003) “Lymphangiogenesis in tumors: what do we know?”, Microsc. Res. Tech. 60: 171-180; I. Van der Auwera et al. (2004) “Increased angiogenesis and lymphangiogenesis in inflammatory versus noninflammatory breast cancer by real-time reverse transcriptase-PCR gene expression quantification”, Clin. Cancer Res. 10: 7965-7971; M. A. Al-Rawi et al. (2005) “Lymphangiogenesis and its role in cancer”, Histol. Histopathol. 20: 283-298).

Inhibitors of PROX1 expression as described herein can effect the expansion of MHCs.

AHR(NCBI Gene ID: 196; Aryl hydrocarbon receptor) is a cytosolic ligand-activated transcription factor that translocates to the nucleus after ligand binding. Ahr is known to be transcribed into one mRNA transcript variant (NCBI Accession No: NM_{—}001621; SEQ ID NO: 3) Inhibition of AHR can expand HSC populations (US Patent Publication 2007/0048313) while AHR ligands have been disclosed as dermatological or cosmetic medicaments (US Patent Publication 2010/0324109). AHR is known to mediate a large number of toxic and carcinogenic effects in animals and possibly in humans (Safe S 2001 Toxicol Lett 120:1-7). As a consequence of AHR activation by its ligands, many detoxification genes are transcriptionally induced, including those coding for phase I xenobiotic-metabolizing enzymes, such as the cytochromes P450 CYP1A1, CYP1A2, CYP1B1 and CYP2S1, and the phase II enzymes UDP-glucuronosyltransferase UGT1A6, NAD(P)H-dependent quinone oxidoreductase-1 (NQO1), the aldehyde dehydrogenase ALDH3A1, and several glutathione-S-transferase.

Ligands of AHR include 2,3,7,8-tetrachlorodibenzo-p-dioxin (TCDD), an environmental toxin that is a by-product of industrial processes (Poland et al., J Biol Chem, 1976, 251, 4936-4946); polycyclic aromatic hydrocarbons, found in cigarette smoke and smog (Reisz-Porszasz et al., Mol Cell Biol, 1994, 14, 6075-6086); and heterocyclic amines, found in some cooked meats (Reisz-Porszasz et al., Mol Cell Biol, 1994, 14, 60756086). After ligand-binding and nuclear translocation, AHR forms a dimer with HIF1-beta, resulting in the activation of a number of genes involved in drug metabolism, such as the cytochromes P450, CYP1A1, CYP1A2, and CYP1B1. AHR/HIF1-beta dimers are capable of activating a range of other genes regulated by the dioxin response element (DRE), resulting in some of the toxic and carcinogenic effects associated with many of the AHR ligands, such as immunotoxicity, developmental and reproductive toxicity, disruption of endocrine pathways, a wasting syndrome, and tumor promotion (Safe, Toxicol Lett, 2001, 120, 1-7). Ohtake and colleagues (Ohtake et al., Nature, 2003, 423, 545-550) demonstrated that the AHR/HIF1-beta heterodimer directly associates with the estrogen receptors ER-alpha and ER-beta. They showed that this association results in the recruitment of unliganded estrogen receptor and coactivator p300 to estrogen-responsive gene promoters, leading to activation of transcription and estrogenic effects and giving rise to the adverse estrogen-related actions of dioxin-type environmental contaminants.

Inhibitors of Ahr expression as described herein can effect the expansion of MHCs.

Described herein are iRNA agents that inhibit the expression of a gene or genes encoding a negative regulator(s) of MHC expansion. In one embodiment, the iRNA agent includes double-stranded ribonucleic acid (dsRNA) molecules for inhibiting the expression of a gene encoding a negative regulator of MHC expansion in a cell or mammal, e.g., a cell in a population of human MHCs obtained from UCB, where the dsRNA includes an antisense strand having a region of complementarity which is complementary to at least a part of an mRNA formed in the expression of a gene encoding a negative regulator of MHC expansion, and where the region of complementarity is 30 nucleotides or less in length, generally 19-24 nucleotides in length, and where the dsRNA, upon contact with or introduction to a cell expressing the gene encoding a negative regulator of MHC expansion, inhibits the expression of the gene by at least 10% as assayed by, for example, a PCR or branched DNA (bDNA)-based method, or by a protein-based method, such as by immunoassay or Western blot. Expression of a negative regulator of MHC expansion in cell culture, such as in MHCs or in a biological sample from a subject, can be assayed by measuring mRNA levels of a gene encoding a negative regulator of MHC expansion, such as by bDNA or TaqMan assay, or by measuring protein levels, such as by immunofluorescence analysis, using, for example, Western Blotting or flowcytometric techniques.

A dsRNA includes two RNA strands that are complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of a gene encoding a negative regulator of MHC expansion. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

One of skill in the art will also recognize that the duplex region is a primary functional portion of a dsRNA, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an RNA molecule or complex of RNA molecules having a duplex region greater than 30 base pairs is a dsRNA. Thus, an ordinarily skilled artisan will recognize that in one embodiment, then, an miRNA is a dsRNA. In another embodiment, a dsRNA is not a naturally occurring miRNA. In another embodiment, an iRNA agent useful to target expression of a gene encoding a negative regulator of MHC expansion is not generated in the target cell by cleavage of a larger dsRNA.

A dsRNA as described herein can further include one or more single-stranded nucleotide overhangs. The dsRNA can be synthesized by standard methods known in the art as further discussed below, e.g., by use of an automated DNA synthesizer, such as are commercially available from, for example, Biosearch, Applied Biosystems, Inc. In one embodiment, a gene encoding a negative regulator of MHC expansion is a human gene. In another embodiment the gene encoding a negative regulator of MHC expansion is a mouse or rat gene. In specific embodiments, the first sequence is a sense strand of a dsRNA that includes a sense sequence from one of Tables 2-7, and the second sequence is selected from the group consisting of the antisense sequences of one of Tables 2-7. Alternative dsRNA agents that target elsewhere in the target sequence provided in Tables 2-7 can readily be determined using the target sequence and the flanking sequence.

In one aspect, a dsRNA will include at least two nucleotide sequences, a sense and an anti-sense sequence, wherein the sense strand is selected from the groups of sequences provided in Tables 2-7, and the corresponding antisense strand of the sense strand selected from Tables 2-7. In this aspect, one of the two sequences is complementary to the other of the two sequences, with one of the sequences being substantially complementary to a sequence of an mRNA generated in the expression of a gene encoding a negative regulator of MHC expansion. As such, in this aspect, a dsRNA will include two oligonucleotides, where one oligonucleotide is described as the sense strand in Tables 2-7, and the second oligonucleotide is described as the corresponding antisense strand of the sense strand from Tables 2-7. As described elsewhere herein and as known in the art, the complementary sequences of a dsRNA can also be contained as self-complementary regions of a single nucleic acid molecule, as opposed to being on separate oligonucleotides.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer RNA duplex structures can be effective as well. In the embodiments described above, by virtue of the nature of the oligonucleotide sequences identified by sequence identifiers provided in Tables 2-7, dsRNAs described herein can include at least one strand of a length of minimally 21 nt. It can be reasonably expected that shorter duplexes having one of the sequences of Tables 2-7 minus only a few nucleotides on one or both ends can be similarly effective as compared to the dsRNAs described above. Hence, dsRNAs having a partial sequence of at least 15, 16, 17, 18, 19, 20, or more contiguous nucleotides from one of the sequences of Tables 2-7, and differing in their ability to inhibit the expression of a gene encoding a negative regulator of MHC expansion by not more than 5, 10, 15, 20, 25, or 30% inhibition from a dsRNA comprising the full sequence, are contemplated according to the technology described herein.

In addition, the RNAs provided in Tables 2-7 identify a site in a transcript encoding a negative regulator of MHC expansion that is susceptible to RISC-mediated cleavage. As such, the technology described herein further features iRNAs that target within one of such sequences. As used herein, an iRNA is said to target within a particular site of an RNA transcript if the iRNA promotes cleavage of the transcript anywhere within that particular site. Such an iRNA will generally include at least 15 contiguous nucleotides from one of the sequences provided in Tables 2-7 coupled to additional nucleotide sequences taken from the region contiguous to the selected sequence in a gene encoding a negative regulator of MHC expansion.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression. Thus, while the sequences identified, for example, by sequence identifiers in Tables 2-7 represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified by a sequence identifier, e.g., in Tables 2-78, further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of iRNAs based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch not be located in the center of the region of complementarity. If the antisense strand of the iRNA contains mismatches to the target sequence, it is preferable that the mismatch be restricted to be within the last 5 nucleotides from either the 5′ or 3′ end of the region of complementarity. For example, for a 23 nucleotide iRNA agent RNA strand which is complementary to a region of a gene encoding a negative regulator of MHC expansion, the RNA strand generally does not contain any mismatch within the central 13 nucleotides. The methods described herein or methods known in the art can be used to determine whether an iRNA containing a mismatch to a target sequence is effective in inhibiting the expression of a gene encoding a negative regulator of MHC expansion. Consideration of the efficacy of iRNAs with mismatches in inhibiting expression of a gene encoding a negative regulator of MHC expansion is important, especially if the particular region of complementarity in a gene encoding a negative regulator of MHC expansion is known to have polymorphic sequence variation within the population.

In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts. In yet another embodiment, the RNA of an iRNA, e.g., a dsRNA, is chemically modified to enhance stability or other beneficial characteristics. The nucleic acids featured in the technology described herein can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other embodiments, suitable RNA mimetics suitable are contemplated for use in iRNAs, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the technology described herein include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy(2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy(2′-OCH₃), 2′-aminopropoxy(2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the technology described herein. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-β-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the technology described herein involves chemically linking to the RNA one or more ligands, moieties or conjugates that enhance the activity, cellular distribution or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-5-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In one embodiment, a ligand alters the distribution, targeting or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B 12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a HSC. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic (PK) modulator. As used herein, a “PK modulator” refers to a pharmacokinetic modulator. PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the technology described herein as ligands (e.g. as PK modulating ligands). In addition, aptamers that bind serum components (e.g. serum proteins) are also suitable for use as PK modulating ligands in the embodiments described herein.

RNA effector molecules are ribonucleotide agents that are capable of reducing the expression level of a target gene within a host cell, or ribonucleotide agents capable of forming a molecule that can reduce the expression level of a target gene within a host cell. A portion of a RNA effector molecule, wherein the portion is at least 10, at least 12, at least 15, at least 17, at least 18, at least 19, or at least 20 nucleotide long, is substantially complementary to the target gene. The complementary region may be the coding region, the promoter region, the 3′ untranslated region (3′-UTR), and/or the 5′-UTR of the target gene. Preferably, at least 16 contiguous nucleotides of the RNA effector molecule are complementary to the target sequence (e.g., at least 17, at least 18, at least 19, or more contiguous nucleotides of the RNA effector molecule are complementary to the target sequence). The RNA effector molecules interact with RNA transcripts of target genes and mediate their selective degradation or otherwise prevent their translation. In some embodiments, the target gene is selected from the group consisting of: a gene for Itch (e.g. SEQ ID NO: 23), a gene for SH2B3/Lnk (e.g. SEQ ID NO:22), a gene for Ahr (e.g. SEQ ID NO: 21), and a gene for Prox 1 (e.g. SEQ ID NO: 20). The RNA effector molecules may interact with RNA transcripts of target genes and mediate their selective degradation or otherwise prevent their translation. In some embodiments, the target gene sequence is a mRNA transcript sequence selected from SEQ ID NO's: 1-3, or 8-12.

RNA effector molecules can comprise a single RNA strand or more than one RNA strand. Examples of RNA effector molecules include, e.g., double stranded RNA (dsRNA), microRNA (miRNA), antisense RNA, promoter-directed RNA (pdRNA), Piwi-interacting RNA (piRNA), expressed interfering RNA (eiRNA), short hairpin RNA (shRNA), antagomirs, decoy RNA, DNA, plasmids and aptamers. The RNA effector molecule can be single-stranded or double-stranded. A single-stranded RNA effector molecule can have double-stranded regions and a double-stranded RNA effector can have single-stranded regions. Preferably, the RNA effector molecules are double-stranded RNA, wherein the antisense strand comprises a sequence that is substantially complementary to the target gene.

Complementary sequences within a RNA effector molecule, e.g., within a dsRNA (a double-stranded ribonucleic acid) may be fully complementary or substantially complementary. Generally, for a for a duplex up to 30 base pairs, the dsRNA comprises no more than 5, 4, 3 or 2 mismatched base pairs upon hybridization, while retaining the ability to regulation the expression of its target gene.

In some embodiments, the RNA effector molecule comprises a single-stranded oligonucleotide that interacts with and directs the cleavage of RNA transcripts of a target gene. For example, single stranded RNA effector molecules comprise a 5′ modification including one or more phosphate groups or analogs thereof to protect the effector molecule from nuclease degradation. The RNA effector molecule can be a single-stranded antisense nucleic acid having a nucleotide sequence that is complementary to a “sense” nucleic acid of a target gene, e.g., the coding strand of a double-stranded cDNA molecule or a RNA sequence, e.g., a pre-mRNA, mRNA, miRNA, or pre-miRNA. Accordingly, an antisense nucleic acid can form hydrogen bonds with a sense nucleic acid target.

Given a coding strand sequence (e.g., the sequence of a sense strand of a cDNA molecule), antisense nucleic acids can be designed according to the rules of Watson-Crick base pairing. The antisense nucleic acid can be complementary to the coding or noncoding region of a RNA, e.g., the region surrounding the translation start site of a pre-mRNA or mRNA, e.g., the 5′ UTR. An antisense oligonucleotide can be, for example, about 10 to 25 nucleotides in length (e.g., 10, 11, 12, 13, 14, 15, 16, 17, 18, 19, 20, 21, 22, 23, or 24 nucleotides in length). In some embodiments, the antisense oligonucleotide comprises one or more modified nucleotides, e.g., phosphorothioate derivatives and/or acridine substituted nucleotides, designed to increase its biological stability of the molecule and/or the physical stability of the duplexes formed between the antisense and target nucleic acids. Antisense oligonucleotides can comprise ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA and prevent access of the translation machinery to the target RNA transcript, thereby preventing protein synthesis. An antisense molecule including only deoxyribonucleotides, or deoxyribonucleotides and ribonucleotides, can hybridize to a complementary RNA and the RNA target can be subsequently cleaved by an enzyme, e.g., RNAse H, to prevent translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

In one embodiment, the ligand or conjugate is a lipid or lipid-based molecule. Such a lipid or lipid-based molecule preferably binds a serum protein, e.g., human serum albumin (HSA). Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. Exemplary vitamins include vitamin A, E, and K. Other exemplary vitamins include are B vitamin, e.g., folic acid, B12, riboflavin, biotin, pyridoxal or other vitamins or nutrients taken up by target cells such as HSC. Also included are HSA and low density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO:4). An RFGF analogue (e.g., amino acid sequence AALLPVLLAAP (SEQ ID NO:5)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:6)) and the Drosophila Antennapedia protein (RQIKIWFQNRRMKWKK (SEQ ID NO:7)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide moiety can be used to target a tumor cell, such as an endothelial tumor cell or a breast cancer tumor cell (Zitzmann et al., Cancer Res., 62:5139-43, 2002). An RGD peptide can facilitate targeting of an dsRNA agent to tumors of a variety of other tissues, including the lung, kidney, spleen, or liver (Aoki et al., Cancer Gene Therapy 8:783-787, 2001). Preferably, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver a iRNA agent to a tumor cell expressing αVβ3 (Haubner et al., Jour. Nucl. Med., 42:326-336, 2001).

Peptides that target markers enriched in proliferating cells can be used, e.g., RGD containing peptides and peptidomimetics can target cancer cells, in particular cells that exhibit an αvβ3 integrin. Thus, one could use RGD peptides, cyclic peptides containing RGD, RGD peptides that include D-amino acids, as well as synthetic RGD mimics. In addition to RGD, one can use other moieties that target the αvβ3 integrin ligand. Generally, such ligands can be used to control proliferating cells and angiogenesis.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

In some embodiments, the iRNA oligonucleotides described herein further comprise carbohydrate conjugates. The carbohydrate conjugates are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C5 and above (preferably C5-C8) sugars; di- and trisaccharides include sugars having two or three monosaccharide units (preferably C5-C8).

In some embodiments, the carbohydrate conjugate further comprises other ligand such as, but not limited to, PK modulator, endosomolytic ligand, and cell permeation peptide.

In some embodiments, the conjugates described herein can be attached to the iRNA oligonucleotide with various linkers that can be cleavable or non cleavable.

The term “linker” or “linking group” means an organic moiety that connects two parts of a compound. Linkers typically comprise a direct bond or an atom such as oxygen or sulfur, a unit such as NR8, C(O), C(O)NH, SO, SO2, SO2NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO2, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between 1-24 atoms, preferably 4-24 atoms, preferably 6-18 atoms, more preferably 8-18 atoms, and most preferably 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of an RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

The delivery of an iRNA to a MHC can be achieved in a number of different ways. Ex vivo delivery can be performed by contacting a cell with a composition comprising an iRNA, eg. dsRNA. iRNA formulations and methods for delivery to cells in culture is well known to those of skill in the art. Alternatively, delivery can be performed by contacting a cell with one or more vectors that encode and direct the expression of the iRNA.

While a preferred aspect described herein involves in vitro manipulation of MHCs before transplantation, the administration of iRNA formulations to a subject undergoing to in need of an MHC transplant is also specifically contemplated. In vivo delivery can be performed directly by administering a composition comprising an iRNA, e.g. a dsRNA, to a subject. Alternatively, delivery can be performed indirectly by administering one or more vectors that encode and direct the expression of the iRNA. These alternatives are discussed further below.

In general, any method of delivering a nucleic acid molecule can be adapted for use with an iRNA (see e.g., Akhtar S, and Julian R L. (1992) Trends Cell. Biol. 2(5):139-144 and WO94/02595, which are incorporated herein by reference in their entireties). However, there are three factors that are important to consider in order to successfully deliver an iRNA molecule in vivo: (a) biological stability of the delivered molecule, (2) preventing non-specific effects, and (3) accumulation of the delivered molecule in the target tissue. The non-specific effects of an iRNA can be minimized by local administration, for example by direct injection or implantation into a tissue (as a non-limiting example, a tumor) or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered. Several studies have shown successful knockdown of gene products when an iRNA is administered locally. For example, intraocular delivery of a VEGF dsRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an iRNA systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA composition to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an iRNA to an aptamer has been shown to inhibit tumor growth and mediate tumor regression in a mouse model of prostate cancer (McNamara, J O., et al (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H., et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R., et al (2003) J. Mol. Biol. 327:761-766; Verma, U N., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety.

In another aspect, iRNA targeting a gene encoding a negative regulator of MHC expansion can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

The individual strand or strands of an iRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

iRNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell.

iRNA expression plasmids can be transfected into target cells as a complex with cationic lipid carriers (e.g., Oligofectamine) or non-cationic lipid-based carriers (e.g., Transit-TKO™). Multiple lipid transfections for iRNA-mediated knockdowns targeting different regions of a target RNA over a period of a week or more are also contemplated by the technology described herein. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are further described below.

Vectors useful for the delivery of an iRNA will include regulatory elements (promoter, enhancer, etc.) sufficient for expression of the iRNA in the desired target cell or tissue. The regulatory elements can be chosen to provide either constitutive or regulated/inducible expression.

Expression of the iRNA can be precisely regulated, for example, by using an inducible regulatory sequence that is sensitive to certain physiological regulators, e.g., circulating glucose levels, or hormones (Docherty et al., 1994, FASEB J. 8:20-24). Such inducible expression systems, suitable for the control of dsRNA expression in cells or in mammals include, for example, regulation by ecdysone, by estrogen, progesterone, tetracycline, chemical inducers of dimerization, and isopropyl-beta-D1-thiogalactopyranoside (IPTG). A person skilled in the art would be able to choose the appropriate regulatory/promoter sequence based on the intended use of the iRNA transgene.

In a specific embodiment, viral vectors that contain nucleic acid sequences encoding an iRNA can be used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding an iRNA are cloned into one or more vectors, which facilitates delivery of the nucleic acid into a patient. More detail about retroviral vectors can be found, for example, in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Lentiviral vectors contemplated for use include, for example, the HIV based vectors described in U.S. Pat. Nos. 6,143,520; 5,665,557; and 5,981,276, which are herein incorporated by reference.

Adenoviruses are also contemplated for use in delivery of iRNAs. Adenoviruses are especially attractive vehicles, e.g., for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). A suitable AV vector for expressing an iRNA featured in the technology described herein, a method for constructing the recombinant AV vector, and a method for delivering the vector into target cells, are described in Xia H et al. (2002), Nat. Biotech. 20: 1006-1010.

Use of Adeno-associated virus (AAV) vectors is also contemplated (Walsh et al., Proc. Soc. Exp. Biol. Med. 204:289-300 (1993); U.S. Pat. No. 5,436,146). In one embodiment, the iRNA can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the technology described herein, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

Another preferred viral vector is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes; see, e.g., Rabinowitz J E et al. (2002), J Virol 76:791-801, the entire disclosure of which is herein incorporated by reference.

The pharmaceutical preparation of a vector can include the vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, provided herein are pharmaceutical compositions containing an iRNA and a pharmaceutically acceptable carrier. The pharmaceutical composition containing the iRNA is useful for treating a disease or disorder that benefits from MHC transplantation or expansion. The iRNA compositions described herein can enhance MHC expansion and/or engraftment and thereby enhance the potential of cells to differentiate to the necessary or desired hematopoietic lineage cell types. Such pharmaceutical compositions are formulated based on the mode of delivery. One example is compositions that are formulated for systemic administration via parenteral delivery, e.g., by intravenous (IV) delivery.

The pharmaceutical compositions featured herein are administered in dosages sufficient to inhibit expression of genes encoding negative regulators of MHC expansion. In general, a suitable dose of iRNA will be in the range of 0.01 to 200.0 milligrams per kilogram body weight of the recipient per day, generally in the range of 1 to 50 mg per kilogram body weight per day. For example, the dsRNA can be administered at 0.05 mg/kg, 0.5 mg/kg, 1 mg/kg, 1.5 mg/kg, 2 mg/kg, 3 mg/kg, 10 mg/kg, 20 mg/kg, 30 mg/kg, 40 mg/kg, or 50 mg/kg per single dose. The pharmaceutical composition can be administered once daily, or the iRNA can be administered as two, three, or more sub-doses at appropriate intervals throughout the day or even using continuous infusion or delivery through a controlled release formulation. In that case, the iRNA contained in each sub-dose must be correspondingly smaller in order to achieve the total daily dosage. The dosage unit can also be compounded for delivery over several days, e.g., using a conventional sustained release formulation which provides sustained release of the iRNA over a several day period. Sustained release formulations are well known in the art and are particularly useful for delivery of agents at a particular site, such as could be used with the agents of the technology described herein. In this embodiment, the dosage unit contains a corresponding multiple of the daily dose.

The effect of a single dose on expression levels of a gene encoding a negative regulator of MHC expansion can be long lasting, such that subsequent doses are administered at not more than 3, 4, or 5 day intervals, or at not more than 1, 2, 3, or 4 week intervals.

The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNAs encompassed by the technology described herein can be made using conventional methodologies or on the basis of in vivo testing using an appropriate animal model, as described elsewhere herein.

Advances in mouse genetics have generated a number of mouse models for the study of various human diseases, such as hematopoietic diseases treated by MHC transplantation. Such models can be used for in vivo testing of iRNA, as well as for determining a therapeutically effective dose. A suitable mouse model is, for example, a mouse with leukemia.

The technology described herein also includes pharmaceutical compositions and formulations that include the iRNA compounds featured herein. The pharmaceutical compositions of the technology described herein can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The iRNA can be delivered in a manner to target a particular tissue, such as the bone marrow (e.g., the MHCs in the bone marrow).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the iRNAs featured in the technology described herein are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). iRNAs featured in the technology described herein can be encapsulated within liposomes or can form complexes thereto, in particular to cationic liposomes. Alternatively, iRNAs can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present technology described herein, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to traverse intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent can act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged RNA molecules to form a stable complex. The positively charged RNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap nucleic acids rather than complex with it. Since both the nucleic acid and the lipid are similarly charged, repulsion rather than complex formation occurs. Nevertheless, some nucleic acid is entrapped within the aqueous interior of these liposomes. pH-sensitive liposomes have been used to deliver nucleic acids encoding the thymidine kinase gene to cell monolayers in culture. Expression of the exogenous gene was detected in the target cells (Thou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilauratekholesterol/polyoxyethylene-10-stearyl ether) and Novasome™ II (glyceryl distearatekholesterol/polyoxyethylene-10-stearyl ether) were used to deliver cyclosporin-A into the dermis of mouse skin. Results indicated that such non-ionic liposomal systems were effective in facilitating the deposition of cyclosporine-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describes PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes can include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

In one embodiment, an dsRNA featured in the technology described herein is fully encapsulated in the lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles of the technology described herein typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the technology described herein are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid can be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-(2-(bis(2-hydroxydodecypamino)ethyl)(2-hydroxydodecypamino)ethyl)piperazin-1-yl)ethylazanediyl)didodecan-2-ol (Tech G1), or a mixture thereof. The cationic lipid can comprise from about 20 mol % to about 50 mol % or about 40 mol % of the total lipid present in the particle.

In another embodiment, the compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

In one embodiment, the lipid-siRNA particle includes 40% 2,2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 siRNA/Lipid Ratio.

The non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the technology described herein are administered in conjunction with one or more penetration enhancer surfactants and chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the technology described herein can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, US Publn. No. 20030027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the technology described herein include, but are not limited to, solutions, emulsions, and liposome-containing formulations. These compositions can be generated from a variety of components that include, but are not limited to, preformed liquids, self-emulsifying solids and self-emulsifying semisolids.

The pharmaceutical formulations of the technology described herein, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the technology described herein can be formulated into any of many possible dosage forms such as, but not limited to, tablets, capsules, gel capsules, liquid syrups, soft gels, suppositories, and enemas. The compositions of the technology described herein can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

The compositions of the technology described herein can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the technology described herein, the compositions of iRNAs and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or iRNAs. Microemulsions have also been effective in the transdermal delivery of active components in both cosmetic and pharmaceutical applications. It is expected that the microemulsion compositions and formulations of the technology described herein will facilitate the increased systemic absorption of iRNAs and nucleic acids from the gastrointestinal tract, as well as improve the local cellular uptake of iRNAs and nucleic acids.

Microemulsions of the technology described herein can also contain additional components and additives such as sorbitan monostearate (Grill 3), Labrasol, and penetration enhancers to improve the properties of the formulation and to enhance the absorption of the iRNAs and nucleic acids of the technology described herein. Penetration enhancers used in the microemulsions of the technology described herein can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

In one embodiment, the technology described herein employs various penetration enhancers to effect the efficient delivery of nucleic acids, particularly iRNAs, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, N.Y., 2002; Lee et al., Critical Reviews in Therapeutic Drug Carrier Systems, 1991, p. 92). Each of the above mentioned classes of penetration enhancers are described below in greater detail.

Surfactants: In connection with the technology described herein, surfactants (or “surface-active agents”) are chemical entities which, when dissolved in an aqueous solution, reduce the surface tension of the solution or the interfacial tension between the aqueous solution and another liquid, with the result that absorption of iRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, N.Y., 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, Mass., 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (see e.g., Malmsten, M. Surfactants and polymers in drug delivery, Informa Health Care, New York, N.Y., 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, N.Y., 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agent: Chelating agents, as used in connection with the technology described herein, can be defined as compounds that remove metallic ions from solution by forming complexes therewith, with the result that absorption of iRNAs through the mucosa is enhanced. With regards to their use as penetration enhancers in the technology described herein, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (see e.g., Katdare, A. et al., Excipient development for pharmaceutical, biotechnology, and drug delivery, CRC Press, Danvers, Mass., 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of iRNAs through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of iRNAs at the cellular level can also be added to the pharmaceutical and other compositions of the technology described herein. For example, cationic lipids, such as lipofectin (Junichi et al, U.S. Pat. No. 5,705,188), cationic glycerol derivatives, and polycationic molecules, such as polylysine (Lotto et al., PCT Application WO 97/30731), are also known to enhance the cellular uptake of dsRNAs. Examples of commercially available transfection reagents include, for example Lipofectamine™ (Invitrogen; Carlsbad, Calif.), Lipofectamine 2000™ (Invitrogen; Carlsbad, Calif.), 293Fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPass^{a} D1 Transfection Reagent (New England Biolabs; Ipswich, Mass., USA), LyoVec™/LipoGen™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TroganPORTER™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

Other agents can be utilized to enhance the penetration of the administered nucleic acids, including glycols such as ethylene glycol and propylene glycol, pyrrols such as 2-pyrrol, azones, and terpenes such as limonene and menthone.

Certain compositions of the technology described herein also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′ isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the technology described herein. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the technology described herein can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the technology described herein, such as dyes, flavoring agents, preservatives, antioxidants, opacifiers, thickening agents and stabilizers. However, such materials, when added, should not unduly interfere with the biological activities of the components of the compositions of the technology described herein. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions can contain substances that increase the viscosity of the suspension including, for example, sodium carboxymethylcellulose, sorbitol and/or dextran. The suspension can also contain stabilizers.

In some embodiments, pharmaceutical compositions featured in the technology described herein include (a) one or more iRNA compounds and (b) one or more other agents which function by a non-RNAi mechanism. Examples of such other agents include but are not limited to growth factors (e.g. bone morphogenic protein, basic fibroblast growth factor, platelet-derived growth factor and epidermal growth factor, Stem cell factor, thrombopoietin, Flt3Ligand and 1′-3. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; each incorporated by reference herein in its entirety), hormones (e.g. D-aldosterone, diethylstilbestrol (DES), dexamethasone, β-estradiol, hydrocortisone, insulin, prolactin, progesterone, somatostatin/human growth hormone (HGH), thyrotropin, thyroxine and L-thyronine) and ligands (e.g. c-kit ligand, IL-3, G-CSF, GM-CSF, IL-1, IL-6, IL-11, and flt-3 ligand).

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions described herein lies generally within a range of circulating concentrations that include the ED50 with little or no toxicity. The dosage can vary within this range depending upon the dosage form employed and the route of administration utilized. For any compound used in the methods featured in the technology described herein, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the iRNAs featured in the technology described herein can be administered in combination with other known agents effective in expanding MHCs for transplantation, increasing the population of MHCs ex vivo or in vivo, and increasing MHC transplantation success. In any event, one skilled in the art can adjust the amount and timing of iRNA administration on the basis of results observed using standard measures of efficacy known in the art or described herein.

The technology described herein relates in particular to the use of iRNA targeting a gene or genes encoding a negative regulator(s) of MHC expansion and compositions containing at least one such iRNA for the treatment of a disease requiring a MHC transplantation. The compositions described herein can be used to treat MHC and/or progenitor cells in vitro, thereby enhancing the expansion or engraftment potential of those cells which can then be transplanted into a patient. In another aspect described herein, the MHC and/or progenitor cells and the iRNA compositions described herein are both administered to the patient. In another aspect described herein, the iRNA compositions described herein are administered to a patient in need of MHC expansion and/or engraftment.

For example, a composition containing an iRNA targeting a gene encoding a negative regulator of MHC expansion is used to enhance MHC expansion, engraftment, and hematopoietic repopulation of a patient receiving a UCB MHC transplant where the patient received the transplant to treat one of the following, which are offered by way of example only: leukemia; AML; ALL; CML; Hodgkins' disease; neutropenia; myelodysplastic syndrome; Fanconi's anemia; Blackfan Diamond anemia; severe aplastic anemia; severe combined immunodeficiency; Wiskott-Aldrich syndrome; osteopetrosis; Hurler syndrome; adrenoleukodystrophy; sickle-cell anemia; HIV; Ewing's sarcoma; Gaucher's disease; and thalassemia. By “enhance” in this context is meant a statistically significant increase in such level. The increase can be, for example, at least 10%, at least 20%, at least 30%, at least 40% or more.

The technology described herein further relates to the use of an iRNA or a pharmaceutical composition thereof, e.g., for treating a patient receiving a MHC transplantation, in combination with other pharmaceuticals and/or other therapeutic methods, e.g., with known pharmaceuticals and/or known therapeutic methods, such as, for example, those which can be beneficial to patients receiving a MHC transplant. Examples include, but are not limited to infusions of platelets, infusions of red blood cells, antibiotics (e.g. vancomycin, amphotericin, cyclosporine, ganciclovir, micafungin, fluconazole), anti-nausea medications, growth factors (e.g. bone morphogenic protein, basic fibroblast growth factor, tumor necrosis factor, platelet-derived growth factor and epidermal growth factor, Stem cell factor, thrombopoietin, granulocyte-colony stimulating factor (G-CSF), GM-CSF, Flt3Ligand and 1′-3. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; each incorporated by reference herein in its entirety), hormones (e.g. D-aldosterone, diethylstilbestrol (DES), dexamethasone, β-estradiol, hydrocortisone, insulin, prolactin, progesterone, somatostatin/human growth hormone (HGH), thyrotropin, thyroxine and L-thyronine), ligands (e.g. c-kit ligand, IL-3, G-CSF, GM-CSF, IL-1, IL-1β, IL-6, IL-11, and flt-3 ligand), aminoglycosides, intravenous immune globin, parathyroid hormone (PTH) (e.g. FORTEO® or a peptide as disclosed in US Patent Publication 2008/0051332 which is incorporated herein by reference in its entirety), modulators of the nitric oxide pathway (e.g. sildenafil, vardenafil, tadalafil, apoplipoprotein-E, nitroglycerine, L-arginine, nitrate esters, isoamylynitrite, SIN-1, cysteine, dithiothreitol, N-acetylcysteine, mercaptosuccinic acid, thiosalicylic acid, and methylthiosalicylic acid), dithiocarbamates, disulfram, or prostaglandin E2.

The iRNA and an additional therapeutic agent can be administered in the same combination, e.g., parenterally, or the additional therapeutic agent can be administered as part of a separate composition or by another method described herein.

Efficacy of treatment or prevention of disease can be assessed, for example by measuring neutrophil and/or platelet recovery, engraftment, relapse, survival or any other measurable parameter appropriate. It is well within the ability of one skilled in the art to monitor efficacy of treatment or prevention by measuring any one of such parameters, or any combination of parameters.

A successful treatment is evident when there is a statistically significant improvement in one or more parameters of transplantation recovery status, or by a failure to worsen or to develop symptoms where they would otherwise be anticipated. As an example, a favorable change of at least 10% in a measurable parameter of recovery, and preferably at least 20%, 30%, 40%, 50% or more can be indicative of effective treatment. Efficacy for a given iRNA drug or formulation of that drug can also be judged using an experimental animal model for MHC transplantation as known in the art. When using an experimental animal model, efficacy of treatment is evidenced when a statistically significant increase in a marker is observed.

Patients can be administered a therapeutic amount of iRNA, such as 0.5 mg/kg, 1.0 mg/kg, 1.5 mg/kg, 2.0 mg/kg, or 2.5 mg/kg dsRNA. The iRNA can be administered by intravenous infusion over a period of time, such as over a 5 minute, 10 minute, 15 minute, 20 minute, or 25 minute period. The administration is repeated, for example, on a regular basis, such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Administration of the iRNA can reduce levels of a gene encoding a negative regulator of MHC expansion, e.g., in a cell, tissue, blood, urine or other compartment of the patient by at least 10%, at least 15%, at least 20%, at least 25%, at least 30%, at least 40%, at least 50%, at least 60%, at least 70%, at least 80% or at least 90% or more.

Before administration of a full dose of the iRNA, patients can be administered a smaller dose, such as a 5% infusion reaction, and monitored for adverse effects, such as an allergic reaction.

Owing to the effects on MHC expansion, a composition according to the technology described herein or a pharmaceutical composition prepared therefrom can enhance the quality of life.

Engraftment after lethal ablation of the bone marrow can be assessed by measuring hematopoietic blood cell counts; in particular white blood cell counts. Following lethal ablation, recovery of normal white blood cell counts is a functional measure of successful engraftment. In a clinical context, this can be accompanied by the measurement of cellularity in the bone marrow through serial bone marrow punctions/biopsies and/or by human leukocyte antigen (HLA) typing of circulating white blood cells. Bone marrow aspirates can also be assessed for donor chimerism as a measure of engraftment.

All blood cell types can be indicative of engraftment, but depending on their half lives, provide a more or less sensitive measure of engraftment. Neutrophils have a very short half life (just hours in the blood), and thus are a very good measure of early engraftment. Platelets also have a short half life, but they are usually the last blood element to recover to pre-transplant levels, which can not make them suitable as a marker of early engraftment.

Thus, it is noted herein that cells useful for determining engraftment of hematopoietic progenitor cells are those that recover relatively rapidly following transplantation and have a relatively short half-life (e.g., neutrophils). In one embodiment of the methods described herein, hematopoietic progenitor cell engraftment is assessed by detecting and/or measuring the level of recovery of neutrophils in an individual.

The efficacy of a given treatment to enhance hematopoietic cell engraftment can be determined by the skilled clinician. However, a treatment is considered “effective treatment,” as the term is used herein, if any one or all of the signs or symptoms of e.g., poor hematopoietic cell function or engraftment are altered in a beneficial manner, or other clinically accepted symptoms are improved, or even ameliorated, e.g., by at least 10% following treatment with a compound as described herein. Efficacy can also be measured by a failure of an individual to worsen as assessed by hospitalization, need for medical interventions (i.e., progression of the disease is halted), or incidence of engraftment failure. Methods of measuring these indicators are known to those of skill in the art and/or are described herein. Treatment includes any treatment of a disease in an individual or an animal (some non-limiting examples include a human, or a mammal) and includes: (1) inhibiting the disease, e.g., preventing engraftment failure; or (2) relieving the disease, e.g., causing regression of one or more symptoms. An effective amount for the treatment of a disease means that amount which, when administered to a mammal in need thereof, is sufficient to result in effective treatment as that term is defined herein, for that disease. Efficacy of an agent can be determined by assessing physical indicators of, for example hematopoietic cell engraftment, such as e.g., neutrophil production, white blood cell count, hematopoietic cell numbers, presence/absence of anemia etc. Efficacy can be assessed in animal models of bone marrow transplantation, for example treatment of a rodent following bone marrow transplantation, and any treatment or administration of the compositions or formulations that leads to an increase of at least one symptom of hematopoietic cell engraftment.

In yet another aspect, the technology described herein provides a method for inhibiting the expression of a gene encoding a negative regulator of MHC expansion in a mammal.

In one embodiment, the method includes administering an iRNA composition as described herein to the mammal such that expression of the target gene encoding a negative regulator of MHC expansion is decreased, such as for an extended duration, e.g., at least two, three, four days or more, e.g., one week, two weeks, three weeks, or four weeks or longer. The effect of the decreased target gene preferably results in a increase in neutrophil and/or platelet recovery, engraftment or survival or a decrease in relapse as compared to mammals not receiving the composition.

Preferably, the iRNAs useful for the methods and compositions as described herein specifically target RNAs (primary or processed) of the target gene encoding a negative regulator of MHC expansion. Compositions and methods for inhibiting the expression of these genes using iRNAs can be prepared and performed as described elsewhere herein.

In one embodiment, the method includes administering a composition containing an iRNA, where the iRNA includes a nucleotide sequence that is complementary to at least a part of an RNA transcript of a gene encoding a negative regulator of MHC expansion of the mammal to be treated. When the organism to be treated is a mammal such as a human, the composition can be administered by any means known in the art including, but not limited to oral, intraperitoneal, or parenteral routes, including intracranial (e.g., intraventricular, intraparenchymal and intrathecal), intravenous, intramuscular, subcutaneous, transdermal, airway (aerosol), nasal, rectal, and topical (including buccal and sublingual) administration. In certain embodiments, the compositions are administered by intravenous infusion or injection.

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the technology described herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The technology described herein is further illustrated by the following examples which in no way should be construed as being further limiting.

Some embodiments of the technology described herein can be defined according to any of the following numbered paragraphs:
1. A double-stranded ribonucleic acid (dsRNA) that inhibits expression of a gene encoding a negative regulator of MHC expansion, wherein said negative regulator of MHC expansion is gene selected from the group consisting of: Itch, SH2B3, Prox 1 and AhR.
2. The dsRNA of paragraph 1, wherein said dsRNA comprises a sense strand and an antisense strand, wherein the sense strand comprises at least 15 contiguous nucleotides differing by no more than 3 nucleotides from the nucleotide sequence of SEQ ID NO: 1 and the antisense strand comprises at least 15 contiguous nucleotides differing by no more than 3 nucleotides from the nucleotide sequence of SEQ ID NO: 8.
3. The dsRNA of paragraph 1, wherein said dsRNA comprises a sense strand and an antisense strand, wherein the sense strand comprises at least 15 contiguous nucleotides differing by no more than 3 nucleotides from the nucleotide sequence of SEQ ID NO: 2 and the antisense strand comprises at least 15 contiguous nucleotides differing by no more than 3 nucleotides from the nucleotide sequence of SEQ ID NO: 9.
4. The dsRNA of paragraph 1, wherein said dsRNA comprises a sense strand and an antisense strand, wherein the sense strand comprises at least 15 contiguous nucleotides differing by no more than 3 nucleotides from the nucleotide sequence of SEQ ID NO: 3 and the antisense strand comprises at least 15 contiguous nucleotides differing by no more than 3 nucleotides from the nucleotide sequence of SEQ ID NO: 10.
5. The dsRNA of paragraph 1, wherein said dsRNA comprises a sense strand and an antisense strand, wherein the sense strand comprises at least 15 contiguous nucleotides differing by no more than 3 nucleotides from the nucleotide sequence of SEQ ID NO: 11 and the antisense strand comprises at least 15 contiguous nucleotides differing by no more than 3 nucleotides from the nucleotide sequence of SEQ ID NO: 12.
6. The dsRNA of paragraph 1, wherein said dsRNA comprises a sense strand and an antisense strand, the antisense strand comprising a region of complementarity which comprises at least 15 contiguous nucleotides differing by no more than 3 nucleotides from one of the antisense sequences listed in Tables 2-7.
7. The dsRNA of paragraph 1, wherein said dsRNA comprises at least one modified nucleotide.
8. The dsRNA of paragraph 7, wherein at least one of said modified nucleotides is chosen from the group of: a 2′-O-methyl modified nucleotide, a nucleotide comprising a 5′-phosphorothioate group, and a terminal nucleotide linked to a cholesteryl derivative or dodecanoic acid bisdecylamide group.
9. The dsRNA of paragraph 7, wherein said modified nucleotide is chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.
10. The dsRNA of paragraph 6, wherein the region of complementary is at least 17 nucleotides in length.
11. The dsRNA of paragraph 6, wherein the region of complementarity is between 19 and 21 nucleotides in length.
12. The dsRNA of paragraph 11, wherein the region of complementarity is 19 nucleotides in length.
13. The dsRNA of paragraph 1, wherein each strand is no more than 30 nucleotides in length.
14. The dsRNA of paragraph 1, wherein at least one strand comprises a 3′ overhang of at least 1 nucleotide.
15. The dsRNA of paragraph 1, wherein at least one strand comprises a 3′ overhang of at least 2 nucleotides.
16. The dsRNA of paragraph 1, further comprising a ligand.
17. The dsRNA of paragraph 16, wherein the ligand is conjugated to the 3′ end of the sense strand of the dsRNA.
18. The dsRNA of paragraph 6, wherein the region of complementarity consists of one of the antisense sequences of Tables 2-7.
19. The dsRNA of paragraph 6, wherein the sense strand consists of SEQ ID NO: 1 and the antisense strand consists of SEQ ID NO: 8.
20. The dsRNA of paragraph 6, wherein the sense strand consists of SEQ ID NO: 2 and the antisense strand consists of SEQ ID NO: 9.
21. The dsRNA of paragraph 6, wherein the sense strand consists of SEQ ID NO: 3 and the antisense strand consists of SEQ ID NO: 10.
22. The dsRNA of paragraph 1 wherein the dsRNA comprises a sense strand consisting of a sense strand sequence selected from Tables 2-7, and an antisense strand consisting of an antisense sequence selected from Tables 2-7.
23. A cell containing the dsRNA of paragraph 1.
24. An isolated or cultured cell containing the dsRNA of paragraph 1.
25. A vector encoding at least one strand of a dsRNA of any of paragraphs 1-22.
26. The vector of paragraph 25, wherein the region of complementarity is at least 15 nucleotides in length.
27. The vector of paragraph 25, wherein the region of complementarity is 19 to 21 nucleotides in length.
28. A cell comprising the vector of paragraph 25.
29. An isolated or cultured cell containing the vector of paragraph 25.
30. A method of inhibiting expression of a gene encoding a negative regulator of MHC expansion in a cell, the method comprising:
   (a) introducing into the cell the dsRNA of any of paragraphs 1-22; and
   (b) maintaining the cell produced in step (a) for a time sufficient to obtain degradation of the mRNA transcript of a gene encoding a negative regulator of MHC expansion, thereby inhibiting expression of the gene encoding a negative regulator of MHC expansion in the cell.
31. The method of paragraph 30, wherein the expression of the gene encoding a negative regulator of MHC expansion is inhibited by at least 30%.
32. A cell treated according to the method of paragraph 30.
33. An isolated or cultured cell treated according to the method of paragraph 30.
34. A method of expansion of a multipotent hematopoietic cell, the method comprising introducing into an multipotent hematopoietic cell a dsRNA of any of paragraphs 1-22 and maintaining the cell for a time and under conditions sufficient to permit expansion of the cell.
35. The method of paragraph 34 wherein resulting expanded cells have enhanced ability to engraft and/or differentiate relative to a multipotent hematopoietic cell that has not been treated according to the method of paragraph 34.
36. The method of paragraph 34, wherein said expansion occurs ex vivo.
37. The method of paragraph 34, wherein said expansion occurs in vivo.
38. A cell treated according to the method of paragraph 34.
39. An isolated or cultured cell treated according to the method of paragraph 34.
40. A method of treating a patient in need of enhanced multipotent hematopoietic cell expansion and/or engraftment, wherein said method comprises administering to the patient a treatment comprising one or more of the following:
   a dsRNA of any of paragraphs 1-22; a cell of any of paragraphs 23, 24, 28, 29, 32, 33, 38 and 39; and a vector of any of paragraphs 21-23.

### EXAMPLES

### Example 1

### iRNA Synthesis

### Source of Reagents

Where the source of a reagent is not specifically given herein, such reagent can be obtained from a supplier of reagents for molecular biology at a quality/purity standard for application in molecular biology.

### Oligonucleotide Synthesis

Oligonucleotides are synthesized on an AKTAoligopilot synthesizer. Commercially available controlled pore glass solid support (dT-CPG, 500 Å, Prime Synthesis) and RNA phosphoramidites with standard protecting groups, 5′-O-dimethoxytrityl N6-benzoyl-2′-t-butyldimethylsilyl-adenosine-3′-O—N,N′-diisopropyl-2-cyanoethylphosphoramidite, 5′-O-dimethoxytrityl-N4-acetyl-2′-t-butyldimethylsilyl-cytidine-3′-O—N,N′-diisopropyl-2-cyanoethylphosphoramidite, 5′-O-dimethoxytrityl-N2-isobutryl-2′-t-butyldimethylsilyl-guanosine-3′-O—N,N′-diisopropyl-2-cyanoethylphosphoramidite, and 5′-O-dimethoxytrityl-2′-t-butyldimethylsilyl-uridine-3′-O—N,N′-diisopropyl-2-cyanoethylphosphoramidite (Pierce Nucleic Acids Technologies) were used for the oligonucleotide synthesis. The 2′-F phosphoramidites, 5′-O-dimethoxytrityl-N4-acetyl-2′-fluoro-cytidine-3′-O—N,N′-diisopropyl-2-cyanoethylphosphoramidite and 5′-O-dimethoxytrityl-2′-fluoro-uridine-3′-O—N,N′-diisopropyl-2-cyanoethylphosphoramidite are purchased from (Promega). Phosphoramidites are used at a concentration of 0.2M in acetonitrile (CH₃CN) except for guanosine which is used at 0.2M concentration in 10% THF/ANC (v/v). Coupling/recycling time of 16 minutes is used. The activator is 5-ethyl thiotetrazole (0.75M, American International Chemicals); for the PO-oxidation iodine/water/pyridine is used and for the PS-oxidation PADS (2%) in 2,6-lutidine/ACN (1:1 v/v) is used.

3′-ligand conjugated strands are synthesized using solid support containing the corresponding ligand. For example, the introduction of cholesterol unit in the sequence is performed from a hydroxyprolinol-cholesterol phosphoramidite. Cholesterol is tethered to trans-4-hydroxyprolinol via a 6-aminohexanoate linkage to obtain a hydroxyprolinol-cholesterol moiety. 5′-end Cy-3 and Cy-5.5 (fluorophore) labeled iRNAs are synthesized from the corresponding Quasar-570 (Cy-3) phosphoramidite are purchased from Biosearch Technologies. Conjugation of ligands to 5′-end and or internal position is achieved by using appropriately protected ligand-phosphoramidite building block. An extended 15 min coupling of 0.1 M solution of phosphoramidite in anhydrous CH₃CN in the presence of 5-(ethylthio)-1H-tetrazole activator to a solid-support-bound oligonucleotide. Oxidation of the internucleotide phosphite to the phosphate is carried out using standard iodine-water as reported (1) or by treatment with tert-butyl hydroperoxide/acetonitrile/water (10:87:3) with 10 min oxidation wait time conjugated oligonucleotide. Phosphorothioate is introduced by the oxidation of phosphite to phosphorothioate by using a sulfur transfer reagent such as DDTT (purchased from AM Chemicals), PADS and or Beaucage reagent. The cholesterol phosphoramidite is synthesized in house and used at a concentration of 0.1 M in dichloromethane. Coupling time for the cholesterol phosphoramidite is 16 minutes.

### Deprotection I (Nucleobase Deprotection)

After completion of synthesis, the support is transferred to a 100 mL glass bottle (VWR). The oligonucleotide is cleaved from the support with simultaneous deprotection of base and phosphate groups with 80 mL of a mixture of ethanolic ammonia [ammonia:ethanol (3:1)] for 6.5 h at 55° C. The bottle is cooled briefly on ice and then the ethanolic ammonia mixture is filtered into a new 250-mL bottle. The CPG is washed with 2×40 mL portions of ethanol/water (1:1 v/v). The volume of the mixture is then reduced to −30 mL by roto-vap. The mixture is then frozen on dry ice and dried under vacuum on a speed vac.

### Deprotection II (Removal of 2′-TBDMS Group)

The dried residue is resuspended in 26 mL of triethylamine, triethylamine trihydrofluoride (TEA.3HF) or pyridine-HF and DMSO (3:4:6) and heated at 60° C. for 90 minutes to remove the tert-butyldimethylsilyl (TBDMS) groups at the 2′ position. The reaction is then quenched with 50 mL of 20 mM sodium acetate and the pH is adjusted to 6.5. Oligonucleotide is stored in a freezer until purification.

### Analysis

The oligonucleotides are analyzed by high-performance liquid chromatography (HPLC) prior to purification and selection of buffer and column depends on nature of the sequence and or conjugated ligand.

### HPLC Purification

The ligand-conjugated oligonucleotides are purified by reverse-phase preparative HPLC. The unconjugated oligonucleotides are purified by anion-exchange HPLC on a TSK gel column packed in house. The buffers are 20 mM sodium phosphate (pH 8.5) in 10% CH₃CN (buffer A) and 20 mM sodium phosphate (pH 8.5) in 10% CH₃CN, 1M NaBr (buffer B). Fractions containing full-length oligonucleotides are pooled, desalted, and lyophilized. Approximately 0.15 OD of desalted oligonucleotides are diluted in water to 150 μL and then pipetted into special vials for CGE and LC/MS analysis. Compounds are then analyzed by LC-ESMS and CGE.

iRNA Preparation

For the general preparation of iRNA, equimolar amounts of sense and antisense strand are heated in 1×PBS at 95° C. for 5 mM and slowly cooled to room temperature. Integrity of the duplex is confirmed by HPLC analysis.

Nucleic acid sequences are represented below using standard nomenclature, and specifically the abbreviations of Table 1.

TABLE 1
Abbreviations of nucleotide monomers used in nucleic
acid sequence representation. It will be understood that
these monomers, when present in an oligonucleotide, are
mutually linked by 5′-3′-phosphodiester bonds.

| Abbreviation | Nucleotide(s) |
|---|---|
| A | adenosine |
| C | cytidine |
| G | guanosine |
| T | thymidine |
| U | uridine |
| N | any nucleotide (G, A, C, T or U) |
| a | 2′-O-methyladenosine |
| c | 2′-O-methylcytidine |
| g | 2′-O-methylguanosine |
| u | 2′-O-methyluridine |
| dT | 2′-deoxythymidine |
| s | phosphorothioate linkage |

### Example 2

### siRNA Design

### Transcripts

siRNAs targeting Itch, SH2B3, Ahr, and Prox1 were designed and synthesized. The design used human transcripts from the NCBI Refseq collection. siRNA duplexes were designed with 100% identity to the target gene.

A total of 186 sense and 186 antisense human derived siRNA oligos were synthesized and formed into duplexes. The oligos are presented in Tables 2-7.

Synthesis of Sequences for iRNA Agents

Sequences were synthesized on MerMade 192 synthesizer at 1 μmol scale. For all the sequences in Table 3, ‘endolight’ chemistry was applied as detailed below.

All pyrimidines (cytosine and uridine) in the sense strand were replaced with corresponding 2′-O-Methyl bases (2′ O-Methyl C and 2′-O-Methyl U)
In the antisense strand, pyrimidines adjacent to (towards 5′ position) ribo A nucleoside were replaced with their corresponding 2-O-Methyl nucleosides
A two base dTsdT extension at 3′ end of both sense and anti sense sequences was introduced
The sequence file was converted to a text file to make it compatible for loading in the MerMade 192 synthesis software

The synthesis of the polynucleotides described herein used solid supported oligonucleotide synthesis using phosphoramidite chemistry.

The synthesis of the above sequences was performed at 1 □m scale in 96 well plates. The amidite solutions were prepared at 0.1M concentration and ethyl thio tetrazole (0.6M in Acetonitrile) was used as activator.

The synthesized sequences were cleaved and deprotected in 96 well plates, using methylamine in the first step and pyridine.3HF in the second step. The crude sequences thus obtained were precipitated using acetone: ethanol mix and the pellets were re-suspended in 0.5M sodium acetate buffer. Samples from each sequence were analyzed by LC-MS and the resulting mass data confirmed the identity of the sequences. A selected set of samples were also analyzed by IEX chromatography.

The next step in the process was purification. All sequences were purified on AKTA explorer purification system using Source 15Q column. A single peak corresponding to the full length sequence was collected in the eluent and was subsequently analyzed for purity by ion exchange chromatography.

The purified sequences were desalted on a Sephadex G25 column using AKTA purifier. The desalted sequences were analyzed for concentration and purity. The single strands were then submitted for annealing

### Example 3

### Screening of SH2B3 siRNA in Cultured Cells

### Cell Culture and Transfections

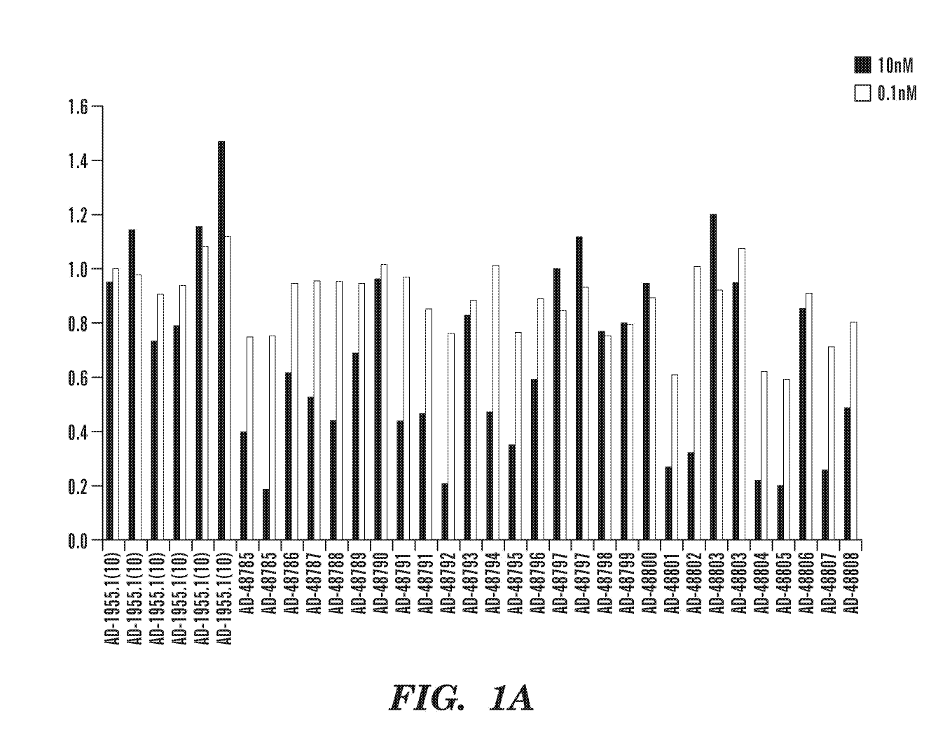

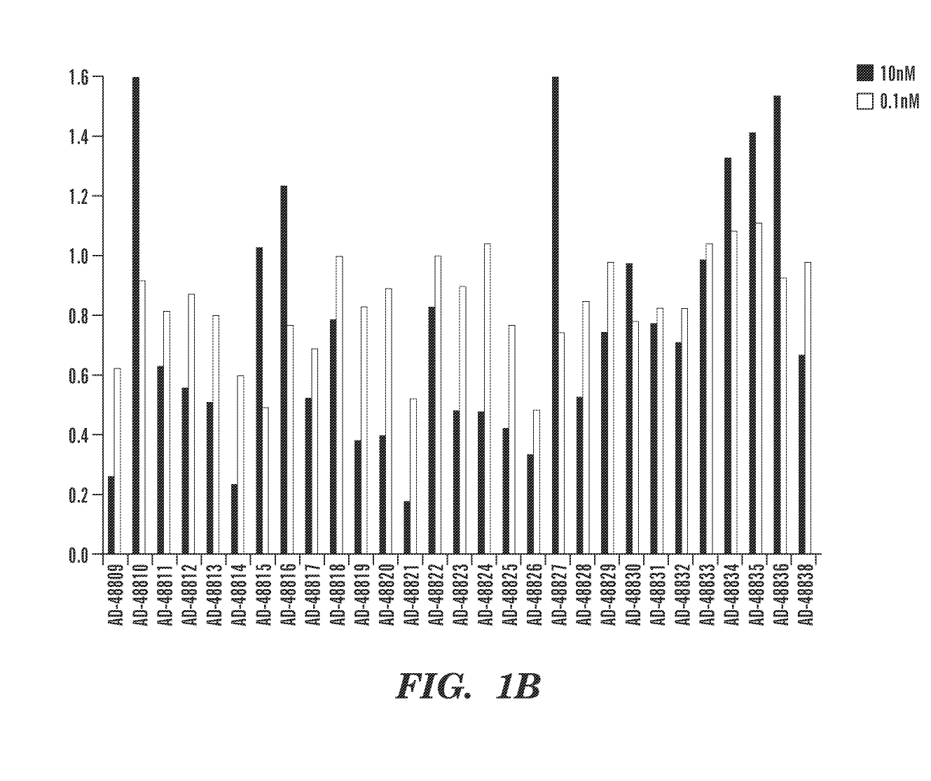

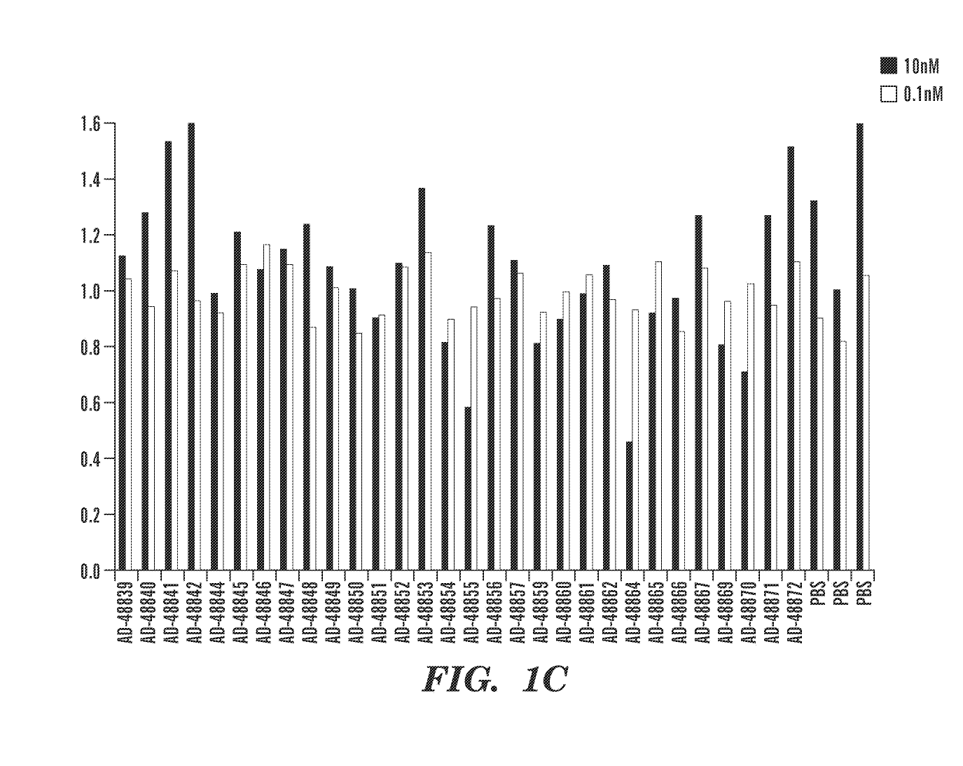

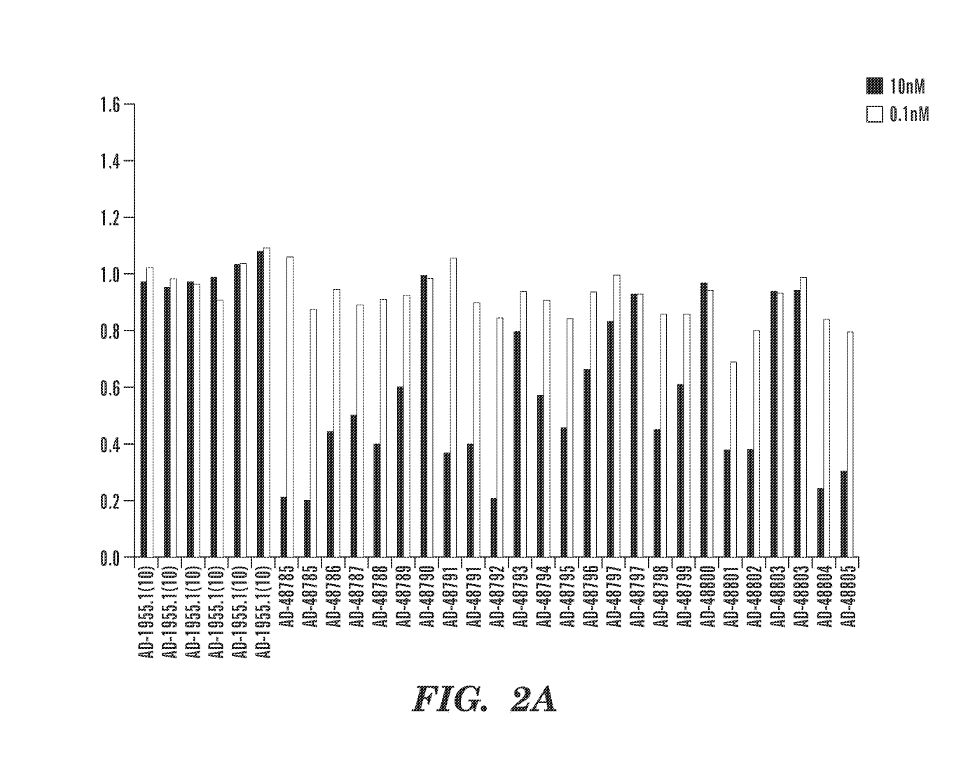

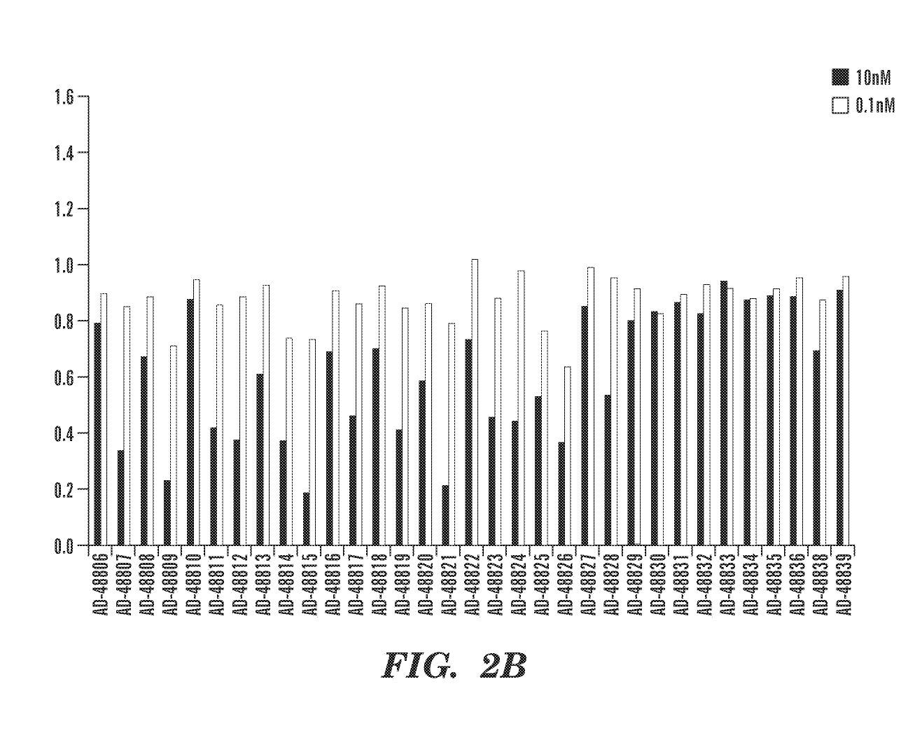

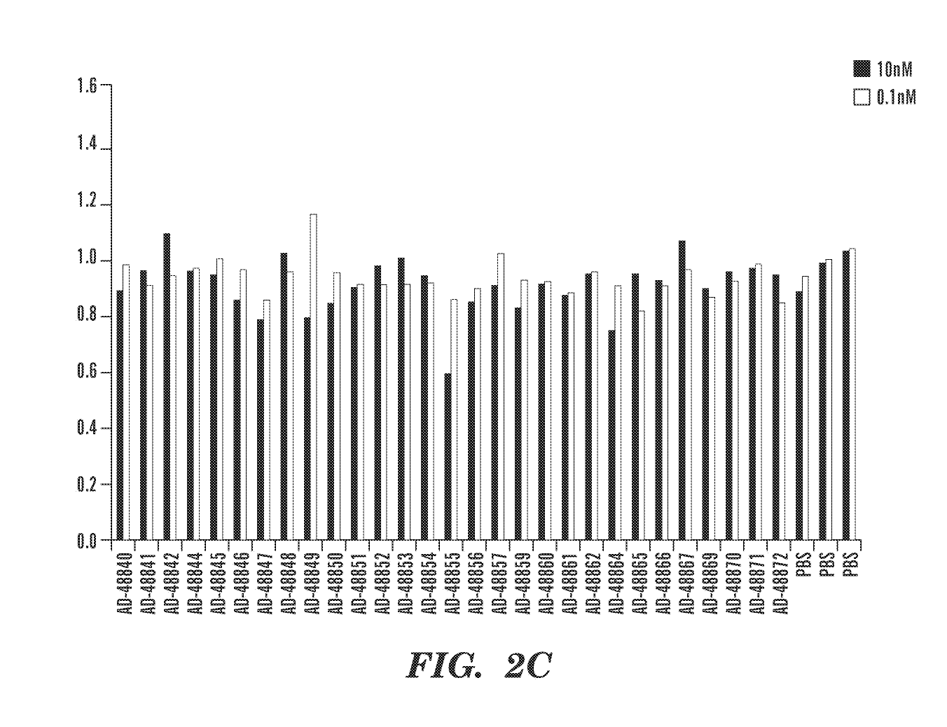

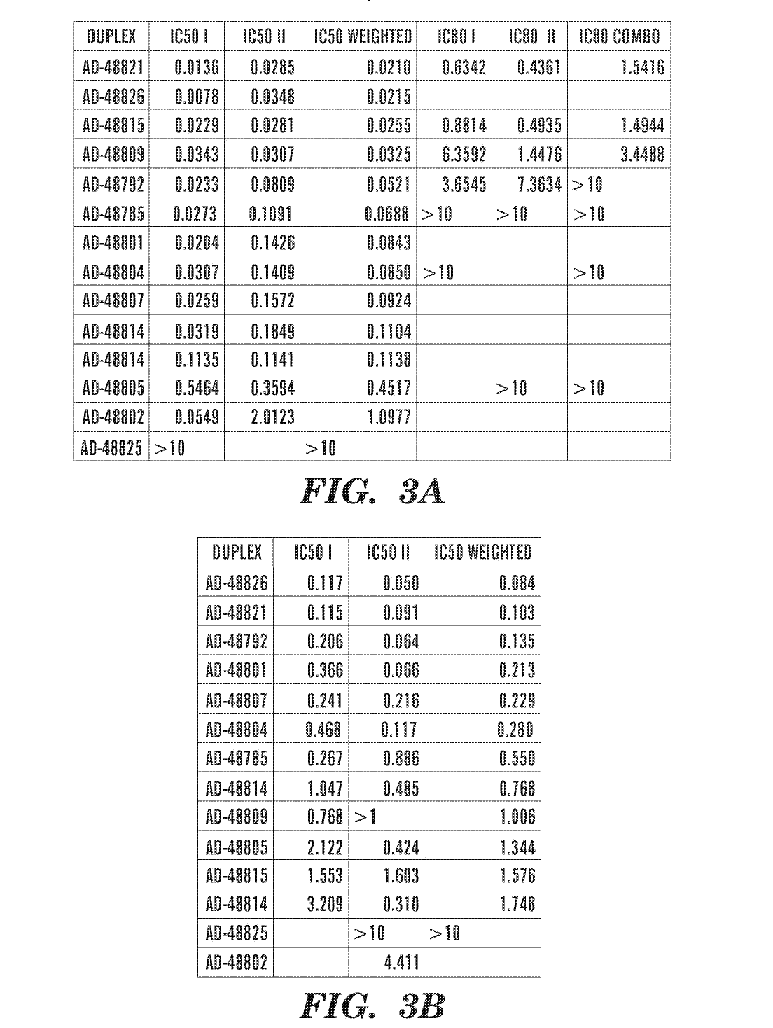

RKO and HEp3b cells were grown to near confluence at 37° C. in an atmosphere of 5% CO2 in MEM (INVITROGEN™) supplemented with 10% FBS before being released from the plate by trypsinization. Transfection was carried out by mixing 5 μl of siRNA duplexes per well into a 96-well plate along with 44.75 μl of Opti-MEM plus 0.25 μl of Lipofectamine RNAiMax per well (Invitrogen™, Carlsbad Calif. cat #13778-150) and incubated at room temperature for 15 minutes. Subsequently 20000 cells (50 ul) were added to the transfection mix and incubated for 24 hours prior to RNA purification. Experiments were performed at 10 nM or 0.1 nM final duplex concentration for single dose screens with each of the SH2B3 duplexes (FIGS. 1A-1C and 2A-2C). A subset of 14 duplexes that showed robust silencing in the single dose screens were assayed over a range of concentrations from 10 nM to 10 fM using serial dilutions to determine their IC₅₀ (FIGS. 3A-3B).

Total RNA was isolated using Dynabeads™ mRNA Isolation protocol (Life Technologies, Carlsbard Calif., part #: 610-12)

cDNA was synthesized using ABI High capacity cDNA reverse transcription kit (Applied Biosystems, Foster City, Calif., Cat #4368813)

A master mix of 2 μl 10× Buffer, 0.8 μl 25×dNTPs, 2 μl Random primers, 1 μl Reverse Transcriptase, 1 μl RNase inhibitor and 3.2 μl of H2O per reaction were added into 10 μl total RNA. cDNA was generated using a MJ Research or Bio-Rad C-1000 or S-1000 thermal cycler (Hercules, Calif.) through the following steps: 25° C. 10 min, 37° C. 120 min, 85° C. 5 sec, 4° C. hold.

### Real Time PCR

2 μl of cDNA were added to a master mix containing 0.5 μl GAPDH TaqMan Probe (Applied Biosystems Cat #4326317E), 0.5 μl SH2B3 TaqMan probe (Applied Biosystems Cat#Hs00193878 ml) and 5 μl Roche Probes Master Mix (Roche Cat #04887301001) in a total of 10 μl per well in a LightCycler 480 384 well plate (Roche cat #0472974001). Real time PCR was done in a LightCycler 480 Real Time PCR machine (Roche). Each duplex was tested in at least two independent transfections. Each transfection was assayed by qPCR in duplicate.

Real time data were analyzed using the ΔΔCt method. Each sample was normalized to GAPDH expression and knockdown was assessed relative to cells transfected with the non-targeting duplex AD-1955. IC₅₀s were defined using a 4 parameter fit model in XLfit. As shown in FIGS. 1A-1C (Hep3b cells) and FIGS. 2A-2C (RKO cells), the efficacy of single doses of SH2B3 siRNA was examined in vitro.

Other embodiments are in the claims.

TABLE 2
siRNAs specific for human Ahr. Start position is position of 5′ sense
base on transcript NM_001621 (SEQ ID NO: 3).

| | | SEQ | | SEQ | |
|---|---|---|---|---|---|
| | Start | ID | | ID | |
| oligoName | Position | NO: | sense (5′-3′) | NO: | antis (5′-3′) |
| NM_001621.4_727- | 727 | 100 | gcauagagaccgacuuaau | 150 | auuaagucggucucuaugc |
| 745_s | | | | | |
| NM_001621.4_1104- | 1104 | 101 | ccgaagaccgagcugaauu | 151 | aauucagcucggucuucgg |
| 1122_s | | | | | |
| NM_001621.4_1205- | 1205 | 102 | cagacaguagucuguuaua | 152 | uauaacagacuacugucug |
| 1223_s | | | | | |
| NM_001621.4_1371- | 1371 | 103 | ggaaagauggaucaauacu | 153 | aguauugauccaucuuucc |
| 1389_s | | | | | |
| NM_001621.4_1495- | 1495 | 104 | cuucacaccuauugguugu | 154 | acaaccaauaggugugaag |
| 1513_s | | | | | |
| NM_001621.4_1561- | 1561 | 105 | cacgagaggcucagguuau | 155 | auaaccugagccucucgug |
| 1579_s | | | | | |
| NM_001621.4_1563- | 1563 | 106 | cgagaggcucagguuauca | 156 | ugauaaccugagccucucg |
| 1581_s | | | | | |
| NM_001621.4_1613- | 1613 | 107 | gccgagucccauauccgaa | 157 | uucggauaugggacucggc |
| 1631_s | | | | | |
| NM_001621.4_1614- | 1614 | 108 | ccgagucccauauccgaau | 158 | auucggauaugggacucgg |
| 1632_s | | | | | |
| NM_001621.4_1620- | 1620 | 109 | cccauauccgaaugauuaa | 159 | uuaaucauucggauauggg |
| 1638_s | | | | | |
| NM_001621.4_1646- | 1646 | 110 | gaaaguggcaugauaguuu | 160 | aaacuaucaugccacuuuc |
| 1664_s | | | | | |
| NM_001621.4_1793- | 1793 | 111 | cauuuacgaaaacgaaaua | 161 | uauuucguuuucguaaaug |
| 1811_s | | | | | |
| NM_001621.4_1958- | 1958 | 112 | gacucucucaauccuaguu | 162 | aacuaggauugagagaguc |
| 1976_s | | | | | |
| NM_001621.4_1997- | 1997 | 113 | caacaagaugagucuauuu | 163 | aaauagacucaucuuguug |
| 2015_s | | | | | |
| NM_001621.4_2020- | 2020 | 114 | cuauccugcuucaaguacu | 164 | aguacuugaagcaggauag |
| 2038_s | | | | | |
| NM_001621.4_2062- | 2062 | 115 | caacuuuuucaacgaaucu | 165 | agauucguugaaaaaguug |
| 2080_s | | | | | |
| NM_001621.4_2117- | 2117 | 116 | ccgaugggaaaugauacua | 166 | uaguaucauuucccaucgg |
| 2135_s | | | | | |
| NM_001621.4_2198- | 2198 | 117 | gggcucuuucaagauagua | 167 | uacuaucuugaaagagccc |
| 2216_s | | | | | |
| NM_001621.4_2357- | 2357 | 118 | cugacguauguecaagauu | 168 | aaucuuggacauacgucag |
| 2375_s | | | | | |
| NM_001621.4_2359- | 2359 | 119 | gacguauguccaagauucu | 169 | agaaucuuggacauacguc |
| 2377_s | | | | | |
| NM_001621.4_2585- | 2585 | 120 | caauucgugccuuucaauu | 170 | aauugaaaggcacgaauug |
| 2603_s | | | | | |
| NM_001621.4_2618- | 2618 | 121 | ccacaacaauauauugucu | 171 | agacauuauauuguugugg |
| 2636_s | | | | | |
| NM_001621.4_2644- | 2644 | 122 | cuuacaugggaucagucaa | 172 | uugacugaucccauguaag |
| 2662_s | | | | | |
| NM_001621.4_2913- | 2913 | 123 | gggccgugucgauguauca | 173 | ugauacaucgacacggccc |
| 2931_s | | | | | |
| NM_001621.4_2915- | 2915 | 124 | gccgugucgauguaucagu | 174 | acugauacaucgacacggc |
| 2933_s | | | | | |
| NM_001621.4_3144- | 3144 | 125 | ccaguggauuccuguaauu | 175 | aauuacaggaauccacugg |
| 3162_s | | | | | |
| NM_001621.4_3394- | 3394 | 126 | gugagguaccgucuacauu | 176 | aauguagacgguaccucac |
| 3412_s | | | | | |
| NM_001621.4_3731- | 3731 | 127 | cacucuaggauguauauuu | 177 | aaauauacauccuagagug |
| 3749_s | | | | | |
| NM_001621.4_3833- | 3833 | 128 | cauuaauucaggcauauuu | 178 | aaauaugccugaauuaaug |
| 3851_s | | | | | |
| NM_001621.4_3953- | 3953 | 129 | gcuaugugccuuauguuga | 179 | ucaacauaaggcacauagc |
| 3971_s | | | | | |
| NM_001621.4_4284- | 4284 | 130 | gacuuuauaaauuucgcuu | 180 | aagcgaaauuuauaaaguc |
| 4302_s | | | | | |
| NM_001621.4_4497- | 4497 | 131 | guuggggcuuuacauacuu | 181 | aaguauguaaagccccaac |
| 4515_s | | | | | |
| NM.001621.4_4548- | 4548 | 132 | gaauccaacugcuuaaagu | 182 | acuuuaagcaguuggauuc |
| 4566_s | | | | | |
| NM.001621.4_4553- | 4553 | 133 | caacugcuuaaaguuggua | 183 | uaccaacuuuaagcaguug |
| 4571_s | | | | | |
| NM_001621.4_4558- | 4558 | 134 | gcuuaaaguugguauuaau | 184 | auuaauaccaacuuuaagc |
| 4576_s | | | | | |
| NM_001621.4_4584- | 4584 | 135 | caaccacauaguucguuua | 185 | uaaacgaacuaugugguug |
| 4602_s | | | | | |
| NM_001621.4_4588- | 4588 | 136 | cacauaguucguuuaccuu | 186 | aagguaaacgaaeuaugug |
| 4606_s | | | | | |
| NM_001621.4_5055- | 5055 | 137 | gcuuagacacauuuugcau | 187 | augcaaaaugugucuaagc |
| 5073-s | | | | | |
| NM_001621.4_5474- | 5474 | 138 | ggcuucggacaaaauaucu | 188 | agauauuuuguccgaagcc |
| 5492_s | | | | | |
| NM_001621.4_5611- | 5611 | 139 | gaceagaggucuagauuaa | 189 | uuaaucuagaccucugguc |
| 5629_s | | | | | |
| NM_001621.4_5613- | 5613 | 140 | ccagaugucuagauuaaua | 190 | uauuaaucuagaccucugg |
| 5631_s | | | | | |
| NM_001621.4_5754- | 5754 | 141 | gacaaucaguuauacaguu | 191 | aacuguauaacugauuguc |
| 5772_s | | | | | |
| NM_001621.4_5856- | 5856 | 142 | cguuaaaccaaauacacgu | 192 | acguguauuugguuuaacg |
| 5874_s | | | | | |
| NM_001621.4_5893- | 5893 | 143 | gccaaacaaaggauacuua | 193 | uaaguauccuuuguuuggc |
| 5911_s | | | | | |
| NM_001621.4_5968- | 5968 | 144 | gaugugucuaauuuagucu | 194 | agacuaaauuagacacauc |
| 5986_s | | | | | |
| NM_001621.4_6074- | 6074 | 145 | cuaguguuuuucgauguua | 195 | uaacaucgaaaaacacuag |
| 6092_s | | | | | |
| NM_001621.4_6079- | 6079 | 146 | guuuuucgauguuacuaaa | 196 | uuuaguaacaucgaaaaac |
| 6097_s | | | | | |

TABLE 3
siRNAs specific for human Prox1. Start position is position of 5′ sense
base on transcript NM_002763 (SEQ ID NO: 2).

| | | SEQ | | SEQ | |
|---|---|---|---|---|---|
| | Start | ID | | ID | |
| oligoName | Position | NO: | sense (5′-3′) | NO: | antis (5′-3′) |
| NM_002763.3_56-74_s | 56 | 200 | gagccucccauuacucaga | 250 | ucugaguaaugggaggcuc |
| NM_002763.3_185- | 185 | 201 | cgcuccucccaguuccuaa | 251 | uuaggaacugggaggagcg |
| 203_s | | | | | |
| NM_002763.3_769- | 769 | 202 | gcgcccggguugagaauau | 252 | auauucucaacccgggcgc |
| 787_s | | | | | |
| NM_002763.3_770- | 770 | 203 | cgcccggguugagaauaua | 253 | uauauucucaacccgggcg |
| 788_s | | | | | |
| NM_002763.3_773- | 773 | 204 | ccggguugagaauauaauu | 254 | aauuauauucucaacccgg |
| 791_s | | | | | |
| NM_002763.3_1058- | 1058 | 205 | cagcacugauucggaaaau | 255 | auuuuccgaaucagugcug |
| 1076_s | | | | | |
| NM_002763.3_1144- | 1144 | 206 | cugucggaaggucagauaa | 256 | uuaucugaccuuccgacag |
| 1162_s | | | | | |
| NM_002763.3_1296- | 1296 | 207 | caaccggaaggcaaacauu | 257 | aauguuugccuuccgguug |
| 1314_s | | | | | |
| NM_002763.3_1732- | 1732 | 208 | ccuugauggccuauccauu | 258 | aauggauaggccaucaagg |
| 1750_s | | | | | |
| NM_002763.3_1948- | 1948 | 209 | gcggcgaucuucaagauau | 259 | auaucuugaagaucgccgc |
| 1966_s | | | | | |
| NM_002763.3_2008- | 2008 | 210 | gauugucacccaaucacuu | 260 | aagugauugggugacaauc |
| 2026_s | | | | | |
| NM_002763.3_2138- | 2138 | 211 | gugguuuagcaauuuccgu | 261 | acggaaauugcuaaaccac |
| 2156_s | | | | | |
| NM_002763.3_2209- | 2209 | 212 | gggucaccaguacugaaga | 262 | ucuucaguacuggugaccc |
| 2227_s | | | | | |
| NM_002763.3_2223- | 2223 | 213 | gaagagcugucuauaacca | 263 | ugguuauagacagcucuuc |
| 2241_s | | | | | |
| NM_002763.3_2414- | 2414 | 214 | cugcaagcuggauagugaa | 264 | uucacuauccagcuugcag |
| 2432_s | | | | | |
| NM_002763.3_2416- | 2416 | 215 | gcaagcuggauagugaagu | 265 | acuucacuauccagcuugc |
| 2434_s | | | | | |
| NM_002763.3_2469- | 2469 | 216 | gagcugcuucaugaguaga | 266 | ucuacucaugaagcagcuc |
| 2487_s | | | | | |
| NM_002763.3_2581- | 2581 | 217 | guaugggaggcauggauau | 267 | auauccaugccucccauac |
| 2599_s | | | | | |
| NM_002763.3_2705- | 2705 | 218 | gcccaaggcccuuaacauu | 268 | aauguuaagggccuugggc |
| 2723_s | | | | | |
| NM_002763.3_2726- | 2726 | 219 | gacacuuaaaauaggguua | 269 | uaacccuauuuuaaguguc |
| 2744_s | | | | | |
| NM_002763.3_2869- | 2869 | 220 | gacacagcacaacucuaca | 270 | uguagaguugugcuguguc |
| 2887_s | | | | | |
| NM_002763.3_2943- | 2943 | 221 | ccacuucauacauuuaagu | 271 | acuuaaauguaugaagugg |
| 2961_s | | | | | |
| NM_002763.3_2944- | 2944 | 222 | cacuucauacauuuaagua | 272 | uacuuaaauguaugaagug |
| 2962_s | | | | | |

TABLE 4
siRNAs specific for human and murine Prox1. Start position is position
of 5′ sense base on transcript NM_002763 (SEQ ID NO: 2).

| | | SEQ | | SEQ | |
|---|---|---|---|---|---|
| | Start | ID | | ID | |
| oligoName | Position | NO: | sense (5′-3′) | NO: | antis (5′-3′) |
| NM_002763.3_227- | 227 | 300 | ugcccagcugacgagcuuu | 350 | aaagcucgucagcugggca |
| 245_s | | | | | |
| NM_002763.3_228- | 228 | 301 | gcccagcugacgagcuuuu | 351 | aaaagcucgucagcugggc |
| 246_s | | | | | |
| NM_002763.3_230- | 230 | 302 | ccagcugacgagcuuuuga | 352 | ucaaaagcucgucagcugg |
| 248_s | | | | | |
| NM_002763.3_231- | 231 | 303 | cagcugacgagcuuuugaa | 353 | uucaaaagcucgucagcug |
| 249_s | | | | | |
| NM_002763.3_233- | 233 | 304 | gcugacgagcuuuugaaga | 354 | ucuucaaaagcucgucagc |
| 251_s | | | | | |
| NM_002763.3_234- | 234 | 305 | cugacgagcuuuugaagau | 355 | aucuucaaaagcucgucag |
| 252_s | | | | | |
| NM_002763.3_298- | 298 | 306 | ucuuaagccggcaaaccaa | 356 | uugguuugccggcuuaaga |
| 316_s | | | | | |
| NM_002763.3_352- | 352 | 307 | uagggacagcaucugcauu | 357 | aaugcagaugcugucccua |
| 370_s | | | | | |
| NM_002763.3_628- | 628 | 308 | caggcuccgaaguacauca | 358 | ugauguacuucggagccug |
| 646_s | | | | | |
| NM_002763.3_690- | 690 | 309 | ugucuuuccccuuuuggca | 359 | ugccaaaaggggaaagaca |
| 708_s | | | | | |
| NM_002763.3_712- | 712 | 310 | cuacuaugagccaguuuga | 360 | ucaaacuggcucauaguag |
| 730_s | | | | | |
| NM_002763.3_747- | 747 | 311 | gaugagcaccugagagcaa | 361 | uugcucucaggugcucauc |
| 765_s | | | | | |
| NM_002763.3_748- | 748 | 312 | augagcaccugagagcaaa | 362 | uuugcucucaggugcucau |
| 766_s | | | | | |
| NM_002763.3_849- | 849 | 313 | auggccccgcagucuguga | 363 | ucacagacugcggggccau |
| 867_s | | | | | |
| NM_002763.3_867- | 867 | 314 | aguccccgagaaaguuaca | 364 | uguaacuuucucggggacu |
| 885_s | | | | | |
| NM_002763.3_869- | 869 | 315 | uccccgagaaaguuacaga | 365 | ucuguaacuuucucgggga |
| 887_s | | | | | |
| NM_002763.3_882- | 882 | 316 | uacagagaaaacaaacgca | 366 | ugcguuuguuuucucugua |
| 900_s | | | | | |
| NM_002763.3_883- | 883 | 317 | acagagaaaacaaacgcaa | 367 | uugcguuuguuuucucugu |
| 901_s | | | | | |
| NM_002763.3_943- | 943 | 318 | ugguuucagcccgaaaaga | 368 | ucuuuucgggcugaaacca |
| 961_s | | | | | |
| NM_002763.3_944- | 944 | 319 | gguuucagcccgaaaagaa | 369 | uucuuuucgggcugaaacc |
| 962_s | | | | | |
| NM_002763.3_2013- | 2013 | 320 | ucacccaaucacuugaaaa | 370 | uuuucaagugauuggguga |
| 2031_s | | | | | |
| NM_002763.3_2145- | 2145 | 321 | agcaauuuccgugaguuuu | 371 | aaaacucacggaaauugcu |
| 2163_s | | | | | |
| NM_002763.3_2148- | 2148 | 322 | aauuuccgugaguuuuacu | 372 | aguaaaacucacggaaauu |
| 2166_s | | | | | |

TABLE 5
siRNAs specific for human SH2B3. Start position is position of 5′ sense
base on transcript NM_005475 (SEQ ID NO: 1).

| | | SEQ | | SEQ | |
|---|---|---|---|---|---|
| | Start | ID | | ID | |
| oligoName | Position | NO: | sense (5′-3′) | NO: | antis (5′-3′) |
| NM_005475.2_1078- | 1078 | 400 | gacccacccaagaguucaa | 450 | uugaacucuuggguggguc |
| 1096_s | | | | | |
| NM_005475.2_1162- | 1162 | 401 | ccugacaaccuuuacaccu | 451 | agguguaaagguugucagg |
| 1180_s | | | | | |
| NM_005475.2_1300- | 1300 | 402 | gcagagaugcauauucccu | 452 | agggaauaugcaucucugc |
| 1318_s | | | | | |
| NM_005475.2_1358- | 1358 | 403 | gcacagauucccuuaacca | 453 | ugguuaagggaaucugugc |
| 1376_s | | | | | |
| NM_005475.2_1412- | 1412 | 404 | gccagaagacggaccauuu | 454 | aaaugguccgucuucuggc |
| 1430_s | | | | | |
| NM_005475.2_2008- | 2008 | 405 | cgggacucggacuacgaaa | 455 | uuucguaguccgagucccg |
| 2026_s | | | | | |
| NM_005475.2_2009- | 2009 | 406 | gggacucggacuacgaaau | 456 | auuucguaguccgaguccc |
| 2027_s | | | | | |
| NM_005475.2_2016- | 2016 | 407 | ggacuacgaaauggacuca | 457 | ugaguccauuucguagucc |
| 2034_s | | | | | |
| NM_005475.2_2204- | 2204 | 408 | cugcucgugccaguuugga | 458 | uccaaacuggcacgagcag |
| 2222_s | | | | | |
| NM_005475.2_2361- | 2361 | 409 | cccguuacucuuagagaaa | 459 | uuucucuaagaguaacggg |
| 2379_s | | | | | |
| NM_005475.2_2509- | 2509 | 410 | cgguucucccaucuaccgu | 460 | acgguagaugggagaaccg |
| 2527_s | | | | | |
| NM_005475.2_2567- | 2567 | 411 | gaccacaucuaauccugcu | 461 | agcaggauuagaugugguc |
| 2585_s | | | | | |
| NM_005475.2_2715- | 2715 | 412 | cagugcacuguuucaacua | 462 | uaguugaaacagugcacug |
| 2733_s | | | | | |
| NM_005475.2_2928- | 2928 | 413 | ggguggcaagaacaguucu | 463 | agaacuguucuugccaccc |
| 2946_s | | | | | |
| NM_005475.2_2949- | 2949 | 414 | ccuggugccuuacgaauaa | 464 | uuauucguaaggcaccagg |
| 2967_s | | | | | |
| NM_005475.2_2992- | 2992 | 415 | gcuuuacagugauaguuaa | 465 | uuaacuaucacuguaaagc |
| 3010_s | | | | | |
| NM_005475.2_3057- | 3057 | 416 | ccuaggcccuuucuaguaa | 466 | uuacuagaaagggccuagg |
| 3075_s | | | | | |
| NM_005475.2_3063- | 3063 | 417 | cccuuucuaguaaauccuu | 467 | aaggauuuacuagaaaggg |
| 3081_s | | | | | |
| NM_005475.2_3109- | 3109 | 418 | caagcaucuaauggcuuau | 468 | auaagccauuagaugcuug |
| 3127_s | | | | | |
| NM_005475.2_3121- | 3121 | 419 | ggcuuauuaaauuauccca | 469 | ugggauaauuuaauaagcc |
| 3139_s | | | | | |
| NM_005475.2_3138- | 3138 | 420 | cacaaguggguuuuaggcu | 470 | agccuaaaacccacuugug |
| 3156_s | | | | | |
| NM_005475.2_3224- | 3224 | 421 | ccacaaucaguguucuguu | 471 | aacagaacacugauugugg |
| 3242_s | | | | | |
| NM_005475.2_3269- | 3269 | 422 | gagaaucuaucuaucuccu | 472 | aggagauagauagauucuc |
| 3287_s | | | | | |
| NM_005475.2_3560- | 3560 | 423 | gccuaaaacucauaggacu | 473 | aguccuaugaguuuuaggc |
| 3578_s | | | | | |
| NM_005475.2_3592- | 3592 | 424 | gaggguuaggucugcuucu | 474 | agaagcagaccuaacccuc |
| 3610_s | | | | | |
| NM_005475.2_3605- | 3605 | 425 | gcuucuuccacuuauacuu | 475 | aaguauaaguggaagaagc |
| 3623_s | | | | | |
| NM_005475.2_4148- | 4148 | 426 | gcuaauuagugugaaccaa | 476 | uugguucacacuaauuagc |
| 4166_s | | | | | |
| NM_005475.2_4156- | 4156 | 427 | gugugaaccaaaagaguaa | 477 | uuacucuuuugguucacac |
| 4174_s | | | | | |
| NM_005475.2_4160- | 4160 | 428 | gaaccaaaagaguaaguaa | 478 | uuacuuacucuuuugguuc |
| 4178_s | | | | | |
| NM_005475.2_4308- | 4308 | 429 | cugggauaugacgucuuca | 479 | ugaagacgucauaucccag |
| 4326_s | | | | | |
| NM_005475.2_4325- | 4325 | 430 | caugccaggggcacucauu | 480 | aaugagugccccuggcaug |
| 4343_s | | | | | |
| NM_005475.2_4333- | 4333 | 431 | gggcacucauuucuuagca | 481 | ugcuaagaaaugagugccc |
| 4351_s | | | | | |
| NM_005475.2_4353- | 4353 | 432 | ccucucuacauacaucucu | 482 | agagauguauguagagagg |
| 4371_s | | | | | |
| NM_005475.2_4532- | 4532 | 433 | gaaaacucccauggcucua | 483 | uagagccaugggaguuuuc |
| 4550_s | | | | | |
| NM_005475.2_4744- | 4744 | 434 | caccaaacuggacaguaga | 484 | ucuacuguccaguuuggug |
| 4762_s | | | | | |
| NM_005475.2_4930- | 4930 | 435 | ccauauggcuuuaucuggu | 485 | accagauaaagccauaugg |
| 4948_s | | | | | |
| NM_005475.2_4931- | 4931 | 436 | cauauggcuuuaucuggua | 486 | uaccagauaaagccauaug |
| 4949_s | | | | | |
| NM_005475.2_5118- | 5118 | 437 | gacuaagucccuuacugca | 487 | ugcaguaagggacuuaguc |
| 5136_s | | | | | |
| NM_005475.2_5169- | 5169 | 438 | gaggaacuugcgaguauau | 488 | auauacucgcaaguuccuc |
| 5187_s | | | | | |
| NM_005475.2_5269- | 5269 | 439 | cauuauaggcagaagguau | 489 | auaccuucugccuauaaug |
| 5287_s | | | | | |
| NM_005475.2_5330- | 5330 | 440 | cuuauucuugaauaaugca | 490 | ugcauuauucaagaauaag |
| 5348_s | | | | | |

TABLE 6
siRNAs specific for human and murine SH2B3. Start position is
position of 5′ sense base on transcript NM_005475 (SEQ ID NO: 1).

| | | SEQ | | SEQ | |
|---|---|---|---|---|---|
| | Start | ID | | ID | |
| oligoName | Position | NO: | sense (5′-3′) | NO: | antis (5′-3′) |
| NM_005475.2_1205- | 1205 | 500 | cagacaucaucuuugaggu | 550 | accucaaagaugaugucug |
| 1223_s | | | | | |
| NM_005475.2_1795- | 1795 | 501 | ucccuuccucacugggauu | 551 | aaucccagugaggaaggga |
| 1813_s | | | | | |
| NM_005475.2_1929- | 1929 | 502 | gaucuuccaccuggugccu | 552 | aggcaccagguggaagauc |
| 1947_s | | | | | |
| NM_005475.2_1930- | 1930 | 503 | aucuuccaccuggugccuu | 553 | aaggcaccagguggaagau |
| 1948_s | | | | | |
| NM_005475.2_2295- | 2295 | 504 | uguuuacagauguaguucu | 554 | agaacuacaucuguaaaca |
| 2313_s | | | | | |
| NM_005475.2_2296- | 2296 | 505 | guuuacagauguaguucuu | 555 | aagaacuacaucuguaaac |
| 2314_s | | | | | |
| NM_005475.2_2298- | 2298 | 506 | uuacagauguaguucuugu | 556 | acaagaacuacaucuguaa |
| 2316_s | | | | | |

TABLE 7
siRNAs specific for human and rhesus Itch. Some sequences may also
hybridize with murine Itch mRNAs. Start position is position of 5′ sense base
on transcript NM_031483 (SEQ ID NO: 11).

| | | SEQ | | SEQ | |
|---|---|---|---|---|---|
| | Start | ID | | ID | |
| oligoName | Position | NO: | sense (5′-3′) | NO: | antis (5′-3′) |
| NM_031483.4_89-107_s | 89 | 600 | gucccgguaccaugcauuu | 650 | aaaugcaugguaccgggac |
| NM_031483.4_138- | 138 | 601 | cccaaggaagugacucaaa | 651 | uuugagucacuuccuuggg |
| 156_s | | | | | |
| NM_031483.4_182- | 182 | 602 | cuauuggugguaugucuga | 652 | ucagacauaccaccaauag |
| 200_s | | | | | |
| NM_031483.4_230- | 230 | 603 | gccucaccaugaaaucaca | 653 | ugugauuucauggugaggc |
| 248_s | | | | | |
| NM_031483.4_260- | 260 | 604 | cugucaucucagcaaaacu | 654 | aguuuugcugagaugacag |
| 278_s | | | | | |
| NM_031483.4_413- | 413 | 605 | ccccugugaguaaauuaca | 655 | uguaauuuacucacagggg |
| 431_s | | | | | |
| NM_031483.4_414- | 414 | 606 | cccugugaguaaauuacau | 656 | auguaauuuacucacaggg |
| 432_s | | | | | |
| NM_031483.4_422- | 422 | 607 | guaaauuacauuuucgugu | 657 | acacgaaaauguaauuuac |
| 440_s | | | | | |
| NM_031483.4_477- | 477 | 608 | gggaacugcugcauuagau | 658 | aucuaaugcagcaguuccc |
| 495_s | | | | | |
| NM_031483.4_478- | 478 | 609 | ggaacugcugcauuagaua | 659 | uaucuaaugcagcaguucc |
| 496_s | | | | | |
| NM_031483.4_482- | 482 | 610 | cugcugcauuagauauuua | 660 | uaaauaucuaaugcagcag |
| 500_s | | | | | |
| NM_031483.4_506- | 506 | 611 | cauuaaagucaaacaauau | 661 | auauuguuugacuuuaaug |
| 524_s | | | | | |
| NM_031483.4_690- | 690 | 612 | cuccagauccaaggaugaa | 662 | uucauccuuggaucuggag |
| 708_s | | | | | |
| NM_031483.4_862- | 862 | 613 | cguagaccagcaucuguca | 663 | ugacagaugcuggucuacg |
| 880_s | | | | | |
| NM_031483.4_970- | 970 | 614 | gcaacaucuggauuaauaa | 664 | uuauuaauccagauguugc |
| 988_s | | | | | |
| NM_031483.4_1025- | 1025 | 615 | cauuaaauccuguaacuca | 665 | ugaguuacaggauuuaaug |
| 1043_s | | | | | |
| NM_031483.4_1124- | 1124 | 616 | caacaugggauagaccaga | 666 | ucuggucuaucccauguug |
| 1142_s | | | | | |
| NM_031483.4_1174- | 1174 | 617 | gacaacaugggacguauuu | 667 | aaauacgucccauguuguc |
| 1192_s | | | | | |
| NM_031483.4_1272- | 1272 | 618 | gcuacagcguagucagcuu | 668 | aagcugacuacgcuguagc |
| 1290_s | | | | | |
| NM_031483.4_1315- | 1315 | 619 | cagagauucauuuauggga | 669 | ucccauaaaugaaucucug |
| 1333_s | | | | | |
| NM_031483.4_1423- | 1423 | 620 | ggcagaguauauuucguca | 670 | ugacgaaauauacucugcc |
| 1441_s | | | | | |
| NM_031483.4_1424- | 1424 | 621 | gcagaguauauuucgucaa | 671 | uugacgaaauauacucugc |
| 1442_s | | | | | |
| NM_031483.4_1475- | 1475 | 622 | ccagaagucaaggucaauu | 672 | aauugaccuugacuucugg |
| 1493_s | | | | | |
| NM_031483.4_1574- | 1574 | 623 | gaacuaccaccuauauaga | 673 | ucuauauaggugguaguuc |
| 1592_s | | | | | |
| NM_031483.4_1701- | 1701 | 624 | gccacagcacauaaagauu | 674 | aaucuuuaugugcuguggc |
| 1719_s | | | | | |
| NM_031483.4_1831- | 1831 | 625 | gauuauggagguguagcaa | 675 | uugcuacaccuccauaauc |
| 1849_s | | | | | |
| NM_031483.4_1916- | 1916 | 626 | ggaaggauaacuacugcuu | 676 | aagcaguaguuauccuucc |
| 1934_s | | | | | |
| NM_031483.4_1947- | 1947 | 627 | cgcuucuuacaucaaucca | 677 | uggauugauguaagaagcg |
| 1965_s | | | | | |
| NM_031483.4_1969- | 1969 | 628 | caccugaaauauuuucguu | 678 | aacgaaaauauuucaggug |
| 1987_s | | | | | |
| NM_031483.4_2053- | 2053 | 629 | ccauucuauaagcguaucu | 679 | agauacgcuuauagaaugg |
| 2071_s | | | | | |
| NM_031483.4_2178- | 2178 | 630 | guacuucuccguugacaaa | 680 | uuugucaacggagaaguac |
| 2196_s | | | | | |
| NM_031483.4_2217- | 2217 | 631 | gagucaugaucugaaaccu | 681 | agguuucagaucaugacuc |
| 2235_s | | | | | |
| NM_031483.4_2234- | 2234 | 632 | cuaaugguggcaauauucu | 682 | agaauauugccaccauuag |
| 2252_s | | | | | |
| NM_031483.4_2303- | 2303 | 633 | gguugucucgagguguuga | 683 | ucaacaccucgagacaacc |
| 2321_s | | | | | |
| NM_031483.4_2455- | 2455 | 634 | caugccaucuaccgucauu | 684 | aaugacgguagauggcaug |
| 2473_s | | | | | |
| NM_031483.4_2765- | 2765 | 635 | gauuuggacaagaguaacu | 685 | aguuacucuuguccaaauc |
| 2783_s | | | | | |
| NM_031483.4_2806- | 2806 | 636 | gggcaagaacuuauuugca | 686 | ugcaaauaaguucuugccc |
| 2824_s | | | | | |
| NM_031483.4_2849- | 2849 | 637 | cacaucuuguaaaauugga | 687 | uccaauuuuacaagaugug |
| 2867_s | | | | | |
| NM_031483.4_2883- | 2883 | 638 | gaguuaucugaguguaagu | 688 | acuuacacucagauaacuc |
| 2901_s | | | | | |
| NM_031483.4_2885- | 2885 | 639 | guuaucugaguguaaguaa | 689 | uuacuuacacucagauaac |
| 2903_s | | | | | |
| NM_031483.4_2915- | 2915 | 640 | cauuuagauuuaucuccca | 690 | ugggagauaaaucuaaaug |
| 2933_s | | | | | |
| NM_031483.4_2965- | 2965 | 641 | gucugcaaaugacuaguca | 691 | ugacuagucauuugcagac |
| 2983_s | | | | | |
| NM_031483.4_3261- | 3261 | 642 | cccucucauuuuagucuga | 692 | ucagacuaaaaugagaggg |
| 3279_s | | | | | |
| NM_031483.4_3274- | 3274 | 643 | gucugacuugacuauuguu | 693 | aacaauagucaagucagac |
| 3292_s | | | | | |
| NM_031483.4_3310- | 3310 | 644 | gaauccauacaucauuccu | 694 | aggaaugauguauggauuc |
| 3328_s | | | | | |

1. A double-stranded ribonucleic acid (dsRNA) that inhibits expression of a gene encoding a negative regulator of multipotent hematopoietic cell (MHC) expansion, wherein said negative regulator of MHC expansion is gene selected from the group consisting of: Itch, SH2B3, Prox1 and AhR.

2-40. (canceled)

41. The dsRNA of claim 1, wherein said dsRNA comprises a sense strand and an antisense strand, wherein the sense strand comprises at least 15 contiguous nucleotides differing by no more than 3 nucleotides from the nucleotide sequence of SEQ ID NO: 1 and the antisense strand comprises at least 15 contiguous nucleotides differing by no more than 3 nucleotides from the nucleotide sequence of SEQ ID NO: 8.

42. The dsRNA of claim 1, wherein said dsRNA comprises a sense strand and an antisense strand, wherein the sense strand comprises at least 15 contiguous nucleotides differing by no more than 3 nucleotides from the nucleotide sequence of SEQ ID NO: 2 and the antisense strand comprises at least 15 contiguous nucleotides differing by no more than 3 nucleotides from the nucleotide sequence of SEQ ID NO: 9.

43. The dsRNA of claim 1, wherein said dsRNA comprises a sense strand and an antisense strand, wherein the sense strand comprises at least 15 contiguous nucleotides differing by no more than 3 nucleotides from the nucleotide sequence of SEQ ID NO: 3 and the antisense strand comprises at least 15 contiguous nucleotides differing by no more than 3 nucleotides from the nucleotide sequence of SEQ ID NO: 10.

44. The dsRNA of claim 1, wherein said dsRNA comprises a sense strand and an antisense strand, wherein the sense strand comprises at least 15 contiguous nucleotides differing by no more than 3 nucleotides from the nucleotide sequence of SEQ ID NO: 11 and the antisense strand comprises at least 15 contiguous nucleotides differing by no more than 3 nucleotides from the nucleotide sequence of SEQ ID NO: 12.

45. The dsRNA of claim 1, wherein said dsRNA comprises a sense strand and an antisense strand, the antisense strand comprising a region of complementarity which comprises at least 15 contiguous nucleotides differing by no more than 3 nucleotides from one of the antisense sequences listed in Tables 2-7.

46. The dsRNA of claim 1, wherein said dsRNA comprises at least one modified nucleotide.

47. The dsRNA of claim 46, wherein at least one of said modified nucleotides is chosen from the group consisting of:
a 2′-O-methyl modified nucleotide; a nucleotide comprising a 5′-phosphorothioate group; a terminal nucleotide linked to a cholesteryl derivative or dodecanoic acid bisdecylamide group; a 2′-deoxy-2′-fluoro modified nucleotide; a 2′-deoxy-modified nucleotide; a locked nucleotide; an abasic nucleotide; a 2′-amino-modified nucleotide; 2′-alkyl-modified nucleotide; a morpholino nucleotide; a phosphoramidate; and a non-natural base comprising nucleotide.

48. The dsRNA of claim 1, wherein each strand of said double-stranded RNA is no more than 30 nucleotides in length.

49. The dsRNA of claim 1, wherein at least one strand of said double-stranded RNA comprises a 3′ overhang of at least 1 nucleotide.

50. The dsRNA of claim 1, further comprising a ligand.

51. The dsRNA of claim 45, wherein the region of complementarity has sequence consisting of one of the antisense sequences of Tables 2-7.

52. The dsRNA of claim 45, wherein the sense strand has sequence consisting of SEQ ID NO: 1 and the antisense strand consists of SEQ ID NO: 8.

53. The dsRNA of claim 45, wherein the sense strand has sequence consisting of SEQ ID NO: 2 and the antisense strand consists of SEQ ID NO: 9.

54. The dsRNA of claim 45, wherein the sense strand has sequence consisting of SEQ ID NO: 3 and the antisense strand consists of SEQ ID NO: 10.

55. The dsRNA of claim 1 wherein the dsRNA comprises a sense strand with sequence consisting of a sense strand sequence selected from Tables 2-7, and an antisense strand with sequence consisting of an antisense sequence selected from Tables 2-7.

56. A vector encoding at least one strand of a dsRNA of claim 1.

57. A method of inhibiting expression of a gene encoding a negative regulator of MHC expansion in a cell, the method comprising:
(a) introducing into the cell the dsRNA of claim 1; and
(b) maintaining the cell produced in step (a) for a time sufficient to obtain degradation of the mRNA transcript of a gene encoding a negative regulator of MHC expansion, thereby inhibiting expression of the gene encoding a negative regulator of MHC expansion in the cell.

58. The method of claim 57, wherein the expression of the gene encoding a negative regulator of MHC expansion is inhibited by at least 30%.

59. A method of expansion of a multipotent hematopoietic cell, the method comprising introducing into an multipotent hematopoietic cell a dsRNA of claim 1 and maintaining the cell for a time and under conditions sufficient to permit expansion of the cell.

60. The method of claim 59 wherein resulting expanded cells have enhanced ability to engraft and/or differentiate relative to a multipotent hematopoietic cell that has not been treated according to the method of claim 59.

61. The method of claim 59, wherein said expansion occurs ex vivo.

62. The method of claim 59, wherein said expansion occurs in vivo.

63. A method of treating a patient in need of enhanced multipotent hematopoietic cell expansion and/or engraftment, wherein said method comprises administering to the patient a treatment comprising one or more of the following:
a dsRNA of claim 1; a cell comprising a dsRNA of claim 1; a vector of claim 56; or a cell comprising a vector of claim 56.
